(19) United States
(12) Patent Application Publication
(10) Pub. No.: US 2023/0391856 A1
(43) Pub. Date: Dec. 7, 2023

(54) SARS-COV-2 ANTIBODIES AND USES THEREOF

(71) Applicant: Icahn School of Medicine at Mount Sinai, New York, NY (US)

(72) Inventors: Thomas Moran, New York, NY (US); Thomas Kraus, New York, NY (US); Domenico Tortorella, New York, NY (US); J. Andrew Duty, New York, NY (US)

(73) Assignee: Icahn School of Medicine at Mount Sinai, New York, NY (US)

(21) Appl. No.: 18/249,999

(22) PCT Filed: Oct. 22, 2021
(86) PCT No.: PCT/US21/56225

§ 371 Date: Apr. 21, 2023

Related U.S. Application Data
(60) Provisional application No. 63/151,570, filed on Feb. 19, 2021.
(60) Provisional application No. 63/105,190, filed on Oct. 23, 2020.

(51) Int. Cl. C07K 16/10; A61P 31/14; G01N 33/569

(52) U.S. Cl. CPC C07K 16/1003; A61P 31/14; G01N 33/56983; C07K 2317/565; C07K 2317/622

(57) ABSTRACT

Provided herein are antibodies that bind to SARS-CoV-2 spike protein or a fragment thereof (e.g., receptor binding domain (RBD)), host cells for producing such antibodies, and kits comprising such antibodies. Also provided herein are compositions comprising antibodies that bind to SARS-CoV-2 spike protein or a fragment thereof (e.g., receptor binding domain) and methods of using such antibodies to diagnose, prevent or treat a SARS-CoV-2 infection, or COVID-19.

### 1. CROSS REFERENCE TO RELATED APPLICATION

This application claims priority under 35 U.S.C. § 119(e) to U.S. Provisional Patent Application No. 63/105,190, filed Oct. 23, 2020 and U.S. Provisional Application No. 63/151,570 filed Feb. 19, 2021, the disclosures of which are incorporated herein by reference in their entireties.

### 2. SEQUENCE LISTING

The instant application contains a Sequence Listing which has been submitted electronically in ASCII format and is hereby incorporated by reference in its entirety. Said ASCII copy, created on Oct. 22, 2021, is named 084284_00238_SL.txt and is 194,120 bytes in size.

### 3. BACKGROUND

Coronavirus disease 2019 (COVID-19) is a contagious disease caused by severe acute respiratory syndrome coronavirus-2 (SARS-CoV-2). The disease has spread world-wide, with symptoms ranging from mild to severe illness. There is an urgent need to develop therapeutics to treat COVID-19 and diagnostics to detect SARS-CoV-2).

### 4. SUMMARY

In one aspect, provided herein is an antibody that binds to the spike protein of severe acute respiratory syndrome coronavirus-2 (SARS-CoV-2) or a fragment thereof (e.g., receptor binding domain (RBD)) and compositions comprising such an antibody. In one embodiment, an antibody described herein comprises the amino acid sequences of the variable heavy chain region and variable light chain region of antibody 1, 2, 3, 4, 5, 6, 7, 8, 9, 10, 11, 12, 13, 14, 15, 16, 17, 18, 19, 20, 21, 22, 23, 24, 25 or 26 (see Tables 1 and 2).

In one embodiment, an antibody provided herein comprises the amino acid sequences of the variable heavy chain region (Table 1) and the variable light chain region (Table 2) of the antibody SARS2-1C12_f3. In another embodiment, an antibody provided herein comprises the amino acid sequences of the variable heavy chain region (Table 1) and the variable light chain region (Table 2) of the antibody SARS2-1D5_f3. In one embodiment, an antibody provided herein comprises the amino acid sequences of the variable heavy chain region (Table 1) and the variable light chain region (Table 2) of the antibody SARS2-1D10_f3. In one embodiment, an antibody provided herein comprises the amino acid sequences of the variable heavy chain region (Table 1) and the variable light chain region (Table 2) of the antibody SARS2-1E2_f3. In one embodiment, an antibody provided herein comprises the amino acid sequences of the variable heavy chain region (Table 1) and the variable light chain region (Table 2) of the antibody SARS2-1G9_f3. In one embodiment, an antibody provided herein comprises the amino acid sequences of the variable heavy chain region (Table 1) and the variable light chain region (Table 2) of the antibody SARS2-5D7_f3. In one embodiment, an antibody provided herein comprises the amino acid sequences of the variable heavy chain region (Table 1) and the variable light chain region (Table 2) of the antibody SARS2-5H12_f3. In one embodiment, an antibody provided herein comprises the amino acid sequences of the variable heavy chain region (Table 1) and the variable light chain region (Table 2) of the antibody SARS2-7C10_f3. In one embodiment, an antibody provided herein comprises the amino acid sequences of the variable heavy chain region (Table 1) and the variable light chain region (Table 2) of the antibody SARS2-8H4_f3. In one embodiment, an antibody provided herein comprises the amino acid sequences of the variable heavy chain region (Table 1) and the variable light chain region (Table 2) of the antibody SARS2-9C6_f3. In one embodiment, an antibody provided herein comprises the amino acid sequences of the variable heavy chain region (Table 1) and the variable light chain region (Table 2) of the antibody SARS2-10D6_f3. In one embodiment, an antibody provided herein comprises the amino acid sequences of the variable heavy chain region (Table 1) and the variable light chain region (Table 2) of the antibody SARS2-11D5_f3. In one embodiment, an antibody provided herein comprises the amino acid sequences of the variable heavy chain region (Table 1) and the variable light chain region (Table 2) of the antibody SARS2-11G2_S. Ina specific embodiment, an antibody provided herein comprises the amino acid sequences of the variable heavy chain region (Table 1) and the variable light chain region (Table 2) of the antibody SARS2-11G7_f2. In one embodiment, an antibody provided herein comprises the amino acid sequences of the variable heavy chain region (Table 1) and the variable light chain region (Table 2) of the antibody SARS2-16C5_f3. In one embodiment, an antibody provided herein comprises the amino acid sequences of the variable heavy chain region (Table 1) and the variable light chain region (Table 2) of the antibody SARS2-16C12_f3. In one embodiment, an antibody provided herein comprises the amino acid sequences of the variable heavy chain region (Table 1) and the variable light chain region (Table 2) of the antibody SARS2-17A8_f3. In one embodiment, an antibody provided herein comprises the amino acid sequences of the variable heavy chain region (Table 1) and the variable light chain region (Table 2) of the antibody SARS2-17F2_f3. In one embodiment, an antibody provided herein comprises the amino acid sequences of the variable heavy chain region (Table 1) and the variable light chain region (Table 2) of the antibody SARS2-18E7_f3. In one embodiment, an antibody provided herein comprises the amino acid sequences of the variable heavy chain region (Table 1) and the variable light chain region (Table 2) of the antibody SARS2-19C4_f3. In one embodiment, an antibody provided herein comprises the amino acid sequences of the variable heavy chain region (Table 1) and the variable light chain region (Table 2) of the antibody SARS2_2C1_f4. In one embodiment, an antibody provided herein comprises the amino acid sequences of the variable heavy chain region (Table 1) and the variable light chain region (Table 2) of the antibody SARS2_4E3_f4. In one embodiment, an antibody provided herein comprises the amino acid sequences of the variable heavy chain region (Table 1) and the variable light chain region (Table 2) of the antibody SARS2_7D2_f4. In one embodiment, an antibody provided herein comprises the amino acid sequences of the variable heavy chain region (Table 1) and the variable light chain region (Table 2) of the antibody SASR2_9C5_f4. In one embodiment, an antibody provided herein comprises the amino acid sequences of the variable heavy chain region (Table 1) and the variable light chain region (Table 2) of the antibody SASR2_10A3_f4. In one embodiment, an antibody provided herein comprises the amino acid sequences of the variable heavy chain region (Table 1) and the variable light chain region (Table 2) of the antibody SASR2_14G5_f4. In one embodiment, an antibody provided herein comprises the amino acid sequences of the variable heavy chain region (Table 1) and the variable light chain region (Table 2) of the antibody SASR2_13A12_f3. In one embodiment, an antibody provided herein comprises the amino acid sequences of the variable heavy chain region (Table 1) and the variable light chain region (Table 2) of the antibody SASR2_5B6_f3. In one embodiment, an antibody provided herein comprises the amino acid sequences of the variable heavy chain region (Table 1) and the variable light chain region (Table 2) of the antibody SASR2_2G6_f4.

In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 1 in Table 1; or (b) a variable light chain region comprising the variable light chain region CDRs of antibody number 1 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 2 in Table 1; or (b) a variable light chain region comprising the variable light chain region CDRs of antibody number 2 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 3 in Table 1; or (b) a variable light chain region comprising the variable light chain region CDRs of antibody number 3 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 4 in Table 1; or (b) a variable light chain region comprising the variable light chain region CDRs of antibody number 4 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 5 in Table 1; or (b) a variable light chain region comprising the variable light chain region CDRs of antibody number 5 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 6 in Table 1; or (b) a variable light chain region comprising the variable light chain region CDRs of antibody number 6 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 7 in Table 1; or (b) a variable light chain region comprising the variable light chain region CDRs of antibody number 7 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 8 in Table 1; or (b) a variable light chain region comprising the variable light chain region CDRs of antibody number 8 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 9 in Table 1; or (b) a variable light chain region comprising the variable light chain region CDRs of antibody number 9 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 10 in Table 1; or (b) a variable light chain region comprising the variable light chain region CDRs of antibody number 10 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 11 in Table 1; or (b) a variable light chain region comprising the variable light chain region CDRs of antibody number 11 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 12 in Table 1; or (b) a variable light chain region comprising the variable light chain region CDRs of antibody number 12 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 13 in Table 1; or (b) a variable light chain region comprising the variable light chain region CDRs of antibody number 13 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 14 in Table 1; or (b) a variable light chain region comprising the variable light chain region CDRs of antibody number 14 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 15 in Table 1; or (b) a variable light chain region comprising the variable light chain region CDRs of antibody number 15 in Table 1. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 16 in Table 1; or (b) a variable light chain region comprising the variable light chain region CDRs of antibody number 16 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 17 in Table 1; or (b) a variable light chain region comprising the variable light chain region CDRs of antibody number 17 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 18 in Table 1; or (b) a variable light chain region comprising the variable light chain region CDRs of antibody number 19 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 20 in Table 1; or (b) a variable light chain region comprising the variable light chain region CDRs of antibody number 20 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 21 in Table 1; or (b) a variable light chain region comprising the variable light chain region CDRs of antibody number 21 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 22 in Table 1; or (b) a variable light chain region comprising the variable light chain region CDRs of antibody number 22 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 23 in Table 1; or (b) a variable light chain region comprising the variable light chain region CDRs of antibody number 23 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 24 in Table 1; or (b) a variable light chain region comprising the variable light chain region CDRs of antibody number 24 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 25 in Table 1; or (b) a variable light chain region comprising the variable light chain region CDRs of antibody number 25 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 26 in Table 1; or (b) a variable light chain region comprising the variable light chain region CDRs of antibody number 26 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 27 in Table 1; or (b) a variable light chain region comprising the variable light chain region CDRs of antibody number 27 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 28 in Table 1; or (b) a variable light chain region comprising the variable light chain region CDRs of antibody number 28 in Table 2. In one embodiment, an antibody that binds to SARS-CoV-2 spike protein or a fragment thereof (e.g., RBD) comprises: (a) a variable heavy chain region comprising the variable heavy chain region complementarity determining regions (CDRs) of antibody number 29 in Table 1; or (b) a variable light chain region comprising the variable light chain region CDRs of antibody number 29 in Table 2.

In another embodiment, provided herein is an antibody that binds to SARS-CoV-2 spike protein, wherein the antibody comprises a variable heavy chain region comprising the amino acid sequence of antibody number 1 in Table 1 and a variable light chain region comprising the amino acid sequence of antibody number 1 in Table 2. In another embodiment, provided herein is an antibody that binds to SARS-CoV-2 spike protein, wherein the antibody comprises a variable heavy chain region comprising the amino acid sequence of antibody number 2 in Table 1 and a variable light chain region comprising the amino acid sequence of antibody number 2 in Table 2. In another embodiment, provided herein is an antibody that binds to SARS-CoV-2 spike protein, wherein the antibody comprises a variable heavy chain region comprising the amino acid sequence of antibody number 3 in Table 1 and a variable light chain region comprising the amino acid sequence of antibody number 3 in Table 2. In another embodiment, provided herein is an antibody that binds to SARS-CoV-2 spike protein, wherein the antibody comprises a variable heavy chain region comprising the amino acid sequence of antibody number 4 in Table 1 and a variable light chain region comprising the amino acid sequence of antibody number 4 in Table 2. In another embodiment, provided herein is an antibody that binds to SARS-CoV-2 spike protein, wherein the antibody comprises a variable heavy chain region comprising the amino acid sequence of antibody number 5 in Table 1 and a variable light chain region comprising the amino acid sequence of antibody number 5 in Table 2. In another embodiment, provided herein is an antibody that binds to SARS-CoV-2 spike protein, wherein the antibody comprises a variable heavy chain region comprising the amino acid sequence of antibody number 6 in Table 1 and a variable light chain region comprising the amino acid sequence of antibody number 6 in Table 2. In another embodiment, provided herein is an antibody that binds to SARS-CoV-2 spike protein, wherein the antibody comprises a variable heavy chain region comprising the amino acid sequence of antibody number 7 in Table 1 and a variable light chain region comprising the amino acid sequence of antibody number 7 in Table 2. In another embodiment, provided herein is an antibody that binds to SARS-CoV-2 spike protein, wherein the antibody comprises a variable heavy chain region comprising the amino acid sequence of antibody number 8 in Table 1 and a variable light chain region comprising the amino acid sequence of antibody number 8 in Table 2. In another embodiment, provided herein is an antibody that binds to SARS-CoV-2 spike protein, wherein the antibody comprises a variable heavy chain region comprising the amino acid sequence of antibody number 9 in Table 1 and a variable light chain region comprising the amino acid sequence of antibody number 9 in Table 2. In another embodiment, provided herein is an antibody that binds to SARS-CoV-2 spike protein, wherein the antibody comprises a variable heavy chain region comprising the amino acid sequence of antibody number 10 in Table 1 and a variable light chain region comprising the amino acid sequence of antibody number 10 in Table 2. In another embodiment, provided herein is an antibody that binds to SARS-CoV-2 spike protein, wherein the antibody comprises a variable heavy chain region comprising the amino acid sequence of antibody number 11 in Table 1 and a variable light chain region comprising the amino acid sequence of antibody number 11 in Table 2. In another embodiment, provided herein is an antibody that binds to SARS-CoV-2 spike protein, wherein the antibody comprises a variable heavy chain region comprising the amino acid sequence of antibody number 12 in Table 1 and a variable light chain region comprising the amino acid sequence of antibody number 12 in Table 2. In another embodiment, provided herein is an antibody that binds to SARS-CoV-2 spike protein, wherein the antibody comprises a variable heavy chain region comprising the amino acid sequence of antibody number 13 in Table 1 and a variable light chain region comprising the amino acid sequence of antibody number 13 in Table 2. In another embodiment, provided herein is an antibody that binds to SARS-CoV-2 spike protein, wherein the antibody comprises a variable heavy chain region comprising the amino acid sequence of antibody number 14 in Table 1 and a variable light chain region comprising the amino acid sequence of antibody number 14 in Table 2. In another embodiment, provided herein is an antibody that binds to SARS-CoV-2 spike protein, wherein the antibody comprises a variable heavy chain region comprising the amino acid sequence of antibody number 15 in Table 1 and a variable light chain region comprising the amino acid sequence of antibody number 15 in Table 2. In another embodiment, provided herein is an antibody that binds to SARS-CoV-2 spike protein, wherein the antibody comprises a variable heavy chain region comprising the amino acid sequence of antibody number 16 in Table 1 and a variable light chain region comprising the amino acid sequence of antibody number 16 in Table 2. In another embodiment, provided herein is an antibody that binds to SARS-CoV-2 spike protein, wherein the antibody comprises variable heavy chain region comprising the amino acid sequence of antibody number 17 in Table 1 and a variable light chain region comprising the amino acid sequence of antibody number 17 in Table 2. In another embodiment, provided herein is an antibody that binds to SARS-CoV-2 spike protein, wherein the antibody comprises a variable heavy chain region comprising the amino acid sequence of antibody number 18 in Table 1 and a variable light chain region comprising the amino acid sequence of antibody number 18 in Table 2. In another embodiment, provided herein is an antibody that binds to SARS-CoV-2 spike protein, wherein the antibody comprises a variable heavy chain region comprising the amino acid sequence of antibody number 19 in Table 1 and a variable light chain region comprising the amino acid sequence of antibody number 19 in Table 2. In another embodiment, provided herein is an antibody that binds to SARS-CoV-2 spike protein, wherein the antibody comprises variable heavy chain region comprising the amino acid sequence of antibody number 20 in Table 1 and a variable light chain region comprising the amino acid sequence of antibody number 20 in Table 2. In another embodiment, provided herein is an antibody that binds to SARS-CoV-2 spike protein, wherein the antibody comprises variable heavy chain region comprising the amino acid sequence of antibody number 21 in Table 1 and a variable light chain region comprising the amino acid sequence of antibody number 21 in Table 2. In another embodiment, provided herein is an antibody that binds to SARS-CoV-2 spike protein, wherein the antibody comprises variable heavy chain region comprising the amino acid sequence of antibody number 22 in Table 1 and a variable light chain region comprising the amino acid sequence of antibody number 22 in Table 2. In another embodiment, provided herein is an antibody that binds to SARS-CoV-2 spike protein, wherein the antibody comprises variable heavy chain region comprising the amino acid sequence of antibody number 23 in Table 1 and a variable light chain region comprising the amino acid sequence of antibody number 23 in Table 2. In another embodiment, provided herein is an antibody that binds to SARS-CoV-2 spike protein, wherein the antibody comprises variable heavy chain region comprising the amino acid sequence of antibody number 24 in Table 1 and a variable light chain region comprising the amino acid sequence of antibody number 24 in Table 2. In another embodiment, provided herein is an antibody that binds to SARS-CoV-2 spike protein, wherein the antibody comprises variable heavy chain region comprising the amino acid sequence of antibody number 25 in Table 1 and a variable light chain region comprising the amino acid sequence of antibody number 25 in Table 2. In another embodiment, provided herein is an antibody that binds to SARS-CoV-2 spike protein, wherein the antibody comprises variable heavy chain region comprising the amino acid sequence of antibody number 26 in Table 1 and a variable light chain region comprising the amino acid sequence of antibody number 26 in Table 2. In another embodiment, provided herein is an antibody that binds to SARS-CoV-2 spike protein, wherein the antibody comprises variable heavy chain region comprising the amino acid sequence of antibody number 27 in Table 1 and a variable light chain region comprising the amino acid sequence of antibody number 27 in Table 2. In another embodiment, provided herein is an antibody that binds to SARS-CoV-2 spike protein, wherein the antibody comprises variable heavy chain region comprising the amino acid sequence of antibody number 28 in Table 1 and a variable light chain region comprising the amino acid sequence of antibody number 28 in Table 2. In another embodiment, provided herein is an antibody that binds to SARS-CoV-2 spike protein, wherein the antibody comprises variable heavy chain region comprising the amino acid sequence of antibody number 29 in Table 1 and a variable light chain region comprising the amino acid sequence of antibody number 29 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 1 in Table 1; or (b) a variable light chain region that is at least 80%, at least 85%, at least 90%, at least 95%, at least 96%, at least 97%, at least 98%, or at least 99% identical to the amino acid sequence of antibody number 1 in Table 2. In one embodiment, the CDRs of the variable heavy chain region of the antibody are identical to the CDRs of the variable heavy chain region of antibody number 1 in Table 1. In one embodiment, the CDRs of the variable light chain region of the antibody are identical to the CDRs of the variable heavy light region of antibody number 1 in Table 2. In one embodiment, the CDRs of the variable heavy chain region of the antibody are identical to the CDRs of the variable heavy chain region of antibody number 1 in Table 1 and the CDRs of the variable light chain region of the antibody are identical to the CDRs of the variable heavy light region of antibody number 1 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 2 in Table 1; or (b) a variable light chain region that is at least 80%, at least 85%, at least 90%, at least 95%, at least 96%, at least 97%, at least 98%, or at least 99% identical to the amino acid sequence of antibody number 2 in Table 2. In one embodiment, the CDRs of the variable heavy chain region of the antibody are identical to the CDRs of the variable heavy chain region of antibody number 2 in Table 1. In one embodiment, the CDRs of the variable light chain region of the antibody are identical to the CDRs of the variable heavy light region of antibody number 2 in Table 2. In one embodiment, the CDRs of the variable heavy chain region of the antibody are identical to the CDRs of the variable heavy chain region of antibody number 2 in Table 1 and the CDRs of the variable light chain region of the antibody are identical to the CDRs of the variable heavy light region of antibody number 2 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 3 in Table 1; or (b) a variable light chain region that is at least 80%, at least 85%, at least 90%, at least 95%, at least 96%, at least 97%, at least 98%, or at least 99% identical to the amino acid sequence of antibody number 3 in Table 2. In one embodiment, the CDRs of the variable heavy chain region of the antibody are identical to the CDRs of the variable heavy chain region of antibody number 3 in Table 1. In one embodiment, the CDRs of the variable light chain region of the antibody are identical to the CDRs of the variable heavy light region of antibody number 3 in Table 2. In one embodiment, the CDRs of the variable heavy chain region of the antibody are identical to the CDRs of the variable heavy chain region of antibody number 3 in Table 1 and the CDRs of the variable light chain region of the antibody are identical to the CDRs of the variable heavy light region of antibody number 3 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 4 in Table 1; or (b) a variable light chain region that is at least 80%, at least 85%, at least 90%, at least 95%, at least 96%, at least 97%, at least 98%, or at least 99% identical to the amino acid sequence of antibody number 4 in Table 2. In one embodiment, the CDRs of the variable heavy chain region of the antibody are identical to the CDRs of the variable heavy chain region of antibody number 4 in Table 1. In one embodiment, the CDRs of the variable light chain region of the antibody are identical to the CDRs of the variable heavy light region of antibody number 4 in Table 2. In one embodiment, the CDRs of the variable heavy chain region of the antibody are identical to the CDRs of the variable heavy chain region of antibody number 4 in Table 1 and the CDRs of the variable light chain region of the antibody are identical to the CDRs of the variable heavy light region of antibody number 4 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 5 in Table 1; or (b) a variable light chain region that is at least 80%, at least 85%, at least 90%, at least 95%, at least 96%, at least 97%, at least 98%, or at least 99% identical to the amino acid sequence of antibody number 5 in Table 2. In one embodiment, the CDRs of the variable heavy chain region of the antibody are identical to the CDRs of the variable heavy chain region of antibody number 5 in Table 1. In one embodiment, the CDRs of the variable light chain region of the antibody are identical to the CDRs of the variable heavy light region of antibody number 5 in Table 2. In one embodiment, the CDRs of the variable heavy chain region of the antibody are identical to the CDRs of the variable heavy chain region of antibody number 5 in Table 1 and the CDRs of the variable light chain region of the antibody are identical to the CDRs of the variable heavy light region of antibody number 5 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 6 in Table 1; or (b) a variable light chain region that is at least 80%, at least 85%, at least 90%, at least 95%, at least 96%, at least 97%, at least 98%, or at least 99% identical to the amino acid sequence of antibody number 6 in Table 2. In one embodiment, the CDRs of the variable heavy chain region of the antibody are identical to the CDRs of the variable heavy chain region of antibody number 6 in Table 1. In one embodiment, the CDRs of the variable light chain region of the antibody are identical to the CDRs of the variable heavy light region of antibody number 6 in Table 2. In one embodiment, the CDRs of the variable heavy chain region of the antibody are identical to the CDRs of the variable heavy chain region of antibody number 6 in Table 1 and the CDRs of the variable light chain region of the antibody are identical to the CDRs of the variable heavy light region of antibody number 6 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 7 in Table 1; or (b) a variable light chain region that is at least 80%, at least 85%, at least 90%, at least 95%, at least 96%, at least 97%, at least 98%, or at least 99% identical to the amino acid sequence of antibody number 7 in Table 2. In one embodiment, the CDRs of the variable heavy chain region of the antibody are identical to the CDRs of the variable heavy chain region of antibody number 7 in Table 1. In one embodiment, the CDRs of the variable light chain region of the antibody are identical to the CDRs of the variable heavy light region of antibody number 7 in Table 2. In one embodiment, the CDRs of the variable heavy chain region of the antibody are identical to the CDRs of the variable heavy chain region of antibody number 7 in Table 1 and the CDRs of the variable light chain region of the antibody are identical to the CDRs of the variable heavy light region of antibody number 7 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 8 in Table 1; or (b) a variable light chain region that is at least 80%, at least 85%, at least 90%, at least 95%, at least 96%, at least 97%, at least 98%, or at least 99% identical to the amino acid sequence of antibody number 8 in Table 2. In one embodiment, the CDRs of the variable heavy chain region of the antibody are identical to the CDRs of the variable heavy chain region of antibody number 8 in Table 1. In one embodiment, the CDRs of the variable light chain region of the antibody are identical to the CDRs of the variable heavy light region of antibody number 8 in Table 2. In one embodiment, the CDRs of the variable heavy chain region of the antibody are identical to the CDRs of the variable heavy chain region of antibody number 8 in Table 1 and the CDRs of the variable light chain region of the antibody are identical to the CDRs of the variable heavy light region of antibody number 8 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 9 in Table 1; or (b) a variable light chain region that is at least 80%, at least 85%, at least 90%, at least 95%, at least 96%, at least 97%, at least 98%, or at least 99% identical to the amino acid sequence of antibody number 9 in Table 2. In one embodiment, the CDRs of the variable heavy chain region of the antibody are identical to the CDRs of the variable heavy chain region of antibody number 9 in Table 1. In one embodiment, the CDRs of the variable light chain region of the antibody are identical to the CDRs of the variable heavy light region of antibody number 9 in Table 2. In one embodiment, the CDRs of the variable heavy chain region of the antibody are identical to the CDRs of the variable heavy chain region of antibody number 9 in Table 1 and the CDRs of the variable light chain region of the antibody are identical to the CDRs of the variable heavy light region of antibody number 9 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 10 in Table 1; or (b) a variable light chain region that is at least 80%, at least 85%, at least 90%, at least 95%, at least 96%, at least 97%, at least 98%, or at least 99% identical to the amino acid sequence of antibody number 10 in Table 2. In one embodiment, the CDRs of the variable heavy chain region of the antibody are identical to the CDRs of the variable heavy chain region of antibody number 10 in Table 1. In one embodiment, the CDRs of the variable light chain region of the antibody are identical to the CDRs of the variable heavy light region of antibody number 10 in Table 2. In one embodiment, the CDRs of the variable heavy chain region of the antibody are identical to the CDRs of the variable heavy chain region of antibody number 10 in Table 1 and the CDRs of the variable light chain region of the antibody are identical to the CDRs of the variable heavy light region of antibody number 10 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 11 in Table 1; or (b) a variable light chain region that is at least 80%, at least 85%, at least 90%, at least 95%, at least 96%, at least 97%, at least 98%, or at least 99% identical to the amino acid sequence of antibody number 11 in Table 2. In one embodiment, the CDRs of the variable heavy chain region of the antibody are identical to the CDRs of the variable heavy chain region of antibody number 11 in Table 1. In one embodiment, the CDRs of the variable light chain region of the antibody are identical to the CDRs of the variable heavy light region of antibody number 11 in Table 2. In one embodiment, the CDRs of the variable heavy chain region of the antibody are identical to the CDRs of the variable heavy chain region of antibody number 11 in Table 1 and the CDRs of the variable light chain region of the antibody are identical to the CDRs of the variable heavy light region of antibody number 11 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 12 in Table 1; or (b) a variable light chain region that is at least 80%, at least 85%, at least 90%, at least 95%, at least 96%, at least 97%, at least 98%, or at least 99% identical to the amino acid sequence of antibody number 12 in Table 2. In one embodiment, the CDRs of the variable heavy chain region of the antibody are identical to the CDRs of the variable heavy chain region of antibody number 12 in Table 1. In one embodiment, the CDRs of the variable light chain region of the antibody are identical to the CDRs of the variable heavy light region of antibody number 12 in Table 2. In one embodiment, the CDRs of the variable heavy chain region of the antibody are identical to the CDRs of the variable heavy chain region of antibody number 12 in Table 1 and the CDRs of the variable light chain region of the antibody are identical to the CDRs of the variable heavy light region of antibody number 12 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 13 in Table 1; or (b) a variable light chain region that is at least 80%, at least 85%, at least 90%, at least 95%, at least 96%, at least 97%, at least 98%, or at least 99% identical to the amino acid sequence of antibody number 13 in Table 2. In one embodiment, the CDRs of the variable heavy chain region of the antibody are identical to the CDRs of the variable heavy chain region of antibody number 13 in Table 1. In one embodiment, the CDRs of the variable light chain region of the antibody are identical to the CDRs of the variable heavy light region of antibody number 13 in Table 2. In one embodiment, the CDRs of the variable heavy chain region of the antibody are identical to the CDRs of the variable heavy chain region of antibody number 13 in Table 1 and the CDRs of the variable light chain region of the antibody are identical to the CDRs of the variable heavy light region of antibody number 13 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 14 in Table 1; or (b) a variable light chain region that is at least 80%, at least 85%, at least 90%, at least 95%, at least 96%, at least 97%, at least 98%, or at least 99% identical to the amino acid sequence of antibody number 14 in Table 2. In one embodiment, the CDRs of the variable heavy chain region of the antibody are identical to the CDRs of the variable heavy chain region of antibody number 14 in Table 1. In one embodiment, the CDRs of the variable light chain region of the antibody are identical to the CDRs of the variable heavy light region of antibody number 14 in Table 2. In one embodiment, the CDRs of the variable heavy chain region of the antibody are identical to the CDRs of the variable heavy chain region of antibody number 14 in Table 1 and the CDRs of the variable light chain region of the antibody are identical to the CDRs of the variable heavy light region of antibody number 14 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 15 in Table 1; or (b) a variable light chain region that is at least 80%, at least 85%, at least 90%, at least 95%, at least 96%, at least 97%, at least 98%, or at least 99% identical to the amino acid sequence of antibody number 15 in Table 2. In one embodiment, the CDRs of the variable heavy chain region of the antibody are identical to the CDRs of the variable heavy chain region of antibody number 15 in Table 1. In one embodiment, the CDRs of the variable light chain region of the antibody are identical to the CDRs of the variable heavy light region of antibody number 15 in Table 2. In one embodiment, the CDRs of the variable heavy chain region of the antibody are identical to the CDRs of the variable heavy chain region of antibody number 15 in Table 1 and the CDRs of the variable light chain region of the antibody are identical to the CDRs of the variable heavy light region of antibody number 15 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 16 in Table 1; or (b) a variable light chain region that is at least 80%, at least 85%, at least 90%, at least 95%, at least 96%, at least 97%, at least 98%, or at least 99% identical to the amino acid sequence of antibody number 16 in Table 2. In one embodiment, the CDRs of the variable heavy chain region of the antibody are identical to the CDRs of the variable heavy chain region of antibody number 16 in Table 1. In one embodiment, the CDRs of the variable light chain region of the antibody are identical to the CDRs of the variable heavy light region of antibody number 16 in Table 2. In one embodiment, the CDRs of the variable heavy chain region of the antibody are identical to the CDRs of the variable heavy chain region of antibody number 16 in Table 1 and the CDRs of the variable light chain region of the antibody are identical to the CDRs of the variable heavy light region of antibody number 16 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 17 in Table 1; or (b) a variable light chain region that is at least 80%, at least 85%, at least 90%, at least 95%, at least 96%, at least 97%, at least 98%, or at least 99% identical to the amino acid sequence of antibody number 17 in Table 2. In one embodiment, the CDRs of the variable heavy chain region of the antibody are identical to the CDRs of the variable heavy chain region of antibody number 17 in Table 1. In one embodiment, the CDRs of the variable light chain region of the antibody are identical to the CDRs of the variable heavy light region of antibody number 17 in Table 2. In one embodiment, the CDRs of the variable heavy chain region of the antibody are identical to the CDRs of the variable heavy chain region of antibody number 17 in Table 1 and the CDRs of the variable light chain region of the antibody are identical to the CDRs of the variable heavy light region of antibody number 17 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 18 in Table 1; or (b) a variable light chain region that is at least 80%, at least 85%, at least 90%, at least 95%, at least 96%, at least 97%, at least 98%, or at least 99% identical to the amino acid sequence of antibody number 18 in Table 2. In one embodiment, the CDRs of the variable heavy chain region of the antibody are identical to the CDRs of the variable heavy chain region of antibody number 18 in Table 1. In one embodiment, the CDRs of the variable light chain region of the antibody are identical to the CDRs of the variable heavy light region of antibody number 18 in Table 2. In one embodiment, the CDRs of the variable heavy chain region of the antibody are identical to the CDRs of the variable heavy chain region of antibody number 18 in Table 1 and the CDRs of the variable light chain region of the antibody are identical to the CDRs of the variable heavy light region of antibody number 18 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 19 in Table 1; or (b) a variable light chain region that is at least 80%, at least 85%, at least 90%, at least 95%, at least 96%, at least 97%, at least 98%, or at least 99% identical to the amino acid sequence of antibody number 19 in Table 2. In one embodiment, the CDRs of the variable heavy chain region of the antibody are identical to the CDRs of the variable heavy chain region of antibody number 19 in Table 1. In one embodiment, the CDRs of the variable light chain region of the antibody are identical to the CDRs of the variable heavy light region of antibody number 19 in Table 2. In one embodiment, the CDRs of the variable heavy chain region of the antibody are identical to the CDRs of the variable heavy chain region of antibody number 19 in Table 1 and the CDRs of the variable light chain region of the antibody are identical to the CDRs of the variable heavy light region of antibody number 19 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 20 in Table 1; or (b) a variable light chain region that is at least 80%, at least 85%, at least 90%, at least 95%, at least 96%, at least 97%, at least 98%, or at least 99% identical to the amino acid sequence of antibody number 20 in Table 2. In one embodiment, the CDRs of the variable heavy chain region of the antibody are identical to the CDRs of the variable heavy chain region of antibody number 20 in Table 1. In one embodiment, the CDRs of the variable light chain region of the antibody are identical to the CDRs of the variable heavy light region of antibody number 20 in Table 2. In one embodiment, the CDRs of the variable heavy chain region of the antibody are identical to the CDRs of the variable heavy chain region of antibody number 20 in Table 1 and the CDRs of the variable light chain region of the antibody are identical to the CDRs of the variable heavy light region of antibody number 20 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 21 in Table 1; or (b) a variable light chain region that is at least 80%, at least 85%, at least 90%, at least 95%, at least 96%, at least 97%, at least 98%, or at least 99% identical to the amino acid sequence of antibody number 21 in Table 2. In one embodiment, the CDRs of the variable heavy chain region of the antibody are identical to the CDRs of the variable heavy chain region of antibody number 21 in Table 1. In one embodiment, the CDRs of the variable light chain region of the antibody are identical to the CDRs of the variable heavy light region of antibody number 21 in Table 2. In one embodiment, the CDRs of the variable heavy chain region of the antibody are identical to the CDRs of the variable heavy chain region of antibody number 21 in Table 1 and the CDRs of the variable light chain region of the antibody are identical to the CDRs of the variable heavy light region of antibody number 21 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 22 in Table 1; or (b) a variable light chain region that is at least 80%, at least 85%, at least 90%, at least 95%, at least 96%, at least 97%, at least 98%, or at least 99% identical to the amino acid sequence of antibody number 22 in Table 2. In one embodiment, the CDRs of the variable heavy chain region of the antibody are identical to the CDRs of the variable heavy chain region of antibody number 22 in Table 1. In one embodiment, the CDRs of the variable light chain region of the antibody are identical to the CDRs of the variable heavy light region of antibody number 22 in Table 2. In one embodiment, the CDRs of the variable heavy chain region of the antibody are identical to the CDRs of the variable heavy chain region of antibody number 22 in Table 1 and the CDRs of the variable light chain region of the antibody are identical to the CDRs of the variable heavy light region of antibody number 22 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 23 in Table 1; or (b) a variable light chain region that is at least 80%, at least 85%, at least 90%, at least 95%, at least 96%, at least 97%, at least 98%, or at least 99% identical to the amino acid sequence of antibody number 23 in Table 2. In one embodiment, the CDRs of the variable heavy chain region of the antibody are identical to the CDRs of the variable heavy chain region of antibody number 23 in Table 1. In one embodiment, the CDRs of the variable light chain region of the antibody are identical to the CDRs of the variable heavy light region of antibody number 23 in Table 2. In one embodiment, the CDRs of the variable heavy chain region of the antibody are identical to the CDRs of the variable heavy chain region of antibody number 23 in Table 1 and the CDRs of the variable light chain region of the antibody are identical to the CDRs of the variable heavy light region of antibody number 23 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 24 in Table 1; or (b) a variable light chain region that is at least 80%, at least 85%, at least 90%, at least 95%, at least 96%, at least 97%, at least 98%, or at least 99% identical to the amino acid sequence of antibody number 24 in Table 2. In one embodiment, the CDRs of the variable heavy chain region of the antibody are identical to the CDRs of the variable heavy chain region of antibody number 24 in Table 1. In one embodiment, the CDRs of the variable light chain region of the antibody are identical to the CDRs of the variable heavy light region of antibody number 24 in Table 2. In one embodiment, the CDRs of the variable heavy chain region of the antibody are identical to the CDRs of the variable heavy chain region of antibody number 24 in Table 1 and the CDRs of the variable light chain region of the antibody are identical to the CDRs of the variable heavy light region of antibody number 24 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 25 in Table 1; or (b) a variable light chain region that is at least 80%, at least 85%, at least 90%, at least 95%, at least 96%, at least 97%, at least 98%, or at least 99% identical to the amino acid sequence of antibody number 25 in Table 2. In one embodiment, the CDRs of the variable heavy chain region of the antibody are identical to the CDRs of the variable heavy chain region of antibody number 25 in Table 1. In one embodiment, the CDRs of the variable light chain region of the antibody are identical to the CDRs of the variable heavy light region of antibody number 25 in Table 2. In one embodiment, the CDRs of the variable heavy chain region of the antibody are identical to the CDRs of the variable heavy chain region of antibody number 25 in Table 1 and the CDRs of the variable light chain region of the antibody are identical to the CDRs of the variable heavy light region of antibody number 25 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 26 in Table 1; or (b) a variable light chain region that is at least 80%, at least 85%, at least 90%, at least 95%, at least 96%, at least 97%, at least 98%, or at least 99% identical to the amino acid sequence of antibody number 26 in Table 2. In one embodiment, the CDRs of the variable heavy chain region of the antibody are identical to the CDRs of the variable heavy chain region of antibody number 26 in Table 1. In one embodiment, the CDRs of the variable light chain region of the antibody are identical to the CDRs of the variable heavy light region of antibody number 26 in Table 2. In one embodiment, the CDRs of the variable heavy chain region of the antibody are identical to the CDRs of the variable heavy chain region of antibody number 26 in Table 1 and the CDRs of the variable light chain region of the antibody are identical to the CDRs of the variable heavy light region of antibody number 26 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 27 in Table 1; or (b) a variable light chain region that is at least 80%, at least 85%, at least 90%, at least 95%, at least 96%, at least 97%, at least 98%, or at least 99% identical to the amino acid sequence of antibody number 27 in Table 2. In one embodiment, the CDRs of the variable heavy chain region of the antibody are identical to the CDRs of the variable heavy chain region of antibody number 27 in Table 1. In one embodiment, the CDRs of the variable light chain region of the antibody are identical to the CDRs of the variable heavy light region of antibody number 27 in Table 2. In one embodiment, the CDRs of the variable heavy chain region of the antibody are identical to the CDRs of the variable heavy chain region of antibody number 27 in Table 1 and the CDRs of the variable light chain region of the antibody are identical to the CDRs of the variable heavy light region of antibody number 27 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 28 in Table 1; or (b) a variable light chain region that is at least 80%, at least 85%, at least 90%, at least 95%, at least 96%, at least 97%, at least 98%, or at least 99% identical to the amino acid sequence of antibody number 28 in Table 2. In one embodiment, the CDRs of the variable heavy chain region of the antibody are identical to the CDRs of the variable heavy chain region of antibody number 28 in Table 1. In one embodiment, the CDRs of the variable light chain region of the antibody are identical to the CDRs of the variable heavy light region of antibody number 28 in Table 2. In one embodiment, the CDRs of the variable heavy chain region of the antibody are identical to the CDRs of the variable heavy chain region of antibody number 28 in Table 1 and the CDRs of the variable light chain region of the antibody are identical to the CDRs of the variable heavy light region of antibody number 28 in Table 2.

In another embodiment, an antibody that binds to SARS-CoV-2 spike protein or a fragment thereof (e.g., RBD) comprises: (a) a variable heavy chain region that is at least 80%, at least 85%, at least 90%, at least 95%, at least 96%, at least 97%, at least 98%, or at least 99% identical to the amino acid sequence of antibody number 29 in Table 1; or (b) a variable light chain region that is at least 80%, at least 85%, at least 90%, at least 95%, at least 96%, at least 97%, at least 98%, or at least 99% identical to the amino acid sequence of antibody number 29 in Table 2. In one embodiment, the CDRs of the variable heavy chain region of the antibody are identical to the CDRs of the variable heavy chain region of antibody number 29 in Table 1. In one embodiment, the CDRs of the variable light chain region of the antibody are identical to the CDRs of the variable heavy light region of antibody number 29 in Table 2. In one embodiment, the CDRs of the variable heavy chain region of the antibody are identical to the CDRs of the variable heavy chain region of antibody number 29 in Table 1 and the CDRs of the variable light chain region of the antibody are identical to the CDRs of the variable heavy light region of antibody number 29 in Table 2.

Provided herein is an antibody that binds to SARS-CoV-2 spike protein or a fragment thereof (e.g., RBD) comprising a variable heavy chain region and a variable light chain region wherein:
a) the variable heavy chain region comprises a sequence that is at least 80%, at least 85%, at least 90%, at least 95%, at least 96%, at least 97%, at last 98%, or least 99% identical to SEQ ID NO:59 and the variable light chain region comprises a sequence that is at least 80%, at least 85%, at least 90%, at least 95%, at least 96%, at least 97%, at last 98%, or least 99% identical to SEQ ID NO:88;
b) the variable heavy chain region comprises a sequence that is at least 80%, at least 85%, at least 90%, at least 95%, at least 96%, at least 97%, at last 98%, or least 99% identical to SEQ ID NO:60 and the variable light chain region comprises a sequence that is at least 80%, at least 85%, at least 90%, at least 95%, at least 96%, at least 97%, at last 98%, or least 99% identical to SEQ ID NO:89;
c) the variable heavy chain region comprises a sequence that is at least 80%, at least 85%, at least 90%, at least 95%, at least 96%, at least 97%, at last 98%, or least 99% identical to SEQ ID NO:61 and the variable light chain region comprises a sequence that is at least 80%, at least 85%, at least 90%, at least 95%, at least 96%, at least 97%, at last 98%, or least 99% identical to SEQ ID NO:90;
d) the variable heavy chain region comprises a sequence that is at least 80%, at least 85%, at least 90%, at least 95%, at least 96%, at least 97%, at last 98%, or least 99% identical to SEQ ID NO:62 and the variable light chain region comprises a sequence that is at least 80%, at least 85%, at least 90%, at least 95%, at least 96%, at least 97%, at last 98%, or least 99% identical to SEQ ID NO:91;
e) the variable heavy chain region comprises a sequence that is at least 80%, at least 85%, at least 90%, at least 95%, at least 96%, at least 97%, at last 98%, or least 99% identical to SEQ ID NO:63 and the variable light chain region comprises a sequence that is at least 80%, at least 85%, at least 90%, at least 95%, at least 96%, at least 97%, at last 98%, or least 99% identical to SEQ ID NO:92;
f) the variable heavy chain region comprises a sequence that is at least 80%, at least 85%, at least 90%, at least 95%, at least 96%, at least 97%, at last 98%, or least 99% identical to SEQ ID NO:64 and the variable light chain region comprises a sequence that is at least 80%, at least 85%, at least 90%, at least 95%, at least 96%, at least 97%, at last 98%, or least 99% identical to SEQ ID NO:93;
g) the variable heavy chain region comprises a sequence that is at least 80%, at least 85%, at least 90%, at least 95%, at least 96%, at least 97%, at last 98%, or least 99% identical to SEQ ID NO:65 and the variable light chain region comprises a sequence that is at least 80%, at least 85%, at least 90%, at least 95%, at least 96%, at least 97%, at last 98%, or least 99% identical to SEQ ID NO:94;
h) the variable heavy chain region comprises a sequence that is at least 80%, at least 85%, at least 90%, at least 95%, at least 96%, at least 97%, at last 98%, or least 99% identical to SEQ ID NO:66 and the variable light chain region comprises a sequence that is at least 80%, at least 85%, at least 90%, at least 95%, at least 96%, at least 97%, at last 98%, or least 99% identical to SEQ ID NO:95;
i) the variable heavy chain region comprises a sequence that is at least 80%, at least 85%, at least 90%, at least 95%, at least 96%, at least 97%, at last 98%, or least 99% identical to SEQ ID NO:67 and the variable light chain region comprises a sequence that is at least 80%, at least 85%, at least 90%, at least 95%, at least 96%, at least 97%, at last 98%, or least 99% identical to SEQ ID NO:96;
j) the variable heavy chain region comprises a sequence that is at least 80%, at least 85%, at least 90%, at least 95%, at least 96%, at least 97%, at last 98%, or least 99% identical to SEQ ID NO:68 and the variable light chain region comprises a sequence that is at least 80%, at least 85%, at least 90%, at least 95%, at least 96%, at least 97%, at last 98%, or least 99% identical to SEQ ID NO:97;
k) the variable heavy chain region comprises a sequence that is at least 80%, at least 85%, at least 90%, at least 95%, at least 96%, at least 97%, at last 98%, or least 99% identical to SEQ ID NO:69 and the variable light chain region comprises a sequence that is at least 80%, at least 85%, at least 90%, at least 95%, at least 96%, at least 97%, at last 98%, or least 99% identical to SEQ ID NO:98;
l) the variable heavy chain region comprises a sequence that is at least 80%, at least 85%, at least 90%, at least 95%, at least 96%, at least 97%, at last 98%, or least 99% identical to SEQ ID NO:70 and the variable light chain region comprises a sequence that is at least 80%, at least 85%, at least 90%, at least 95%, at least 96%, at least 97%, at last 98%, or least 99% identical to SEQ ID NO:99;
m) the variable heavy chain region comprises a sequence that is at least 80%, at least 85%, at least 90%, at least 95%, at least 96%, at least 97%, at last 98%, or least 99% identical to SEQ ID NO:71 and the variable light chain region comprises a sequence that is at least 80%, at least 85%, at least 90%, at least 95%, at least 96%, at least 97%, at last 98%, or least 99% identical to SEQ ID NO:100;
n) the variable heavy chain region comprises a sequence that is at least 80%, at least 85%, at least 90%, at least 95%, at least 96%, at least 97%, at last 98%, or least 99% identical to SEQ ID NO:72 and the variable light chain region comprises a sequence that is at least 80%, at least 85%, at least 90%, at least 95%, at least 96%, at least 97%, at last 98%, or least 99% identical to SEQ ID NO:101;
o) the variable heavy chain region comprises a sequence that is at least 80%, at least 85%, at least 90%, at least 95%, at least 96%, at least 97%, at last 98%, or least 99% identical to SEQ ID NO:73 and the variable light chain region comprises a sequence that is at least 80%, at least 85%, at least 90%, at least 95%, at least 96%, at least 97%, at last 98%, or least 99% identical to SEQ ID NO:102;
p) the variable heavy chain region comprises a sequence that is at least 80%, at least 85%, at least 90%, at least 95%, at least 96%, at least 97%, at last 98%, or least 99% identical to SEQ ID NO:74 and the variable light chain region comprises a sequence that is at least 80%, at least 85%, at least 90%, at least 95%, at least 96%, at least 97%, at last 98%, or least 99% identical to SEQ ID NO:103;
q) the variable heavy chain region comprises a sequence that is at least 80%, at least 85%, at least 90%, at least 95%, at least 96%, at least 97%, at last 98%, or least 99% identical to SEQ ID NO:75 and the variable light chain region comprises a sequence that is at least 80%, at least 85%, at least 90%, at least 95%, at least 96%, at least 97%, at last 98%, or least 99% identical to SEQ ID NO:104;
r) the variable heavy chain region comprises a sequence that is at least 80%, at least 85%, at least 90%, at least 95%, at least 96%, at least 97%, at last 98%, or least 99% identical to SEQ ID NO:76 and the variable light chain region comprises a sequence that is at least 80%, at least 85%, at least 90%, at least 95%, at least 96%, at least 97%, at last 98%, or least 99% identical to SEQ ID NO:105;
s) the variable heavy chain region comprises a sequence that is at least 80%, at least 85%, at least 90%, at least 95%, at least 96%, at least 97%, at last 98%, or least 99% identical to SEQ ID NO:77 and the variable light chain region comprises a sequence that is at least 80%, at least 85%, at least 90%, at least 95%, at least 96%, at least 97%, at last 98%, or least 99% identical to SEQ ID NO:106;
t) the variable heavy chain region comprises a sequence that is at least 80%, at least 85%, at least 90%, at least 95%, at least 96%, at least 97%, at last 98%, or least 99% identical to SEQ ID NO:78 and the variable light chain region comprises a sequence that is at least 80%, at least 85%, at least 90%, at least 95%, at least 96%, at least 97%, at last 98%, or least 99% identical to SEQ ID NO:107;
u) the variable heavy chain region comprises a sequence that is at least 80%, at least 85%, at least 90%, at least 95%, at least 96%, at least 97%, at last 98%, or least 99% identical to SEQ ID NO:79 and the variable light chain region comprises a sequence that is at least 80%, at least 85%, at least 90%, at least 95%, at least 96%, at least 97%, at last 98%, or least 99% identical to SEQ ID NO:108;
v) the variable heavy chain region comprises a sequence that is at least 80%, at least 85%, at least 90%, at least 95%, at least 96%, at least 97%, at last 98%, or least 99% identical to SEQ ID NO:80 and the variable light chain region comprises a sequence that is at least 80%, at least 85%, at least 90%, at least 95%, at least 96%, at least 97%, at last 98%, or least 99% identical to SEQ ID NO:109;
w) the variable heavy chain region comprises a sequence that is at least 80%, at least 85%, at least 90%, at least 95%, at least 96%, at least 97%, at last 98%, or least 99% identical to SEQ ID NO:81 and the variable light chain region comprises a sequence that is at least 80%, at least 85%, at least 90%, at least 95%, at least 96%, at least 97%, at last 98%, or least 99% identical to SEQ ID NO:110;
x) the variable heavy chain region comprises a sequence that is at least 80%, at least 85%, at least 90%, at least 95%, at least 96%, at least 97%, at last 98%, or least 99% identical to SEQ ID NO:82 and the variable light chain region comprises a sequence that is at least 80%, at least 85%, at least 90%, at least 95%, at least 96%, at least 97%, at last 98%, or least 99% identical to SEQ ID NO:111;
y) the variable heavy chain region comprises a sequence that is at least 80%, at least 85%, at least 90%, at least 95%, at least 96%, at least 97%, at last 98%, or least 99% identical to SEQ ID NO:83 and the variable light chain region comprises a sequence that is at least 80%, at least 85%, at least 90%, at least 95%, at least 96%, at least 97%, at last 98%, or least 99% identical to SEQ ID NO:112;
z) the variable heavy chain region comprises a sequence that is at least 80%, at least 85%, at least 90%, at least 95%, at least 96%, at least 97%, at last 98%, or least 99% identical to SEQ ID NO:84 and the variable light chain region comprises a sequence that is at least 80%, at least 85%, at least 90%, at least 95%, at least 96%, at least 97%, at last 98%, or least 99% identical to SEQ ID NO:113;
aa) the variable heavy chain region comprises a sequence that is at least 80%, at least 85%, at least 90%, at least 95%, at least 96%, at least 97%, at last 98%, or least 99% identical to SEQ ID NO:85 and the variable light chain region comprises a sequence that is at least 80%, at least 85%, at least 90%, at least 95%, at least 96%, at least 97%, at last 98%, or least 99% identical to SEQ ID NO:114;
bb) the variable heavy chain region comprises a sequence that is at least 80%, at least 85%, at least 90%, at least 95%, at least 96%, at least 97%, at last 98%, or least 99% identical to SEQ ID NO:86 and the variable light chain region comprises a sequence that is at least 80%, at least 85%, at least 90%, at least 95%, at least 96%, at least 97%, at last 98%, or least 99% identical to SEQ ID NO:115; or
cc) the variable heavy chain region comprises a sequence that is at least 80%, at least 85%, at least 90%, at least 95%, at least 96%, at least 97%, at last 98%, or least 99% identical to SEQ ID NO:87 and the variable light chain region comprises a sequence that is at least 80%, at least 85%, at least 90%, at least 95%, at least 96%, at least 97%, at last 98%, or least 99% identical to SEQ ID NO:116.

Provided herein is an antibody that binds to SARS-CoV-2 spike protein or a fragment thereof (e.g., RBD) comprising a variable heavy chain region and a variable light chain region wherein:
a) the variable heavy chain region comprises SEQ ID NO:59 and the variable light chain region comprises SEQ ID NO:88;
b) the variable heavy chain region comprises SEQ ID NO:60 and the variable light chain region comprises SEQ ID NO:89;
c) the variable heavy chain region comprises SEQ ID NO:61 and the variable light chain region comprises SEQ ID NO:90;
d) the variable heavy chain region comprises SEQ ID NO:62 and the variable light chain region comprises SEQ ID NO:91;
e) the variable heavy chain region comprises SEQ ID NO:63 and the variable light chain region comprises SEQ ID NO:92;
f) the variable heavy chain region comprises SEQ ID NO:64 and the variable light chain region comprises SEQ ID NO:93;
g) the variable heavy chain region comprises SEQ ID NO:65 and the variable light chain region comprises SEQ ID NO:94;
h) the variable heavy chain region comprises SEQ ID NO:66 and the variable light chain region comprises SEQ ID NO:95;
i) the variable heavy chain region comprises SEQ ID NO:67 and the variable light chain region comprises SEQ ID NO:96;
j) the variable heavy chain region comprises SEQ ID NO:68 and the variable light chain region comprises SEQ ID NO:97;
k) the variable heavy chain region comprises SEQ ID NO:69 and the variable light chain region comprises SEQ ID NO:98;
l) the variable heavy chain region comprises SEQ ID NO:70 and the variable light chain region comprises SEQ ID NO:99;
m) the variable heavy chain region comprises SEQ ID NO:71 and the variable light chain region comprises SEQ ID NO:100;
n) the variable heavy chain region comprises SEQ ID NO:72 and the variable light chain region comprises SEQ ID NO:101;
o) the variable heavy chain region comprises SEQ ID NO:73 and the variable light chain region comprises SEQ ID NO:102;
p) the variable heavy chain region comprises SEQ ID NO:74 and the variable light chain region comprises SEQ ID NO:103;
q) the variable heavy chain region comprises SEQ ID NO:75 and the variable light chain region comprises SEQ ID NO:104;
r) the variable heavy chain region comprises SEQ ID NO:76 and the variable light chain region comprises SEQ ID NO:105;
s) the variable heavy chain region comprises SEQ ID NO:77 and the variable light chain region comprises SEQ ID NO:106;
t) the variable heavy chain region comprises SEQ ID NO:78 and the variable light chain region comprises SEQ ID NO:107;
u) the variable heavy chain region comprises SEQ ID NO:79 and the variable light chain region comprises SEQ ID NO:108;
v) the variable heavy chain region comprises SEQ ID NO:80 and the variable light chain region comprises SEQ ID NO:109;
w) the variable heavy chain region comprises SEQ ID NO:81 and the variable light chain region comprises SEQ ID NO:110;
x) the variable heavy chain region comprises SEQ ID NO:82 and the variable light chain region comprises SEQ ID NO:111;
y) the variable heavy chain region comprises SEQ ID NO:83 and the variable light chain region comprises SEQ ID NO:112;
z) the variable heavy chain region comprises SEQ ID NO:84 and the variable light chain region comprises SEQ ID NO:113;
aa) the variable heavy chain region comprises SEQ ID NO:85 and the variable light chain region comprises SEQ ID NO:114;
bb) the variable heavy chain region comprises SEQ ID NO:86 and the variable light chain region comprises SEQ ID NO:115; or
cc) the variable heavy chain region comprises SEQ ID NO:87 and the variable light chain region comprises SEQ ID NO:116.

Provided herein is an antibody that binds to SARS-CoV-2 spike protein or a fragment thereof (e.g., RBD) comprising a variable heavy chain region comprising a CDR1H, a CDR2H, and a CDR3H, and a variable light chain region comprising a CFR1L, a CDR2L, and a CDR3L, wherein:
a) CDR1H comprises SEQ ID NO:204, CDR2H comprises SEQ ID NO:233, CDR3H comprises SEQ ID NO:262, CDR1L comprises SEQ ID NO:378, CDR2L comprises sequence GAS, and CDR3L comprises SEQ ID NO:407;
b) CDR1H comprises SEQ ID NO:205, CDR2H comprises SEQ ID NO:234, CDR3H comprises SEQ ID NO:263, CDR1L comprises SEQ ID NO:379, CDR2L comprises sequence GAS, and CDR3L comprises SEQ ID NO:408;
c) CDR1H comprises SEQ ID NO:206, CDR2H comprises SEQ ID NO:235, CDR3H comprises SEQ ID NO:264, CDR1L comprises SEQ ID NO:380, CDR2L comprises sequence GAS, and CDR3L comprises SEQ ID NO:409;
d) CDR1H comprises SEQ ID NO:207, CDR2H comprises SEQ ID NO:236, CDR3H comprises SEQ ID NO:265, CDR1L comprises SEQ ID NO:381, CDR2L comprises sequence GAS, and CDR3L comprises SEQ ID NO:410;
e) CDR1H comprises SEQ ID NO:208, CDR2H comprises SEQ ID NO:237, CDR3H comprises SEQ ID NO:266, CDR1L comprises SEQ ID NO:382, CDR2L comprises sequence GAS, and CDR3L comprises SEQ ID NO:411;
f) CDR1H comprises SEQ ID NO:209, CDR2H comprises SEQ ID NO:238, CDR3H comprises SEQ ID NO:267, CDR1L comprises SEQ ID NO:383, CDR2L comprises sequence GAS, and CDR3L comprises SEQ ID NO:412;
g) CDR1H comprises SEQ ID NO:210, CDR2H comprises SEQ ID NO:239, CDR3H comprises SEQ ID NO:268, CDR1L comprises SEQ ID NO:384, CDR2L comprises sequence GAS, and CDR3L comprises SEQ ID NO:413;
h) CDR1H comprises SEQ ID NO:211, CDR2H comprises SEQ ID NO:240, CDR3H comprises SEQ ID NO:269, CDR1L comprises SEQ ID NO:385, CDR2L comprises sequence GAS, and CDR3L comprises SEQ ID NO:414;
i) CDR1H comprises SEQ ID NO:212, CDR2H comprises SEQ ID NO:241, CDR3H comprises SEQ ID NO:270, CDR1L comprises SEQ ID NO:386, CDR2L comprises sequence GAS, and CDR3L comprises SEQ ID NO:415;
j) CDR1H comprises SEQ ID NO:213, CDR2H comprises SEQ ID NO:242, CDR3H comprises SEQ ID NO:271, CDR1L comprises SEQ ID NO:387, CDR2L comprises sequence GAS, and CDR3L comprises SEQ ID NO:416;
k) CDR1H comprises SEQ ID NO:214, CDR2H comprises SEQ ID NO:243, CDR3H comprises SEQ ID NO:272, CDR1L comprises SEQ ID NO:388, CDR2L comprises sequence AAS, and CDR3L comprises SEQ ID NO:417;
l) CDR1H comprises SEQ ID NO:215, CDR2H comprises SEQ ID NO:244, CDR3H comprises SEQ ID NO:273, CDR1L comprises SEQ ID NO:389, CDR2L comprises sequence AAS, and CDR3L comprises SEQ ID NO:418;
m) CDR1H comprises SEQ ID NO:216, CDR2H comprises SEQ ID NO:245, CDR3H comprises SEQ ID NO:274, CDR1L comprises SEQ ID NO:390, CDR2L comprises sequence GAS, and CDR3L comprises SEQ ID NO:419;
n) CDR1H comprises SEQ ID NO:217, CDR2H comprises SEQ ID NO:246, CDR3H comprises SEQ ID NO:275, CDR1L comprises SEQ ID NO:391, CDR2L comprises sequence LGS, and CDR3L comprises SEQ ID NO:420;
o) CDR1H comprises SEQ ID NO:218, CDR2H comprises SEQ ID NO:247, CDR3H comprises SEQ ID NO:276, CDR1L comprises SEQ ID NO:392, CDR2L comprises sequence GAS, and CDR3L comprises SEQ ID NO:421;
p) CDR1H comprises SEQ ID NO:219, CDR2H comprises SEQ ID NO:248, CDR3H comprises SEQ ID NO:277, CDR1L comprises SEQ ID NO:393, CDR2L comprises sequence GAS, and CDR3L comprises SEQ ID NO:422;
q) CDR1H comprises SEQ ID NO:220, CDR2H comprises SEQ ID NO:249, CDR3H comprises SEQ ID NO:278, CDR1L comprises SEQ ID NO:394, CDR2L comprises sequence GAS, and CDR3L comprises SEQ ID NO:423;
r) CDR1H comprises SEQ ID NO:221, CDR2H comprises SEQ ID NO:250, CDR3H comprises SEQ ID NO:279, CDR1L comprises SEQ ID NO:395, CDR2L comprises sequence GAS, and CDR3L comprises SEQ ID NO:424;
s) CDR1H comprises SEQ ID NO:222, CDR2H comprises SEQ ID NO:251, CDR3H comprises SEQ ID NO:280, CDR1L comprises SEQ ID NO:396, CDR2L comprises sequence GAS, and CDR3L comprises SEQ ID NO:425;
t) CDR1H comprises SEQ ID NO:223, CDR2H comprises SEQ ID NO:252, CDR3H comprises SEQ ID NO:281, CDR1L comprises SEQ ID NO:397, CDR2L comprises sequence AAS, and CDR3L comprises SEQ ID NO:426;
u) CDR1H comprises SEQ ID NO:224, CDR2H comprises SEQ ID NO:253, CDR3H comprises SEQ ID NO:282, CDR1L comprises SEQ ID NO:398, CDR2L comprises sequence GAS, and CDR3L comprises SEQ ID NO:427;
v) CDR1H comprises SEQ ID NO:225, CDR2H comprises SEQ ID NO:254, CDR3H comprises SEQ ID NO:283, CDR1L comprises SEQ ID NO:399, CDR2L comprises sequence AAS, and CDR3L comprises SEQ ID NO:428;
w) CDR1H comprises SEQ ID NO:226, CDR2H comprises SEQ ID NO:255, CDR3H comprises SEQ ID NO:284, CDR1L comprises SEQ ID NO:400, CDR2L comprises sequence GAS, and CDR3L comprises SEQ ID NO:429;
x) CDR1H comprises SEQ ID NO:227, CDR2H comprises SEQ ID NO:256, CDR3H comprises SEQ ID NO:285, CDR1L comprises SEQ ID NO:401, CDR2L comprises sequence TS, and CDR3L comprises SEQ ID NO:430;
y) CDR1H comprises SEQ ID NO:228, CDR2H comprises SEQ ID NO:257, CDR3H comprises SEQ ID NO:286, CDR1L comprises SEQ ID NO:402, CDR2L comprises sequence AAS, and CDR3L comprises SEQ ID NO:431;
z) CDR1H comprises SEQ ID NO:229, CDR2H comprises SEQ ID NO:258, CDR3H comprises SEQ ID NO:287, CDR1L comprises SEQ ID NO:403, CDR2L comprises sequence GAS, and CDR3L comprises SEQ ID NO:432;
aa) CDR1H comprises SEQ ID NO:230, CDR2H comprises SEQ ID NO:259, CDR3H comprises SEQ ID NO:288, CDR1L comprises SEQ ID NO:404, CDR2L comprises sequence GAS, and CDR3L comprises SEQ ID NO:433;
bb) CDR1H comprises SEQ ID NO:231, CDR2H comprises SEQ ID NO:260, CDR3H comprises SEQ ID NO:289, CDR1L comprises SEQ ID NO:405, CDR2L comprises sequence AAS, and CDR3L comprises SEQ ID NO:434; or
cc) CDR1H comprises SEQ ID NO:232, CDR2H comprises SEQ ID NO:261, CDR3H comprises SEQ ID NO:290, CDR1L comprises SEQ ID NO:406, CDR2L comprises sequence AAS, and CDR3L comprises SEQ ID NO:435.

In one embodiment, an antibody provided herein comprises human-derived heavy and light chain constant regions. In one embodiment, the heavy chain constant region has an isotype selected from the group consisting of gamma1, gamma2, gamma3, and gamma4. In one embodiment, the light chain constant region has an isotype selected from the group consisting of kappa and lambda.

In one embodiment, an antibody provided herein is an immunoglobulin comprising two identical heavy chains and two identical light chains.

In one embodiment, an antibody provided herein is a monoclonal antibody. In one embodiment, an antibody provided herein is an antigen-binding fragment. In one embodiment, an antibody provided herein is a single-chain variable fragment (scFv).

In one embodiment, an antibody provided herein is conjugated to a detectable agent or a therapeutic agent.

In one embodiment, an antibody provide herein is isolated.

In another aspect, provided herein are polynucleotide sequences encoding antibodies described herein. See Tables 1 and 2 for the nucleotide sequences of the variable heavy chain region and variable light chain region of antibodies 1, 2, 3, 4, 5, 6, 7, 8, 9, 10, 11, 12, 13, 14, 15, 16, 17, 18, 19, 20, 21, 22, 23, 24, 25 26, 27, 28, and 29. In one embodiment, an antibody described herein is encoded by a nucleic acid sequence comprising the nucleotide sequences of the variable heavy chain region and variable light chain region of antibody 1, 2, 3, 4, 5, 6, 7, 8, 9, 10, 11, 12, 13, 14, 15, 16, 17, 18, 19, 20, 21, 22, 23, 24, 25, 26, 27, 28, or 29 (see Tables 1 and 2). In one embodiment, the nucleic acid sequences are isolated.

Provided herein is a nucleic acid encoding an antibody that binds to SARS-CoV-2 spike protein or a fragment thereof (e.g., RBD), wherein the nucleic acid comprises one or more of SEQ ID NOs:1-58. Provided herein is a nucleic acid encoding an antibody that binds to SARS-CoV-2 spike protein or a fragment thereof (e.g., RBD), wherein the nucleic acid comprises one or more of SEQ ID NOs:117-203 or SEQ ID NOs:291-377.

In another aspect, provided herein are expression vectors comprising a nucleotide encoding an antibody described herein. In one embodiment, an expression vector provided herein is operably linked to one or more regulatory regions.

In another aspect, provided herein are host cells comprising a nucleotide encoding an antibody described herein. In one embodiment, provided herein are host cells engineered to express an antibody described herein. The host cells may be used to produce the antibody using techniques known to one of skill in the art or described herein.

In another aspect, provided herein are compositions comprising an antibody described herein. The compositions described herein may be used in the methods of prevention, treatment, or diagnosis described herein. In one embodiment, the compositions may be used to prevent COVID-19 In another embodiment, the compositions may be used to treat a SARS-CoV-2 infection or COVID-19.

In another aspect, provided herein are methods for preventing COVID-19 comprising administering to a subject in need thereof an antibody described herein, or a composition comprising such an antibody. In one embodiment, the subject is a human. In one embodiment, the subject is a human infant or human toddler. In one embodiment, the subject is an elderly human.

In another aspect, provided herein are methods for treating SARS-CoV-2 infection or COVID-19 comprising administering to a subject in need thereof an antibody described herein, or composition comprising such an antibody. In one embodiment, the subject is diagnosed with SARS-CoV-2 virus infection or COVID-19. In one embodiment, the subject is diagnosed as SARS-CoV-2 infection or COVID-19. In one embodiment, the subject is a human. In one embodiment, the subject is a human infant or human toddler. In one embodiment, the subject is an elderly human.

In another aspect, provided herein are methods for detecting SARS-CoV-2, or diagnosing SARS-CoV-2 infection using an antibody described herein.

In another aspect, provided herein are kits comprising an antibody described herein. In one embodiment, provided herein is a kit comprising an antibody described herein, and optionally instructions for use of the antibody in the prevention or treatment of SARS-CoV-2 infection, or COVID-19, or in the detection of SARS-CoV-2 infection.

In another aspect, provided herein is an isolated antigenic peptide comprising an epitope of an antibody described herein. In one embodiment, the peptide of the SARS-CoV-21 spike protein may be used to actively immunize a patient, or in a diagnostic to detect SARS-CoV-2.

### 5. DESCRIPTION OF THE DRAWINGS

FIG. 1 is a heat map that shows the diversity of mAb binding to the spike protein. The clones were screened in several assays that included binding to membrane bound whole spike on the cell surface (MFI/flow cytometry), binding to receptor binding domain (RBD) of spike protein (RBD ELISA), RBD/ACE2 competition assay (ACE2 inhibition), and pseudovirus neutralization assay (pseudovirus neutralization).

FIG. 2 is a graph illustrating the results of neutralizing titer testing. Mabs were evaluated for neutralization potency using SARS-CoV-2 Spike expressing/VSV pseudovirus assay system. Twenty-four hours post infection, cells were analyzed by flow cytometry. Max infection (100%) is the percent of cells infected with virus alone.

FIGS. 3A, 3B, 3C, and 3D are genetic trees illustrating grouping of antibodies in families based on CDR3 identity Antibody sequences were determined to belong to two sequence families based on CDR3 homologies, designated A (FIG. 3A), B (FIG. 3B), E (FIG. 3C) and G (FIG. 3D). Individual clones with variances from members within each family are shown in the above genetic trees, with identical clones (100% homology) in shaded boxes. All clones with some degree of uniqueness were chosen to move forward. Only one clone was chosen from the identical cluster sets.

FIGS. 4A, 4B, 4C, and 4D show results from a competitive ELISA. SARS-CoV RBD coated plates were exposed to supernatants (either undiluted (neat) or at a 1:10 dilution in sera free media) comprising clones from each family or rat IgG isotype control (1 μg/ml). Biotinylated purified antibody from family A (10D6) or B (16C5) was added after the supernatants. The plates were developed with streptavidin HRP. Representative clones included 19C4 (family A), 16C12 (family B), 2C1 (family D), 14G5 (family E), 7D2 (family F), and 10A3 (family G) (FIGS. 4A and 4B). Three family E clones (3E10, 14G5, and 14G11) were examined for competition with family A (FIG. 4C) and B (FIG. 4D) biotinylated monoclonals.

FIGS. 5A, 5B, and 5C show neutralization experiments with WT (FIG. 5A) and mutant-VSV pseudovriuses N501Y (FIG. 5B) and E484K (FIG. 5C). Representative clones included 19C4 (family A), 16C12 (family B), 2C1 (family D), 14G5 (family E), 7D2 (family F), and 10A3 (family G).

FIGS. 6A, 6B, 6C, 6D, 6E, 6F, 6G, and 6H show an ELISA experiment in which fusion proteins representing the RBD domains of the SARS-CoV2 Spike protein from the wild type, N501Y single mutant, or E484K single mutant (South African variant SA) were coated to ELISA plates and screened for binding as described in the materials and methods. Monoclonal antibodies representing each of the genetic families were added to coated ELISA plates at concentrations of 2 μg/ml, 200 ng/ml, 20 ng/ml, and 2 ng/ml (from left to right).

FIGS. 7A, 7B, and 7C illustrate that anti-SARS-CoV2 RBD mAbs block virus proliferation in vivo. The timeline of sensivizing BALB/c mice with Ad5-hACE2, treatment with anti-RBD monoclonal antibodies, and challenge with SARS-CoV-2 (FIG. 7A). Monoclonal antibodies 5H12, 10D6 (FIG. 7B), 1D10, 16C5, and 19C4 (FIG. 7C) were administered intraperitoneally into BALB/c mice that had been sensitized with Ad5-hACE2 at different doses. The SARS-CoV2 viral titer (PFU/ml) from harvested lungs two days post-infection was analyzed. PBS and human IgG (hIgG) were used as controls. The panel of mAbs effectively blocked virus replication in the lungs in contrast to human IgG negative control and PBS, with almost no detectable titers in some groups.

### 6. DETAILED DESCRIPTION

6.1 Antibodies

Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecule, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′) fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG2) or subclass (e.g., IgG2a) thereof. In one embodiment, an antibody described herein is isolated or purified.

In one embodiment, an antibody includes any molecule with an antigen-binding site that binds an antigen. In some embodiments, an antibody includes an antigen-binding fragment (e.g., the region(s) of an immunoglobulin that binds to an antigen or an epitope, such as a sequence comprising complementarity determining regions (e.g., the heavy and/or light chain variable regions)). In other embodiments, an antibody does not include antigen-binding fragments.

In one embodiment, an antibody described herein is a monoclonal antibody. As used herein, the term “monoclonal antibody” refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In some embodiments, a “monoclonal antibody,” as used herein, is an antibody produced by a single cell (e.g., hybridoma or host cell producing a recombinant antibody), wherein the antibody binds to SARS-CoV-2 spike protein or a fragment thereof (e.g., RBD) as determined, e.g., by ELISA or other antigen-binding or competitive binding assay known in the art or in the Examples provided herein. In some embodiments, a monoclonal antibody can be a chimeric antibody, a human antibody, or a humanized antibody.

In certain embodiments, a monoclonal antibody is a monovalent antibody or multivalent (e.g., bivalent) antibody. In some embodiments, a monoclonal antibody is a monospecific or multispecific antibody (e.g., bispecific antibody). Monoclonal antibodies described herein can, for example, be made by the hybridoma method as described in Kohler et al.; Nature, 256:495 (1975) or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York).

In one embodiment, an antibody described herein is an immunoglobulin, such as an IgG, IgE, IgM, IgD, IgA or IgY. In one embodiment, an antibody described herein is an IgG2a. In some embodiments, an antibody described herein is an IgG1 or IgG2a. In another embodiment, antibody described herein is an antigen-binding fragment, such as, e.g., a Fab fragment or F(ab′)₂ fragment. In another embodiment, an antibody described herein is an scFv.

As used herein, the terms “SARS-CoV-2 spike protein” and “spike protein of SARS-CoV-2” refer to a SARS-CoV-2 spike protein known to those of skill in the art. In certain embodiments, the spike protein comprises the amino acid or nucleic acid sequence found at GenBank Accession No. MN908947.3. A typical spike protein comprises domains known to those of skill in the art including an S1 domain, a receptor binding domain, an S2 domain, a transmembrane domain and a cytoplasmic domain. See, e.g., Wrapp et al., 2020, Science 367: 1260-1263 for a description of SARS-CoV-2 spike protein (in particular, the structure of such protein). The spike protein may be characterized has having a signal peptide (e.g., a signal peptide of 1-14 amino acid residues of the amino acid sequence of GenBank Accession No. MN908947.3), a receptor binding domain (e.g., a receptor binding domain of 319-541 amino acid residues of GenBank Accession No. MN908947.3), an ectodomain (e.g., an ectodomain of 15-1213 amino acid residues of GenBank Accession No. MN908947.3), and a transmembrane and endodomain (e.g., a transmembrane and endodomain of 1214-1273 amino acid residues of GenBank Accession No. MN908947.3).

In certain embodiments, a fragment of a SARS-CoV-2 spike protein is at least 8, 10, 12, 15, or 20 amino acid residues in length. In some embodiments, a fragment of a SARS-CoV-2 spike protein comprises or consists of the receptor binding domain (RBD) of the spike protein. In certain embodiments, fragment of a SARS-CoV-2 spike protein consists of the RBD of the spike protein and 1, 2, 3, 4, 5, 6, 7, 8, 9, or 10 amino acid residues at the N-terminus, C-terminus or both. In some embodiments, fragment of a SARS-CoV-2 spike protein comprises or consists of the S1 domain, S2 domain or ectodomain of the spike protein. In certain embodiments, fragment of a SARS-CoV-2 spike protein consists of the S1 domain, S2 domain or ectodomain of the spike protein and 1, 2, 3, 4, 5, 6, 7, 8, 9, or 10 amino acid residues at the N-terminus, C-terminus or both.

In another aspect, the antibodies provided herein bind to SARS-CoV-2 spike protein with a certain affinity. “Binding affinity” generally refers to the strength of the sum total of non-covalent interactions between a single binding site of a molecule (e.g., an antibody) and its binding partner (e.g., an antigen). In one embodiment, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), equilibrium association constant (K_{A}), and IC₅₀. The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIAcore™, Kinexa, or biolayer interferometry.

Affinity can be measured by common methods known in the art, including those described herein. For example, individual association (kₒₙ) and dissociation (k_{off}) rate constants can be calculated from the resulting binding curves using the BIAevaluation software available through the vendor. Data can then be fit to a 1:1 binding model, which includes a term to correct for mass transport limited binding, should it be detected. From these rate constants, the apparent dissociation binding constant (K_{D}) for the interaction of the antibody (e.g., IgG) with the antigen (e.g., SARS-CoV-2 spike) can be calculated from the quotient of k_{off}/kₒₙ. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the described herein.

In another embodiment, an antibody described herein binds to SARS-CoV-2 spike protein present in the virion particle. In one embodiment, an antibody described herein binds to a SARS-CoV-2 spike protein on the surface of a cell infected with SARS-CoV-2.

In some embodiments, an antibody described herein binds to SARS-CoV-2 spike protein as assessed by techniques known in the art, e.g., ELISA, Western blot, biolayer interferometry, FACS or BIACore, or described herein. In other embodiments, an antibody described herein does not cross-react with spike protein from another type of coronavirus (e.g., other betacoronaviruses) as assessed by techniques known in the art, e.g., ELISA, Western blot, biolayer interferometry, FACS or BIACore, or described herein. In certain embodiments, provided herein is an antibody that selectively binds to SARS-CoV-2 spike protein relative to a spike protein of another type of coronavirus as assessed by techniques known in the art, e.g., ELISA, Western blot, biolayer interferometry, FACS or BIACore, or described herein. In some embodiments, provided herein is an antibody that selectively binds to SARS-CoV-2 spike protein relative to a spike protein of other betacoronaviruses as assessed by techniques known in the art, e.g., ELISA, Western blot, biolayer interferometry, FACS or BIACore, or described herein. In certain embodiments, provided herein is an antibody that selectively binds to SARS-CoV-2 spike protein relative to a surface protein of a non-coronavirus as assessed by techniques known in the art, e.g., ELISA, Western blot, biolayer interferometry, FACS or BIACore, or described herein.

In certain embodiments, an antibody that binds to a SARS-CoV-2 spike protein inhibits the binding of the spike protein to a host cell receptor. In certain embodiments, an antibody that binds to a SARS-CoV-2 spike protein inhibits the binding of the spike protein to a host cell receptor (e.g., human receptor angiotensin converting enzyme 2 (ACE2)). The inhibition of binding may be complete or partial as assessed by a technique known to one of skill in the art or described herein.

In another aspect, an antibody provided herein has one, two or more, or all of the characteristics/properties of one of the antibodies described herein (e.g., an antibody described in Section 5, infra). For example, in certain embodiments, an antibody described herein has neutralizing activity as assessed by a technique known to one of skill in the art.

In another aspect, an antibody described herein binds to the receptor binding domain of SARS-CoV-2 spike protein or a fragment thereof as assessed by a technique known to one of skill in the art or described herein. For example, an antibody described herein binds to a fragment of the SARS-CoV-2 spike protein comprising the receptor binding domain. Such fragment may comprise amino acid resides 258 to 572 or amino acid residues 498-572 of the SARS-CoV-2 spike protein.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids in a mature heavy chain and about the amino-terminal 90 to 100 amino acids in a mature light chain, which differs extensively in sequence among antibodies and is used in the binding and specificity of a particular antibody for its particular antigen. The variability in sequence is concentrated in those regions called complementarity determining regions (CDRs) while the more highly conserved regions in the variable domain are called framework regions (FR). CDRs are flanked by FRs. Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction and specificity of the antibody with antigen. In certain embodiments, the variable region is a rodent (e.g., mouse or rat) variable region. In certain embodiments, the variable region is a human variable region. In certain embodiments, the variable region comprises rodent (e.g., mouse or rat) CDRs and human framework regions (FRs). In some embodiments, the variable region is a primate (e.g., non-human primate) variable region. In certain embodiments, the variable region comprises rodent or murine CDRs and primate (e.g., non-human primate) framework regions (FRs).

In another aspect, an antibody provided herein comprises one, two or three of the complementarity determining regions (CDRs) of the variable heavy chain region (“VH” domain) or one, two or three of the CDRs of the variable light chain region (“VL” domain) of an antibody described herein. In another aspect, an antibody provided herein comprises one, two or three of the complementarity determining regions (CDRs) of the variable heavy chain region (“VH” domain) and one, two or three of the CDRs of the variable light chain region (“VL” domain) of an antibody described herein. In another aspect, an antibody provided herein comprises the complementarity determining regions (CDRs) of the variable heavy chain region (“VH” domain) and the CDRs of the variable light chain region (“VL” domain) of an antibody described herein.

In certain aspects, the CDRs of an antibody can be determined according to the Kabat numbering system. The terms “Kabat numbering,” and like terms are recognized in the art and refer to a system of numbering amino acid residues in the heavy and light chain variable regions of an antibody, or an antigen-binding portion thereof. In certain aspects, the CDRs of an antibody can be determined according to the Kabat numbering system (see, e.g., Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). With respect to the Kabat numbering system, (i) the VH CDR1 is typically present at amino acid positions 31 to 35 of the heavy chain, which can optionally include one or two additional amino acids following amino acid position 35 (referred to in the Kabat numbering scheme as 35A and 35B); (ii) the VH CDR2 is typically present at amino acid positions 50 to 65 of the heavy chain; and (iii) the VH CDR2 is typically present at amino acid positions 95 to 102 of the heavy chain (Kabat, Elvin A. et al., Sequences of Proteins of Immunological Interest. Bethesda: National Institutes of Health, 1983). With respect to the Kabat numbering system, (i) the VL CDR1 is typically present at amino acid positions 24 to 34 of the light chain; (ii) the VL CDR2 is typically present at amino acid positions 50 to 56 of the light chain; and (iii) the VL CDR3 is typically present at amino acid positions 89 to 97 of the light chain (Kabat, Elvin A. et al., Sequences of Proteins of Immunological Interest. Bethesda: National Institutes of Health, 1983). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

In certain aspects, the CDRs of an antibody can be determined according to the Chothia numbering scheme, which refers to the location of immunoglobulin structural loops (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1):175-82; and U.S. Pat. No. 7,709,226). The Chothia definition is based on the location of the structural loop regions (Chothia et al., (1987) J Mol Biol 196: 901-917; and U.S. Pat. No. 7,709,226). The term “Chothia CDRs,” and like terms are recognized in the art and refer to antibody CDR sequences as determined according to the method of Chothia and Lesk, 1987, J. Mol. Biol., 196:901-917, which will be referred to herein as the “Chothia CDRs” (see also, e.g., U.S. Pat. No. 7,709,226 and Martin, A., “Protein Sequence and Structure Analysis of Antibody Variable Domains,” in Antibody Engineering, Kontermann and DObe, eds., Chapter 31, pp. 422-439, Springer-Verlag, Berlin (2001)). With respect to the Chothia numbering system, using the Kabat numbering system of numbering amino acid residues in the VH region, (i) the VH CDR1 is typically present at amino acid positions 26 to 32 of the heavy chain; (ii) the VH CDR2 is typically present at amino acid positions 53 to 55 of the heavy chain; and (iii) the VH CDR3 is typically present at amino acid positions 96 to 101 of the heavy chain. In one embodiment, with respect to the Chothia numbering system, using the Kabat numbering system of numbering amino acid residues in the VH region, (i) the VH CDR1 is typically present at amino acid positions 26 to 32 or 34 of the heavy chain; (ii) the VH CDR2 is typically present at amino acid positions 52 to 56 (in one embodiment, CDR2 is at positions 52A-56, wherein 52A follows position 52) of the heavy chain; and (iii) the VH CDR3 is typically present at amino acid positions 95 to 102 of the heavy chain (in one embodiment, there is no amino acid at positions numbered 96-100). With respect to the Chothia numbering system, using the Kabat numbering system of numbering amino acid residues in the VL region, (i) the VL CDR1 is typically present at amino acid positions 26 to 33 of the light chain; (ii) the VL CDR2 is typically present at amino acid positions 50 to 52 of the light chain; and (iii) the VL CDR3 is typically present at amino acid positions 91 to 96 of the light chain. In one embodiment, with respect to the Chothia numbering system, using the Kabat numbering system of numbering amino acid residues in the VL region, (i) the VL CDR1 is typically present at amino acid positions 24 to 34 of the light chain; (ii) the VL CDR2 is typically present at amino acid positions 50 to 56 of the light chain; and (iii) the VL CDR3 is typically present at amino acid positions 89 to 97 of the light chain (in one embodiment, there is no amino acid at positions numbered 96-100). These Chothia CDR positions may vary depending on the antibody, and may be determined according to methods known in the art.

In certain aspects, the CDRs of an antibody can be determined according to the IMGT numbering system as described in Lefranc, M.-P., 1999, The Immunologist, 7:132-136 and Lefranc, M.-P. et al., 1999, Nucleic Acids Res., 27:209-212. The IMGT definition is from the IMGT (“IMGT®, the international ImMunoGeneTics information System® website imgt.org, founder and director: Marie-Paule Lefranc, Montpellier, France; see, e.g., Lefranc, M.-P., 1999, The Immunologist, 7:132-136 and Lefranc, M.-P. et al., 1999, Nucleic Acids Res., 27:209-212, both of which are incorporated herein by reference in their entirety). With respect to the IMGT numbering system, (i) the VH CDR1 is typically present at amino acid positions 25 to 35 of the heavy chain; (ii) the VH CDR2 is typically present at amino acid positions 51 to 57 of the heavy chain; and (iii) the VH CDR2 is typically present at amino acid positions 93 to 102 of the heavy chain. With respect to the IMGT numbering system, (i) the VL CDR1 is typically present at amino acid positions 27 to 32 of the light chain; (ii) the VL CDR2 is typically present at amino acid positions 50 to 52 of the light chain; and (iii) the VL CDR3 is typically present at amino acid positions 89 to 97 of the light chain.

In certain aspects, the CDRs of an antibody can be determined according to MacCallum et al., 1996, J. Mol. Biol., 262:732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001).

In certain aspects, the CDRs of an antibody can be determined according to the AbM numbering scheme, which refers AbM hypervariable regions which represent a compromise between the Kabat CDRs and Chothia structural loops, and are used by Oxford Molecular's AbM antibody modeling software. A person of ordinary skill in the art would be able to determine the CDRs and framework regions of the variable regions of the 2B3, 1C7C7 or 22C7C4 antibody sequence based on known numbering systems, such as the Kabat numbering system, Chothia system, Oxford's AbM system, and/or contact system.

A person skilled in the art would be able to identify the CDRs of the provided variable region sequences using techniques known to a person skilled in the art, such as described herein. In one embodiment, the position of a CDR along the VH and/or VL domain of an antibody described herein may vary by one, two, three or four amino acid positions so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). For example, in one embodiment, the position defining a CDR of antibody described herein may vary by shifting the N-terminal and/or C-terminal boundary of the CDR by one, two, three, or four amino acids, relative to the CDR position, so long as binding to herein SARS-CoV-2 spike protein is maintained or substantially maintained (for example, by at least 50%, at least 60%, at least 70%, at least 80%, at least 90%, at least 95% in an assay known in the art or described herein, such as an ELISA).

In another aspect, provided herein are antibodies that bind to SARS-CoV-2 spike protein comprising one, two or three complementarity determining regions (CDRs) of the variable heavy chain region of an antibody described herein (e.g., antibody number 1 in Table 1) and one, two or three CDRs of the variable light chain region of that antibody (e.g., antibody number 1 in Table 2). In certain embodiments, an antibody that binds to a SARS-CoV-2 spike protein, comprises (or alternatively, consists of) a VH CDR1 and a VL CDR1; a VH CDR1 and a VL CDR2; a VH CDR1 and a VL CDR3; a VH CDR2 and a VL CDR1; VH CDR2 and a VL CDR2; a VH CDR2 and a VL CDR3; a VH CDR3 and a VL CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1; a VH CDR2, a VH CDR3 and a VL CDR2; a VH CDR2, a VH CDR3 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; a VH CDR1, VH CDR2, a VH CDR3, a VL CDR1, a VL CDR2, and a VL CDR3; or any combination thereof of the VH CDRs and VL CDRs of an antibody described herein (e.g., antibody number 1 in Tables 1 and 2).

In one embodiment, provided herein is an antibody that binds to SARS-CoV-2 spike protein comprising the three CDRs of the variable heavy chain region of antibody number 1 in Table 1 and the three CDRs of the variable light chain region of antibody number 1 in Table 2. In another embodiment, provided herein is an antibody that binds to SARS-CoV-2 spike protein comprising the three CDRs of the variable heavy chain region of antibody number 2 in Table 1 and the three CDRs of the variable light chain region of antibody number 2 in Table 2. In another embodiment, provided herein is an antibody that binds to SARS-CoV-2 spike protein comprising the three CDRs of the variable heavy chain region of antibody number 3 in Table 1 and the three CDRs of the variable light chain region of antibody number 3 in Table 2. In another embodiment, provided herein is an antibody that binds to SARS-CoV-2 spike protein comprising the three CDRs of the variable heavy chain region of antibody number 4 in Table 1 and the three CDRs of the variable light chain region of antibody number 4 in Table 2. In another embodiment, provided herein is an antibody that binds to SARS-CoV-2 spike protein comprising the three CDRs of the variable heavy chain region of antibody number 5 in Table 1 and the three CDRs of the variable light chain region of antibody number 5 in Table 2. In another embodiment, provided herein is an antibody that binds to SARS-CoV-2 spike protein comprising the three CDRs of the variable heavy chain region of antibody number 6 in Table 1 and the three CDRs of the variable light chain region of antibody number 6 in Table 2. In another embodiment, provided herein is an antibody that binds to SARS-CoV-2 spike protein comprising the three CDRs of the variable heavy chain region of antibody number 7 in Table 1 and the three CDRs of the variable light chain region of antibody number 7 in Table 2. In another embodiment, provided herein is an antibody that binds to SARS-CoV-2 spike protein comprising the three CDRs of the variable heavy chain region of antibody number 8 in Table 1 and the three CDRs of the variable light chain region of antibody number 8 in Table 2. In another embodiment, provided herein is an antibody that binds to SARS-CoV-2 spike protein comprising the three CDRs of the variable heavy chain region of antibody number 9 in Table 1 and the three CDRs of the variable light chain region of antibody number 9 in Table 2. In another embodiment, provided herein is an antibody that binds to SARS-CoV-2 spike protein comprising the three CDRs of the variable heavy chain region of antibody number 10 in Table 1 and the three CDRs of the variable light chain region of antibody number 10 in Table 2. In another embodiment, provided herein is an antibody that binds to SARS-CoV-2 spike protein comprising the three CDRs of the variable heavy chain region of antibody number 11 in Table 1 and the three CDRs of the variable light chain region of antibody number 11 in Table 2. In another embodiment, provided herein is an antibody that binds to SARS-CoV-2 spike protein comprising the three CDRs of the variable heavy chain region of antibody number 12 in Table 1 and the three CDRs of the variable light chain region of antibody number 12 in Table 2. In another embodiment, provided herein is an antibody that binds to SARS-CoV-2 spike protein comprising the three CDRs of the variable heavy chain region of antibody number 13 in Table 1 and the three CDRs of the variable light chain region of antibody number 13 in Table 2. In another embodiment, provided herein is an antibody that binds to SARS-CoV-2 spike protein comprising the three CDRs of the variable heavy chain region of antibody number 14 in Table 1 and the three CDRs of the variable light chain region of antibody number 14 in Table 2. In another embodiment, provided herein is an antibody that binds to SARS-CoV-2 spike protein comprising the three CDRs of the variable heavy chain region of antibody number 15 in Table 1 and the three CDRs of the variable light chain region of antibody number 15 in Table 2. In another embodiment, provided herein is an antibody that binds to SARS-CoV-2 spike protein comprising the three CDRs of the variable heavy chain region of antibody number 16 in Table 1 and the three CDRs of the variable light chain region of antibody number 16 in Table 2. In another embodiment, provided herein is an antibody that binds to SARS-CoV-2 spike protein comprising the three CDRs of the variable heavy chain region of antibody number 17 in Table 1 and the three CDRs of the variable light chain region of antibody number 17 in Table 2. In another embodiment, provided herein is an antibody that binds to SARS-CoV-2 spike protein comprising the three CDRs of the variable heavy chain region of antibody number 18 in Table 1 and the three CDRs of the variable light chain region of antibody number 18 in Table 2. In another embodiment, provided herein is an antibody that binds to SARS-CoV-2 spike protein comprising the three CDRs of the variable heavy chain region of antibody number 19 in Table 1 and the three CDRs of the variable light chain region of antibody number 19 in Table 2. In another embodiment, provided herein is an antibody that binds to SARS-CoV-2 spike protein comprising the three CDRs of the variable heavy chain region of antibody number 20 in Table 1 and the three CDRs of the variable light chain region of antibody number 20 in Table 2. In another embodiment, provided herein is an antibody that binds to SARS-CoV-2 spike protein comprising the three CDRs of the variable heavy chain region of antibody number 21 in Table 1 and the three CDRs of the variable light chain region of antibody number 21 in Table 2. In another embodiment, provided herein is an antibody that binds to SARS-CoV-2 spike protein comprising the three CDRs of the variable heavy chain region of antibody number 22 in Table 1 and the three CDRs of the variable light chain region of antibody number 22 in Table 2. In another embodiment, provided herein is an antibody that binds to SARS-CoV-2 spike protein comprising the three CDRs of the variable heavy chain region of antibody number 23 in Table 1 and the three CDRs of the variable light chain region of antibody number 23 in Table 2. In another embodiment, provided herein is an antibody that binds to SARS-CoV-2 spike protein comprising the three CDRs of the variable heavy chain region of antibody number 24 in Table 1 and the three CDRs of the variable light chain region of antibody number 24 in Table 2. In another embodiment, provided herein is an antibody that binds to SARS-CoV-2 spike protein comprising the three CDRs of the variable heavy chain region of antibody number 25 in Table 1 and the three CDRs of the variable light chain region of antibody number 25 in Table 2. In another embodiment, provided herein is an antibody that binds to SARS-CoV-2 spike protein comprising the three CDRs of the variable heavy chain region of antibody number 26 in Table 1 and the three CDRs of the variable light chain region of antibody number 26 in Table 2. In another embodiment, provided herein is an antibody that binds to SARS-CoV-2 spike protein comprising the three CDRs of the variable heavy chain region of antibody number 27 in Table 1 and the three CDRs of the variable light chain region of antibody number 27 in Table 2. In another embodiment, provided herein is an antibody that binds to SARS-CoV-2 spike protein comprising the three CDRs of the variable heavy chain region of antibody number 28 in Table 1 and the three CDRs of the variable light chain region of antibody number 28 in Table 2. In another embodiment, provided herein is an antibody that binds to SARS-CoV-2 spike protein comprising the three CDRs of the variable heavy chain region of antibody number 29 in Table 1 and the three CDRs of the variable light chain region of antibody number 29 in Table 2.

In another embodiment, provided herein is an antibody that binds to SARS-CoV-2 spike protein comprising CDR1, CDR2, or CDR3 of the variable heavy chain region of antibody number 1 in Table 6 and CDR1, CDR2, or CDR3 of the variable light chain region of antibody number 1 in Table 8. In another embodiment, provided herein is an antibody that binds to SARS-CoV-2 spike protein comprising two of CDR1, CDR2, and CDR3 of the variable heavy chain region of antibody number 1 in Table 6 and two of CDR1, CDR2, and CDR3 of the variable light chain region of antibody number 1 in Table 8. In another embodiment, provided herein is an antibody that binds to SARS-CoV-2 spike protein comprising CDR1, CDR2, and CDR3 of the variable heavy chain region of antibody number 1 in Table 6 and CDR1, CDR2, and CDR3 of the variable light chain region of antibody number 1 in Table 8. In another embodiment, provided herein is an antibody that binds to SARS-CoV-2 spike protein comprising CDR1, CDR2, and CDR3 of the variable heavy chain region of antibody number 1 in Table 6 and CDR1, CDR2, and CDR3 of the variable light chain region of antibody number 1 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 1 in Table 6 and CDR1, CDR2, and CDR3 of the variable light chain region of antibody number 1 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 2 in Table 6 and CDR1, CDR2, or CDR3 of the variable light chain region of antibody number 2 in Table 8. In another embodiment, provided herein is an antibody that binds to SARS-CoV-2 spike protein comprising two of CDR1, CDR2, and CDR3 of the variable heavy chain region of antibody number 2 in Table 6 and two of CDR1, CDR2, and CDR3 of the variable light chain region of antibody number 2 in Table 8. In another embodiment, provided herein is an antibody that binds to SARS-CoV-2 spike protein comprising CDR1, CDR2, and CDR3 of the variable heavy chain region of antibody number 2 in Table 6 and CDR1, CDR2, and CDR3 of the variable light chain region of antibody number 2 in Table 8. In another embodiment, provided herein is an antibody that binds to SARS-CoV-2 spike protein comprising CDR1, CDR2, and CDR3 of the variable heavy chain region of antibody number 2 in Table 6 and CDR1, CDR2, and CDR3 of the variable light chain region of antibody number 2 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 2 in Table 6 and CDR1, CDR2, and CDR3 of the variable light chain region of antibody number 2 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 3 in Table 6 and CDR1, CDR2, or CDR3 of the variable light chain region of antibody number 3 in Table 8. In another embodiment, provided herein is an antibody that binds to SARS-CoV-2 spike protein comprising two of CDR1, CDR2, and CDR3 of the variable heavy chain region of antibody number 3 in Table 6 and two of CDR1, CDR2, and CDR3 of the variable light chain region of antibody number 3 in Table 8. In another embodiment, provided herein is an antibody that binds to SARS-CoV-2 spike protein comprising CDR1, CDR2, and CDR3 of the variable heavy chain region of antibody number 3 in Table 6 and CDR1, CDR2, and CDR3 of the variable light chain region of antibody number 3 in Table 8. In another embodiment, provided herein is an antibody that binds to SARS-CoV-2 spike protein comprising CDR1, CDR2, and CDR3 of the variable heavy chain region of antibody number 3 in Table 6 and CDR1, CDR2, and CDR3 of the variable light chain region of antibody number 3 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 3 in Table 6 and CDR1, CDR2, and CDR3 of the variable light chain region of antibody number 3 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 4 in Table 6 and CDR1, CDR2, or CDR3 of the variable light chain region of antibody number 4 in Table 8. In another embodiment, provided herein is an antibody that binds to SARS-CoV-2 spike protein comprising two of CDR1, CDR2, and CDR3 of the variable heavy chain region of antibody number 4 in Table 6 and two of CDR1, CDR2, and CDR3 of the variable light chain region of antibody number 4 in Table 8. In another embodiment, provided herein is an antibody that binds to SARS-CoV-2 spike protein comprising CDR1, CDR2, and CDR3 of the variable heavy chain region of antibody number 4 in Table 6 and CDR1, CDR2, and CDR3 of the variable light chain region of antibody number 4 in Table 8. In another embodiment, provided herein is an antibody that binds to SARS-CoV-2 spike protein comprising CDR1, CDR2, and CDR3 of the variable heavy chain region of antibody number 4 in Table 6 and CDR1, CDR2, and CDR3 of the variable light chain region of antibody number 4 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 4 in Table 6 and CDR1, CDR2, and CDR3 of the variable light chain region of antibody number 4 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 5 in Table 6 and CDR1, CDR2, or CDR3 of the variable light chain region of antibody number 5 in Table 8. In another embodiment, provided herein is an antibody that binds to SARS-CoV-2 spike protein comprising two of CDR1, CDR2, and CDR3 of the variable heavy chain region of antibody number 5 in Table 6 and two of CDR1, CDR2, and CDR3 of the variable light chain region of antibody number 5 in Table 8. In another embodiment, provided herein is an antibody that binds to SARS-CoV-2 spike protein comprising CDR1, CDR2, and CDR3 of the variable heavy chain region of antibody number 5 in Table 6 and CDR1, CDR2, and CDR3 of the variable light chain region of antibody number 5 in Table 8. In another embodiment, provided herein is an antibody that binds to SARS-CoV-2 spike protein comprising CDR1, CDR2, and CDR3 of the variable heavy chain region of antibody number 5 in Table 6 and CDR1, CDR2, and CDR3 of the variable light chain region of antibody number 5 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 5 in Table 6 and CDR1, CDR2, and CDR3 of the variable light chain region of antibody number 5 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 6 in Table 6 and CDR1, CDR2, or CDR3 of the variable light chain region of antibody number 6 in Table 8. In another embodiment, provided herein is an antibody that binds to SARS-CoV-2 spike protein comprising two of CDR1, CDR2, and CDR3 of the variable heavy chain region of antibody number 6 in Table 6 and two of CDR1, CDR2, and CDR3 of the variable light chain region of antibody number 6 in Table 8. In another embodiment, provided herein is an antibody that binds to SARS-CoV-2 spike protein comprising CDR1, CDR2, and CDR3 of the variable heavy chain region of antibody number 6 in Table 6 and CDR1, CDR2, and CDR3 of the variable light chain region of antibody number 6 in Table 8. In another embodiment, provided herein is an antibody that binds to SARS-CoV-2 spike protein comprising CDR1, CDR2, and CDR3 of the variable heavy chain region of antibody number 6 in Table 6 and CDR1, CDR2, and CDR3 of the variable light chain region of antibody number 6 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 6 in Table 6 and CDR1, CDR2, and CDR3 of the variable light chain region of antibody number 6 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 7 in Table 6 and CDR1, CDR2, or CDR3 of the variable light chain region of antibody number 7 in Table 8. In another embodiment, provided herein is an antibody that binds to SARS-CoV-2 spike protein comprising two of CDR1, CDR2, and CDR3 of the variable heavy chain region of antibody number 7 in Table 6 and two of CDR1, CDR2, and CDR3 of the variable light chain region of antibody number 7 in Table 8. In another embodiment, provided herein is an antibody that binds to SARS-CoV-2 spike protein comprising CDR1, CDR2, and CDR3 of the variable heavy chain region of antibody number 7 in Table 6 and CDR1, CDR2, and CDR3 of the variable light chain region of antibody number 7 in Table 8. In another embodiment, provided herein is an antibody that binds to SARS-CoV-2 spike protein comprising CDR1, CDR2, and CDR3 of the variable heavy chain region of antibody number 7 in Table 6 and CDR1, CDR2, and CDR3 of the variable light chain region of antibody number 7 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 7 in Table 6 and CDR1, CDR2, and CDR3 of the variable light chain region of antibody number 7 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 8 in Table 6 and CDR1, CDR2, or CDR3 of the variable light chain region of antibody number 8 in Table 8. In another embodiment, provided herein is an antibody that binds to SARS-CoV-2 spike protein comprising two of CDR1, CDR2, and CDR3 of the variable heavy chain region of antibody number 8 in Table 6 and two of CDR1, CDR2, and CDR3 of the variable light chain region of antibody number 8 in Table 8. In another embodiment, provided herein is an antibody that binds to SARS-CoV-2 spike protein comprising CDR1, CDR2, and CDR3 of the variable heavy chain region of antibody number 8 in Table 6 and CDR1, CDR2, and CDR3 of the variable light chain region of antibody number 8 in Table 8. In another embodiment, provided herein is an antibody that binds to SARS-CoV-2 spike protein comprising CDR1, CDR2, and CDR3 of the variable heavy chain region of antibody number 8 in Table 6 and CDR1, CDR2, and CDR3 of the variable light chain region of antibody number 8 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 8 in Table 6 and CDR1, CDR2, and CDR3 of the variable light chain region of antibody number 8 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 9 in Table 6 and CDR1, CDR2, or CDR3 of the variable light chain region of antibody number 9 in Table 8. In another embodiment, provided herein is an antibody that binds to SARS-CoV-2 spike protein comprising two of CDR1, CDR2, and CDR3 of the variable heavy chain region of antibody number 9 in Table 6 and two of CDR1, CDR2, and CDR3 of the variable light chain region of antibody number 9 in Table 8. In another embodiment, provided herein is an antibody that binds to SARS-CoV-2 spike protein comprising CDR1, CDR2, and CDR3 of the variable heavy chain region of antibody number 9 in Table 6 and CDR1, CDR2, and CDR3 of the variable light chain region of antibody number 9 in Table 8. In another embodiment, provided herein is an antibody that binds to SARS-CoV-2 spike protein comprising CDR1, CDR2, and CDR3 of the variable heavy chain region of antibody number 9 in Table 6 and CDR1, CDR2, and CDR3 of the variable light chain region of antibody number 9 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 9 in Table 6 and CDR1, CDR2, and CDR3 of the variable light chain region of antibody number 9 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 10 in Table 6 and CDR1, CDR2, or CDR3 of the variable light chain region of antibody number 10 in Table 8. In another embodiment, provided herein is an antibody that binds to SARS-CoV-2 spike protein comprising two of CDR1, CDR2, and CDR3 of the variable heavy chain region of antibody number 10 in Table 6 and two of CDR1, CDR2, and CDR3 of the variable light chain region of antibody number 10 in Table 8. In another embodiment, provided herein is an antibody that binds to SARS-CoV-2 spike protein comprising CDR1, CDR2, and CDR3 of the variable heavy chain region of antibody number 10 in Table 6 and CDR1, CDR2, and CDR3 of the variable light chain region of antibody number 10 in Table 8. In another embodiment, provided herein is an antibody that binds to SARS-CoV-2 spike protein comprising CDR1, CDR2, and CDR3 of the variable heavy chain region of antibody number 10 in Table 6 and CDR1, CDR2, and CDR3 of the variable light chain region of antibody number 10 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 10 in Table 6 and CDR1, CDR2, and CDR3 of the variable light chain region of antibody number 10 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 11 in Table 6 and CDR1, CDR2, or CDR3 of the variable light chain region of antibody number 11 in Table 8. In another embodiment, provided herein is an antibody that binds to SARS-CoV-2 spike protein comprising two of CDR1, CDR2, and CDR3 of the variable heavy chain region of antibody number 11 in Table 6 and two of CDR1, CDR2, and CDR3 of the variable light chain region of antibody number 11 in Table 8. In another embodiment, provided herein is an antibody that binds to SARS-CoV-2 spike protein comprising CDR1, CDR2, and CDR3 of the variable heavy chain region of antibody number 11 in Table 6 and CDR1, CDR2, and CDR3 of the variable light chain region of antibody number 11 in Table 8. In another embodiment, provided herein is an antibody that binds to SARS-CoV-2 spike protein comprising CDR1, CDR2, and CDR3 of the variable heavy chain region of antibody number 11 in Table 6 and CDR1, CDR2, and CDR3 of the variable light chain region of antibody number 11 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 11 in Table 6 and CDR1, CDR2, and CDR3 of the variable light chain region of antibody number 11 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 12 in Table 6 and CDR1, CDR2, or CDR3 of the variable light chain region of antibody number 12 in Table 8. In another embodiment, provided herein is an antibody that binds to SARS-CoV-2 spike protein comprising two of CDR1, CDR2, and CDR3 of the variable heavy chain region of antibody number 12 in Table 6 and two of CDR1, CDR2, and CDR3 of the variable light chain region of antibody number 12 in Table 8. In another embodiment, provided herein is an antibody that binds to SARS-CoV-2 spike protein comprising CDR1, CDR2, and CDR3 of the variable heavy chain region of antibody number 12 in Table 6 and CDR1, CDR2, and CDR3 of the variable light chain region of antibody number 12 in Table 8. In another embodiment, provided herein is an antibody that binds to SARS-CoV-2 spike protein comprising CDR1, CDR2, and CDR3 of the variable heavy chain region of antibody number 12 in Table 6 and CDR1, CDR2, and CDR3 of the variable light chain region of antibody number 12 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 12 in Table 6 and CDR1, CDR2, and CDR3 of the variable light chain region of antibody number 12 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 13 in Table 6 and CDR1, CDR2, or CDR3 of the variable light chain region of antibody number 13 in Table 8. In another embodiment, provided herein is an antibody that binds to SARS-CoV-2 spike protein comprising two of CDR1, CDR2, and CDR3 of the variable heavy chain region of antibody number 13 in Table 6 and two of CDR1, CDR2, and CDR3 of the variable light chain region of antibody number 13 in Table 8. In another embodiment, provided herein is an antibody that binds to SARS-CoV-2 spike protein comprising CDR1, CDR2, and CDR3 of the variable heavy chain region of antibody number 13 in Table 6 and CDR1, CDR2, and CDR3 of the variable light chain region of antibody number 13 in Table 8. In another embodiment, provided herein is an antibody that binds to SARS-CoV-2 spike protein comprising CDR1, CDR2, and CDR3 of the variable heavy chain region of antibody number 13 in Table 6 and CDR1, CDR2, and CDR3 of the variable light chain region of antibody number 13 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 13 in Table 6 and CDR1, CDR2, and CDR3 of the variable light chain region of antibody number 13 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 14 in Table 6 and CDR1, CDR2, or CDR3 of the variable light chain region of antibody number 14 in Table 8. In another embodiment, provided herein is an antibody that binds to SARS-CoV-2 spike protein comprising two of CDR1, CDR2, and CDR3 of the variable heavy chain region of antibody number 14 in Table 6 and two of CDR1, CDR2, and CDR3 of the variable light chain region of antibody number 14 in Table 8. In another embodiment, provided herein is an antibody that binds to SARS-CoV-2 spike protein comprising CDR1, CDR2, and CDR3 of the variable heavy chain region of antibody number 14 in Table 6 and CDR1, CDR2, and CDR3 of the variable light chain region of antibody number 14 in Table 8. In another embodiment, provided herein is an antibody that binds to SARS-CoV-2 spike protein comprising CDR1, CDR2, and CDR3 of the variable heavy chain region of antibody number 14 in Table 6 and CDR1, CDR2, and CDR3 of the variable light chain region of antibody number 14 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 14 in Table 6 and CDR1, CDR2, and CDR3 of the variable light chain region of antibody number 14 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 15 in Table 6 and CDR1, CDR2, or CDR3 of the variable light chain region of antibody number 15 in Table 8. In another embodiment, provided herein is an antibody that binds to SARS-CoV-2 spike protein comprising two of CDR1, CDR2, and CDR3 of the variable heavy chain region of antibody number 15 in Table 6 and two of CDR1, CDR2, and CDR3 of the variable light chain region of antibody number 15 in Table 8. In another embodiment, provided herein is an antibody that binds to SARS-CoV-2 spike protein comprising CDR1, CDR2, and CDR3 of the variable heavy chain region of antibody number 15 in Table 6 and CDR1, CDR2, and CDR3 of the variable light chain region of antibody number 15 in Table 8. In another embodiment, provided herein is an antibody that binds to SARS-CoV-2 spike protein comprising CDR1, CDR2, and CDR3 of the variable heavy chain region of antibody number 15 in Table 6 and CDR1, CDR2, and CDR3 of the variable light chain region of antibody number 15 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 15 in Table 6 and CDR1, CDR2, and CDR3 of the variable light chain region of antibody number 15 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 16 in Table 6 and CDR1, CDR2, or CDR3 of the variable light chain region of antibody number 16 in Table 8. In another embodiment, provided herein is an antibody that binds to SARS-CoV-2 spike protein comprising two of CDR1, CDR2, and CDR3 of the variable heavy chain region of antibody number 16 in Table 6 and two of CDR1, CDR2, and CDR3 of the variable light chain region of antibody number 16 in Table 8. In another embodiment, provided herein is an antibody that binds to SARS-CoV-2 spike protein comprising CDR1, CDR2, and CDR3 of the variable heavy chain region of antibody number 16 in Table 6 and CDR1, CDR2, and CDR3 of the variable light chain region of antibody number 16 in Table 8. In another embodiment, provided herein is an antibody that binds to SARS-CoV-2 spike protein comprising CDR1, CDR2, and CDR3 of the variable heavy chain region of antibody number 16 in Table 6 and CDR1, CDR2, and CDR3 of the variable light chain region of antibody number 16 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 16 in Table 6 and CDR1, CDR2, and CDR3 of the variable light chain region of antibody number 16 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 17 in Table 6 and CDR1, CDR2, or CDR3 of the variable light chain region of antibody number 17 in Table 8. In another embodiment, provided herein is an antibody that binds to SARS-CoV-2 spike protein comprising two of CDR1, CDR2, and CDR3 of the variable heavy chain region of antibody number 17 in Table 6 and two of CDR1, CDR2, and CDR3 of the variable light chain region of antibody number 17 in Table 8. In another embodiment, provided herein is an antibody that binds to SARS-CoV-2 spike protein comprising CDR1, CDR2, and CDR3 of the variable heavy chain region of antibody number 17 in Table 6 and CDR1, CDR2, and CDR3 of the variable light chain region of antibody number 17 in Table 8. In another embodiment, provided herein is an antibody that binds to SARS-CoV-2 spike protein comprising CDR1, CDR2, and CDR3 of the variable heavy chain region of antibody number 17 in Table 6 and CDR1, CDR2, and CDR3 of the variable light chain region of antibody number 17 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 17 in Table 6 and CDR1, CDR2, and CDR3 of the variable light chain region of antibody number 17 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 18 in Table 6 and CDR1, CDR2, or CDR3 of the variable light chain region of antibody number 18 in Table 8. In another embodiment, provided herein is an antibody that binds to SARS-CoV-2 spike protein comprising two of CDR1, CDR2, and CDR3 of the variable heavy chain region of antibody number 18 in Table 6 and two of CDR1, CDR2, and CDR3 of the variable light chain region of antibody number 18 in Table 8. In another embodiment, provided herein is an antibody that binds to SARS-CoV-2 spike protein comprising CDR1, CDR2, and CDR3 of the variable heavy chain region of antibody number 18 in Table 6 and CDR1, CDR2, and CDR3 of the variable light chain region of antibody number 18 in Table 8. In another embodiment, provided herein is an antibody that binds to SARS-CoV-2 spike protein comprising CDR1, CDR2, and CDR3 of the variable heavy chain region of antibody number 18 in Table 6 and CDR1, CDR2, and CDR3 of the variable light chain region of antibody number 18 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 18 in Table 6 and CDR1, CDR2, and CDR3 of the variable light chain region of antibody number 18 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 19 in Table 6 and CDR1, CDR2, or CDR3 of the variable light chain region of antibody number 19 in Table 8. In another embodiment, provided herein is an antibody that binds to SARS-CoV-2 spike protein comprising two of CDR1, CDR2, and CDR3 of the variable heavy chain region of antibody number 19 in Table 6 and two of CDR1, CDR2, and CDR3 of the variable light chain region of antibody number 19 in Table 8. In another embodiment, provided herein is an antibody that binds to SARS-CoV-2 spike protein comprising CDR1, CDR2, and CDR3 of the variable heavy chain region of antibody number 19 in Table 6 and CDR1, CDR2, and CDR3 of the variable light chain region of antibody number 19 in Table 8. In another embodiment, provided herein is an antibody that binds to SARS-CoV-2 spike protein comprising CDR1, CDR2, and CDR3 of the variable heavy chain region of antibody number 19 in Table 6 and CDR1, CDR2, and CDR3 of the variable light chain region of antibody number 19 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 19 in Table 6 and CDR1, CDR2, and CDR3 of the variable light chain region of antibody number 19 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 20 in Table 6 and CDR1, CDR2, or CDR3 of the variable light chain region of antibody number 20 in Table 8. In another embodiment, provided herein is an antibody that binds to SARS-CoV-2 spike protein comprising two of CDR1, CDR2, and CDR3 of the variable heavy chain region of antibody number 20 in Table 6 and two of CDR1, CDR2, and CDR3 of the variable light chain region of antibody number 20 in Table 8. In another embodiment, provided herein is an antibody that binds to SARS-CoV-2 spike protein comprising CDR1, CDR2, and CDR3 of the variable heavy chain region of antibody number 20 in Table 6 and CDR1, CDR2, and CDR3 of the variable light chain region of antibody number 20 in Table 8. In another embodiment, provided herein is an antibody that binds to SARS-CoV-2 spike protein comprising CDR1, CDR2, and CDR3 of the variable heavy chain region of antibody number 20 in Table 6 and CDR1, CDR2, and CDR3 of the variable light chain region of antibody number 20 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 20 in Table 6 and CDR1, CDR2, and CDR3 of the variable light chain region of antibody number 20 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 21 in Table 6 and CDR1, CDR2, or CDR3 of the variable light chain region of antibody number 21 in Table 8. In another embodiment, provided herein is an antibody that binds to SARS-CoV-2 spike protein comprising two of CDR1, CDR2, and CDR3 of the variable heavy chain region of antibody number 21 in Table 6 and two of CDR1, CDR2, and CDR3 of the variable light chain region of antibody number 21 in Table 8. In another embodiment, provided herein is an antibody that binds to SARS-CoV-2 spike protein comprising CDR1, CDR2, and CDR3 of the variable heavy chain region of antibody number 21 in Table 6 and CDR1, CDR2, and CDR3 of the variable light chain region of antibody number 21 in Table 8. In another embodiment, provided herein is an antibody that binds to SARS-CoV-2 spike protein comprising CDR1, CDR2, and CDR3 of the variable heavy chain region of antibody number 21 in Table 6 and CDR1, CDR2, and CDR3 of the variable light chain region of antibody number 21 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 21 in Table 6 and CDR1, CDR2, and CDR3 of the variable light chain region of antibody number 21 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 22 in Table 6 and CDR1, CDR2, or CDR3 of the variable light chain region of antibody number 22 in Table 8. In another embodiment, provided herein is an antibody that binds to SARS-CoV-2 spike protein comprising two of CDR1, CDR2, and CDR3 of the variable heavy chain region of antibody number 22 in Table 6 and two of CDR1, CDR2, and CDR3 of the variable light chain region of antibody number 22 in Table 8. In another embodiment, provided herein is an antibody that binds to SARS-CoV-2 spike protein comprising CDR1, CDR2, and CDR3 of the variable heavy chain region of antibody number 22 in Table 6 and CDR1, CDR2, and CDR3 of the variable light chain region of antibody number 22 in Table 8. In another embodiment, provided herein is an antibody that binds to SARS-CoV-2 spike protein comprising CDR1, CDR2, and CDR3 of the variable heavy chain region of antibody number 22 in Table 6 and CDR1, CDR2, and CDR3 of the variable light chain region of antibody number 22 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 22 in Table 6 and CDR1, CDR2, and CDR3 of the variable light chain region of antibody number 22 in Table 8, wherein one, two or three of the CDRs of the variable heavy chain region, the variable light chain region, or both contain are one, two or three amino acid residues longer or shorter at the N-terminus, C-terminus, or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 23 in Table 6 and CDR1, CDR2, or CDR3 of the variable light chain region of antibody number 23 in Table 8. In another embodiment, provided herein is an antibody that binds to SARS-CoV-2 spike protein comprising two of CDR1, CDR2, and CDR3 of the variable heavy chain region of antibody number 23 in Table 6 and two of CDR1, CDR2, and CDR3 of the variable light chain region of antibody number 23 in Table 8. In another embodiment, provided herein is an antibody that binds to SARS-CoV-2 spike protein comprising CDR1, CDR2, and CDR3 of the variable heavy chain region of antibody number 23 in Table 6 and CDR1, CDR2, and CDR3 of the variable light chain region of antibody number 23 in Table 8. In another embodiment, provided herein is an antibody that binds to SARS-CoV-2 spike protein comprising CDR1, CDR2, and CDR3 of the variable heavy chain region of antibody number 23 in Table 6 and CDR1, CDR2, and CDR3 of the variable light chain region of antibody number 23 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 23 in Table 6 and CDR1, CDR2, and CDR3 of the variable light chain region of antibody number 23 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 24 in Table 6 and CDR1, CDR2, or CDR3 of the variable light chain region of antibody number 24 in Table 8. In another embodiment, provided herein is an antibody that binds to SARS-CoV-2 spike protein comprising two of CDR1, CDR2, and CDR3 of the variable heavy chain region of antibody number 24 in Table 6 and two of CDR1, CDR2, and CDR3 of the variable light chain region of antibody number 24 in Table 8. In another embodiment, provided herein is an antibody that binds to SARS-CoV-2 spike protein comprising CDR1, CDR2, and CDR3 of the variable heavy chain region of antibody number 24 in Table 6 and CDR1, CDR2, and CDR3 of the variable light chain region of antibody number 24 in Table 8. In another embodiment, provided herein is an antibody that binds to SARS-CoV-2 spike protein comprising CDR1, CDR2, and CDR3 of the variable heavy chain region of antibody number 24 in Table 6 and CDR1, CDR2, and CDR3 of the variable light chain region of antibody number 24 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 24 in Table 6 and CDR1, CDR2, and CDR3 of the variable light chain region of antibody number 24 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 25 in Table 6 and CDR1, CDR2, or CDR3 of the variable light chain region of antibody number 25 in Table 8. In another embodiment, provided herein is an antibody that binds to SARS-CoV-2 spike protein comprising two of CDR1, CDR2, and CDR3 of the variable heavy chain region of antibody number 25 in Table 6 and two of CDR1, CDR2, and CDR3 of the variable light chain region of antibody number 25 in Table 8. In another embodiment, provided herein is an antibody that binds to SARS-CoV-2 spike protein comprising CDR1, CDR2, and CDR3 of the variable heavy chain region of antibody number 25 in Table 6 and CDR1, CDR2, and CDR3 of the variable light chain region of antibody number 25 in Table 8. In another embodiment, provided herein is an antibody that binds to SARS-CoV-2 spike protein comprising CDR1, CDR2, and CDR3 of the variable heavy chain region of antibody number 25 in Table 6 and CDR1, CDR2, and CDR3 of the variable light chain region of antibody number 25 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 25 in Table 6 and CDR1, CDR2, and CDR3 of the variable light chain region of antibody number 25 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 26 in Table 6 and CDR1, CDR2, or CDR3 of the variable light chain region of antibody number 26 in Table 8. In another embodiment, provided herein is an antibody that binds to SARS-CoV-2 spike protein comprising two of CDR1, CDR2, and CDR3 of the variable heavy chain region of antibody number 26 in Table 6 and two of CDR1, CDR2, and CDR3 of the variable light chain region of antibody number 26 in Table 8. In another embodiment, provided herein is an antibody that binds to SARS-CoV-2 spike protein comprising CDR1, CDR2, and CDR3 of the variable heavy chain region of antibody number 26 in Table 6 and CDR1, CDR2, and CDR3 of the variable light chain region of antibody number 26 in Table 8. In another embodiment, provided herein is an antibody that binds to SARS-CoV-2 spike protein comprising CDR1, CDR2, and CDR3 of the variable heavy chain region of antibody number 26 in Table 6 and CDR1, CDR2, and CDR3 of the variable light chain region of antibody number 26 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 26 in Table 6 and CDR1, CDR2, and CDR3 of the variable light chain region of antibody number 26 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 27 in Table 6 and CDR1, CDR2, or CDR3 of the variable light chain region of antibody number 27 in Table 8. In another embodiment, provided herein is an antibody that binds to SARS-CoV-2 spike protein comprising two of CDR1, CDR2, and CDR3 of the variable heavy chain region of antibody number 27 in Table 6 and two of CDR1, CDR2, and CDR3 of the variable light chain region of antibody number 27 in Table 8. In another embodiment, provided herein is an antibody that binds to SARS-CoV-2 spike protein comprising CDR1, CDR2, and CDR3 of the variable heavy chain region of antibody number 27 in Table 6 and CDR1, CDR2, and CDR3 of the variable light chain region of antibody number 27 in Table 8. In another embodiment, provided herein is an antibody that binds to SARS-CoV-2 spike protein comprising CDR1, CDR2, and CDR3 of the variable heavy chain region of antibody number 27 in Table 6 and CDR1, CDR2, and CDR3 of the variable light chain region of antibody number 27 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 27 in Table 6 and CDR1, CDR2, and CDR3 of the variable light chain region of antibody number 27 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 28 in Table 6 and CDR1, CDR2, or CDR3 of the variable light chain region of antibody number 28 in Table 8. In another embodiment, provided herein is an antibody that binds to SARS-CoV-2 spike protein comprising two of CDR1, CDR2, and CDR3 of the variable heavy chain region of antibody number 28 in Table 6 and two of CDR1, CDR2, and CDR3 of the variable light chain region of antibody number 28 in Table 8. In another embodiment, provided herein is an antibody that binds to SARS-CoV-2 spike protein comprising CDR1, CDR2, and CDR3 of the variable heavy chain region of antibody number 28 in Table 6 and CDR1, CDR2, and CDR3 of the variable light chain region of antibody number 28 in Table 8. In another embodiment, provided herein is an antibody that binds to SARS-CoV-2 spike protein comprising CDR1, CDR2, and CDR3 of the variable heavy chain region of antibody number 28 in Table 6 and CDR1, CDR2, and CDR3 of the variable light chain region of antibody number 28 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 28 in Table 6 and CDR1, CDR2, and CDR3 of the variable light chain region of antibody number 28 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In another embodiment, provided herein is an antibody that binds to SARS-CoV-2 spike protein comprising CDR1, CDR2, or CDR3 of the variable heavy chain region of antibody number 29 in Table 6 and CDR1, CDR2, or CDR3 of the variable light chain region of antibody number 29 in Table 8. In another embodiment, provided herein is an antibody that binds to SARS-CoV-2 spike protein comprising two of CDR1, CDR2, and CDR3 of the variable heavy chain region of antibody number 29 in Table 6 and two of CDR1, CDR2, and CDR3 of the variable light chain region of antibody number 29 in Table 8. In another embodiment, provided herein is an antibody that binds to SARS-CoV-2 spike protein comprising CDR1, CDR2, and CDR3 of the variable heavy chain region of antibody number 29 in Table 6 and CDR1, CDR2, and CDR3 of the variable light chain region of antibody number 29 in Table 8. In another embodiment, provided herein is an antibody that binds to SARS-CoV-2 spike protein comprising CDR1, CDR2, and CDR3 of the variable heavy chain region of antibody number 29 in Table 6 and CDR1, CDR2, and CDR3 of the variable light chain region of antibody number 29 in Table 8, wherein one, two or three of the CDRs of the variable heavy chain region, the variable light chain region, or both contain one, two, three or four amino acid substitutions (e.g., a conservative amino acid substitution) so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA). In another embodiment, provided herein is an antibody that binds to SARS-CoV-2 spike protein comprising CDR1, CDR2, and CDR3 of the variable heavy chain region of antibody number 29 in Table 6 and CDR1, CDR2, and CDR3 of the variable light chain region of antibody number 29 in Table 8, wherein one, two or three of the CDRs of the variable heavy chain region, the variable light chain region, or both contain are one, two or three amino acid residues longer or shorter at the N-terminus, C-terminus or both so long as binding to SARS-CoV-2 spike protein is maintained or substantially maintained (for example, by at least 50%, at least 60%, at least 70%, at least 80%, at least 90%, at least 95% in an assay known in the art or described herein, such as an ELISA).

In certain embodiments, an antibody described herein, which binds to SARS-CoV-2 spike protein or a fragment thereof (e.g., RBD), comprises a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of an antibody described herein (e.g., an antibody in Table 1). In some embodiments, an antibody described herein, which binds to SARS-CoV-2 spike protein or a fragment thereof (e.g., RBD), comprises a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of an antibody described herein (e.g., an antibody in Table 2). In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of an antibody described herein (e.g., an antibody in Table 1) and (b) a variable light chain region comprising an amino acid sequence that is at least 80%, 85%, 86%, 87%, 88%, 89%, 90%, 91%, 92%, 93%, 94%95%, 96%, 97% or 98% identical to the amino acid sequence of a variable light chain region of an antibody described herein (e.g., an antibody in Table 2). In accordance with these embodiments, the CDRs of the antibody may, in certain embodiments, be identical to one, two, three, four, five, or all six of the CDRs of the antibody described herein.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 1 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 1 in Table 2. In accordance with these embodiments, the CDRs of the antibody may, in certain embodiments, be identical to one, two, three, four, five, or all six of the CDRs of antibody number 1. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 1 in Table 1; and (b) a variable light chain region comprising the amino acid sequence of a variable light chain region of antibody number 1 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 2 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 2 in Table 2. In accordance with these embodiments, the CDRs of the antibody may, in certain embodiments, be identical to one, two, three, four, five, or all six of the CDRs of antibody number 2. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 2 in Table 1; and (b) a variable light chain region comprising the amino acid sequence of a variable light chain region of antibody number 2 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 3 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 3 in Table 2. In accordance with these embodiments, the CDRs of the antibody may, in certain embodiments, be identical to one, two, three, four, five, or all six of the CDRs of antibody number 3. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 3 in Table 1; and (b) a variable light chain region comprising the amino acid sequence of a variable light chain region of antibody number 3 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 4 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 4 in Table 2. In accordance with these embodiments, the CDRs of the antibody may, in certain embodiments, be identical to one, two, three, four, five, or all six of the CDRs of antibody number 4. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 4 in Table 1; and (b) a variable light chain region comprising the amino acid sequence of a variable light chain region of antibody number 4 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 5 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 5 in Table 2. In accordance with these embodiments, the CDRs of the antibody may, in certain embodiments, be identical to one, two, three, four, five, or all six of the CDRs of antibody number 5. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 5 in Table 1; and (b) a variable light chain region comprising the amino acid sequence of a variable light chain region of antibody number 5 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 6 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 6 in Table 2. In accordance with these embodiments, the CDRs of the antibody may, in certain embodiments, be identical to one, two, three, four, five, or all six of the CDRs of antibody number 6. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 6 in Table 1; and (b) a variable light chain region comprising the amino acid sequence of a variable light chain region of antibody number 6 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 7 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 7 in Table 2. In accordance with these embodiments, the CDRs of the antibody may, in certain embodiments, be identical to one, two, three, four, five, or all six of the CDRs of antibody number 7. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 7 in Table 1; and (b) a variable light chain region comprising the amino acid sequence of a variable light chain region of antibody number 7 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 8 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 8 in Table 2. In accordance with these embodiments, the CDRs of the antibody may, in certain embodiments, be identical to one, two, three, four, five, or all six of the CDRs of antibody number 8. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 8 in Table 1; and (b) a variable light chain region comprising the amino acid sequence of a variable light chain region of antibody number 8 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 9 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 9 in Table 2. In accordance with these embodiments, the CDRs of the antibody may, in certain embodiments, be identical to one, two, three, four, five, or all six of the CDRs of antibody number 9. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 9 in Table 1; and (b) a variable light chain region comprising the amino acid sequence of a variable light chain region of antibody number 9 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 10 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 10 in Table 2. In accordance with these embodiments, the CDRs of the antibody may, in certain embodiments, be identical to one, two, three, four, five, or all six of the CDRs of the antibody number 10. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 10 in Table 1; and (b) a variable light chain region comprising the amino acid sequence of a variable light chain region of antibody number 10 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 11 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 11 in Table 2. In accordance with these embodiments, the CDRs of the antibody may, in certain embodiments, be identical to one, two, three, four, five, or all six of the CDRs of the antibody number 11. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 11 in Table 1; and (b) a variable light chain region comprising the amino acid sequence of a variable light chain region of antibody number 11 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 12 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 12 in Table 2. In accordance with these embodiments, the CDRs of the antibody may, in certain embodiments, be identical to one, two, three, four, five, or all six of the CDRs of the antibody number 12. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 12 in Table 1; and (b) a variable light chain region comprising the amino acid sequence of a variable light chain region of antibody number 12 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 13 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 13 in Table 2. In accordance with these embodiments, the CDRs of the antibody may, in certain embodiments, be identical to one, two, three, four, five, or all six of the CDRs of the antibody number 13. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 13 in Table 1; and (b) a variable light chain region comprising the amino acid sequence of a variable light chain region of antibody number 13 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 14 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 14 in Table 2. In accordance with these embodiments, the CDRs of the antibody may, in certain embodiments, be identical to one, two, three, four, five, or all six of the CDRs of the antibody number 14. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 14 in Table 1; and (b) a variable light chain region comprising the amino acid sequence of a variable light chain region of antibody number 14 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 15 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 15 in Table 2. In accordance with these embodiments, the CDRs of the antibody may, in certain embodiments, be identical to one, two, three, four, five, or all six of the CDRs of the antibody number 15. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 15 in Table 1; and (b) a variable light chain region comprising the amino acid sequence of a variable light chain region of antibody number 15 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 16 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 16 in Table 2. In accordance with these embodiments, the CDRs of the antibody may, in certain embodiments, be identical to one, two, three, four, five, or all six of the CDRs of the antibody number 16. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 16 in Table 1; and (b) a variable light chain region comprising the amino acid sequence of a variable light chain region of antibody number 16 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 17 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 17 in Table 2. In accordance with these embodiments, the CDRs of the antibody may, in certain embodiments, be identical to one, two, three, four, five, or all six of the CDRs of the antibody number 17. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 17 in Table 1; and (b) a variable light chain region comprising the amino acid sequence of a variable light chain region of antibody number 17 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 18 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 18 in Table 2. In accordance with these embodiments, the CDRs of the antibody may, in certain embodiments, be identical to one, two, three, four, five, or all six of the CDRs of the antibody number 18. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 18 in Table 1; and (b) a variable light chain region comprising the amino acid sequence of a variable light chain region of antibody number 18 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 19 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 19 in Table 2. In accordance with these embodiments, the CDRs of the antibody may, in certain embodiments, be identical to one, two, three, four, five, or all six of the CDRs of the antibody number 19. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 19 in Table 1; and (b) a variable light chain region comprising the amino acid sequence of a variable light chain region of antibody number 19 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 20 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 20 in Table 2. In accordance with these embodiments, the CDRs of the antibody may, in certain embodiments, be identical to one, two, three, four, five, or all six of the CDRs of the antibody number 20. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 20 in Table 1; and (b) a variable light chain region comprising the amino acid sequence of a variable light chain region of antibody number 20 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 21 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 21 in Table 2. In accordance with these embodiments, the CDRs of the antibody may, in certain embodiments, be identical to one, two, three, four, five, or all six of the CDRs of the antibody number 21. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 21 in Table 1; and (b) a variable light chain region comprising the amino acid sequence of a variable light chain region of antibody number 21 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 22 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 22 in Table 2. In accordance with these embodiments, the CDRs of the antibody may, in certain embodiments, be identical to one, two, three, four, five, or all six of the CDRs of the antibody number 22. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 22 in Table 1; and (b) a variable light chain region comprising the amino acid sequence of a variable light chain region of antibody number 22 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 23 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 23 in Table 2. In accordance with these embodiments, the CDRs of the antibody may, in certain embodiments, be identical to one, two, three, four, five, or all six of the CDRs of the antibody number 23. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 23 in Table 1; and (b) a variable light chain region comprising the amino acid sequence of a vaable light chain region of antibody number 23 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 24 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 24 in Table 2. In accordance with these embodiments, the CDRs of the antibody may, in certain embodiments, be identical to one, two, three, four, five, or all six of the CDRs of the antibody number 24. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 24 in Table 1; and (b) a variable light chain region comprising the amino acid sequence of a variable light chain region of antibody number 24 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 25 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 25 in Table 2. In accordance with these embodiments, the CDRs of the antibody may, in certain embodiments, be identical to one, two, three, four, five, or all six of the CDRs of the antibody number 25. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 25 in Table 1; and (b) a variable light chain region comprising the amino acid sequence of a variable light chain region of antibody number 25 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 26 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 26 in Table 2. In accordance with these embodiments, the CDRs of the antibody may, in certain embodiments, be identical to one, two, three, four, five, or all six of the CDRs of the antibody number 26. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 26 in Table 1; and (b) a variable light chain region comprising the amino acid sequence of a variable light chain region of antibody number 26 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 27 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 27 in Table 2. In accordance with these embodiments, the CDRs of the antibody may, in certain embodiments, be identical to one, two, three, four, five, or all six of the CDRs of the antibody number 27. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 27 in Table 1; and (b) a variable light chain region comprising the amino acid sequence of a variable light chain region of antibody number 27 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 28 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 28 in Table 2. In accordance with these embodiments, the CDRs of the antibody may, in certain embodiments, be identical to one, two, three, four, five, or all six of the CDRs of the antibody number 28. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 28 in Table 1; and (b) a variable light chain region comprising the amino acid sequence of a variable light chain region of antibody number 28 in Table 2.

In certain embodiments, an antibody described herein, which binds to SARS-CoV-2 spike protein or a fragment thereof (e.g., RBD), comprises (a) a variable heavy chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable heavy chain region of antibody number 29 in Table 1; and (b) a variable light chain region comprising an amino acid sequence that is at least 80%, 85%, 86%, 87%, 88%, 89%, 90%, 91%, 92%, 93%, 94% 95%, 96%, 97% or 98% identical to the amino acid sequence of a variable light chain region of antibody number 29 in Table 2. In accordance with these embodiments, the CDRs of the antibody may, in certain embodiments, be identical to one, two, three, four, five, or all six of the CDRs of the antibody number 29. In one embodiment, an antibody described herein, which binds to SARS-CoV-2 spike protein or a fragment thereof (e.g., RBD), comprises (a) a variable heavy chain region comprising the amino acid sequence of a variable heavy chain region of antibody number 29 in Table 1; and (b) a variable light chain region comprising the amino acid sequence of a variable light chain region of antibody number 29 in Table 2.

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length. In a certain embodiment, the percent identity is determined over the entire length of an amino acid sequence or nucleotide sequence.

The determination of percent identity between two sequences (e.g., amino acid sequences or nucleic acid sequences) can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In some embodiments, an antibody described herein, which binds to SARS-CoV-2 spike protein or a fragment thereof (e.g., RBD), comprises the variable heavy chain region or variable light chain region of an antibody described herein (e.g., a variable heavy chain region or variable light chain region an antibody in Table 1 or 2, respectively) with one or more (e.g., 1, 2, 3, 4, 5, 6, 7, 8, 9, 10, 15 or 20) amino acid substitutions (e.g., conservative amino acid substitutions), deletions, or additions. In certain embodiments, an antibody described herein, which binds to SARS-CoV-2 spike protein or a fragment thereof (e.g., RBD), comprises the variable heavy chain region and variable light chain region of an antibody described herein (e.g., a variable heavy chain region and variable light chain region an antibody with the same name in Tables 1 and 2) with one or more (e.g., 1, 2, 3, 4, 5, 6, 7, 8, 9, 10, 15, or 20) amino acid substitutions (e.g., conservative amino acid substitutions), deletions, or additions. In specific embodiments, none of the amino acid substitutions are located within the CDRs. In specific embodiments, all of the amino acid substitutions are in the framework regions.

As used herein, a “conservative amino acid substitution” has the meaning known to one of skill in the art. In one embodiment, a conservative amino acid substitution is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In one embodiment, an antibody provided herein comprises a variable heavy chain region and a variable light chain region, wherein the variable heavy chain region comprises the amino acid sequence encoded by the nucleotide sequence of antibody number 1 in Table 1 and the variable light chain region comprises the amino acid sequence encoded by the nucleotide sequence of antibody 1 in Table 2. In one embodiment, an antibody provided herein comprises a variable heavy chain region and a variable light chain region, wherein the variable heavy chain region comprises the amino acid sequence encoded by the nucleotide sequence of antibody number 2 in Table 1 and the variable light chain region comprises the amino acid sequence encoded by the nucleotide sequence of antibody 2 in Table 2. In one embodiment, an antibody provided herein comprises a variable heavy chain region and a variable light chain region, wherein the variable heavy chain region comprises the amino acid sequence encoded by the nucleotide sequence of antibody number 3 in Table 1 and the variable light chain region comprises the amino acid sequence encoded by the nucleotide sequence of antibody 3 in Table 2. In one embodiment, an antibody provided herein comprises a variable heavy chain region and a variable light chain region, wherein the variable heavy chain region comprises the amino acid sequence encoded by the nucleotide sequence of antibody number 3 in Table 1 and the variable light chain region comprises the amino acid sequence encoded by the nucleotide sequence of antibody 3 in Table 2. In one embodiment, an antibody provided herein comprises a variable heavy chain region and a variable light chain region, wherein the variable heavy chain region comprises the amino acid sequence encoded by the nucleotide sequence of antibody number 4 in Table 1 and the variable light chain region comprises the amino acid sequence encoded by the nucleotide sequence of antibody 4 in Table 2. In one embodiment, an antibody provided herein comprises a variable heavy chain region and a variable light chain region, wherein the variable heavy chain region comprises the amino acid sequence encoded by the nucleotide sequence of antibody number 5 in Table 1 and the variable light chain region comprises the amino acid sequence encoded by the nucleotide sequence of antibody 5 in Table 2. In one embodiment, an antibody provided herein comprises a variable heavy chain region and a variable light chain region, wherein the variable heavy chain region comprises the amino acid sequence encoded by the nucleotide sequence of antibody number 6 in Table 1 and the variable light chain region comprises the amino acid sequence encoded by the nucleotide sequence of antibody 6 in Table 2. In one embodiment, an antibody provided herein comprises a variable heavy chain region and a variable light chain region, wherein the variable heavy chain region comprises the amino acid sequence encoded by the nucleotide sequence of antibody number 7 in Table 1 and the variable light chain region comprises the amino acid sequence encoded by the nucleotide sequence of antibody 7 in Table 2. In one embodiment, an antibody provided herein comprises a variable heavy chain region and a variable light chain region, wherein the variable heavy chain region comprises the amino acid sequence encoded by the nucleotide sequence of antibody number 8 in Table 1 and the variable light chain region comprises the amino acid sequence encoded by the nucleotide sequence of antibody 8 in Table 2. In one embodiment, an antibody provided herein comprises a variable heavy chain region and a variable light chain region, wherein the variable heavy chain region comprises the amino acid sequence encoded by the nucleotide sequence of antibody number 9 in Table 1 and the variable light chain region comprises the amino acid sequence encoded by the nucleotide sequence of antibody 9 in Table 2. In one embodiment, an antibody provided herein comprises a variable heavy chain region and a variable light chain region, wherein the variable heavy chain region comprises the amino acid sequence encoded by the nucleotide sequence of antibody number 10 in Table 1 and the variable light chain region comprises the amino acid sequence encoded by the nucleotide sequence of antibody 10 in Table 2. In one embodiment, an antibody provided herein comprises a variable heavy chain region and a variable light chain region, wherein the variable heavy chain region comprises the amino acid sequence encoded by the nucleotide sequence of antibody number 11 in Table 1 and the variable light chain region comprises the amino acid sequence encoded by the nucleotide sequence of antibody 11 in Table 2. In one embodiment, an antibody provided herein comprises a variable heavy chain region and a variable light chain region, wherein the variable heavy chain region comprises the amino acid sequence encoded by the nucleotide sequence of antibody number 11 in Table 1 and the variable light chain region comprises the amino acid sequence encoded by the nucleotide sequence of antibody 11 in Table 2. In one embodiment, an antibody provided herein comprises a variable heavy chain region and a variable light chain region, wherein the variable heavy chain region comprises the amino acid sequence encoded by the nucleotide sequence of antibody number 12 in Table 1 and the variable light chain region comprises the amino acid sequence encoded by the nucleotide sequence of antibody 12 in Table 2. In one embodiment, an antibody provided herein comprises a variable heavy chain region and a variable light chain region, wherein the variable heavy chain region comprises the amino acid sequence encoded by the nucleotide sequence of antibody number 13 in Table 1 and the variable light chain region comprises the amino acid sequence encoded by the nucleotide sequence of antibody 13 in Table 2. In one embodiment, an antibody provided herein comprises a variable heavy chain region and a variable light chain region, wherein the variable heavy chain region comprises the amino acid sequence encoded by the nucleotide sequence of antibody number 14 in Table 1 and the variable light chain region comprises the amino acid sequence encoded by the nucleotide sequence of antibody 14 in Table 2. In one embodiment, an antibody provided herein comprises a variable heavy chain region and a variable light chain region, wherein the variable heavy chain region comprises the amino acid sequence encoded by the nucleotide sequence of antibody number 15 in Table 1 and the variable light chain region comprises the amino acid sequence encoded by the nucleotide sequence of antibody 15 in Table 2. In one embodiment, an antibody provided herein comprises a variable heavy chain region and a variable light chain region, wherein the variable heavy chain region comprises the amino acid sequence encoded by the nucleotide sequence of antibody number 16 in Table 1 and the variable light chain region comprises the amino acid sequence encoded by the nucleotide sequence of antibody 16 in Table 2. In one embodiment, an antibody provided herein comprises a variable heavy chain region and a variable light chain region, wherein the variable heavy chain region comprises the amino acid sequence encoded by the nucleotide sequence of antibody number 17 in Table 1 and the variable light chain region comprises the amino acid sequence encoded by the nucleotide sequence of antibody 17 in Table 2. In one embodiment, an antibody provided herein comprises a variable heavy chain region and a variable light chain region, wherein the variable heavy chain region comprises the amino acid sequence encoded by the nucleotide sequence of antibody number 18 in Table 1 and the variable light chain region comprises the amino acid sequence encoded by the nucleotide sequence of antibody 18 in Table 2. In one embodiment, an antibody provided herein comprises a variable heavy chain region and a variable light chain region, wherein the variable heavy chain region comprises the amino acid sequence encoded by the nucleotide sequence of antibody number 19 in Table 1 and the variable light chain region comprises the amino acid sequence encoded by the nucleotide sequence of antibody 19 in Table 2. In one embodiment, an antibody provided herein comprises a variable heavy chain region and a variable light chain region, wherein the variable heavy chain region comprises the amino acid sequence encoded by the nucleotide sequence of antibody number 20 in Table 1 and the variable light chain region comprises the amino acid sequence encoded by the nucleotide sequence of antibody 20 in Table 2. In one embodiment, an antibody provided herein comprises a variable heavy chain region and a variable light chain region, wherein the variable heavy chain region comprises the amino acid sequence encoded by the nucleotide sequence of antibody number 21 in Table 1 and the variable light chain region comprises the amino acid sequence encoded by the nucleotide sequence of antibody 21 in Table 2. In one embodiment, an antibody provided herein comprises a variable heavy chain region and a variable light chain region, wherein the variable heavy chain region comprises the amino acid sequence encoded by the nucleotide sequence of antibody number 22 in Table 1 and the variable light chain region comprises the amino acid sequence encoded by the nucleotide sequence of antibody 22 in Table 2. In one embodiment, an antibody provided herein comprises a variable heavy chain region and a variable light chain region, wherein the variable heavy chain region comprises the amino acid sequence encoded by the nucleotide sequence of antibody number 23 in Table 1 and the variable light chain region comprises the amino acid sequence encoded by the nucleotide sequence of antibody 23 in Table 2. In one embodiment, an antibody provided herein comprises a variable heavy chain region and a variable light chain region, wherein the variable heavy chain region comprises the amino acid sequence encoded by the nucleotide sequence of antibody number 24 in Table 1 and the variable light chain region comprises the amino acid sequence encoded by the nucleotide sequence of antibody 24 in Table 2. In one embodiment, an antibody provided herein comprises a variable heavy chain region and a variable light chain region, wherein the variable heavy chain region comprises the amino acid sequence encoded by the nucleotide sequence of antibody number 25 in Table 1 and the variable light chain region comprises the amino acid sequence encoded by the nucleotide sequence of antibody 25 in Table 2. In one embodiment, an antibody provided herein comprises a variable heavy chain region and a variable light chain region, wherein the variable heavy chain region comprises the amino acid sequence encoded by the nucleotide sequence of antibody number 26 in Table 1 and the variable light chain region comprises the amino acid sequence encoded by the nucleotide sequence of antibody 26 in Table 2. In one embodiment, an antibody provided herein comprises a variable heavy chain region and a variable light chain region, wherein the variable heavy chain region comprises the amino acid sequence encoded by the nucleotide sequence of antibody number 27 in Table 1 and the variable light chain region comprises the amino acid sequence encoded by the nucleotide sequence of antibody 27 in Table 2. In one embodiment, an antibody provided herein comprises a variable heavy chain region and a variable light chain region, wherein the variable heavy chain region comprises the amino acid sequence encoded by the nucleotide sequence of antibody number 28 in Table 1 and the variable light chain region comprises the amino acid sequence encoded by the nucleotide sequence of antibody 28 in Table 2. In one embodiment, an antibody provided herein comprises a variable heavy chain region and a variable light chain region, wherein the variable heavy chain region comprises the amino acid sequence encoded by the nucleotide sequence of antibody number 29 in Table 1 and the variable light chain region comprises the amino acid sequence encoded by the nucleotide sequence of antibody 29 in Table 2.

In another aspect, provided herein are antibodies that bind to the same or an overlapping epitope of an antibody described herein. e.g., antibodies that compete for binding to SARS-CoV-2 spike protein with an antibody described herein, or antibodies which bind to an epitope which overlaps with an epitope to which an antibody described herein binds. As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain aspects, epitope mapping assays, well known to one of skill in the art, can be performed to ascertain the epitope (e.g., conformational epitope) to which an antibody described herein binds. In certain embodiments, the epitope can be determined by, e.g., structural mapping using negative electron microscopy, X-ray diffraction crystallography studies (see, e.g., Blechman et al., 1993, J. Biol. Chem. 268:4399-4406; Cho et al., 2003, Nature, 421:756-760), ELISA assays, hydrogen/deuterium exchange coupled with mass spectrometry (e.g., MALDI mass spectrometry), array-based oligo-peptide scanning assays, mutagenesis mapping (e.g., site-directed mutagenesis mapping) and/or escape binding assays.

Antibodies that recognize such epitopes can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as SARS-CoV-2 spike protein. Numerous types of competitive binding assays are known, for example: solid phase direct or indirect radioimmunoassay (RIA), solid phase direct or indirect enzyme immunoassay (EIA), sandwich competition assay (see Stahli et al., (1983) Methods in Enzymology 9:242); solid phase direct biotin-avidin EIA (see Kirkland et al., (1986) J. Immunol. 137:3614); solid phase direct labeled assay, solid phase direct labeled sandwich assay (see Harlow and Lane, (1988) Antibodies: A Laboratory Manual, Cold Spring Harbor Press); solid phase direct label RIA using I-125 label (see Morel et al., (1988) Mol. Immunol. 25(1):7); solid phase direct biotin-avidin EIA (Cheung et al., (1990) Virology 176:546); and direct labeled RIA. (Moldenhauer et al., (1990) Scand J. Immunol. 32:77). Typically, such an assay involves the use of purified antigen (e.g., SARS-CoV-2 spike proteinfragment thereof (e.g., RBD)) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, for example, Wagener et al., J. Immunol., 1983, 130:2308-2315; Wagener et al., J. Immunol. Methods, 1984, 68:269-274; Kuroki et al., Cancer Res., 1990, 50:4872-4879; Kuroki et al., Immunol. Invest., 1992, 21:523-538; Kuroki et al., Hybridoma, 1992, 11:391-407, and Using Antibodies: A Laboratory Manual, Ed Harlow and David Lane editors (Cold Springs Harbor Laboratory Press, Cold Springs Harbor, N.Y., 1999), pp. 386-389.

In certain aspects, competition binding assays can be used to determine whether an antibody is competitively blocked, e.g., in a dose dependent manner, by another antibody for example, an antibody binds essentially the same epitope, or overlapping epitopes, as a reference antibody, when the two antibodies recognize identical or sterically overlapping epitopes in competition binding assays such as competition ELISA assays, which can be configured in all number of different formats, using either labeled antigen or labeled antibody. In one embodiment, an antibody can be tested in competition binding assays with an antibody described herein.

In specific embodiments, an antibody described herein, which binds to SARS-CoV-2 spike protein or a fragment thereof (e.g. RBD), comprises framework regions (e.g., framework regions of the VL domain and/or VH domain) that are human framework regions or derived from human framework regions. The framework region may be naturally occurring or consensus framework regions (see, e.g., Sui et al., 2009, Nature Structural & Molecular Biology 16:265-273). Non-limiting examples of human framework regions are described in the art, e.g., see Kabat et al. (1991) Sequences of Proteins of Immunological Interest, Fifth Edition, U.S. Department of Health and Human Services, NIH Publication No. 91-3242). In certain embodiment, an antibody described herein comprises framework regions (e.g., framework regions of the VL domain and/or VH domain) that are primate (e.g., non-human primate) framework regions or derived from primate (e.g., non-human primate) framework regions.

In specific aspects, provided herein is an antibody comprising an antibody light chain and heavy chain, e.g., a separate light chain and heavy chain.

With respect to the light chain, in one embodiment, the light chain of an antibody described herein is a kappa light chain. In one embodiment, the light chain of an antibody described herein is a lambda light chain. In yet one embodiment, the light chain of an antibody described herein is a human kappa light chain or a human lambda light chain. In one embodiment, an antibody described herein, which binds to SARS-CoV-2 spike protein or a fragment thereof (e.g., RBD), comprises a light chain wherein the amino acid sequence of the variable light chain region can comprise any amino acid sequence described herein (e.g., variable light chain region of an antibody in Table 2), and wherein the constant region of the light chain comprises the amino acid sequence of a human kappa or lamda light chain constant region. Non-limiting examples of human constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

In one embodiment, an antibody described herein comprises (i) a heavy chain comprising a variable heavy chain region described herein and a constant region; or (ii) a light chain comprising a variable light chain region described herein and a constant region. In one embodiment, an antibody described herein comprises comprises (i) a heavy chain comprising a variable heavy chain region described herein and a constant region; and (ii) a light chain comprising a variable light chain region described herein and a constant region. As used herein, the term “constant region” or “constant domain” is interchangeable and has its meaning common in the art. The constant region refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

As used herein, the term“heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

As used herein, the term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

With respect to the heavy chain, in one embodiment, the heavy chain of an antibody described herein can be an alpha (α), delta (δ), epsilon (ε), gamma (γ) or mu (μ) heavy chain. In one embodiment, the heavy chain of an antibody described can comprise a human alpha (α), delta (δ), epsilon (ε), gamma (γ) or mu (μ) heavy chain. In one embodiment, an antibody described herein, which binds to SARS-CoV-2 spike protein or a fragment thereof (e.g., RBD), comprises a heavy chain wherein the amino acid sequence of the variable heavy chain region can comprise any amino acid sequence described herein (e.g., variable heavy chain region of an antibody in Table 1), and wherein the constant region of the heavy chain comprises the amino acid sequence of a human gamma (γ) heavy chain constant region. Non-limiting examples of human constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

In one embodiment, an antibody described herein, which binds to SARS-CoV-2 spike protein or a fragment thereof (e.g., RBD), comprises a variable heavy chain region and a variable light chain region comprising any amino acid sequences described herein, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In one embodiment, an antibody described herein, which binds SARS-CoV-2 spike protein or a fragment thereof (e.g., RBD), comprises a variable heavy chain region and a variable light chain region comprising any amino acid sequences described herein, and wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. In some embodiments, an antibody described herein is an IgG2a antibody, and optionally comprises a kappa light chain.

The antibodies described herein can be affinity matured using techniques known to one of skill in the art.

The antibodies provided herein include derivatives that are chemically modified, i.e., by the covalent attachment of any type of molecule to the antibody. For example, but not by way of limitation, the antibody derivatives include antibodies that have been chemically modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In some embodiments, the glycosylation of antibodies described herein, in particular glycosylation of a variable region of an antibody described herein, is modified. For example, an aglycoslated antibody can be made (i.e., the antibody lacks glycosylation) or an antibody comprising a mutation or substitution at one or more glycosylation sites to eliminate glycosylation at the one or more glycosylation sites can be be made. Glycosylation can be altered to, for example, increase the affinity of the antibody for SARS-CoV-2 spike protein or a fragment thereof (e.g., RBD). Such carbohydrate modifications can be accomplished by, for example, altering one or more sites of glycosylation within the antibody sequence. For example, one or more amino acid substitutions can be made that result in elimination of one or more variable region (e.g., variable heavy chain region CDRS and/or variable light chain region CDRs or variable heavy chain region FRs and/or variable light chain region FRs) glycosylation sites to thereby eliminate glycosylation at that site. Such aglycosylation can increase the affinity of the antibody for SARS-CoV-2 spike protein or a fragment thereof (e.g., RBD). Such an approach is described in further detail in U.S. Pat. Nos. 5,714,350 and 6,350,861.

Glycosylation can occur via N-linked (or asparagine-linked) glycosylation or O-linked glycosylation. N-linked glycosylation involves carbohydrate modification at the side-chain NH₂ group of an asparagine amino acid in a polypeptide. O-linked glycosylation involves carbohydrate modification at the hydroxyl group on the side chain of a serine, threonine, or hydroxylysine amino acid.

In certain embodiments, aglycosylated antibodies can be produced in bacterial cells which lack the necessary glycosylation machinery. Cells with altered glycosylation machinery have been described in the art and can be used as host cells in which to express recombinant antibodies described herein to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342.

Antibodies with reduced fucose content have been reported to have an increased affinity for Fc receptors, such as, e.g., FcγRIIIa. Accordingly, in certain embodiments, the antibodies described herein have reduced fucose content or no fucose content. Such antibodies can be produced using techniques known to one skilled in the art. For example, the antibodies can be expressed in cells deficient or lacking the ability to fucosylate. In one example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

In certain embodiments, one, two or more mutations (e.g., amino acid substitutions) are introduced into the Fc region of an antibody described herein or a fragment thereof (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to increase the affinity of the antibody for an Fc receptor (e.g., an activated Fc receptor) on the surface of an effector cell. Mutations in the Fc region of an antibody or fragment thereof that increase the affinity of an antibody for an Fc receptor and techniques for introducting such mutations into the Fc receptor or fragment thereof are known to one of skill in the art. Examples of mutations in the Fc receptor of an antibody that can be made to increase the affinity of the antibody for an Fc receptor are described in, e.g., Smith, P., et al. (2012) PNAS. 109:6181-6186, which is incorporated herein by reference.

In some aspects, provided herein are antibodies, conjugated or recombinantly fused to a diagnostic, detectable or therapeutic agent or any other molecule. The conjugated or recombinantly fused antibodies can be useful, e.g., for monitoring or prognosing the onset, development, progression and/or severity of COVID-19 as part of a clinical testing procedure, such as determining the efficacy of a particular therapy. In certain aspects, the conjugated or recombinantly fused antibodies can be useful in preventing, treating, or both COVID-19. Antibodies described herein can also be conjugated to a molecule (e.g., polyethylene glycol) which can affect one or more biological and/or molecular properties of the antibodies, for example, stability (e.g., in serum), half-life, solubility, and antigenicity.

In some embodiments, a conjugate comprises an antibody described herein and a molecule (e.g., therapeutic or drug moiety), wherein the antibody is linked directly to the molecule, or by way of one or more linkers. In certain embodiments, an antibody is covalently conjugated to a molecule. In one embodiment, an antibody is noncovalently conjugated to a molecule. In one embodiment, provided herein is an antibody drug conjugate comprising an antibody moiety and a drug (e.g., therapeutic or prophylactic agent), wherein the antibody moiety is an antibody described herein and wherein the conjugate may comprise one or more linkers.

In certain embodiments, an antibody described herein is conjugated to one or more molecules (e.g., therapeutic or drug moiety) directly or indirectly via one or more linker molecules. In one embodiments, a linker comprises 1, 2, 3, 4, 5, 6, 7, 8, 9, 10, 11, 12, 13, 14, 15, or 20 amino acid residues. In certain embodiments, a linker consists of 1 to 10 amino acid residues, 1 to 15 amino acid residues, 5 to 20 amino acid residues, 10 to 25 amino acid residues, 10 to 30 amino acid residues, or 10 to 50 amino acid residues. In some embodiments, a linker is an enzyme-cleavable linker or a disulfide linker. In one embodiment, the cleavable linker is cleavable via an enzyme such an aminopeptidase, an aminoesterase, a dipeptidyl carboxy peptidase, or a protease of the blood clotting cascade. In one embodiment, the linker that may be conjugated to the antibody does not interfere with the antibody binding to either recombinant SARS-CoV-2 spike protein or a fragment thereof (e.g., RBD), the virion of SARS-CoV-2, or both, using techniques known in the art or described herein.

In certain aspects, diagnosis and detection can be accomplished, for example, by coupling the antibody to a detectable substance(s) including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ¹¹³Sn, and ¹¹⁷Sn; and positron emitting metals using various positron emission tomographies, and non-radioactive paramagnetic metal ions.

Provided are antibodies described herein conjugated or recombinantly fused to a therapeutic moiety (or one or more therapeutic moieties) and uses of such antibodies. The antibody can be conjugated or recombinantly fused to a therapeutic moiety, such as a cytotoxin, e.g., a cytostatic or cytocidal agent, a therapeutic agent or a radioactive metal ion, e.g., alpha-emitters.

Further, provided herein are uses of the antibodies conjugated or recombinantly fused to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity.

In addition, an antibody described herein can be conjugated to therapeutic moieties such as a radioactive metal ion, such as alpha-emitters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides.

Moreover, antibodies can be fused to marker sequences, such as a peptide to facilitate purification. In some embodiments, the marker amino acid sequence is a hexa-histidine peptide (i.e., His-tag), such as the tag provided in a pQE vector (QIAGEN, Inc.), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

Methods for fusing or conjugating therapeutic moieties (including polypeptides) to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-10539, 1991; Traunecker et al., Nature, 331:84-86, 1988; Zheng et al., J. Immunol., 154:5590-5600, 1995; Vil et al., Proc. Natl. Acad. Sci. USA, 89:11337-11341, 1992; which are incorporated herein by reference in their entireties.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the monoclonal antibodies described herein (or an antigen-binding fragment thereof) (e.g., antibodies with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies, or the encoded antibodies, may be altered by being subjected to random mutagenesis by error-prone PCR, random nucleotide insertion or other methods prior to recombination. A polynucleotide encoding a monoclonal antibody described herein (or an antigen-binding fragment thereof) may be recombined with one or more components, motifs, sections, parts, domains, fragments, etc. of one or more heterologous molecules.

An antibody can also be conjugated to a second antibody to form an antibody heteroconjugate as described by Segal in U.S. Pat. No. 4,676,980, which is incorporated herein by reference in its entirety.

An antibody can also be linked directly or indirectly to one or more antibodies to produce bispecific/multispecific antibodies.

An antibody can also be attached to solid supports, which are particularly useful for immunoassays or purification of an antigen. Such solid supports include, but are not limited to, glass, cellulose, polyacrylamide, nylon, polystyrene, polyvinyl chloride or polypropylene.

6.2 Polynucleotides

In certain aspects, provided herein are polynucleotides comprising a nucleotide sequence encoding an antibody described herein or a fragment thereof (e.g., a variable heavy chain region and/or variable light chain region) that binds to SARS-CoV-2 spike protein or a fragment thereof (e.g. RBD), and vectors, e.g., vectors comprising such polynucleotides for recombinant expression in host cells (e.g., E. coli and mammalian cells). Provided herein are polynucleotides comprising nucleotide sequences encoding any of the antibodies provided herein, as well as vectors comprising such polynucleotide sequences, e.g., expression vectors for their efficient expression in host cells, e.g., mammalian cells.

In one embodiment, an “isolated” polynucleotide or nucleic acid molecule is one that is separated from other nucleic acid molecules that are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5% or 0.1% (in particular less than about 10%) of other material, e.g., cellular material, culture medium, other nucleic acid molecules, chemical precursors and/or other chemicals. In one embodiment, a nucleic acid molecule(s) encoding an antibody described herein is isolated or purified.

As used herein, the terms “polynucleotide(s)” “nucleic acid” and “nucleotide” include deoxyribonucleotides, deoxyribonucleic acids, ribonucleotides, and ribonucleic acids, and polymeric forms thereof, and includes either single- or double-stranded forms. In certain embodiments, the terms “polynucleotide(s)” “nucleic acid” and “nucleotide” include known analogues of natural nucleotides, for example, peptide nucleic acids (“PNA”s), that have similar binding properties as the reference nucleic acid. In some embodiments, the terms “polynucleotide(s)” “nucleic acid” and “nucleotide” refer to deoxyribonucleic acids (e.g., cDNA or DNA). In other embodiments, the terms “polynucleotide(s)” “nucleic acid” and “nucleotide” refer to ribonucleic acids (e.g., mRNA or RNA).

In some aspects, provided herein are polynucleotides comprising nucleotide sequences encoding antibodies, which binds to SARS-CoV-2 spike protein or a fragment thereof (e.g., RBD) and comprises an amino acid sequence as described herein, as well as antibodies which compete with such antibodies for binding to SARS-CoV-2 spike protein or a fragment thereof (e.g., in a dose-dependent manner), or which binds to the same epitope as that of such antibodies. In one embodiments, a polynucleotide described herein an antibody which comprises a variable heavy chain region and/or variable light chain region of an antibody with the same name in Tables 1 and 2.
In one embodiment, a polynucleotide described herein comprises a nucleotide sequence encoding an antibody which comprises a variable heavy chain region comprising the amino acid sequence of an antibody in Table 1 and/or a variable light chain region comprising the amino acid sequence of an antibody in Table 2 with the same name. In some embodiments, a polynucleotide described herein comprises a nucleotide sequence encoding for an antibody that binds to SARS-CoV-2 spike protein or a fragment thereof (e.g., RBD), wherein the antibody comprises 1, 2, or 3 VH CDRs and/or 1, 2, or 3 VL CDRs of an antibody in Tables 1 and 2.

In certain aspects, provided herein are polynucleotides comprising a nucleotide sequence encoding the light chain or heavy chain of an antibody described herein. The polynucleotides can comprise nucleotide sequences encoding a light chain or a VL domain, comprising the VL FRs and CDRs of an antibody described herein. The polynucleotides can comprise nucleotide sequences encoding a heavy chain, or a VH domain, comprising the VH FRs and CDRs of antibodies described herein.

In some embodiments, a polynucleotide described herein encodes a variable heavy chain region, wherein the polynucleotide comprises a nucleic acid sequence that is at least 80%, 85%, 90%, 91%, 92%, 93%, 94%, 95%, 96%, 97%, or 98% identical to the nucleic acid sequence of a variable heavy chain region of an antibody in Table 1. In some embodiments, a polynucleotide described herein encodes a variable light chain region, wherein the polynucleotide comprises a nucleic acid sequence that is at least 80%, 85%, 90%, 91%, 92%, 93%, 94%, 95%, 96%, 97%, or 98% identical to the nucleic acid sequence of a variable light chain region of an antibody in Table 1. In one embodiment, the variable heavy chain region and/or variable light chain region encoded the polynucleotide(s) does not affect the amino acid sequence of the variable heavy chain region and/or variable light chain region.

In some embodiments, a polynucleotide described herein encodes a variable heavy chain region and a variable light chain region, wherein the polynucleotide comprises (i) a nucleic acid sequence that is at least 80%, 85%, 90%, 91%, 92%, 93%, 94%, 95%, 96%, 97%, or 98% identical to the nucleic acid sequence of a variable heavy chain region of an antibody in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of an antibody in Table 2. In one embodiment, the variable heavy chain region and/or variable light chain region encoded by the polynucleotide(s) does not affect the amino acid sequence of the variable heavy chain region and/or variable light chain region.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 1 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 1 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 1 in Tables 1 or 6, and the CDRs of the variable light chain region are identical to the CDRs of antibody number 1 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 1 in Table 1, and (ii) a nucleic acid sequence identical to the nucleic acid sequence of a variable light chain region of antibody number 1 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 2 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 2 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 2 in Tables 1 or 6, and the CDRs of the variable light chain region are identical to the CDRs of antibody number 2 in Tables 2 or 8. In another embodiment, a polynucleotide(s) described herein encodes a variable heavy chain region and a variable light the nucleic acid sequence of a variable heavy chain region of antibody number 2 in Table 1, and (ii) a nucleic acid sequence identical to the nucleic acid sequence of a variable light chain region of antibody number 2 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 3 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 3 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 3 in Tables 1 or 6, and the CDRs of the variable light chain region are identical to the CDRs of antibody number 3 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 3 in Table 1, and (ii) a nucleic acid sequence identical to the nucleic acid sequence of a variable light chain region of antibody number 3 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 4 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 4 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 4 in Tables 1 or 6, and the CDRs of the variable light chain region are identical to the CDRs of antibody number 4 in Tables 2 or 8. In another embodiment, a polynucleotide(s) described herein encodes a variable heavy chain region and a variable light the nucleic acid sequence of a variable heavy chain region of antibody number 4 in Table 1, and (ii) a nucleic acid sequence identical to the nucleic acid sequence of a variable light chain region of antibody number 4 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 5 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 5 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 5 in Tables 1 or 6, and the CDRs of the variable light chain region are identical to the CDRs of antibody number 5 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 5 in Table 1, and (ii) a nucleic acid sequence identical to the nucleic acid sequence of a variable light chain region of antibody number 5 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 6 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 6 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 6 in Tables 1 or 6, and the CDRs of the variable light chain region are identical to the CDRs of antibody number 6 in Tables 2 or 8. In another embodiment, a polynucleotide(s) described herein encodes a variable heavy chain region and a variable light the nucleic acid sequence of a variable heavy chain region of antibody number 6 in Table 1, and (ii) a nucleic acid sequence identical to the nucleic acid sequence of a variable light chain region of antibody number 6 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 7 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 7 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 7 in Tables 1 or 6, and the CDRs of the variable light chain region are identical to the CDRs of antibody number 7 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 7 in Table 1, and (ii) a nucleic acid sequence identical to the nucleic acid sequence of a variable light chain region of antibody number 7 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 8 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 8 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 8 in Tables 1 or 6, and the CDRs of the variable light chain region are identical to the CDRs of antibody number 8 in Tables 2 or 8. In another embodiment, a polynucleotide(s) described herein encodes a variable heavy chain region and a variable light the nucleic acid sequence of a variable heavy chain region of antibody number 8 in Table 1, and (ii) a nucleic acid sequence identical to the nucleic acid sequence of a variable light chain region of antibody number 8 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 9 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 9 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 9 in Tables 1 or 6, and the CDRs of the variable light chain region are identical to the CDRs of antibody number 9 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 9 in Table 1, and (ii) a nucleic acid sequence identical to the nucleic acid sequence of a variable light chain region of antibody number 9 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 10 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 10 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 10 in Tables 1 or 6, and the CDRs of the variable light chain region are identical to the CDRs of antibody number 10 in Tables 2 or 8. In another embodiment, a polynucleotide(s) described herein encodes a variable heavy chain region and a variable light the nucleic acid sequence of a variable heavy chain region of antibody number 10 in Table 1, and (ii) a nucleic acid sequence identical to the nucleic acid sequence of a variable light chain region of antibody number 10 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 11 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 11 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 11 in Tables 1 or 6, and the CDRs of the variable light chain region are identical to the CDRs of antibody number 11 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 11 in Table 1, and (ii) a nucleic acid sequence identical to the nucleic acid sequence of a variable light chain region of antibody number 11 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 12 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 12 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 12 in Tables 1 or 6, and the CDRs of the variable light chain region are identical to the CDRs of antibody number 12 in Tables 2 or 8. In another embodiment, a polynucleotide(s) described herein encodes a variable heavy chain region and a variable light the nucleic acid sequence of a variable heavy chain region of antibody number 12 in Table 1, and (ii) a nucleic acid sequence identical to the nucleic acid sequence of a variable light chain region of antibody number 12 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 13 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 13 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 13 in Tables 1 or 6, and the CDRs of the variable light chain region are identical to the CDRs of antibody number 13 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 13 in Table 1, and (ii) a nucleic acid sequence identical to the nucleic acid sequence of a variable light chain region of antibody number 13 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 14 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 14 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 14 in Tables 1 or 6, and the CDRs of the variable light chain region are identical to the CDRs of antibody number 14 in Tables 2 or 8. In another embodiment, a polynucleotide(s) described herein encodes a variable heavy chain region and a variable light the nucleic acid sequence of a variable heavy chain region of antibody number 14 in Table 1, and (ii) a nucleic acid sequence identical to the nucleic acid sequence of a variable light chain region of antibody number 14 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 15 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 15 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 15 in Tables 1 or 6, and the CDRs of the variable light chain region are identical to the CDRs of antibody number 15 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 15 in Table 1, and (ii) a nucleic acid sequence identical to the nucleic acid sequence of a variable light chain region of antibody number 15 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 16 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 16 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 16 in Tables 1 or 6, and the CDRs of the variable light chain region are identical to the CDRs of antibody number 16 in Tables 2 or 8. In another embodiment, a polynucleotide(s) described herein encodes a variable heavy chain region and a variable light the nucleic acid sequence of a variable heavy chain region of antibody number 16 in Table 1, and (ii) a nucleic acid sequence identical to the nucleic acid sequence of a variable light chain region of antibody number 16 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 17 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 17 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 17 in Tables 1 or 6, and the CDRs of the variable light chain region are identical to the CDRs of antibody number 17 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 17 in Table 1, and (ii) a nucleic acid sequence identical to the nucleic acid sequence of a variable light chain region of antibody number 17 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 18 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 18 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 18 in Tables 1 or 6, and the CDRs of the variable light chain region are identical to the CDRs of antibody number 18 in Tables 2 or 8. In another embodiment, a polynucleotide(s) described herein encodes a variable heavy chain region and a variable light the nucleic acid sequence of a variable heavy chain region of antibody number 18 in Table 1, and (ii) a nucleic acid sequence identical to the nucleic acid sequence of a variable light chain region of antibody number 18 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 19 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 19 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 19 in Tables 1 or 6, and the CDRs of the variable light chain region are identical to the CDRs of antibody number 19 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 19 in Table 1, and (ii) a nucleic acid sequence identical to the nucleic acid sequence of a variable light chain region of antibody number 19 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 20 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 20 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 20 in Tables 1 or 6, and the CDRs of the variable light chain region are identical to the CDRs of antibody number 20 in Tables 2 or 8. In another embodiment, a polynucleotide(s) described herein encodes a variable heavy chain region and a variable light the nucleic acid sequence of a variable heavy chain region of antibody number 20 in Table 1, and (ii) a nucleic acid sequence identical to the nucleic acid sequence of a variable light chain region of antibody number 20 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 21 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 21 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 21 in Tables 1 or 6, and the CDRs of the variable light chain region are identical to the CDRs of antibody number 21 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 21 in Table 1, and (ii) a nucleic acid sequence identical to the nucleic acid sequence of a variable light chain region of antibody number 21 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 22 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 22 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 22 in Tables 1 or 6, and the CDRs of the variable light chain region are identical to the CDRs of antibody number 22 in Tables 2 or 8. In another embodiment, a polynucleotide(s) described herein encodes a variable heavy chain region and a variable light the nucleic acid sequence of a variable heavy chain region of antibody number 22 in Table 1, and (ii) a nucleic acid sequence identical to the nucleic acid sequence of a variable light chain region of antibody number 22 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 23 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 23 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 23 in Tables 1 or 6, and the CDRs of the variable light chain region are identical to the CDRs of antibody number 23 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 23 in Table 1, and (ii) a nucleic acid sequence identical to the nucleic acid sequence of a variable light chain region of antibody number 23 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 24 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 24 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 24 in Tables 1 or 6, and the CDRs of the variable light chain region are identical to the CDRs of antibody number 24 in Tables 2 or 8. In another embodiment, a polynucleotide(s) described herein encodes a variable heavy chain region and a variable light the nucleic acid sequence of a variable heavy chain region of antibody number 24 in Table 1, and (ii) a nucleic acid sequence identical to the nucleic acid sequence of a variable light chain region of antibody number 24 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 215 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 25 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 25 in Tables 1 or 6, and the CDRs of the variable light chain region are identical to the CDRs of antibody number 25 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 25 in Table 1, and (ii) a nucleic acid sequence identical to the nucleic acid sequence of a variable light chain region of antibody number 25 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 26 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 26 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 26 in Tables 1 or 6, and the CDRs of the variable light chain region are identical to the CDRs of antibody number 26 in Tables 2 or 8. In another embodiment, a polynucleotide(s) described herein encodes a variable heavy chain region and a variable light the nucleic acid sequence of a variable heavy chain region of antibody number 26 in Table 1, and (ii) a nucleic acid sequence identical to the nucleic acid sequence of a variable light chain region of antibody number 26 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 27 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 27 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 27 in Tables 1 or 6, and the CDRs of the variable light chain region are identical to the CDRs of antibody number 27 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 27 in Table 1, and (ii) a nucleic acid sequence identical to the nucleic acid sequence of a variable light chain region of antibody number 27 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 28 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 28 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 28 in Tables 1 or 6, and the CDRs of the variable light chain region are identical to the CDRs of antibody number 28 in Tables 2 or 8. In another embodiment, a polynucleotide(s) described herein encodes a variable heavy chain region and a variable light the nucleic acid sequence of a variable heavy chain region of antibody number 28 in Table 1, and (ii) a nucleic acid sequence identical to the nucleic acid sequence of a variable light chain region of antibody number 28 in Table 2.

In some embodiments, a polynucleotide(s) described herein encodes a variable heavy chain region and a variable light chain region, wherein the polynucleotide(s) comprises (i) a nucleic acid sequence that is at least 80%, 85%, 90%, 91%, 92%, 93%, 94%, 95%, 96%, 97%, or 98% identical to the nucleic acid sequence of a variable heavy chain region of antibody number 29 in Table 1, and (ii) a nucleic acid sequence that is at least 80%, 85%, 90%, 91%, 92%, 93%, 94%, 95%, 96%, 97%, or 98% identical to the nucleic acid sequence of a variable light chain region of antibody number 29 in Table 2. In one embodiment, the variable heavy chain region and/or variable light chain region encoded by the polynucleotide(s)(s) does not affect the amino acid sequence of the variable heavy chain region and/or variable light chain region. In one embodiment, the CDRs of the variable heavy chain region are identical to the CDRs of antibody number 29 in Tables 1 or 6, and the CDRs of the variable light chain region are identical to the CDRs of antibody number 29 in Tables 2 or 8. In another embodiment, a polynucleotide(s) described herein encodes a variable heavy chain region and a variable light chain region, wherein the polynucleotide(s) comprises (i) a nucleic acid sequence identical to the nucleic acid sequence of a variable heavy chain region of antibody number 29 in Table 1, and (ii) a nucleic acid sequence identical to the nucleic acid sequence of a variable light chain region of antibody number 29 in Table 2.

In one embodiment, a polynucleotide provided herein comprises a nucleotide sequence encoding a kappa light chain (e.g., human kappa light chain). In one embodiment, a polynucleotide provided herein comprises a nucleotide sequence encoding a lambda light chain (e.g., human lambda light chain).

In one embodiment, a polynucleotide provided herein comprises a nucleotide sequence encoding an IgG1 heavy chain (e.g., human IgG1 heavy chain) of an antibody described herein. In one embodiment, a polynucleotide provided herein comprises a nucleotide sequence encoding IgG4 heavy chain (e.g., human IgG4 heavy chain). In one embodiment, a polynucleotide provided herein comprises a nucleotide sequence encoding IgG2 heavy chain (e.g., human IgG2 heavy chain).

In one embodiment, a polynucleotide provided herein encodes an antigen-binding domain, e.g., an Fab or F(ab′)₂.

Also provided are polynucleotides that hybridize under high stringency, intermediate or lower stringency hybridization conditions to antisense polynucleotides of polynucleotides that encode an antibody described herein or a fragment thereof (e.g., variable heavy chain region and/or variable light chain region). In specific embodiments, a polynucleotide described herein hybridizes under high stringency, or intermediate stringency hybridization conditions to an antisense polynucleotide of a polynucleotide encoding a variable light chain region, provided herein. In another specific embodiments, a polynucleotide described herein hybridizes under high stringency, or intermediate stringency hybridization conditions to an antisense polynucleotide of a polynucleotide encoding a variable heavy chain region, provided herein.

Hybridization conditions have been described in the art and are known to one of skill in the art. For example, hybridization under stringent conditions can involve hybridization to filter-bound DNA in 6×sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3.

Also provided herein are polynucleotides encoding an antibody that are optimized, e.g., by codon/RNA optimization, replacement with heterologous signal sequences, and elimination of mRNA instability elements. Methods to generate optimized nucleic acids encoding an antibody or a fragment thereof (e.g., light chain, heavy chain, a variable heavy chain region, or a variable light chain region) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid. Such methods can increase expression of an antibody or fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an antibody encoded by polynucleotides that have not been optimized.

In certain embodiments, an optimized polynucleotide sequence encoding an antibody described herein or a fragment thereof (e.g., a variable light chain region and/or a variable heavy chain region) can hybridize to an antisense polynucleotide of an unoptimized polynucleotide encoding an antibody described herein or a fragment thereof (e.g., a variable light chain region and/or a variable heavy chain region). In specific embodiments, an optimized nucleotide sequence encoding an antibody described herein or a fragment thereof (e.g., a variable light chain region and/or a variable heavy chain region) hybridizes under high stringency conditions to an antisense polynucleotide of an unoptimized polynucleotide encoding an antibody described herein or a fragment thereof (e.g., a variable light chain region and/or a variable heavy chain region). In one embodiment, an optimized nucleotide sequence encoding an antibody described herein or a fragment thereof (e.g., a variable light chain region and/or a variable heavy chain region) hybridizes under intermediate or lower stringency hybridization conditions to an antisense polynucleotide of an unoptimized polynucleotide encoding an antibody described herein or a fragment thereof (e.g., a variable light chain region and/or a variable heavy chain region). Information regarding hybridization conditions have been described, see, e.g., U.S. Patent Application Publication No. US 2005/0048549 (e.g., paragraphs 72-73), which is incorporated herein by reference in its entirety.

The polynucleotides can be obtained, and the nucleotide sequence of the polynucleotides determined, by any method known in the art. Nucleotide sequences encoding antibodies described herein, and modified forms of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light domain and/or the variable heavy domain of an antibody. The amplified nucleic acids can be cloned into vectors for expression in host cells and for further cloning, for example, to generate chimeric and humanized antibodies.

If a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library or a cDNA library generated from, or nucleic acid, preferably poly A+RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding an antibody can be readily isolated and sequenced using conventional procedures (e.g., by using oligonucleotide probes that are capable of binding specifically to genes encoding the heavy and light chains of the antibody). Hybridoma cells can serve as a source of such DNA. Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of antibodies in the recombinant host cells.

In phage display methods, functional antibody domains are displayed on the surface of phage particles which carry the polynucleotide sequences encoding them. In particular, a library of DNA sequences encoding a variable light chain region and/or a variable heavy chain region are generated (e.g., amplified from animal cDNA libraries such as human cDNA libraries or random libraries are generated by chemical synthesis). The DNA encoding the variable light chain region and a variable heavy chain region are recombined together with an scFv linker by PCR and cloned into a phagemid vector. The vector is electroporated in E. coli and the E. coli is infected with helper phage. Phage expressing an antigen-binding domain that binds to a particular antigen can be selected or identified with antigen, e.g., using labeled antigen or antigen bound or captured to a solid surface or bead. After phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produced Fab, Fab′ and F(ab′)₂ fragments can also be employed using methods known in the art such as those disclosed in PCT Publication No. WO 92/22324; Mullinax et al., 1992, BioTechniques, 12(6):864-869; Sawai et al., 1995, AJRI, 34:26-34; and Better et al., 1988, Science, 240:1041-1043.

Antibodies can be isolated from antibody phage libraries generated using the techniques described in McCafferty et al., Nature, 348:552-554 (1990). Clackson et al., Nature, 352:624-628 (1991). Marks et al., J Mol. Biol., 222:581-597 (1991) describe the isolation of murine and human antibodies, respectively, using phage libraries. Chain shuffling can be used in the production of high affinity (nM range) human antibodies (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)).

To generate whole antibodies, PCR primers including a variable light chain region and a variable heavy chain region nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise a promoter, a secretion signal, a cloning site for the variable domain, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

In one embodiment, provided herein are two vectors (e.g., plasmids or viruses), wherein one vector comprises the variable heavy chain region of an antibody described herein, and the second vector comprises the variable light chain region of an antibody described herein.

In a non-limiting example, the Dyax (Cambridge, MA) technology platform can be used to convert Fab-phage or Fabs to complete IgG antibodies, such as the Dyax pR rapid reformatting vectors (RR). Briefly, by PCR, a Fab-encoding DNA fragment is inserted into a Dyax pR-RRV between a eukaryotic leader sequence and an IgG heavy chain constant region cDNA. Antibody expression is driven by the human cytomegalovirus (hCMV). In a second cloning step, bacterial regulatory elements are replaced by the appropriate eukaryotic sequences (i.e., the IRES (internal ribosome entry site) motif). The expression vector can also include the SV40 origin of replication. The Dyax pRh1(a,z), pRh1(f), pRh4 and pRm2a are expression vectors allowing expression of reformatted FAbs as human IgG1 (isotype a,z), human IgG1 (isotype F), human IgG4, and mouse IgG2a, respectively. Expressing vectors can be introduced into a suitable host cell (e.g., HEK293T cells, CHO cells)) for expression and purification.

In some embodiments, a polynucleotide(s) encoding an antibody provided herein is isolated. In other embodiments, a polynucleotide(s) encoding an antibody provided herein is not isolated. In yet other embodiments, a polynucleotide(s) encoding an antibody provided herein is integrated, e.g., into chromosomal DNA or an expression vector. In specific embodiments, a polynucleotide(s) encoding an antibody provided herein is not integrated into chromosomal DNA.

6.3 Antibody Production

In one aspect, provided herein are methods for making an antibody described herein, which binds to SARS-CoV-2 spike protein or a fragment thereof (e.g. RBD). In one embodiment, an antibody described herein (e.g., an antigen-binding fragment), which binds to SARS-CoV-2 spike protein or a fragment thereof (e.g. RBD), may be prepared, expressed, created or isolated by any means that involves creation, e.g., via synthesis or genetic engineering of sequences. In one embodiment, such an antibody comprises sequences that are encoded by DNA sequences that do not naturally exist withing the antibody germline repertoire of an animal or mammal (e.g., a human).

In certain aspects, a method for making an antibody described herein, which binds to SARS-CoV-2 or fragment thereof (e.g. RBD), comprises the step of culturing a cell (e.g., host cell or hybridoma cell) that expresses the antibody. In certain embodiments, the method for making an antibody described herein further comprises the step of purifying the antibody expressed by the cell. In certain aspects, a method for making an antibody described herein (e.g., an antigen-binding fragment thereof), which binds to SARS-CoV-2 spike protein or a fragment thereof (e.g. RBD), comprises the step of culturing a cell (e.g., host cell or hybridoma cell) that comprises polynucleotides or vectors encoding the antibody. In one aspect, provided herein are methods for producing an antibody described herein (e.g., an antigen-binding fragment thereof), comprising expressing such antibody from a host cell.

In certain aspects, provided herein are cells (e.g., host cells) expressing (e.g., recombinantly expressing) the antibodies described herein (e.g., an antigen-binding fragment thereof) and related expression vectors. In another aspect, provided herein are vectors (e.g., expression vectors) comprising polynucleotides comprising nucleotide sequences encoding antibodies (e.g., an antigen-binding fragment) for recombinant expression in host cells, preferably in mammalian cells. Also provided herein are host cells comprising a polynucleotide encoding an antibody, or vectors comprising a polynucleotide encoding an antibody for recombinantly expressing an antibody described herein. In one embodiment, provided herein is a host cell comprising two vectors, wherein the first vector comprises a polynucleotide of an antibody described herein, and the second vector comprises a polynucleotide encoding an antibody for recombinantly expressing an antibody described herein. The cells may be primary cells or cell lines. In one aspect, provided herein are hybridoma cells expressing an antibody described herein. In one embodiment, the host cell is isolated from other cells. In another embodiment, the host cell is not found within the body of a subject.

Antibodies described herein (e.g., monoclonal antibodies, such as chimeric or humanized antibodies, or an antigen-binding fragment thereof) that bind to SARS-CoV-2 spike protein or a fragment thereof (e.g. RBD) can be produced by any method known in the art for the synthesis of antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981). The term “monoclonal antibody” as used herein is not limited to antibodies produced through hybridoma technology.

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will bind to the protein (e.g., SARS-CoV-2 spike protein or a fragment thereof (e.g., RBD)) used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

Additionally, a RIMMS (repetitive immunization multiple sites) technique can be used to immunize an animal (Kilptrack et al., 1997 Hybridoma 16:381-9, incorporated by reference in its entirety).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against SARS-CoV-2 spike protein or a fragment thereof (e.g., RBD). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI 1640 medium. Alternatively, clonal cells can be isolated using a semi-solid agar supplemented with HAT (Stemcell Technologies). In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

In some embodiments, mice (or other animals, such as rats, monkeys, donkeys, pigs, sheep, goats, hamsters, or dogs) can be immunized with an antigen (e.g., SARS-CoV-2 spike proteina fragment thereof (e.g., RBD)) and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP2/0 available from the American Type Culture Collection (ATCC®) (Manassas, VA), to form hybridomas. Hybridomas are selected and cloned by limited dilution.

In certain embodiments, lymph nodes of the immunized mice are harvested and fused with NS0 myeloma cells.

The hybridoma clones are then assayed by methods known in the art for cells that secrete antibodies capable of binding a polypeptide of the antigen. Ascites fluid, which generally contains high levels of antibodies, can be generated by immunizing mice with positive hybridoma clones.

Accordingly, described herein are methods of making antibodies described herein by culturing a hybridoma cell secreting an antibody. In certain embodiments, the method of making an antibody described herein further comprises the step of purifying the antibody.

In some embodiments, the hybridoma is generated by fusing splenocytes isolated from a mouse (or other animal, such as rat, monkey, donkey, pig, sheep, or dog) immunized with SARS-CoV-2 spike proteina fragment thereof (e.g. RBD) with myeloma cells and then screening the hybridomas resulting from the fusion for hybridoma clones that secrete an antibody able to bind to the SARS-CoV-2 spike protein or a fragment thereof (e.g. RBD). In certain embodiments, the hybridoma is generated by fusing lymph nodes isolated from a mouse (or other animal, such as rat, monkey, donkey, pig, sheep, or dog) immunized with a SARS-CoV-2 spike protein or a fragment thereof (e.g. RBD) with myeloma cells, and then screening the hybridomas resulting from the fusion for hybridoma clones that secrete an antibody able to bind to the SARS-CoV-2 spike protein or a fragment thereof (e.g. RBD).

Antibodies described herein include antibody fragments that recognize SARS-CoV-2 spike protein or a fragment thereof (e.g. RBD) and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein can also be generated using various phage display methods known in the art. Examples of phage display methods that can be used to make the antibodies described herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043.

In one aspect, to generate whole antibodies, PCR primers including variable heavy chain region or variable light chain region nucleotide sequences, a restriction site, and a flanking sequence to protect the restriction site can be used to amplify the variable heavy chain region or variable light chain region sequences from a template, e.g., scFv clones. Utilizing cloning techniques known to those of skill in the art, the PCR amplified variable heavy chain region can be cloned into vectors expressing a heavy chain constant region, and the PCR amplified variable light chain region can be cloned into vectors expressing a light chain constant region, e.g., human kappa or lambda constant regions. The variable heavy chain region and variable light chain region can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it can be preferable to use human, humanized or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741.

Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., SARS-CoV-2 spike protein or a fragment thereof (e.g. RBD). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598.

In some embodiments, human antibodies can be produced using mouse-human hybridomas. In some embodiments, human antibodies can be generated by inserting polynucleotides encoding human CDRs (e.g., VL CDRs and/or VH CDRs) of an antibody into an expression vector containing nucleotide sequences encoding human framework region sequences. In certain embodiments, such expression vectors further comprise nucleotide sequences encoding a constant region of a human light and/or heavy chain. In some embodiments, human antibodies can be generated by inserting human CDRs (e.g., VL CDRs and/or VH CDRs) of an antibody obtained from a phage library into such human expression vectors.

In certain embodiments, a human antibody can be generated by selecting human CDR sequences that are homologous (or substantially homologous) to non-human CDR sequences of a non-human antibody and selecting human framework sequences that are homologous (or substantially homologous) to non-human framework sequences of a non-human antibody.

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301.

Bispecific antibodies are antibodies that have binding specificities for at least two different epitopes. Exemplary bispecific antibodies may bind to two different epitopes of an antigen or to two different epitopes of two different antigens. In specific embodiments, a bispecific antibody has two distinct antigen-binding domains, wherein each domain specifically binds to a different antigen. Other such antibodies may bind a first antigen (e.g., SARS-CoV-2 spike protein or a fragment thereof (e.g., RBD)) and further bind a second antigen. Bispecific antibodies can be prepared as full-length antibodies or antibody fragments (e.g., F(ab′): bispecific antibodies).

Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl. Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731,168; 4,676,980; and 4,676,980, WO 94/04690; WO 91/00360; WO 92/200373; WO 93/17715; WO 92/08802; and EP 03089.)

Further, antibodies that bind to SARS-CoV-2 spike protein or a fragment thereof (e.g., RBD) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

Recombinant expression of an antibody described herein (e.g., a full-length antibody, heavy and/or light chain of an antibody, or a single chain antibody described herein) that binds to SARS-CoV-2 spike protein or a fragment thereof (e.g., RBD), can for example, involve construction of vectors (e.g., expression vectors) containing a polynucleotide that encodes the antibody or fragments thereof (e.g., VL domain and/or VH domain). Once a polynucleotide encoding an antibody molecule, heavy and/or light chain of an antibody, or antigen-binding fragment thereof described herein has been obtained, a vector for the production of the antibody molecule can be produced by recombinant DNA technology using techniques well-known in the art. Methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464) and the variable domain of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein or a fragment thereof. Thus, provided herein are host cells containing a polynucleotide encoding an antibody described herein or fragments thereof, or a heavy or light chain thereof, or antigen-binding fragment thereof, or a single chain antibody described herein, operably linked to a promoter for expression of such sequences in the host cell. In certain embodiments, e.g., for the expression of double-chained antibodies, vectors encoding both the heavy and light chains individually can be co-expressed in the host cell for expression of the entire immunoglobulin molecule, as detailed below. In certain embodiments, a host cell contains a vector comprising a polynucleotide encoding both the heavy chain and light chain of an antibody described herein, or a fragment thereof. In specific embodiments, a host cell contains two different vectors, a first vector comprising a polynucleotide encoding a heavy chain of an antibody described herein, or a fragment thereof, and a second vector comprising a polynucleotide encoding a light chain of an antibody described herein, or a fragment thereof. In other embodiments, a first host cell comprises a first vector comprising a polynucleotide encoding a heavy chain of an antibody described herein, or a fragment thereof, and a second host cell comprises a second vector comprising a polynucleotide encoding a light chain of an antibody described herein.

A variety of host-expression vector systems can be utilized to express antibody molecules described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, MDCK, HEK 293, NS0, PER.C6, VERO, CRL7O3O, HsS78Bst, HeLa, and NIH 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In one embodiment, a mammalian expression vector is pOptiVEC™ or pcDNA3.3. Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary (CHO) cells, in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In certain embodiments, antibodies described herein are produced by CHO cells or NS0 cells. In one embodiment, the expression of nucleotide sequences encoding antibodies described herein (or fragments thereof) which bind to SARS-CoV-2 spike protein or a fragment thereof (e.g., RBD) is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruther et al., 1983, EMBO 12:1791), in which the antibody coding sequence can be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

As used herein, the term “host cell” refers to any type of cell, e.g., a primary cell or a cell from a cell line. In specific embodiments, the term “host cell” refers a cell transfected with a polynucleotide and the progeny or potential progeny of such a cell. Progeny of such a cell may not be identical to the parent cell transfected with the polynucleotide due to mutations or environmental influences that may occur in succeeding generations or integration of the polynucleotide into the host cell genome.

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, COS, MDCK, HEK 293, NIH 3T3, W138, BT483, Hs578T, HTB2, BT2O and T47D, NS0 (a murine myeloma cell line that does not endogenously produce any immunoglobulin chains), CRL7O3O and HsS78Bst cells. In certain embodiments, humanized monoclonal antibodies described herein are produced in mammalian cells, such as CHO cells.

For long-term, high-yield production of recombinant proteins, stable expression is preferred. For example, cell lines that stably express the antibody molecule can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the antibody molecule. Such engineered cell lines can be particularly useful in screening and evaluation of compositions that interact directly or indirectly with the antibody molecule.

A number of selection systems can be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215); and hygro, which confers resistance to hygromycin (Santerre et al., 1984, Gene 30:147). Methods commonly known in the art of recombinant DNA technology can be routinely applied to select the desired recombinant clone, and such methods are described, for example, in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds.), Current Protocols in Human Genetics, John Wiley & Sons, N Y (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell can be co-transfected with two or more expression vectors described herein, the first vector encoding a heavy chain derived polypeptide and the second vector encoding a light chain derived polypeptide. In one embodiment, a host cell comprises two expression vectors: one vector comprising a polynucleotide sequence comprising a nucleotide sequence encoding a heavy chain variable region of an antibody described herein and a second vector comprising a polynucleotide sequence comprising a nucleotide sequence encoding a light chain variable region of an antibody described herein.

Alternatively, a single vector can be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES. Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In some embodiments, an antibody (e.g., a monoclonal antibody, such as a humanized or chimeric antibody or an antigen-binding fragment thereof) described herein is isolated or purified. Generally, an isolated antibody is one that is substantially free of other antibodies with different antigenic specificities than the isolated antibody. For example, in one embodiment, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals that are involved in the synthesis of the protein. Accordingly, such preparations of the antibody have less than about 30%, 20%, 10%, 5% (by dry weight) of chemical precursors or compounds other than the antibody of interest. In one embodiment, antibodies described herein are isolated or purified.

6.4 Compositions

Provided herein are compositions (e.g., pharmaceutical compositions) comprising an antibody having the desired degree of purity in a physiologically acceptable carrier, excipient or stabilizer (Remington's Pharmaceutical Sciences (1990) Mack Publishing Co., Easton, PA). In one embodiment, a composition comprises an antibody described herein and an acceptable carrier or excipient. In some embodiments, a composition comprises two or more antibodies described herein an acceptable carrier or excipient. In one embodiment, the compositions comprise an antibody conjugated to a moiety such as described herein. Acceptable carriers, excipients, or stabilizers are nontoxic to recipients at the dosages and concentrations employed, and include buffers such as phosphate, citrate, histidine, and other organic acids; antioxidants; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In one embodiment, pharmaceutical compositions comprise an antibody, and optionally one or more additional prophylactic or therapeutic agents, in a pharmaceutically acceptable carrier. In one embodiment, pharmaceutical compositions comprise an effective amount of an antibody, and optionally one or more additional prophylactic of therapeutic agents, in a pharmaceutically acceptable carrier. In some embodiments, the antibody is the only active ingredient included in the pharmaceutical composition. In one embodiment, pharmaceutical compositions comprise an antibody conjugated to a moiety such as described herein, and optionally one or more additional prophylactic or therapeutic agents, in a pharmaceutically acceptable carrier. In some embodiments, the antibody conjugated to a moiety such as described herein is the only active ingredient included in the pharmaceutical composition. Pharmaceutical compositions described herein can be useful in the prevention and/or treatment of SARS-CoV-2 infection, or disease associated therewith. In one embodiment, a pharmaceutical compositions described herein can be useful in the prevention and/or treatment of COVID-19.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN®80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition may be formulated for any route of administration to a subject. Specific examples of routes of administration include intranasal, oral, pulmonary, transdermal, intradermal, parenteral, and mucosal. In one embodiment, the composition is formulated for intranasal or intramuscular administration. In one embodiment, the composition is formulation for intramuscular administration. In one embodiment, the composition is formulated for mucosal administration. In one embodiment, the composition is formulated for intranasal administration. For example, the composition may be formulated as an aersoal. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

An antibody can be formulated as an aerosol for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in one embodiment, have diameters of less than 50 microns, in one embodiment less than 10 microns.

In certain embodiments, a pharmaceutical composition comprising an antibody is a lyophilized powder, which can be reconstituted for administration as solutions, emulsions and other mixtures. It may also be reconstituted and formulated as solids or gels. The lyophilized powder is prepared by dissolving an antibody provided herein, or a pharmaceutically acceptable derivative thereof, in a suitable solvent. In some embodiments, the lyophilized powder is sterile. The solvent may contain an excipient that improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature. Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

An antibody or a nucleic acid sequence encoding an antibody can, for example, be formulated in liposomes. Liposomes containing the molecule of interest are prepared by methods known in the art, such as described in Epstein et al. (1985) Proc. Natl. Acad. Sci. USA 82:3688; Hwang et al. (1980) Proc. Natl. Acad. Sci. USA 77:4030; and U.S. Pat. Nos. 4,485,045 and 4,544,545. Liposomes with enhanced circulation time are disclosed in U.S. Pat. No. 5,013,556. In one embodiment, liposomal suspensions may also be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound comprising an antibody described herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

An antibody can also be entrapped in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1990) Mack Publishing Co., Easton, PA.

Sustained-release preparations can also be prepared. Suitable examples of sustained-release preparations include semipermeable matrices of solid hydrophobic polymers, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The compositions to be used for in vivo administration can be sterile. This is readily accomplished by filtration through, e.g., sterile filtration membranes.

In one embodiment, nucleic acids comprising sequences encoding an antibody described herein are administered to a subject by way of gene therapy. Gene therapy refers to therapy performed by the administration to a subject of an expressed or expressible nucleic acid. Encompassed herein are any of the methods for gene therapy available in the art. For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. For a review of methods of delivery of transgenes encoding antibodies, see, e.g., Deal, 2015, Curr. Opin. Immunol. 2015 August, 35:113-22; Deal, 2015, Curr Opin HIV AIDS. 2015 May, 10(3):190-7; Marschall, 2015, MAbs. 7(6):1010-35. In one embodiment, an mRNA encoding an antibody described herein is administered to a subject. Techniques known to one of skill in the art may be used to administer an mRNA encoding an antibody to a subject. For methods of delivery of mRNA encoding antibodies, see, e.g., U.S. Patent Application Publication No. US20130244282A1; U.S. Patent Application Publication No. US 2016/0158354A1; and International Patent Application No. WO2016014846A1, each of which is incorporated herein by reference in its entirety.

Methods commonly known in the art of recombinant DNA technology which can be used are described in Ausubel et al. (eds.), Current Protocols in Molecular Biology, John Wiley & Sons, N Y (1993); and Kriegler, Gene Transfer and Expression, A Laboratory Manual, Stockton Press, NY (1990).

6.5 Prophylactic and Therapeutic Uses of Antibodies

In one aspect, provided herein are methods for preventing COVID-19 comprising administering an antibody described herein. In one embodiment, provided herein is a method for preventing COVID-19 in a subject comprising administering to the subject an effective amount of an antibody described herein. In one embodiment, provided herein is a method for preventing COVID-19 in a subject comprising administering to the subject a pharmaceutical composition comprising an effective amount of an antibody described herein. In one embodiment, the antibody is a protein or a protein conjugate. In one embodiment, the antibody is administered to the subjects as polynucleotide sequence comprising a nucleotide sequence encoding the antibody. In one embodiment, the antibody administered to the subject is a conjugated moiety such as described herein. In one embodiment, the administration of an effective amount of the antibody to the subject inhibits or reduces in the development or onset of COVID-19. In one embodiment, provided herein is a method for preventing COVID-19 in a subject comprising administering to the subject an effective amount of an antibody described herein and another therapy, such as known to one of skill in the art or described herein. In one embodiment, the administration of an effective amount of the antibody to the subject inhibits or reduces in the development or onset of COVID-19. In another embodiment, the administration of an effective amount of the antibody to the subject inhibits or reduces onset, development and/or severity of a symptom thereof (e.g., fever, myalgia, cough, difficulty breathing, tiredness) of COVID-19. In another embodiment, the administration of an effective amount of the antibody inhibits or reduces in the recurrence of COVID-19 or a symptom associated therewith.

In some embodiments, the administration of an effective amount of an antibody to a subject results in one, two, three, four, or more of the following: (i) the reduction or inhibition of the spread of SARS-CoV-2 from one cell to another cell; (ii) the reduction or inhibition of the spread of SARS-CoV-2 from one organ or tissue to another organ or tissue; (iii) the reduction or inhibition of the spread of SARS-CoV-2 from one region of an organ or tissue to another region of the organ or tissue (e.g., the reduction in the spread of SARS-CoV-2 from the upper to lower respiratory tract); (iv) the prevention of COVID-19 after exposure to SARS-CoV-2; (v) the reduction or inhibition in SARS-CoV-2 infection and/or replication; and/or (vi) prevention of the onset or development of one or more symptoms associated with COVID-19 or SARS-CoV-2 infection.

In another aspect, provided herein are methods for treating a SARS-CoV-2 infection or COVID-19 comprising administering an antibody described herein. In one embodiment, provided herein is a method for treating SARS-CoV-2 infection or COVID-19 in a subject comprising administering to the subject an effective amount of an antibody described herein. In one embodiment, provided herein is a method for treating SARS-CoV-2 infection or COVD-19 in a subject comprising administering to the subject a pharmaceutical composition comprising an effective amount of an antibody described herein. In one embodiment, provided herein is a method for treating SARS-CoV-2 infection or COVID-19 comprising administering to the subject an effective amount of an antibody described herein and another therapy, such as known to one of skill in the art or described herein. In one embodiment, provided herein is a method for treating SARS-CoV-2 infection or COVID-19 in a subject comprising administering to the subject a pharmaceutical composition comprising an effective amount of an antibody described herein, and another therapy, such as known to one of skill in the art or described herein. In one embodiment, the antibody is administered as a polynucleotide sequence comprising a nucleotide sequence encoding the antibody. In one embodiment, the antibody that is administered to the subject is conjugated to a moiety such as described herein. In one embodiment, the administration of an effective amount of the antibody to the subject inhibits or reduces in the development of COVID-19. In another embodiment, the administration of an effective amount of the antibody to the subject inhibits or reduces onset, development and/or severity of a symptom thereof (e.g., fever, myalgia, cough, difficulty breathing, tiredness) of COVID-19. In another embodiment, the administration of an effective amount of the antibody inhibits or reduces duration of COVID-19 or a symptom associated therewith. In another embodiment, the administration of an effective amount of the antibody reduces organ failure associated with COVID-19. In another embodiment, the administration of an effective amount of the antibody reduces the hospitalization of the subject. In another embodiment, the administration of an effective amount of the antibody reduces the length of hospitalization of the subject. In another embodiment, the administration of an effective amount of the antibody increases the overall survival of subjects with COVID-19. In another embodiment, the administration of an effective amount of the antibody prevents the onset or progression of a secondary infection associated with SARS-CoV-2 infection.

In one embodiment, administration of an antibody(ies) to a subject reduces the incidence of hospitalization by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the incidence of hospitalization in the absence of administration of said antibody(ies).

In one embodiment, administration of an antibody(ies) to a subject reduces mortality by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the mortality in the absence of administration of said antibody(ies).

In one embodiment, administration of an antibody(ies) prevents or inhibits SARS-CoV-2 from binding to its host cell receptor (e.g., ACE-2) by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to SARS-CoV-2 binding to its host cell receptor in the absence of said antibody(ies) or in the presence of a negative control in an assay known to one of skill in the art or described herein.

In one embodiment, administration of an antibody(ies) inhibits or reduces SARS-CoV-2 replication by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to replication of SARS-CoV-2 in the absence of said antibody(ies) or in the presence of a negative control in an assay known to one of skill in the art or described herein. Inhibition of SARS-CoV-2 replication can be determined by detecting the SARS-CoV-2 titer in a biological specimen from a subject using methods known in the art (e.g., Northern blot analysis, RT-PCR, Western Blot analysis, etc.).

In one embodiment, administration of an antibody(ies) results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in SARS-CoV-2 titer in the subject. The fold-reduction in SARS-CoV-2 titer may be as compared to a negative control, as compared to another treatment, or as compared to the titer in the patient prior to antibody administration.

In one embodiment, administration of an antibody(ies) results in a reduction of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 5 logs, 2 to 10 logs, 2 to 5 logs, or 2 to 10 logs in SARS-CoV-2 titer in the subject. The log-reduction in SARS-CoV-2 titer may be as compared to a negative control, as compared to another treatment, or as compared to the titer in the patient prior to antibody administration.

In one embodiment, administration of an antibody(ies) inhibits or reduces SARS-CoV-2 infection of a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to SARS-CoV-2 infection of a subject in the absence of said antibody(ies) or in the presence of a negative control in an assay known to one of skill in the art or described herein.

In one embodiment, administration of an antibody(ies) inhibits or reduces the spread of SARS-CoV-2 in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the spread of SARS-CoV-2 in a subject in the absence of said an antibody(ies) or in the presence of a negative control in an assay known to one of skill in the art or described herein.

In one embodiment, administration of an antibody(ies) inhibits or reduces the spread of SARS-CoV-2 between a subject and at least one other subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the spread of SARS-CoV-2 between a subject and at least one other subject in the absence of said antibody(ies) or in the presence of a negative control in an assay known to one of skill in the art or described herein.

In one embodiment, administration of an antibody(ies) to a subject reduces the number of and/or the frequency of symptoms of in the subject (exemplary symptoms of a SARS-CoV-2 include, but are not limited to, body aches (especially joints and throat), fever, nausea, headaches, fatigue, sore throat, and difficulty breathing).

In one embodiment, administration of an antibody(ies) to a subject reduces the number of and/or the frequency of symptoms of COVID-19 in the subject (exemplary symptoms of COVID-19 include, but are not limited to, body aches (especially joints and throat), fever, nausea, headaches, fatigue, sore throat, and difficulty breathing).

An antibody(ies) may be administered alone or in combination with another/other type of therapy known in the art.

In specific embodiment, an antibody described herein may be used as any line of therapy, including, but not limited to, a first, second, third, fourth and/or fifth line of therapy. Encompassed herein are methods for administering one or more antibodies described herein to prevent the onset of a disease associated with SARS-CoV-2 infection and/or to treat or lessen the recurrence of a disease associated with SARS-CoV-2 infection.

In specific embodiment, an antibody described herein may be used as any line of therapy, including, but not limited to, a first, second, third, fourth and/or fifth line of therapy. Encompassed herein are methods for administering one or more antibodies described herein to prevent the onset of COVID-19 and/or to treat or lessen the recurrence of COVID-19.

Further encompassed herein are methods for preventing and/or treating a disease associated with SARS-CoV-2 infection (e.g., COVID-19) and/or a symptom relating thereto for which no other antiviral therapy is available.

6.5.1 Routes of Administration and Dosage

An antibody (e.g., a monoclonal antibody, such as a chimeric or humanized antibody, or an antigen-binding fragment thereof) or composition described herein may be delivered to a subject by a variety of routes. In one embodiment, an antibody conjugated to a moiety such as described herein, or a polynucleotide encoding a sequence encoding an antibody may be administered to a subject by a variety of routes. These include, but are not limited to, intranasal, intratracheal, oral, intradermal, intramuscular, intraperitoneal, transdermal, intravenous, conjunctival and subcutaneous routes. Pulmonary administration can also be employed, e.g., by use of an inhaler or nebulizer, and formulation with an aerosolizing agent for use as a spray. In one embodiment, an antibody described herein is administered to a subject intranasally or intramuscularly.

The amount of an antibody (e.g., a monoclonal antibody, such as a chimeric or humanized antibody, or an antigen-binding fragment thereof), antibody conjugate or composition which will be effective in the treatment and/or prevention of SARS-CoV-2 infection, or a disease associated therewith (e.g., COVID-19) will depend on the nature of the disease. The precise dose to be employed in a composition will also depend on the route of administration, and the seriousness of the infection or disease caused by it, and should be decided according to the judgment of the practitioner and each subject's circumstances. For passive immunization with an antibody (e.g., a monoclonal antibody, such as a chimeric or humanized antibody, or an antigen-binding fragment thereof), the dosage ranges from about 0.0001 to 100 mg/kg, and more usually 0.01 to 5 mg/kg, of the patient body weight. Generally, human antibodies have a longer half-life within the human body than antibodies from other species due to the immune response to the foreign polypeptides. Thus, lower dosages of human antibodies and less frequent administration is often possible.

An exemplary treatment regime entails administration once per every two weeks or once a month or once every 3 to 6 months for a period of one year or over several years, or over several year-intervals. In some methods, two or more antibodies with different binding specificities are administered simultaneously to a subject. An antibody is usually administered on multiple occasions. Intervals between single dosages can be weekly, monthly, every 3 months, every 6 months or yearly. Intervals can also be irregular as indicated by measuring blood levels of antibody to the SARS-CoV-2 antigen in the patient.

In some embodiments, the plasma level of an antibody described herein in a patient is measured prior to administration of a subsequent dose of an antibody described herein, or a composition thereof. The plasma level of the antibody may be considered in determining the eligibility of a patient to receive a subsequent dose of an antibody described herein. For example, a patient's plasma level of an antibody described herein may suggest not administering an antibody described herein; alternatively, a patient's plasma level of an antibody described herein may suggest administering an antibody described herein at a particular dosage, at a particular frequency, and/or for a certain period of time.

In certain embodiments, the route of administration for a dose of an antibody described herein, or a composition thereof to a patient is intranasal, intramuscular, intravenous, or a combination thereof, but other routes described herein are also acceptable. Each dose may or may not be administered by an identical route of administration. In some embodiments, an antibody described herein, or composition thereof, may be administered via multiple routes of administration simultaneously or subsequently to other doses of the same or a different antibody described herein.

6.5.2 Combination Therapy

In various embodiments, an antibody described herein or a nucleic acid encoding such an antibody may be administered to a subject in combination with one or more other therapies (e.g., antiviral or immunomodulatory therapies). In one embodiment, an antibody conjugated to a moiety such as described herein may be administered to a subject with one or more other therapies. In some embodiments, a pharmaceutical composition described herein may be administered to a subject in combination with one or more therapies. The one or more other therapies may be in the same composition or a different composition as an antibody described herein.

In some embodiments, the one or more other therapies that are supportive measures, such as pain relievers, anti-fever medications, or therapies that alleviate or assist with breathing. Specific examples of supportive measures include humidification of the air by an ultrasonic nebulizer, aerolized racemic epinephrine, oral dexamethasone, intravenous fluids, intubation, fever reducers (e.g., ibuprofen, acetometaphin), and antibiotic and/or antifungal therapy (i.e., to prevent or treat secondary bacterial and/or fungal infections).

In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In specific embodiments, two or more therapies are administered within the same patient visit. In some embodiments, two or more therapies are administered concurrently. The two or more therapies can be administered in the same composition or a different composition. Further, the two or more therapies can be administered by the same route of administration of a different route of administration.

6.5.3 Patient Populations

As used herein, the terms “subject” and “patient” are used interchangeably to refer to an animal (e.g., birds, reptiles, and mammals). In one embodiment, a patient treated or prevented in accordance with the methods provided herein is a naïve subject, i.e., a subject that does not have COVID-19 or has not been and is not currently infected with SARS-CoV-2. In another embodiment, a patient treated or prevented in accordance with the methods provided herein is a subject that is at risk of acquiring SARS-CoV-2 infection. In another embodiment, a patient treated or prevented in accordance with the methods provided herein is a patient suffering from or expected to suffer from COVID-19. In another embodiment, a patient treated or prevented in accordance with the methods provided herein is a patient diagnosed with SARS-CoV-2 infection or COVID-19. In some embodiments, a patient treated or prevented in accordance with the methods provided herein is a patient infected with SARS-CoV-2 that does not manifest any symptoms of COVID-19. In certain embodiments, a patient treated or prevented in accordance with the methods provided herein is a patient infected with SARS-CoV-2 that manifests moderate to severe symptoms of COVID-19.

In another embodiment, a patient treated or prevented in accordance with the methods provided herein is a patient experiencing one or more symptoms of COVID-19. Symptoms of COVID-19 include, but are not limited to, body aches (especially joints and throat), fever, nausea, headaches, fatigue, sore throat, and difficulty breathing. In another embodiment, a patient treated or prevented in accordance with the methods provided herein is a patient with COVID-19 who does not manifest symptoms of the disease that are severe enough to require hospitalization.

In one embodiment, a patient treated or prevented in accordance with the methods provided herein is a human. In certain embodiments, a patient treated or prevented in accordance with the methods provided herein is a human infant. In some embodiments, a patient treated or prevented in accordance with the methods provided herein is a human toddler. In certain embodiments, a patient treated or prevented in accordance with the methods provided herein is a human child. In other embodiments, a patient treated or prevented in accordance with the methods provided herein is a human adult. In some embodiments, a patient treated or prevented in accordance with the methods provided herein is an elderly human. In certain embodiments, a patient treated or prevented in accordance with the methods provided herein is patient that is pregnant.

As used herein, the term “human adult” refers to a human that is 18 years or older. As used herein, the term “human child” refers to a human that is 1 year to 18 years old. As used herein, the term “human infant” refers to a newborn to 1 year old human. As used herein, the term “human toddler” refers to a human that is 1 years to 3 years old. As used herein, the term “elderly human” refers to a human that is 65 years old and older.

In some embodiments, a patient treated or prevented in accordance with the methods provided herein is any subject at increased risk of SARS-CoV-2 infection or COVID-19 (e.g., an immunocompromised or immunodeficient individual). In some embodiments, a patient treated or prevented in accordance with the methods provided herein is any subject in close contact with an individual with increased risk of SARS-CoV-2 infection or COVID-19 (e.g., immunocompromised or immunosuppressed individuals).

In some embodiments, a patient treated or prevented in accordance with the methods provided herein is a subject affected by any condition that increases susceptibility to SARS-CoV-2 infection or complications or COVID-19. In other embodiments, a patient treated or prevented in accordance with the methods provided herein is a subject in which SARS-CoV-2 infection has the potential to increase complications of another condition that the individual is affected by, or for which they are at risk. In some embodiments, such conditions that increase susceptibility to SARS-CoV-2 complications or for which SARS-CoV-2 increases complications associated with the condition are, e.g., conditions that affect the lung, such as cystic fibrosis, asthma, chronic obstructive pulmonary disease, emphysema, or bacterial infections; cardiovascular disease; or diabetes. Other conditions that may increase SARS-CoV-2 complications include kidney disorders; blood disorders (including anemia or sickle cell disease); or weakened immune systems (including immunosuppression caused by medications, malignancies such as cancer, organ transplant, or HIV infection).

In some embodiments, a patient treated or prevented in accordance with the methods provided herein is a subject that resides in a group home, such as a nursing home or orphanage. In some embodiments, a patient treated or prevented in accordance with the methods provided herein is subject that works in, or spends a significant amount of time in, a group home, e.g., a nursing home or orphanage. In some embodiments, a patient treated or prevented in accordance with the methods provided herein is a health care worker (e.g., a doctor or nurse). In some embodiments, a patient treated or prevented in accordance with the methods provided herein resides in a dormitory (e.g., a college dormitory). In some embodiments, a patient treated or prevented in accordance with the methods provided herein is a member of the military. In some embodiments, a patient treated or prevented in accordance with the methods provided herein is a child that attends school or daycare.

In certain embodiments, patients treated or prevented in accordance with the methods provided herein are patients already being treated with antibiotics, antivirals, antifungals, or other biological therapy/immunotherapy.

6.6 Diagnostic Uses

The antibodies described herein (e.g., a monoclonal antibody, such as a chimeric or humanized antibody, or an antigen-binding fragment thereof) can be used for diagnostic purposes to detect SARS-CoV-2 as well as detect, diagnose, or monitor a SARS-CoV-2 infection.

Provided herein are methods for the detection of SARS-CoV-2 infection comprising: (a) detecting the expression of SARS-CoV-2 spike protein or a fragment thereof (e.g., RBD) in a biological specimen (e.g., sputum, nasal drippings, cells or tissue samples) from a subject using an antibody described herein (e.g., a monoclonal antibody, such as a chimeric or humanized antibody, or an antigen-binding fragment thereof); and (b) comparing the level of the SARS-CoV-2 spike protein or a fragment thereof (e.g., RBD) with a control level, e.g., levels in a biological specimen from a subject not infected with SARS-CoV-2, wherein an increase in the assayed level of SARS-CoV-2 spike protein c or a fragment thereof (e.g., RBD) compared to the control level of the SARS-CoV-2 spike protein or a fragment thereof (e.g., RBD) is indicative of SARS-CoV-2 infection.

Provided herein is a diagnostic assay for diagnosing SARS-Co-2 infection comprising: (a) assaying for the level of SARS-CoV-2 spike protein or a fragment thereof (e.g., RBD) in a biological specimen from a subject using an antibody described herein (e.g., a monoclonal antibody, such as a chimeric or humanized antibody, or an antigen-binding fragment thereof); and (b) comparing the level of the SARS-CoV-2 spike protein or a fragment thereof (e.g., RBD) with a control level, e.g., levels in a biological specimen from a subject not infected with SARS-CoV-2, wherein an increase in the assayed SARS-CoV-2 spike protein or a fragment thereof (e.g., RBD) level compared to the control level of the SARS-CoV-2 spike protein or a fragment thereof (e.g., RBD) is indicative of SARS-CoV-2 infection. A more definitive diagnosis of SARS-CoV-2 infection may allow health professionals to employ preventative measures or aggressive treatment earlier thereby preventing the development or further progression of the SARS-CoV-2 infection.

In one embodiment, provided herein is a method for detecting SARS-CoV-2, comprising: (a) contacting a biological sample (e.g., cells, sputum, nasal swab, mucous, etc.) with the antibody described herein; (b) detecting the binding of the antibody to SARS-CoV-2 spike protein or a fragment thereof (e.g., RBD), wherein SARS-CoV-2 is detected if the level of binding of the antibody to SARS-CoV-2 spike protein or a fragment thereof (e.g., RBD) is greater than the level of binding of the antibody to non-SARS-CoV-2 infected cells or a biological sample not infected with SARS-CoV-2. In one embodiment, the detection is done in vitro. In other embodiments, the detection is done in vivo. Techniques known to one of skill in the art may be used to detect the binding of the antibody to the SARS-CoV-2 spike protein or a fragment thereof (e.g., RBD).

Antibodies described herein (e.g., a monoclonal antibody, such as a chimeric or humanized antibody, or an antigen-binding fragment thereof) can be used to assay SARS-CoV-2 spike protein or a fragment thereof (e.g., RBD) levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Antibody-based methods useful for detecting protein expression include immunoassays, such as the enzyme linked immunosorbent assay (ELISA) and the radioimmunoassay (RIA). An antibody described herein or generated in accordance with the methods described herein may be labeled with a detectable label or a secondary antibody that binds to such an antibody may be labeled with a detectable label. Suitable antibody assay labels are known in the art and include enzyme labels, such as, glucose oxidase; radioisotopes, such as iodine (¹²⁵I, ¹²¹I) carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. See, above for examples of antibody conjugates that might be useful in the detection and diagnosis of SARS-CoV-2 infection.

In one embodiment, monitoring of SARS-CoV-2 infection is carried out by repeating the method for diagnosing the SARS-CoV-2 infection, for example, one day, two days, one week, two weeks, or one month after initial diagnosis.

6.7 Biological Assays

An antibody described herein (e.g., a monoclonal antibody, such as a chimeric or humanized antibody, or an antigen-binding fragment thereof) may be characterized using any assay known to one of skill in the art or described herein. In one embodiment, an antibody described herein is characterized as described in Section 5, infra.

6.7.1 Assays for Testing Antibody Activity

An antibody may be characterized in a variety of ways known to one of skill in the art (e.g., ELISA, biolayer interferometry, surface plasmon resonance display (BIAcore kinetic), Western blot, immunofluorescence, immunostaining, plaque reduction assays, and/or microneutralization assays). In some embodiments, an antibody is assayed for its ability to bind to SARS-CoV-2 spike protein or a fragment thereof (e.g., RBD). In certain embodiments, an antibody is assayed for its ability to inhibit or reduce the interaction of SARS-CoV-2 with its host cell receptor (e.g., ACE-2) using techniques known to one of skill in the art. For example, the ability of an antibody to inhibit or reduce the interaction of SARS-CoV-2 spike protein with ACE-2 may be tested using techniques known to one of skill in the art.

The specificity or selectivity of an antibody for SARS-CoV-2 spike protein or a fragment thereof (e.g., RBD) and cross-reactivity with other antigens can be assessed by any method known in the art. Immunoassays which can be used to analyze specific binding and cross-reactivity include, but are not limited to, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety).

The binding affinity of an antibody to SARS-CoV-2 spike protein or a fragment thereof (e.g., RBD) and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody for a SARS-CoV-2 spike protein or a fragment thereof (e.g., RBD) and the binding off-rates can be determined from the data by Scatchard plot analysis. Competition with a second antibody can also be determined using radioimmunoassays. In this case, a a SARS-CoV-2 spike protein or a fragment thereof (e.g., RBD), or or SARS-CoV-2 is incubated with the test antibody conjugated to a detectable labeled (e.g., ³H or ¹²⁵I) in the presence of increasing amounts of an unlabeled second antibody.

In some embodiments, surface plasmon resonance (e.g., BIAcore kinetic) analysis is used to determine the binding on and off rates of an antibody to SARS-CoV-2 spike protein or a fragment thereof (e.g., RBD), or SARS-CoV-2.

In some embodiments, an antibody described herein is tested for its ability to neutralize SARS-CoV-2 or SARS-CoV-2 spike protein expressing pseudotyped viruses, such as described in the Section 5, infra.

6.7.2 Cytotoxicity Assays

Many assays well-known in the art can be used to assess viability of cells (infected or uninfected) or cell lines following exposure to an antibody or composition thereof and, thus, determine the cytotoxicity of the antibody or composition thereof. For example, cell proliferation can be assayed by measuring Bromodeoxyuridine (BrdU) incorporation (See, e.g., Hoshino et al., 1986, Int. J. Cancer 38, 369; Campana et al., 1988, J. Immunol. Meth. 107:79), (3H) thymidine incorporation (See, e.g., Chen, J., 1996, Oncogene 13:1395-403; Jeoung, J., 1995, J. Biol. Chem. 270:18367 73), by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In one embodiment, the level of cellular ATP is measured to determined cell viability.

In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In one embodiment, cell viability can be measured in the neutral red uptake assay. In other embodiments, visual observation for morphological changes may include enlargement, granularity, cells with ragged edges, a filmy appearance, rounding, detachment from the surface of the well, or other changes. These changes may be given a designation of T (100% toxic), PVH (partially toxic-very heavy—80%), PH (partially toxic-heavy—60%), P (partially toxic—40%), Ps (partially toxic-slight—20%), or 0 (no toxicity—0%), conforming to the degree of cytotoxicity seen. A 50% cell inhibitory (cytotoxic) concentration (IC₅₀) is determined by regression analysis of these data.

In one embodiment, the cells used in the cytotoxicity assay are animal cells, including primary cells and cell lines. In some embodiments, the cells are human cells. In certain embodiments, cytotoxicity is assessed in one or more of the following cell lines: U937, a human monocyte cell line; primary peripheral blood mononuclear cells (PBMC); Huh7, a human hepatoblastoma cell line; 293T, a human embryonic kidney cell line; and THP-1, monocytic cells. In certain embodiments, cytotoxicity is assessed in one or more of the following cell lines: MDCK, MEF, Huh 7.5, Detroit, or human tracheobronchial epithelial (HTBE) cells.

An antibody or composition thereof can be tested for in vivo toxicity in animal models. For example, animal models, described herein and/or others known in the art, used to test the activities of an antibody or composition thereof can also be used to determine the in vivo toxicity of these antibodies. For example, animals are administered a range of concentrations of an antibody. Subsequently, the animals are monitored over time for lethality, weight loss or failure to gain weight, and/or levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and/or regimen in addition to dosages.

The toxicity and/or efficacy of an antibody or composition thereof can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. An antibody or composition thereof that exhibits large therapeutic indices is preferred. While an antibody or composition thereof that exhibits toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of an antibody or composition thereof for use in humans. The dosage of such antibodies lies preferably within a range of circulating concentrations that include the ED₅₀ with little or no toxicity. The dosage may vary within this range depending upon the dosage form employed and the route of administration utilized. For an antibody or composition thereof used in a method described herein, the effective dose can be estimated initially from cell culture assays. A dose may be formulated in animal models to achieve a circulating plasma concentration range that includes the IC₅₀ (i.e., the concentration of the antibody that achieves a half-maximal inhibition of symptoms) as determined in cell culture. Such information can be used to more accurately determine useful doses in humans. Levels in plasma may be measured, for example, by high-performance liquid chromatography. Additional information concerning dosage determination is provided herein.

Further, any assays known to those skilled in the art can be used to evaluate the prophylactic and/or therapeutic utility of an antibody or composition thereof, for example, by measuring viral infection or a condition or symptoms associated therewith.

6.7.3 In Vivo Assays

Antibodies and compositions thereof are preferably assayed in vivo for the desired therapeutic or prophylactic activity prior to use in humans. For example, in vivo assays can be used to determine whether it is preferable to administer an antibody or composition thereof and/or another therapy. For example, to assess the use of an antibody or composition thereof to prevent a disease associated with SARS-CoV-2 (e.g., COVID-19), the antibody or composition can be administered before the animal is infected with SARS-CoV-2. Alternatively, or in addition, an antibody or composition thereof can be administered to the animal at the same time that the animal is infected with SARS-CoV-2. To assess the use of an antibody or composition thereof to treat SARS-CoV-2 infection or a disease associated therewith (e.g., COVID-19), the antibody or composition may be administered after infecting the animal with SARS-CoV-2. In one embodiment, an antibody or composition thereof is administered to the animal more than one time.

In general, animals are infected with SARS-CoV-2 and concurrently or subsequently treated with an antibody or composition thereof, or placebo. Alternatively, animals are treated with an antibody or composition thereof or placebo and subsequently infected with SARS-CoV-2. Samples obtained from these animals (e.g., serum, urine, sputum, semen, saliva, plasma, or tissue sample) can be tested for viral replication via well known methods in the art, e.g., those that measure altered viral titers (as determined, e.g., by plaque formation), the production of viral proteins (as determined, e.g., by Western blot, ELISA, or flow cytometry analysis) or the production of viral nucleic acids (as determined, e.g., by RT-PCR or northern blot analysis). For quantitation of virus in tissue samples, tissue samples are homogenized in phosphate-buffered saline (PBS), and dilutions of clarified homogenates are adsorbed for a time period (e.g., 20 minutes or 1 hour) at 37° C. onto monolayers of cells (e.g., Vero, CEF or MDCK cells). In other assays, histopathologic evaluations are performed after infection, preferably evaluations of the organ(s) the virus is known to target for infection. Virus immunohistochemistry can be performed using a viral-specific monoclonal antibody.

The effect of an antibody or composition thereof on the infectious disease process or pathogenicity of a given virus can also be determined using in vivo assays in which the titer of the virus in an infected subject administered an antibody or composition thereof, the length of survival of an infected subject administered an antibody or composition thereof, the immune response in an infected subject administered an antibody or composition thereof, the number, duration and/or severity of the symptoms in an infected subject administered an antibody or composition thereof, and/or the time period before onset of one or more symptoms in an infected subject administered an antibody or composition thereof, is assessed. Techniques known to one of skill in the art can be used to measure such effects.

In yet other assays, histopathologic evaluations are performed after infection of an animal model subject. Nasal turbinates and trachea may be examined for epithelial changes and subepithelial inflammation. The lungs may be examined for bronchiolar epithelial changes and peribronchiolar inflammation in large, medium, and small or terminal bronchioles. The alveoli are also evaluated for inflammatory changes.

Virus immunohistochemistry may be performed using a viral-specific monoclonal antibody (e.g. spike-specific monoclonal antibodies).

In one embodiment, the ability of an antibody or composition thereof to treat SARS-CoV-2 infection or a disease associated therewith (e.g., COVID-19) is assessed by determining the ability of the antibody to confer passive immunity to a disease associated with SARS-CoV-2 infection (e.g., COVID-19) in a subject. The ability of an antibody described herein to confer passive immunity to a disease associated with SARS-CoV-2 infection (e.g., COVID-19) in a subject can be assessed using any methods known in the art.

6.7.4 Assays in Humans

In one embodiment, an antibody or composition thereof that modulates replication of SARS-CoV-2 is assessed in infected human subjects. In accordance with this embodiment, an antibody or composition thereof is administered to the human subject, and the effect of the antibody and/or composition on viral replication is determined by, e.g., analyzing the level of the virus or viral nucleic acids in a biological sample (e.g., serum or plasma). An antibody or composition thereof that alters virus replication can be identified by comparing the level of virus replication in a subject or group of subjects treated with a control antibody to that in a subject or group of subjects treated with an antibody or composition thereof. Alternatively, alterations in viral replication can be identified by comparing the level of the virus replication in a subject or group of subjects before and after the administration of an antibody or composition thereof. Techniques known to those of skill in the art can be used to obtain the biological sample and analyze the mRNA or protein expression.

In another embodiment, the effect of an antibody or composition thereof on the severity of one or more symptoms associated with SARS-CoV-2 infection/COVID-19 are assessed in an infected subject. In accordance with this embodiment, an antibody or composition thereof or a control antibody is administered to a human subject suffering from SARS-CoV-2 infection and the effect of the antibody or composition on one or more symptoms of the virus infection is determined. An antibody or composition thereof that reduces one or more symptoms can be identified by comparing the subjects treated with a control antibody to the subjects treated with the antibody or composition. Techniques known to physicians familiar with infectious diseases can be used to determine whether an antibody or composition thereof reduces one or more symptoms associated with a SARS-CoV-2 infection (e.g., COVID-19).

6.8 Kits

In another aspect, provided herein is a pharmaceutical pack or kit comprising one or more containers filled with one or more of the ingredients of a composition (e.g., a pharmaceutical compositions) described herein, such as one or more antibodies provided herein (e.g., a monoclonal antibody, such as a chimeric or humanized antibody, or an antigen-binding fragment thereof), one or more polynucleotides described herein, or one or more antibody conjugates described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in the above methods. In one embodiment, a kit comprises an antibody described herein, preferably an isolated antibody, in one or more containers. An antibody described herein included in a kit may be attached to a solid support (e.g., a microtiter plate or bead). In one embodiment, the kits encompassed herein contain an isolated SARS-CoV2 antigen that the antibodies encompassed herein react with (e.g., the antibody binds to the antigen) as a control. In one embodiment, the kits provided herein further comprise a control antibody which does not react with SARS-CoV-2 spike protein or a fragment thereof (e.g., RBD) (such as a control IgG). In one embodiment, the kits provided herein contain a means for detecting the binding of an antibody to SARS-CoV-2 spike protein or a fragment thereof (e.g., the antibody may be conjugated to a detectable substrate such as a fluorescent compound, an enzymatic substrate, a radioactive compound, a luminescent compound, or another antibody that is conjugated to a detectable substrate (e.g., the antibody may be conjugated to a second antibody which recognizes/binds to the first antibody)). In certain embodiments, the kits comprise a second antibody which is labeled with a detectable substance and which binds to an antibody described herein. In specific embodiments, the kit may include a recombinantly produced or chemically synthesized SARS-CoV-2 spike protein or a fragment thereof (e.g., RBD). The SARS-CoV-2 spike protein or a fragment thereof (e.g., RBD) provided in the kit may also be attached to a solid support. In a more specific embodiment, the detecting means of the above described kit includes a solid support to which SARS-CoV-2 spike protein or a fragment thereof (e.g., RBD) is attached. Such a kit may also include a non-attached reporter-labeled antibody. In this embodiment, binding of the antibody to the SARS-CoV-2 spike protein or a fragment thereof (e.g., RBD) can be detected by binding of the said reporter-labeled antibody.

### Example 1: Production and Testing of Antibodies

The entry of SARS-CoV2 into cells requires the envelope S glycoprotein engagement with the human angiotensin-converting enzyme 2 gene (ACE2) cell surface protein. SARS-CoV2 S protein is a type I membrane protein with a large extracellular region. The S1 domain binds to ACE2 through its receptor-binding domain (RBD) leading to endocytosis of the virus. Similar to other viruses (e.g., influenza), monoclonal antibodies (mAbs) against regions of the S1 domain including the RBD can prevent infection by blocking virus binding. The S2 region of the spike is involved in fusion and is another target for monoclonal antibody therapeutics. This example describes unique monoclonal antibodies that neutralize the SARs-Cov2 virus in the low pM range.

Immunization: Harbour mice were immunized with either (1) full length spike or (2) a receptor binding domain (RBD) Fc fusion protein in adjuvant. Harbour mice use human VH and VL genes and rat constant region genes to make antibodies that can be rapidly converted to fully human by a simple cloning step. Multiple mice were fused, clones selected and the antibodies they produced tested by ELISA, Flow and inhibition of ACE2 binding.

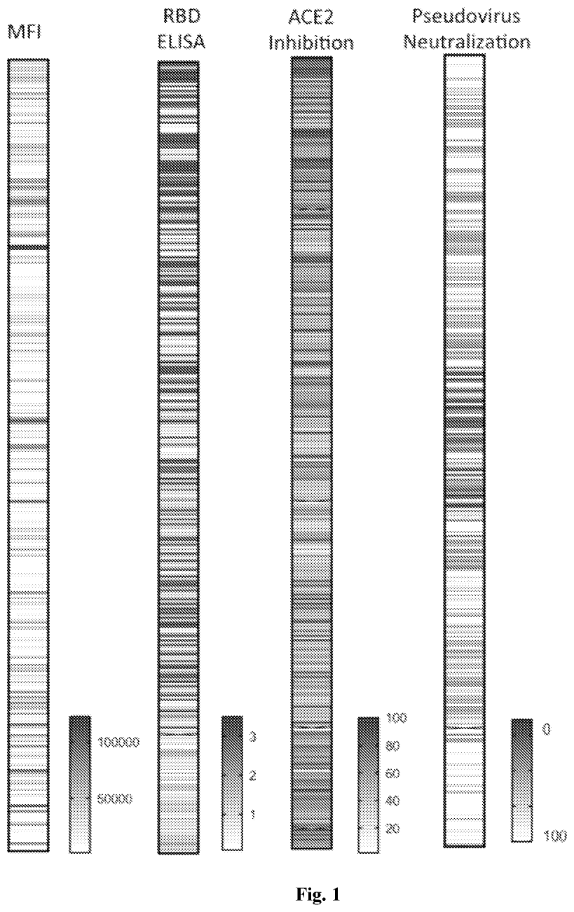

Preliminary screening: The clones were screened in several assays that included binding to membrane bound whole spike on the cell surface (MFI/flow cytometry), binding to receptor binding domain (RBD) of spike protein (RBD ELISA), RBD/ACE2 competition assay (ACE2 inhibition), and pseudovirus neutralization assay (pseudovirus neutralization). In particular, the clones targeting SARS-CoV spike protein were subjected to a flow cytometry assay using HEK-293 cells expressing Spike protein. Clones that demonstrated a mean fluorescence intensity (MFI)>2×background were selected for further analysis. The heat map shows the diversity of mAb binding to the spike protein. The analysis of flow-positive clones using an ELISA in which SARS-CoV-2 RBD was the target protein revealed a significant number of clones that strongly bound to the RBD based on optical density. These clones were also evaluated using an RBD/ACE2 competition assay and calculated as % inhibition. Finally, the clones were subjected to a VsV-pseudovirus neutralization based on no antibody at 0% neutralization. See FIG. 1.

Animal sera and mAb clones were selected if they bound to the surface of Expi293 cells expressing full length spike protein as measured by flow cytometry. In addition, binding to the RBD region was also measured by ELISA and analyzed by an RBD/ACE2 competition assay that measure the inhibition of RBD binding to ACE2 expressed on the surface of HEK-293 cells. In addition, the clones were subjected to inhibition of VSV-Spike pseudovirus infection based on the expression of GFP encoded in the pseudovirus. Briefly, the mAb was preincubated with the pseudovirus and analyzed for infection through measuring virus-infected cells or GFP fluorescence intensity.

The clones for further analysis were selected based on high binding for spike protein and RBD, ACE2 inhibition, and inhibition of SARs-CoV2/VSV-pseudo virus infectivity. These clones were further analyzed using a SARs-CoV2/VSV pseudo virus neutralization assay.

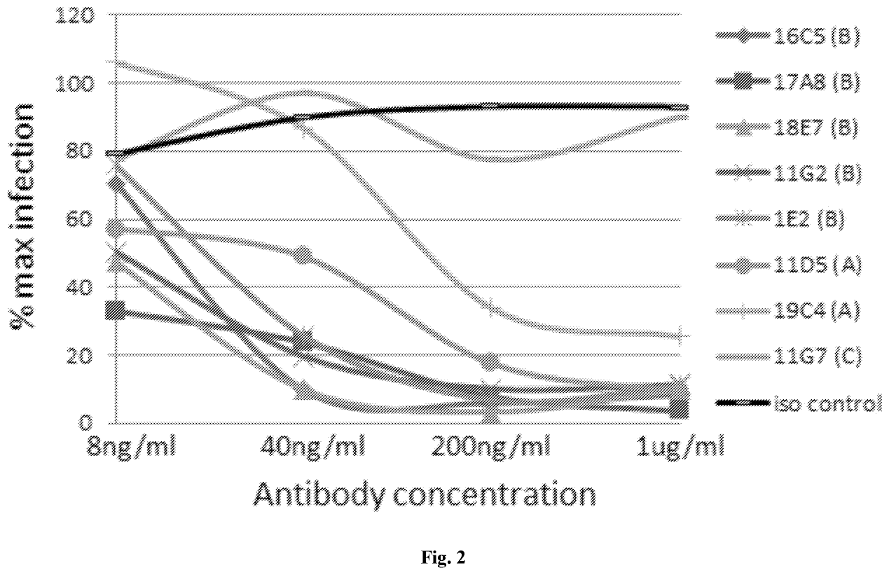

Neutralizing titer testing: Antibodies positive in all four of the above assays were tested further for neutralization titers (EC50s) of a SARs-CoV2 Spike/VSV pseudovirus particle infection of Vero E6 cells based on a dilution range. Dilutions of antibodies were premixed with the virus and blocking of infection was measured by the loss of GFP as measured by flow cytometry. The EC50 values were calculated based on concentration dependent inhibition (FIG. 2) The EC50 values and ACE2 inhibition (Tables 3 and 4) was used as a selection criteria for sequencing and humanization.

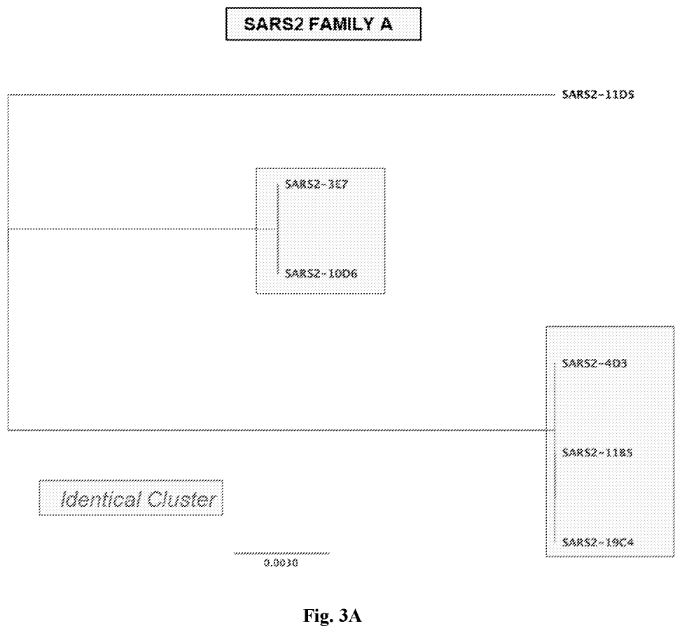

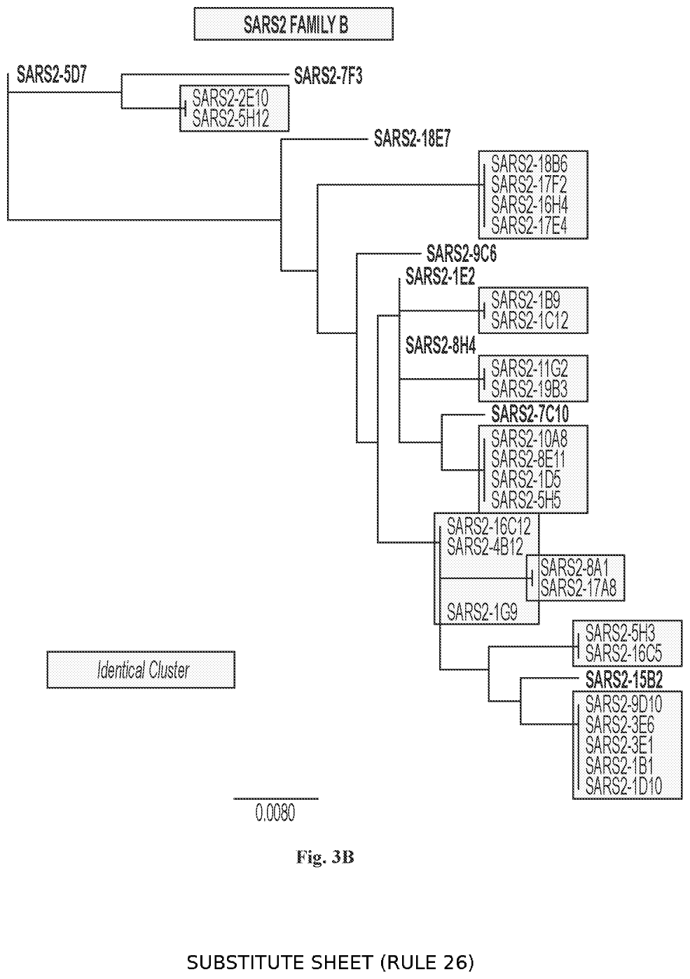

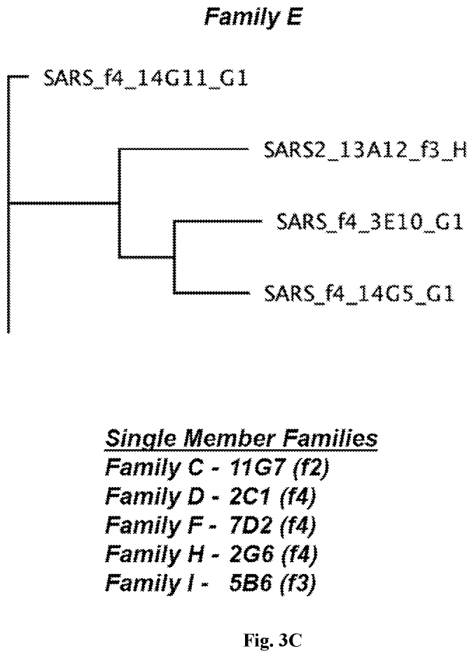

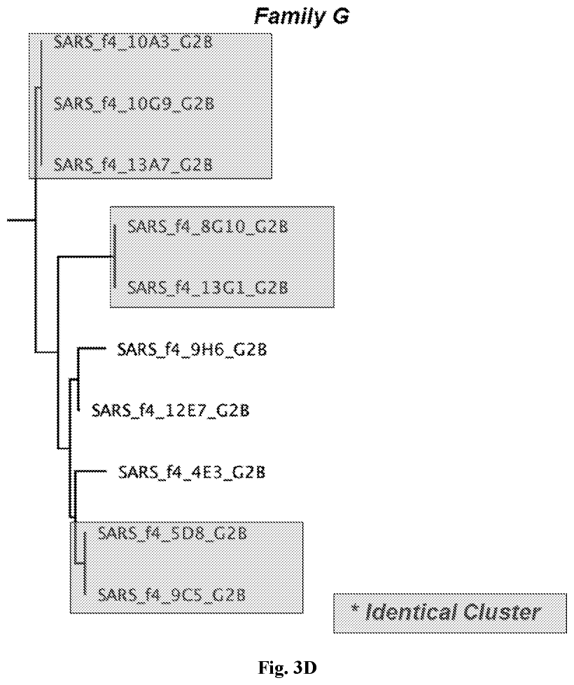

Sequencing: Total RNA was extracted from the hybridomas and converted to cDNA. Antibody specific PCRs were performed to amplify the variable regions of the heavy and light chains. Purified PCR products were submitted for sanger sequencing (GeneWiz). Sequences were blasted using IMGT V-quest (www.imgt.org) to identify matching human variable gene family members, somatic mutation variances, and junctions (CDR3s). Antibodies were grouped together in families based on CDR3 identity, which determines antibody clonality. Clones that had completely identical sequences were considered exact copies and only one candidate was chosen to move forward from such identical clusters (FIG. 3).

Addition of human constant regions: DNA for the variable regions of lead antibodies is synthesized from sequence information and cloned in frame into mammalian expression vectors containing human G1 and kappa constant regions (GenScript). DNA vectors is grown, prepped (mini/maxi) and used for transfection in mammalian cells for antibody production. To validate the antibodies, corresponding heavy/light pairs are transfected into Expi293 cells and supernatants collected for testing and purification (see section “purification and validation of fully human mAb”).

Epitope mapping: Epitope mapping of neutralizing antibodies can be accomplished using site directed mutational analysis. By scanning the Spike/RBD and replacing certain amino acids with alanines, one epitope at a time can be disrupted. This procedure requires approximately 20 clones to be made, and then expressed in cells. Once produced the mutant spike expression plasmids is used to transfect Expi293 cells. Changes in binding are measured by flow cytometry using the 19 antibodies and comparing the binding to control spike expression.

Purification and validation of fully human mAbs: Expression vectors are transfected into Expi293 cells and supernatants collected for testing and purification. Antibodies are purified on protein A/G HiTrap columns using fast protein liquid chromatography (FPLC) on an AKTA Chromatography system (GE). In order to be certain that the specificity has not been altered by the process, purified, fully human antibodies are tested for binding to RBD by ELISA, to full length spike by flow cytometry and in the pseudovirus neutralization assay. In the PsV assay it is expected that the EC50 will not have changed from the pre-humanized versions.

Evaluation of ability of mAb to target immune cells to kill infected cells: The neutralizing mAbs are subjected to an Antibody-dependent cell-mediated cytotoxicity (ADCC) assay to evaluate the anti-SARS-CoV2 antibodies to recognize virus cells and induce immune cell killing. In general, HEK-293 cells expressing spike protein and SARS-CoV2 infected cells are included with increasing concentrations (0-25 μg/ml) of each antibody followed by the addition of ADCC target cell. After co-culture, Cyto-tox reagent (Promega) are used to quantitatively evaluate dead cells in the co-culture.

Neutralization experiments: Dilutions of mAb (500 ng/ml to 1 ng/ml) are mixed with a fixed amount of pseudovirus. Purified antibody alone or equal mixtures of two mAbs are performed and EC50 values determined. Complementary antibodies could show lower EC50 than either of the single mAbs or could provide protection from escape mutations. For identifying escape mutants, SARs-Cov2 spike pseudovirus assays are performed under selection pressure from one mAb, or a combination of mAbs. After 4 days in culture, virus is collected and used to infect a fresh well of Vero E6 cells, again under the selection of suboptimal concentrations of mAb. After another 4 days, virus is collected and subject to deep sequencing.

Affinity and binning analysis: Affinity (K_{D}) for purified antibodies is determined using bio-layer interferometry (BLI) on an Octet RED 96 with a tagged RBD only protein. Antibody pairs are also be binned against each other in similar format to determine if one antibody competes for binding of a second antibody, indicating either competing or non-competing epitope binding. Antibody pairs are chosen based on antibody family designations with pairs representing members of different families. Regeneron antibody pairs did not show a major improvement in neutralization when mixed together even though they bound to different sites. However, when analyzed for suppression of mutant escape such antibodies successfully block the appearance of mutant viruses.

Neutralization of live virus in BSL3 facility: EC50 of neutralization is performed as was done with the pseudovirus assay but under BSL3 conditions and with minor changes. Dilutions of antibodies (0-25 μg/ml) will be mixed and inoculated onto Vero E6 cells. 24 hours later the infected cells are washed and stained with 1C7, a monoclonal antibody specific for the nucleoprotein of the virus followed by an Alexa647 fluorophore tagged mouse IgG specific secondary antibody. The cell monolayers are fixed and fluorescence measured on a Celligo cytometer.

In vivo testing of mAb in hamster model of infection: The efficacy of the mAb is determined using a hamster model of SARsCoV2 infection. Animals are infected by intranasal inoculation with a predetermined dose of virus (100TCID50). One hour post infection the animals receive an intravenous administration of the antibodies to be tested. Two doses, 100 μg and 500 μg of each antibody are injected. Animal weights are monitored daily and on day 5 test animals are euthanized, lungs collected, homogenized and analyzed for viral titers by inoculation onto VeroE6 as described above. Experiments are done with individual lead antibodies and selected combinations of antibodies. Irrelevant human antibody serve as control.

Summary: The antibodies that utilize human V regions, neutralize SARsCoV2 in the pM range (Tables 3 and 4). All mAbs are of a rat IgG2a or 2b isotype. All mAbs have been sequenced (see Tables 1, 2, and 5-10) and show some degree of uniqueness with each other.

### Example 2: Antibodies of Family E Bind to Different Epitopes than Antibodies of Families A and B

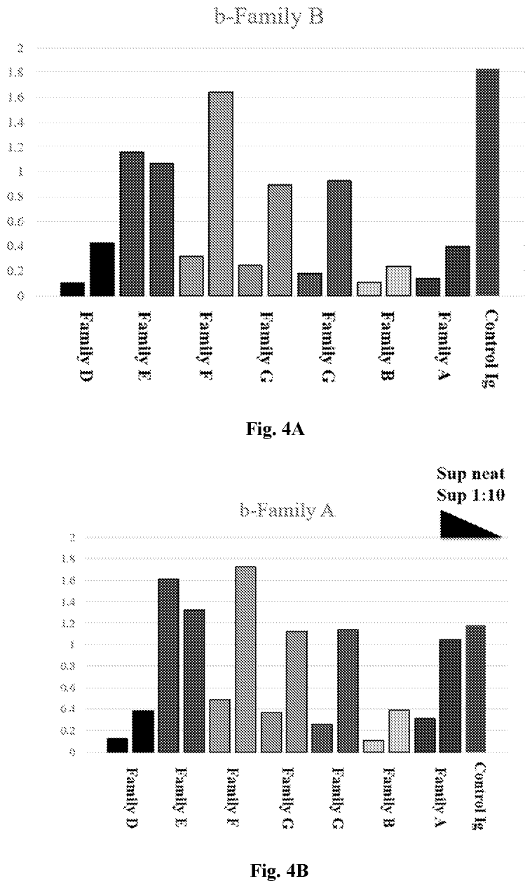

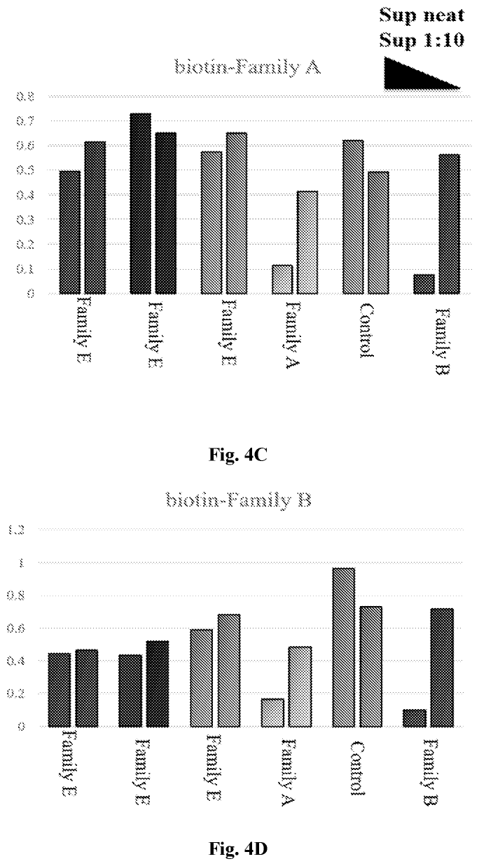

To determine epitope competition between families of antibodies, competitive ELISAs were performed on SARS-CoV RBD coated plates using supernatants (either undiluted (neat) or at a 1:10 dilution in sera free media) representing clones from each family. Rat IgG was used as an isotype control (1 μg/ml). Biotinylated purified antibody from family A (10D6) or B (16C5) was added after the supernatants and developed with streptavidin HRP. Representative clones included 19C4 (family A), 16C12 (family B), 2C1 (family D), 14G5 (family E), 7D2 (family F), and 10A3 (family G) (FIGS. 4A and 4B). Three family E clones (3E10, 14G5, and 14G11) were examined for competition with family A (FIG. 4C) and B (FIG. 4D) biotinylated monoclonals.

None of the family E antibodies competed with family A and B antibodies for epitope binding (FIG. 4), indicating that family E antibodies bind to different epitopes from those epitopes bound by families A and B.

### Example 3: Antibodies Neutralized WT Pseudovirus and Pseudovirus Variants

GFP expressing, VSV based pseudoviruses with either wildtype or major single variant mutation spike proteins N501Y (observed in the α (i.e., UK) mutant virus) and E484K (observed in the β (i.e., the South Africa) mutant virus) were mixed with purified human antibodies from each of the major antibody clonal families a concentration range from 5 μg/ml to 5 ng/ml. Prior to neutralization, hybridoma supernatants grown in SFM (sera free hybridoma media) (Invitrogen) were quantitated using an Octet Red96 by diluting supernatants 1:5 and 1:10 in sera free media and measured for binding against the Anti-Murine IgG Quantitation (AMQ) Biosensors (with cross reactivity to rat IgG Fc) on an Octet Red 96 BLI Instrument (SartoriusAG, Goettingen, Germany). Results were compared to in-lab derived purified rat IgG standards diluted in SFM in the range of 0.5-50 μg/ml. For neutralization, VsV-SARS-spike GFP-expressing reporter virus (PMCID: PMC8313705) was pre-incubated with mouse sera (1:100-1:3200), hybridoma supernatants (1:10-1:10,000), or purified human monoclonal antibodies (0.1 ng/ml-1 μg/ml) and incubated at 4° C. for 1 hr before the inoculum was added either to Vero E6 cells or to HEK-293 cells expressing Transmembrane Serine Protease-2 (PMCID: PMC8313705) overnight at 37° C., 5% CO₂. The cells were resuspended in cold FACS buffer and analyzed by flow cytometry (Intellicyte Corp.) for GFP fluorescence intensity. Cells with a high mean fluorescence intensity were identified using FlowJo software (Tree Star, Inc.) and graphed using GraphPad Prism to create a heat map based on MFI. A human isotype was used as a control (IgG1). Representative clones included 19C4 (family A), 16C12 (family B), 2C1 (family D), 14G5 (family E), 7D2 (family F), and 10A3 (family G).

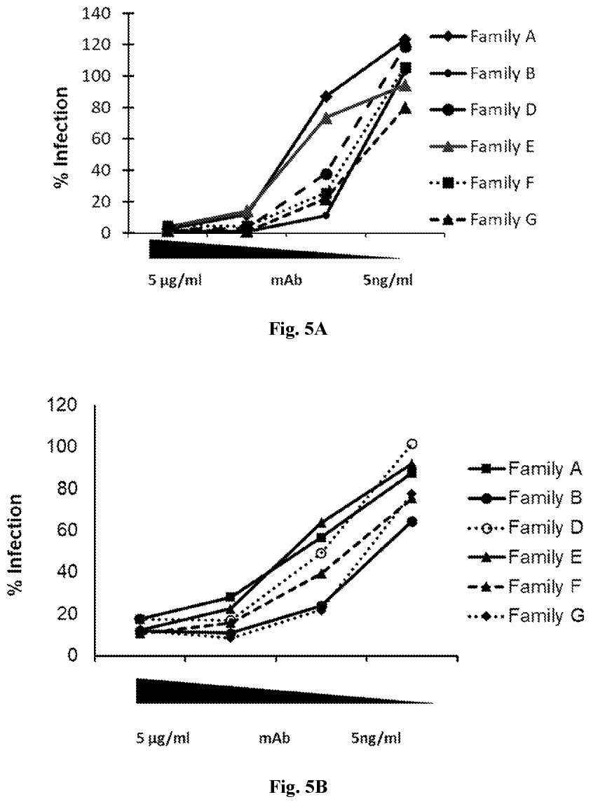

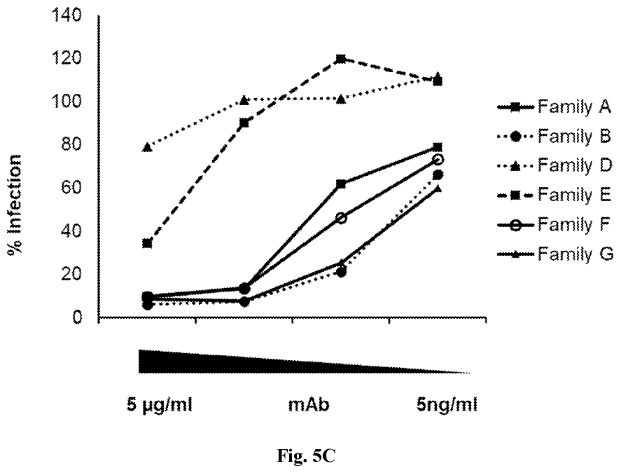

All genetic clonal families of lead antibodies neutralized WT pseudovirus, and N501Y single mutation pseudovirus (FIG. 5).

EC₅₀ values for different antibodies and pseudovirus variants are shown in Table 11.

TABLE 11
Neutralization of different pseudovirus variants by selected antibodies.
B1.1.7 is the UK alpha variant without the E484K mutation (VOC-20DEC-01).

| | | Pseudovirus Mutant Variants Neutralization (EC50 ng/ml) | | | | |
|---|---|---|---|---|---|---|
| Genetic | Clone | J15 | | | | Inhibition |
| family | name | (WT) | 484 | 501 | B1.1.7 | of family B |
| Family I | 5B6 (f3) | 32.4 | 35.2 | 28.2 | 15.3 | yes |
| Family E | 13A12 (f3) | 67.8 | 221 | 29 | 32.8 | no |
| Family H | 2G6 (f4) | 157 | 1457 | 152 | 51.4 | no |

### Example 4: ELISA Binding Analysis of Human Neutralizing mAbs to Wild Type-RBD, South African (SA)-RBD Variant, and N501Y-RBD Point Mutant

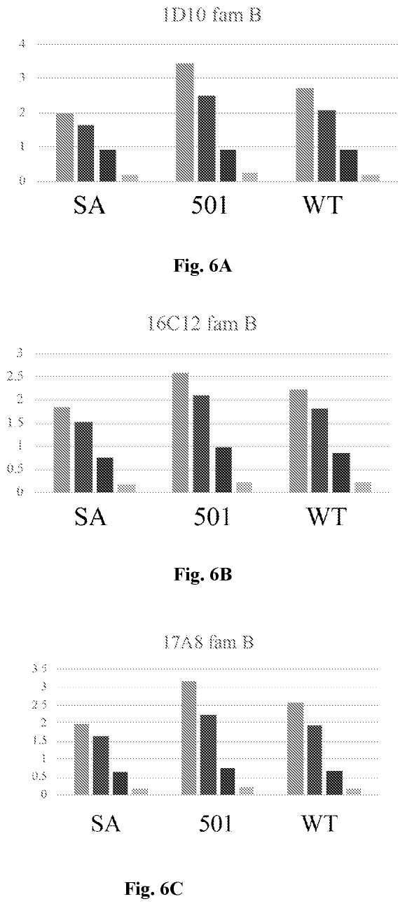

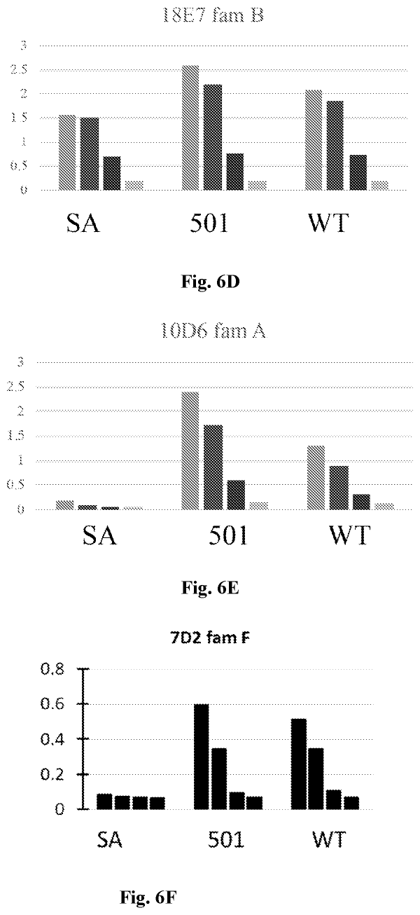

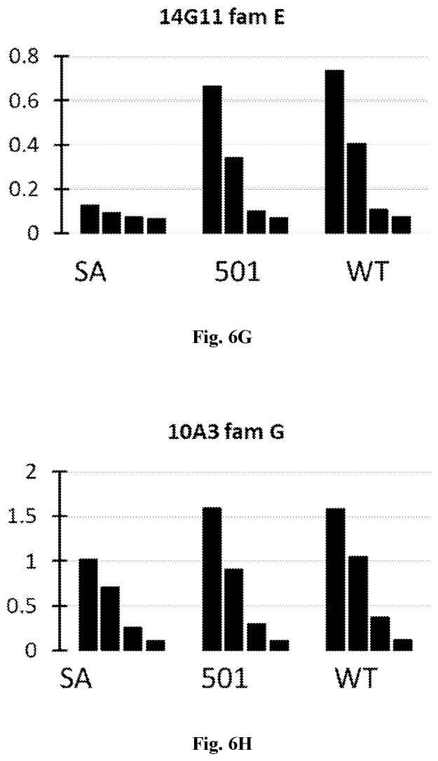

Fusion proteins representing the RBD domains of the SARS-CoV2 Spike protein from the wild type, the N501Y single mutant, or the South African (SA, E484K) variant were coated to ELISA plates and screened for binding as described in the materials and methods. Monoclonal antibodies representing each of the genetic families were added to coated ELISA plates at concentrations between 2 μg/ml to 2 ng/ml. Absorbance was read at 450 nm. All antibody families bound to WT and N501Y RBDs by ELISA (FIG. 6).

### Example 5: Anti-SARS-CoV2 RBD mAbs Block Virus Proliferation In Vivo

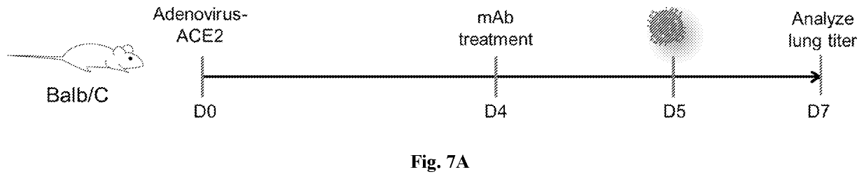

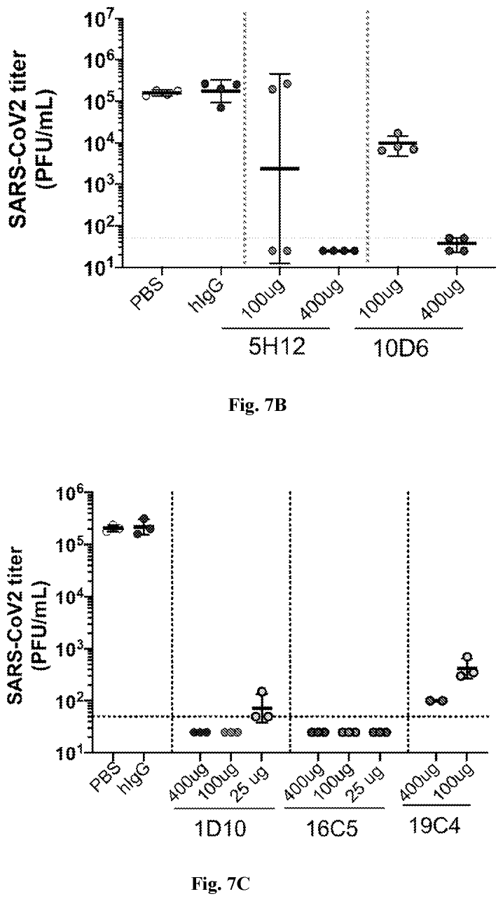

BALB/c mice were sensitized with Ad5-hACE2, treated with anti-RBD monoclonal antibodies, and challenged with SARS-CoV-2 (FIG. 7A). Specifically, nine to twelve-week old female BALB/c mice were sensitized with Ad5-hACE2 via intranasal route at 2.5×10⁸ PFU/animal five days prior to the challenge of SARS-CoV-2. Mice were transferred to BSL-3 facility 2 or 3 days before infection for acclimatization. One day before the challenge, each mouse received 200 μl of antibody (5H12, 10D6, 1D10, 16C5, or 19C4) at the designated amounts via intraperitoneal (IP) route. Two controls groups that received human IgG negative control or PBS were included. Twenty-four hours after the transfer, mice were challenged with 105 PFU of USA-WA1/2020 SARS-CoV-2 strain (day 0). The animals were sacrificed at day 2 post-challenge, when the virus replication peaks, to harvest lungs. Lung lobes from each animal were homogenized in 1 mL PBS. Plaque assays using Vero E6 cells were performed in the biosafety level 3 (BSL-3) facility following institutional guidelines to quantify infectious viral titers in the lung homogenates, in which plaque forming unit (PFU)/mL was used as the readout.

The panel of mAbs effectively and prophylactically blocked virus replication in the lungs in contrast to human IgG negative control and PBS, with almost no detectable titers in some groups (FIGS. 7B and 7C).

TABLE 1
Variable_Heavy Chain Sequences.
Antibodies are also referred to as SARS2-[antibody name] in the specification.
For example, SARS2-1C12_f3_H and 1C12_f3_H refer to the same antibody.

| | | | | Variable Heavy Chain | |
|---|---|---|---|---|---|
| | | Variable_Heavy Chain Nucleotide Sequence | | Amino Acid Sequence | |
| | | SEQ | | SEQ | |
| Ab # | Name | ID NO | Sequence | ID NO | Sequence |
| 1 | 1C12_f3_H | 1 | CAGGTGCAGCTGGTGCAGTCTGGGGCTGAGGTGAAGAAGCCTGGG | 59 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCACCTGGGTGCGACAGGCCACTGGACAAGGGCTTGA | | YDITWVRQATGQGLE |
| | | | GTGGATGGGATGGATGAGCCCTAACAGTGGTAACACAGGCTATAC | | WMGWMSPNSGNTGY |
| | | | ACAGAAGTTCCAGGGCAGAGTCACCATGACCAGGAACACCTCCAT | | TQKFQGRVTMTRNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 2 | 1D5_f3_H | 2 | CAGGTGCAGCTGGTGCAGTCTGGGGCTGAGGTGAAGAAGCCTGGG | 60 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCACCTGGGTGCGACAGGCCACTGGACAAGGGCTTGA | | YDITWVRQATGQGLE |
| | | | GTGGATGGGATGGATGAGCCCTAACAGTGGTAACACAGGCTATGC | | WMGWMSPNSGNTGY |
| | | | ACAGAAGTTCCAGGGCAGAGTCACCATGACCAGGAACACCTCCAT | | AQKFQGRVTMTRNTSI |
| | | | AAGCACAGCCTACATGGAGCTGAACAGCCTGAGATCTGAGGACAC | | STAYMELNSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 3 | 1D10_f3_H | 3 | CAGGTGCAGCTGGTGCAGTCTGGGGCTGAAGTGAAGAAGCCTGGG | 61 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCATCTGGGTGCGACAGGCCTCTGGACAAGGGCTTGA | | YDIIWVRQASGQGLE |
| | | | GTGGATGGGATGGATGAGCCCTAACAGCGGTAACACAGGCTATGC | | WMGWMSPNSGNTGY |
| | | | ACAGAAGTTCCAGGGCAGAGTCACCATGACCAGGAACACCTCCAT | | AQKFQGRVTMTRNTSI |
| | | | AAACACAGCCTACATGGAGCTGAGTAGCCTGAGATCTGAGGACAC | | NTAYMELSSLRSEDTA |
| | | | GGCCGTGTATTATTGTGCGAGATTCGGCTATGGTTCGGGGGCCCTC | | VYYCARFGYGSGALD |
| | | | GACTACTACTATTACGGTTTGGACGTCTGGGGCCAAGGGACCACGG | | YYYYGLDVWGQGTTV |
| | | | TCACCGTCTCCTCA | | TVSS |
| 4 | 1E2_f3_H | 4 | CAGGTGCAGCTGGTGCAGTCTGGGGCTGAGGTGAAGAAGCCTGGG | 62 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCACCTGGGTGCGACAGGCCACTGGACAAGGGCTTGA | | YDITWVRQATGQGLE |
| | | | GTGGATGGGATGGATGAGCCCTAACAGTGGTAACACAGGCTATGC | | WMGWMSPNSGNTGY |
| | | | ACAGAAGTTCCAGGGCAGAGTCACCATGACCAGGAACACCTCCAT | | AQKFQGRVTMTRNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 5 | 1G9_f3_H | 5 | CAGGTGCAGCTGGTGCAGTCTGGGGCTGAGGTGAAGAAGCCTGGG | 63 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCATTTGGGTGCGACAGGCCACTGGACAAGGGCTTGA | | YDIIWVRQATGQGLE |
| | | | GTGGATGGGATGGATGAGCCCTAACAGTGGTAACACAGGCTATGC | | WMGWMSPNSGNTGY |
| | | | ACAGAAGTTCCAGGGCAGAGTCACCATGACCAGGAACACCTCCAT | | AQKFQGRVTMTRNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 6 | 5D7_f3_H | 6 | CAGGTGCAGCTGGTGCAGTCTGGGGCTGAGGTGAAGAAGCCTGGG | 64 | QVQLVQSGAEVKKPG |
| | | | ACCTCAGTGAAAGTCTCCTGCAAGACTTCTGGATACACCTTCACCA | | TSVKVSCKTSGYTFTS |
| | | | GTTATGATATCATCTGGGTGCGACAGGCCACTGGACAAGGGCTTGA | | YDIIWVRQATGQGLE |
| | | | GTGGATGGGATGGATGAGCCCTAAAAATGGTAACACAGGCTATGC | | WMGWMSPKNGNTGY |
| | | | ACAGAGGTTCCAGGGCAGAGTCACCATGACCAGGAACACCTCCAT | | AQRFQGRVTMTRNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 7 | 5H12_f3_H | 7 | CAGGTGCAGCTGGTGCAGTCTGGGGCTGAGGTGAAGAAGCCTGGG | 65 | QVQLVQSGAEVKKPG |
| | | | ACCTCAGTGAAAGTCTCCTGCAAGACTTCTGGATACACCTTCCCCA | | TSVKVSCKTSGYTFPS |
| | | | GTTATGATATCATCTGGGTGCGACAGGCCACTGGACAAGGGCTTGA | | YDIIWVRQATGQGLE |
| | | | GTGGATGGGATGGATGAGCCCTAAAAATGGTAACACAGGCTTTGCA | | WMGWMSPKNGNTGF |
| | | | CAGAGGTTCCAGGGCAGAGTCACCATGACCAGGAACACCTCCATA | | AQRFQGRVTMTRNTSI |
| | | | AGCACAGCCTACATGGAACTGAGCAGCCTGAGATCTGAGGACACG | | STAYMELSSLRSEDTA |
| | | | GCCGTGTATTACTGTGCGAGATTCGGCTATGGGTCGGGGGCCCTCG | | VYYCARFGYGSGALG |
| | | | GATATTACTACTACGGTTTGGACGTCTGGGGCCAAGGGACCACGGT | | YYYYGLDVWGQGTTV |
| | | | CACCGTCTCCTCA | | TVSS |
| 8 | 7C10_f3_H | 8 | CAGGTGCAGCTGGTGCAGTCTGGGGCTGAGGTGAAGAAGCCTGGG | 66 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCACCTGGGTGCGACAGGCCACTGGACAAGGGCTTGA | | YDITWVRQATGQGLE |
| | | | GTGGATGGGGTGGATGAGCCCTAACAGTGGTAACACAGGCTATGC | | WMGWMSPNSGNTGY |
| | | | ACAGAAGTTCCAGGGCAGAGTCACCATGACCAGGAACACCTCCAT | | AQKFQGRVTMTRNTSI |
| | | | AAGCACAGCCTACATGGAGCTGCGCAGCCTGAGGTCTGAGGACAC | | STAYMELRSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 9 | 8H4_f3_H | 9 | CAGGTGCAGCTGGTGCAGTCTGGGGCTGAGGTGAAGAAGCCTGGG | 67 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCACCTGGGTGCGACAGGCCACTGGACAAGGGCTTGA | | YDITWVRQATGQGLE |
| | | | GTGGATGGGATGGATGAGCCCTAACAGTGGTAACACAGGCTATGC | | WMGWMSPNSGNTGY |
| | | | ACAGAAGTTCCAGGGCAGAGTCACCATGACCAGGAACACCTCCAT | | AQKFQGRVTMTRNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 10 | 9C6_f3_H | 10 | CAGGTGCAGCTGGTGCAGTCTGGGGCTGAGGTGAAGAAGCCTGGG | 68 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCACCTGGGTGCGACAGGCCACTGGACAAGGGCTTGA | | YDITWVRQATGQGLE |
| | | | GTGGATGGGATGGATGAGCCCTAAAAGTGGTAACACAGGCTATGC | | WMGWMSPKSGNTGY |
| | | | ACAGAAGTTCCAGGGCAGAGTCACCATGACCAGGAACACCTCCAT | | AQKFQGRVTMTRNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 11 | 10D6_f3_H | 11 | GAGGTGCAGTTGGTGGAGACTGGAGGAGGCTTGATCCAGCCTGGG | 69 | EVQLVETGGGLIQPGG |
| | | | GGGTCCCTGAGACTCTCCTGTGCAGCCTCTGGGATCACCGTCAGTA | | SLRLSCAASGITVSSNY |
| | | | GTAACTACATGAACTGGGTCCGCCAGGCTCCAGGGAAGGGGCTGG | | MNWVRQAPGKGLEW |
| | | | AGTGGGTCTCAGTTATTTATAGCGGTGGTAGCACATACTACGCAGA | | VSVIYSGGSTYYADSV |
| | | | CTCCGTGAAGGGCCGATTCACCATCTCCAGAGACAATTCCAAGAAC | | KGRFTISRDNSKNTLY |
| | | | ACACTGTATCTTCAAATGAACAGCCTGAGAGCCGAGGACACGGCCG | | LQMNSLRAEDTAVYY |
| | | | TGTATTACTGTGCGAGAGATTTAGAACTGGCTGGAGCTTTTGATATC | | CARDLELAGAFDIWG |
| | | | TGGGGCCAAGGGACAATGGTCACCGTCTCTTTA | | QGTMVTVSL |
| 12 | 11D5_f3_H | 12 | GAGGTGCAGTTGGTGGAGACTGGAGGAGGCTTGATCCAGCCTGGG | 70 | EVQLVETGGGLIQPGG |
| | | | GGGTCCCTGAGACTCTCCTGTGCAGCCTCTGGGATCACCGTCAGTA | | SLRLSCAASGITVSSNY |
| | | | GTAACTACATGAGCTGGGTCCGCCAGGCTCCAGGGAAGGGGCTGG | | MSWVRQAPGKGLEW |
| | | | AGTGGGTCTCAGTTATTTATAGCGGTGGTAGCACATACTACGCAGA | | VSVIYSGGSTYYADSV |
| | | | CTCCGTGAAGGGCCGATTCACCATCTCCAGAGACAATTCCAAGAAC | | KGRFTISRDNSKNTLY |
| | | | ACGCTGTATCTTCAAATGAACAGCCTGAGAGCCGAGGACACGGCCG | | LQMNSLRAEDTAVYY |
| | | | TGTATTACTGTGCGAGAGATTTAGCAGTGGCTGGAGCTTTTGATATC | | CARDLAVAGAFDIWG |
| | | | TGGGGCCAAGGGACAATGGTCACCGTCTCTTTA | | QGTMVTVSL |
| 13 | 11G2_f3_H | 13 | CAGGTGCAGCTGGTGCAGTCTGGGGCTGAGGTGAAGAAGCCTGGG | 71 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCACCTGGGTGCGACAGGCCACTGGACAAGGGCTTGA | | YDITWVRQATGQGLE |
| | | | GTGGATGGGATGGATGAGCCCTAACAGTGGTAACACAGGCTATGC | | WMGWMSPNSGNTGY |
| | | | ACAGAAGTTCCAGGGCAGAGTCTCCATGACCAGGAACACCTCCATA | | AQKFQGRVSMTRNTSI |
| | | | AGCACAGCCTACATGGAGCTGAGCAGCCTGAGATCTGAGGACACG | | STAYMELSSLRSEDTA |
| | | | GCCGTGTATTACTGTGCGAGATTCGGCTATGGTTCGGGGGCCCTCG | | VYYCARFGYGSGALD |
| | | | ACTACTACTACTACGGTTTGGACGTCTGGGGCCAAGGGACCACGGT | | YYYYGLDVWGQGTTV |
| | | | CACCGTCTCCTCA | | TVSS |
| 14 | 11G7_f2_H | 14 | CAGGTGCAGCTGCAGGAGTCGGGCCCAGGACTGGTGAAGCCTTCG | 72 | QVQLQESGPGLVKPSG |
| | | | GGGACCCTGTCCCTCACCTGCGCTGTCTCTGGTGGCTCCATCAGCAG | | TLSLTCAVSGGSISSSN |
| | | | TAGTAACTGGTGGAGTTGGGTCCGCCAGCCCCCAGGGAAGGGGCTG | | WWSWVRQPPGKGLE |
| | | | GAGTGGATTGGGGAAATCTTTCATAGTGGGAGCACCAACTACAACC | | WIGEIFHSGSTNYNPSL |
| | | | CGTCCCTCAAGAGTCGAGTCACCATATCAGTAGACAAGTCCAAGAA | | KSRVTISVDKSKNQFS |
| | | | CCAGTTCTCCCTGAAGCTGAACTCTGTGACCGCCGCGGACACGGCC | | LKLNSVTAADTAVYY |
| | | | GTGTATTACTGTGCGAGATTCAGAAGGATAGTGGCTACGAGCTACT | | CARFRRIVATSYYFDY |
| | | | ATTTTGACTACTGGGGCCAGGGAACCCTGGTCACCGTCTCCTCA | | WGQGTLVTVSS |
| 15 | 16C5_f3_H | 15 | CAGGTGCAGCTGGTGCAGTCTGGGGCTGAGGTGAAGAAGCCTGGG | 73 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCATTTGGGTGCGACAGGCCACTGGACAAGGGCTTGA | | YDIIWVRQATGQGLE |
| | | | GTGGATGGGATGGATGAGCCCTAATAGTGGTAACACAGGCTATGCA | | WMGWMSPNSGNTGY |
| | | | CAGAAGTTCCAGGGCAGAGTCACCATGACCAGGAACACCTCCATA | | AQKFQGRVTMTRNTSI |
| | | | GGCACAGCCTACATGGAGCTGAGCAGCCTGAGATCTGAGGACACG | | GTAYMELSSLRSEDTA |
| | | | GCCGTGTATTTCTGTGCGAGATTCGGCTATGGTTCGGGGGCCCTCG | | VYFCARFGYGSGALD |
| | | | ACTACTACTACTACGGTTTGGACGTCTGGGGCCAAGGGACCACGGT | | YYYYGLDVWGQGTTV |
| | | | CACCGTCTCCTCA | | TVSS |
| 16 | 16C12_f3_H | 16 | CAGGTGCAGCTGGTGCAGTCTGGGGCTGAGGTGAAGAAGCCTGGG | 74 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATTATCTGGGTGCGACAGGCCACTGGACAAGGGCTTGA | | YDIIWVRQATGQGLE |
| | | | GTGGATGGGATGGATGAGCCCTAACAGTGGTAACACAGGCTATGC | | WMGWMSPNSGNTGY |
| | | | ACAGAAGTTCCAGGGCAGAGTCACCATGACCAGGAACACCTCCAT | | AQKFQGRVTMTRNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 17 | 17A8_f3_H | 17 | CAGGTGCAGCTGGTGCAGTCTGGGGCTGAGGTGAAGAAGCCTGGG | 75 | QVQLVQSGAEVKKPG |
| | | | GCCTCAGTGAAGGTCTCCTGCAAGGCTTCTGGATACACCTTCACCA | | ASVKVSCKASGYTFTS |
| | | | GTTATGATATCATTTGGGTGCGACAGGCCACTGGACAAGGGCTTGA | | YDIIWVRQATGQGLE |
| | | | GTGGATGGGATGGATGAGCCCTAACAGTGGTAACACAGGCTATGC | | WMGWMSPNSGNTGY |
| | | | ACAGAAGTTCCAGGGCAGAGTCACCATGACCAAGAACACCTCCAT | | AQKFQGRVTMTKNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 18 | 17F2_f3_H | 18 | CAGGTGCAGCTGGTGCAGTCTGGGGCTGAGGTGAAGAGGCCTGGG | 76 | QVQLVQSGAEVKRPG |
| | | | GCCTCAGTGAAGGTCTCCTGCAAGGCTTCTGGATACACCTTCACCA | | ASVKVSCKASGYTFTS |
| | | | GTTATGATATCACCTGGGTGCGACAGGCCACTGGACAAGGGCTTGA | | YDITWVRQATGQGLE |
| | | | GTGGATGGGATGGATGAGCCCTGACAGTGGTAACACAGGCTATGC | | WMGWMSPDSGNTGY |
| | | | ACAGAGGTTCCAGGGCAGAGTCACCATGACCAGGAACACCTCCAT | | AQRFQGRVTMTRNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 19 | 18E7_f3_H | 19 | CAGGTGCAGCTGGTGCAGTCTGGGGCTGAGGTGAAGAAGCCTGGG | 77 | QVQLVQSGAEVKKPG |
| | | | GCCTCAGTGACGGTCTCCTGCAAGGCTTCTGGATACACCTTTACCA | | ASVTVSCKASGYTFTS |
| | | | GTTATGATATCATCTGGGTGCGACAGGCCACTGGACAAGGGCTTGA | | YDIIWVRQATGQGLE |
| | | | GTGGATGGGATGGATGAGCCCTAACAGTGGTAACACAGGCTATGC | | WMGWMSPNSGNTGY |
| | | | ACAGAGGTTCCAGGGCAGAGTCACCATGACCAGGAACACCTCCAT | | AQRFQGRVTMTRNTSI |
| | | | AAGCACAGCCTACATGGAGCTGAGCAGCCTGAGATCTGAGGACAC | | STAYMELSSLRSEDTA |
| | | | GGCCGTGTATTACTGTGCGAGATTCGGCTATGGTTCGGGGGCCCTC | | VYYCARFGYGSGALD |
| | | | GACTACTACTACTACGGTTTGGACGTCTGGGGCCAAGGGACCACGG | | YYYYGLDVWGQGTTV |
| | | | TCACCGTCTCCTCA | | TVSS |
| 20 | 19C4_f3_H | 20 | GAGGTGCAGTTGGTGGAGACTGGAGGAGGCTTGATCCAGCCTGGG | 78 | EVQLVETGGGLIQPGG |
| | | | GGGTCCCTGAGACTCTCCTGTGCAGCCTCTGGGATCACCGTCAGTA | | SLRLSCAASGITVSSNY |
| | | | GTAATTACATGAACTGGGTCCGCCAGGCTCCAGGGAAGGGGCTGG | | MNWVRQAPGKGLEW |
| | | | AGTGGGTCTCAGTTATTTATAGCGGTGGTAGCACATTCTACGCAGA | | VSVIYSGGSTFYADSV |
| | | | CTCCGTGAAGGGCCGATTCACCATCTCCAGAGACAATTCCAAGAAC | | KGRFTISRDNSKNTLY |
| | | | ACGCTGTATCTTCAAATGAACAGCCTGAGAGCCGAGGACACGGCCG | | LQMNSLRAEDTAVYY |
| | | | TATATTACTGTGCGAGAGATTTAGAAGTGGCTGGAGGTTTTGATAT | | CARDLEVAGGFDIWG |
| | | | CTGGGGCCAAGGGACAATGGTCACCGTCTCTTTA | | QGTMVTVSL |
| 21 | 2C1_f4_H | 21 | GAGGTGCAGCTGGTGGAGTCTGGGGGAGGCTTGGTACAGCCAGGA | 79 | EVQLVESGGGLVQPGR |
| | | | CGGTCCCTGAGACTCTCCTGTACAGCTTCTGGATTCACCTTTGGTGA | | SLRLSCTASGFTFGDY |
| | | | TTATACTTTGAGCTGGTTCCGCCAGGCTCCAGGGAAGGGGCTGGAA | | TLSWFRQAPGKGLEW |
| | | | TGGGTAGGTTTCATTAGAAGCAAACCTTTTGGTGGGACAACACAAT | | VGFIRSKPFGGTTQYA |
| | | | ACGCCGCGTCTGTGAAAGGCAGATTCACCATCTCAAGGGATGATTC | | ASVKGRFTISRDDSKSI |
| | | | CAAAAGCATCGCCTATCTGCAAATGAACAGCCTGAAAACCGAGGA | | AYLQMNSLKTEDTAV |
| | | | CACAGCCGTGTATTACTGTACTAGAGTGTCCGGGTATAGCAACATC | | YYCTRVSGYSNIWFFA |
| | | | TGGTTCTTTGCCTACTGGGGCCAGGGAACCCTGGTCACCGTCTCCTC | | YWGQGTLVTVSS |
| | | | A | | |
| 22 | 4E3_f4_H | 22 | CAGGTGCAACTGGTGGAGTCTGGGGGAGGCGTGGTCCAGCCTGGG | 80 | QVQLVESGGGVVQPG |
| | | | AGGTCCCTGAGACTCTCCTGTGCAGCGTCTGGATTCACCTTCAGTAG | | RSLRLSCAASGFTFSSY |
| | | | CTATGGCATGAACTGGGTCCGCCAGGCTCCAGGCAAGGGGCTGGA | | GMNWVRQAPGKGLE |
| | | | GTGGGTGGCAGTTATTTGGTATGATGGAAATAATAAATACTATGCA | | WVAVIWYDGNNKYY |
| | | | GACTCCGTGAAGGGCCGATTCACCATCTCCAGAGACAATTCCAAGA | | ADSVKGRFTISRDNSK |
| | | | ACACGTTGTATCTGCAAATGAACAGCCTGAGAGCCGAGGACACGG | | NTLYLQMNSLRAEDT |
| | | | CTGTGTATTACTGTGCGAGAAAAGATTATTCGAAGACATTTTATGG | | AVYYCARKDYSKTFY |
| | | | ATACTACTTTGACTATTGGGGCCAGGGAACCCTGGTCACCGTCTCCT | | GYYFDYWGQGTLVTV |
| | | | CA | | SS |
| 23 | 7D2_f4_H | 23 | GAGGTGCAGCTGGTGGAGACTGGAGGAGGCTTGATCCAGCCTGGG | 81 | EVQLVETGGGLIQPGG |
| | | | GGGTCCCTGAGACTCTCCTGTGCAGCCTCTGGGTTCACCGTCAGTA | | SLRLSCAASGFTVSSN |
| | | | GCAACTACATGAACTGGGTCCGCCAGGCTCCAGGGAAGGGGCTGG | | YMNWVRQAPGKGLE |
| | | | AGTGGGTCTCAGTTATTTATAGCGGTGGTAGCACATTCTACGCAGA | | WVSVIYSGGSTFY ADS |
| | | | CTCCGTGAAGGGCCGATTCACCATCTCCAGAGACAATTCCTACAAC | | VKGRFTISRDNSYNTL |
| | | | ACGCTGTATCTTCAAATGAACAGCCTGAGAGCCGAGGACACGGCCG | | YLQMNSLRAEDTAVY |
| | | | TGTATTACTGTGCGAGAGATCTAGTCATCTACGGTATGGACGTCTG | | YCARDLVIYGMDVWG |
| | | | GGGCCAAGGGACCACGGTCACCGTCTCCTCA | | QGTTVTVSS |
| 24 | 9C5_f4_H | 24 | CAGGTGCAACTGGTGGAGTCTGGGGGAGGCGTGGTCCAGCCTGGG | 82 | QVQLVESGGGVVQPG |
| | | | AGGTCCCTGAGACTCTCCTGTGTAGCGTCTGGATTCACCTTCAGTAG | | RSLRLSCVASGFTFSSY |
| | | | CTATGGCATGAACTGGGTCCGCCAGGCTCCAGGCAAGGGGCTGGA | | GMNWVRQAPGKGLE |
| | | | GTGGGTGGCAGTTATTTGGTATGATGGAAATAATAAATACTATGCA | | WVAVIWYDGNNKYY |
| | | | GACTCCGTGAAGGGCCGATTCACCATCTCCAGAGACAATTCCAAGA | | ADSVKGRFTISRDNSK |
| | | | ACACGCTGTATCTGCAAATGAACAGTCTGAGAGCCGAGGACACGG | | NTLYLQMNSLRAEDT |
| | | | CTGTGTATTACTGTGCGAGAAAAGATGGTTCGAAGACATATTATGG | | AVYYCARKDGSKTYY |
| | | | ATACTACTTTGACTATTGGGGCCAGGGAACCCTGGTCACCGTCTCCT | | GYYFDYWGQGTLVTV |
| | | | CA | | SS |
| 25 | 10A3_f4_H | 25 | CAGGTGCAACTGGTGGAGTCTGGGGGAGGCGTGGTCCAGCCTGGG | 83 | QVQLVESGGGVVQPG |
| | | | AGGTCCCTGAGACTCTCCTGTGCAGCGTCTGGATTCACCTTCAGTAG | | RSLRLSCAASGFTFSSY |
| | | | TTATGGCATGAACTGGGTCCGCCAGGCTCCAGGCAAGGGGCTGGAG | | GMNWVRQAPGKGLE |
| | | | TGGGTGGCAATTATTTGGTATGATGGAAATAATACATACTATGTAG | | WVAIIWYDGNNTYYV |
| | | | ACTCCGTGAAGGGCCGATTCACCATCTCCAGAGACAATTCCAAGAA | | DSVKGRFTISRDNSKN |
| | | | CACGCTGTATCTGCAAATGAACAGCCTGAGAGCCGAAGACACGGCT | | TLYLQMNSLRAEDTA |
| | | | GTGTATTACTGTGCGAGAAAAGATGGTTCGAAGACATATTATGGAT | | VYYCARKDGSKTYYG |
| | | | ACTACTTTGACTATTGGGGCCAGGGAACCCTGGTCACCGTCTCCTC | | YYFDYWGQGTLVTVS |
| | | | A | | S |
| 26 | 14G5_f4_H | 26 | CAGGTGCAGCTGCAGGAGTCGGGCCCAGGACTGGTGAAGCCTTCG | 84 | QVQLQESGPGLVKPSG |
| | | | GGGACCCTGTCCCTCACCTGCGCTGTCTCTGGTGGCTCCATCAGCAG | | TLSLTCAVSGGSISSNN |
| | | | TAATAACTGGTGGAGTTGGGTCCGCCAGCCCCCAGGGAAGGGGCTG | | WWSWVRQPPGKGLV |
| | | | GTGTGGATTGGGGAAATCTTGCATGGTGAGATCACCAACTACAACC | | WIGEILHGEITNYNPSL |
| | | | CGTCCCTCAAGAGTCGAGTCACCATATCAATAGACAAGTCCAAGAA | | KSRVTISIDKSKNQFSL |
| | | | CCAGTTCTCCCTGAAGCTGAGCTCTGTGACCGCCGCGGACACGGCC | | KLSSVTAADTAVYYC |
| | | | GTGTATTACTGTGCGAGAGATGCGAATTTCTATGGTTCGGGGAGTT | | ARDANFYGSGSSYFDY |
| | | | CTTACTTTGACTACTGGGGCCAGGGAACCCTGGTCACCGTCTCCTCA | | WGQGTLVTVSS |
| 27 | 13A12_f3_H | 27 | CAGGTGCAGCTGCAGGAGTCGGGCCCAGGACTGGTGAAGCCTTCG | 85 | QVQLQESGPGLVKPSG |
| | | | GGGACCCTGTCCCTCACCTGCGCTGTCTCAGGTGGCTCCATCTACAG | | TLSLTCAVSGGSIYSSN |
| | | | TAGTAACTGCTGGAGTTGGGTCCGCCAGCCCCCAGGGAAGGGGCTG | | CWSWVRQPPGKGLEW |
| | | | GAGTGGATTGGGGAAATCTATCATAGTGGGGGCACCAACTACAACC | | IGEIYHSGGTNYNPSLK |
| | | | CGTCCCTCAAGAGTCGAGTCACCATATCATTAGACAAGTCCAAGAA | | SRVTISLDKSKNRFSLR |
| | | | CAGGTTCTCCCTGAGGCTGAGCTCTGTGACCGCCGCGGACACGGCC | | LSSVTAADTAVYYCA |
| | | | GTGTATTACTGTGCGAGAGATCAAGATTACTATGGTTCGGGGAGTT | | RDQDYYGSGSSLFDY |
| | | | CCCTCTTTGACTACTGGGGCCAGGGAACCCTGGTCACCGTCTCCTCA | | WGQGTLVTVSS |
| 28 | 5B6_f3_H | 28 | GAGGTGCAGCTGGTGGAGTCTGGGGGAGGCTTGGTAAAGCCTGGG | 86 | EVQLVESGGGLVKPG |
| | | | GGGTCCCTTAGACTCTCCTGTGCAGCCTCTGGATTCACTTTCAGTAA | | GSLRLSCAASGFTFSN |
| | | | CGCCTGGATGAGCTGGGTCCGCCAGGCTCCAGGGAAGGGGCTGGA | | AWMSWVRQAPGKGL |
| | | | GTGGGTTGGCCGTTTTAAAAGCAAAACTGATGGTGGGACAACAGAC | | EWVGRFKSKTDGGTT |
| | | | TACGCTGCACCCGTGAAAGGCAGATTCACCATCTCAAGAGATGATT | | DYAAPVKGRFTISRDD |
| | | | CAAAAAACACGCTGTATCTGCAAATGAACAGCCTGAAAACCGAGG | | SKNTLYLQMNSLKTED |
| | | | ACACAGCCGTGTATTACTGTACCACCAGCAGTGGCTACTGGGGCCA | | TAVYYCTTSSGYWGQ |
| | | | GGGAACCCTGGTCACCGTCTCCTCA | | GTLVTVSS |
| 29 | 2G6_f4_H | 29 | CAGGTGCAGCTACAGCAGTGGGGCGCAGGACTGTTGAAGCCTTCGG | 87 | QVQLQQWGAGLLKPS |
| | | | AGACCCTGTCCCTCACCTGCACTATCTATGGTGGGTCCTTCAGTGTT | | ETLSLTCTIYGGSFSVY |
| | | | TACTACTGGAACTGGATCCGCCAGCCCCCAGAGAAGGGGCTGGAGT | | YWNWIRQPPEKGLEWI |
| | | | GGATTGGGGAAATCAATCATAGTGGAAACACCAACTACAACCCGTC | | GEINHSGNTNYNPSLK |
| | | | CCTCAAGAGTCGAGTCACCATATCAGTAGACACGTCCAAGAACCAA | | SRVTISVDTSKNQFSLK |
| | | | TTCTCCCTGAAGCTGAGCTCTGTGACCGCCGCGGACACGGCTGTGT | | LSSVTAADTAVYYCA |
| | | | ATTACTGTGCGAGGTATTACTATGATGGTAATGGTTATTACCCCTGG | | RYYYDGNGYYPWGQ |
| | | | GGCCAGGGAACCCTGGTCACCGTCTCCTCA | | GTLVTVSS |

TABLE 2
Variable Light Chain Sequences.

| | | | | Variable Light Chain | |
|---|---|---|---|---|---|
| | | VariABLE Light Chain Nucleotide Sequence | | Amino Acid Sequence | |
| | | SEQ | | SEQ | |
| Ab # | Name | ID NO | Sequence | ID NO | Sequence |
| 1 | 1C12_f3_K | 30 | GAAATTGTGTTGACGCAGTCTCCAGGCACCCTGTCTTTGTCTCCAGG | 88 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCAGCAGAAACCTGGCCAGGCTCCCAGGC | | LAWYQQKPGQAPRLL |
| | | | TCCTCATCTATGGTGCATCCAGCAGGGCCACTGGCATCCCAGACAGG | | IYGASSRATGIPDRFS |
| | | | TTCAGTGGCAGTGGGTCTGGGACAGACTTCACTCTCACCATCAGCAG | | GSGSGTDFTLTISRLEP |
| | | | ACTGGAGCCTGAAGATTTTGCAGTGTATTACTGTCAGCAGTATAATA | | EDFAVYYCQQYNNSP |
| | | | ACTCACCGATCACCTTCGGCCAAGGGACACGACTGGAGATTAAA | | ITFGQGTRLEIK |
| 2 | 1D5_f3_K | 31 | GAAATTGTGTTGACGCAGTCTCCAGGCACCCTGTCTTTGTCTCCAGG | 89 | EIVLTQSPGTLSLSPG |
| | | | GGACAGAGCCACCCTCTCCTGCAGGGCCAGTCAGAGTGTTAGGAGC | | DRATLSCRASQSVRSS |
| | | | AGTCACTTAGCCTGGTACCAGCAGAAACCTGGCCAGGCTCCCAGGC | | HLAWYQQKPGQAPR |
| | | | TCCTCATCTATGGTGCATCCAGCAGGGCCACTGGCATCCCAGACAGG | | LLIYGASSRATGIPDR |
| | | | TTCAGTGGCAGTGGGTCTGGGACAGACTTCTCTCTCACCATCAGCAG | | FSGSGSGTDFSLTISRL |
| | | | ACTGGAGCCTGAGGATTTTGCAGTGTATTACTGTCAGCAGTTTGGTA | | EPEDFAVYYCQQFGS |
| | | | GCTCACCGATCACCTTCGGCCAAGGGACACGGCTGGAGATTAAA | | SPITFGQGTRLEIK |
| 3 | 1D10_f3_K | 32 | GAAATTGTGTTGACGCAGTCTCCAGGCACCCTGTCTTTGTCTCCAGG | 90 | EIVLTQSPGTLSLSPGE |
| | | | GGAAAGAGCCACCCTCTCCTGCAGGGCCAGTCAGAGTGTTAGCAGC | | RATLSCRASQSVSSSH |
| | | | AGCCACTTAGCCTGGTACCAGCAGAAACCTGGCCAGGCTCCCAGGC | | LAWYQQKPGQAPRLL |
| | | | TCCTCATCTATGGTGCATCCAGCAGGGCCACTGGCATCCCAGACAGG | | IYGASSRATGIPDRFS |
| | | | TTCAGTGGCAGTGGGTCTGGAACAGACTTCACTCTCACCATCAGCAG | | GSGSGTDFTLTISRLEP |
| | | | ACTGGAGCCTGAAGATTTTGCAGTGTATTACTGTCAGCAGTATAATA | | EDFAVYYCQQYNSSPI |
| | | | GCTCACCGATCACCTTCGGCCAAGGGACACGACTGGAGATTAAA | | TFGQGTRLEIK |
| 4 | 1E2_f3_K | 33 | GAAATTGTGTTGACGCAGTCTCCAGGCACCCTGTCTTTGTCTCCAGG | 91 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTTGCCTGGTACCAGCAGAAACCTGGCCAGGCTCCCAGGCT | | FAWYQQKPGQAPRLL |
| | | | CCTCATCTATGGTGCATCCAGCAGGGCCACTGGCATCCCAGACAGGT | | IYGASSRATGIPDRFS |
| | | | TCAGTGGCAGTGGGTCTGGGACAGACTTCACTCTCACCATCAGCAG | | GSGSGTDFTLTISRLEP |
| | | | ACTGGAGCCTGAAGATTTTGCAGTGTGTTACTGTCAGCAGTATGGTA | | EDFAVCYCQQYGSSPI |
| | | | GCTCACCGATCACCTTCGGCCAAGGGACACGACTGGAGATTAAA | | TFGQGTRLEIK |
| 5 | 1G9_f3_K | 34 | GAAATTGTGTTGACGCAGTCTCCAGGCACCCTGTCTTTGTCTCCAGG | 92 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCAGCAGAAACCTGGCCAGGCTCCCAGGC | | LAWYQQKPGQAPRLL |
| | | | TCCTCATCTATGGTGCATCCAGCAGGGCCACTGGCATCCCAGACAGG | | IYGASSRATGIPDRFS |
| | | | TTCAGTGGCAGTGGGTCTGGGACAGACTTCACTCTCACCATCAGCAG | | GSGSGTDFTLTISRLEP |
| | | | ACTGGAGCCTGAAGATTTTGCAGTGTATTACTGTCAGCAGTATGGTA | | EDFAVYYCQQYGSSPI |
| | | | GCTCACCGATCACCTTCGGCCAAGGGACACGACTGGAGATTAAA | | TFGQGTRLEIK |
| 6 | 5D7_f3_K | 35 | GAAATTGTGTTGACGCAGTCTCCAGGCACCCTGTCTTTGTCTCCAGG | 93 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCGGCAGAAACCTGGCCAGGCTCCCAGGC | | LAWYRQKPGQAPRLL |
| | | | TCCTCATCTATGGTGCCTCCAGCAGGGCCACTGGCATCCCAGACAGG | | IYGASSRATGIPDRFS |
| | | | TTCAGTGGCCGTGGGTCTGGGACAGACTTCACTCTCACCATCAGCAG | | GRGSGTDFTLTISRLE |
| | | | ACTGGAGCCTGAAGATTTTGCAGTGTATTACTGTCAGCAGTATAATA | | PEDFAVYYCQQYNISP |
| | | | TCTCACCGATCACCTTCGGCCAAGGGACACGACTGGAGATTAAA | | ITFGQGTRLEIK |
| 7 | 5H12_f3_K | 36 | GAAATTGTGTTGACGCAGTCTCCAGGCACCCTGTCTTTGTCTCCAGG | 94 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCGGCAGAAACCTGGCCAGGCTCCCAGGC | | LAWYRQKPGQAPRLL |
| | | | TCCTCATCTATGGTGCCTCCAGCAGGGCCACTGGCATCCCAGACAGG | | IYGASSRATGIPDRFS |
| | | | TTCAGTGGCCGTGGGTCTGGGACAGACTTCACTCTCACCATCAGCAG | | GRGSGTDFTLTISRLE |
| | | | ACTGGAGCCTGAAGATTTTGCAGTGTATTACTGTCAGCAGTATAATT | | PEDFAVYYCQQYNFS |
| | | | TCTCACCGATCACCTTCGGCCAAGGGACACGACTGGAGATTAAA | | PITFGQGTRLEIK |
| 8 | 7C10_f3_K | 37 | GAAATTGTGTTGACGCAGTCTCCAGGCACCCTGTCTTTGTCTCCAGG | 95 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTCCCAGCAGAAACCTGGCCAGGCTCCCAGGC | | LAWSQQKPGQAPRLL |
| | | | TCCTCATCTATGGTGCATCCAGCAGGGCCACTGGCATCCCAGACAGG | | IYGASSRATGIPDRFS |
| | | | TTCAGTGGCAGTGGGTCTGGGACAGACTTCACTCTCACCATCAGCAG | | GSGSGTDFTLTISRLEP |
| | | | ACTGGAGCCTGAAGATTTTGCAGTGTATTACTGTCAGCAGTTTGGTA | | EDFAVYYCQQFGSSPI |
| | | | GCTCACCGATCACCTTCGGCCAAGGGACACGACTGGAGATTAAA | | TFGQGTRLEIK |
| 9 | 8H4_f3_K | 38 | GAAATTGTGTTGACGCAGTCTCCAGGCACCCTGTCTTTGTCTCCAGG | 96 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCAGCAGAAACCTGGCCAGGCTCCCAGGC | | LAWYQQKPGQAPRLL |
| | | | TCCTCATCTATGGTGCATCCAGCAGGGCCACTGGCATCCCAGACAGG | | IYGASSRATGIPDRFS |
| | | | TTCAGTGGCAGTGGGTCTGGGACAGACTTCACTCTCACCATCAGCAG | | GSGSGTDFTLTISRLEP |
| | | | ACTGGAGCCTGAAGATTTTGCAGTGTATTACTGTCAGCAGTTTGGTA | | EDFAVYYCQQFGSSPI |
| | | | GCTCACCGATCACCTTCGGCCAAGGGACACGACTGGAGATTAAG | | TFGQGTRLEIK |
| 10 | 9C6_f3_K | 39 | GAAATTGTGTTGACGCAGTCTCCAGGCACCCTGTCTTTGTCTCCAGG | 97 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCGGCAGAAACCTGGCCAGGCTCCCAGGC | | LAWYRQKPGQAPRLL |
| | | | TCCTCATCTATGGTGCCTCCAGCAGGGCCACTGGCATCCCAGACAGG | | IYGASSRATGIPDRFS |
| | | | TTCAGTGGCCGTGGGTCTGGGACAGACTTCACTCTCACCATCAGCAG | | GRGSGTDFTLTISRLE |
| | | | ACTGGAGCCTGAAGATTTTGCAGTGTATTACTGTCAGCAGTATAATA | | PEDFAVYYCQQYNSS |
| | | | GCTCACCGATCACCTTCGGCCAAGGGACACGACTGGAGATAAAA | | PITFGQGTRLEIK |
| 11 | 10D6_f3_K | 40 | GACATCCAGTTGACCCAGTCTCCATCCTTCCTGTCTGCATCTGTAGG | 98 | DIQLTQSPSFLSASVG |
| | | | AGACAGAGTCACCATCACTTGCCGGGCCAGTCAGGGCATTAGCAGT | | DRVTITCRASQGISSY |
| | | | TATTTAGCCTGGTATCAGCAAAAACCAGGGAAAGCCCCTAAGGTCC | | LAWYQQKPGKAPKV |
| | | | TGATCTATGCTGCATCCACTTTGCAAAGTGGGGTCCCATCAAGGTTC | | LIYAASTLQSGVPSRF |
| | | | AGCGGCAGTGGATCTGGGACAGAATTCACTCTCACAATCAGCAGCC | | SGSGSGTEFTLTISSLQ |
| | | | TGCAGCCTGAAGATTTTGCAACTTATTACTGTCAACAGCTTAATAGT | | PEDFATYYCQQLNSY |
| | | | TACCCTCCGTCCACTTTTGGCCAGGGGACCAAGCTGGAGATCAAA | | PPSTFGQGTKLEIK |
| 12 | 11D5_f3_K | 41 | GACATCCAGTTGACCCAGTCTCCATCCTTCCTGTCTGCATCTGTAGG | 99 | DIQLTQSPSFLSASVG |
| | | | AGACAGAGTCACCATCACTTGCCGGGCCAGTCAGGGCATTAGCAGT | | DRVTITCRASQGISSY |
| | | | TATTTAGCCTGGTATCAGCAAAAACCAGGGAAAGCCCCTAAGGTCC | | LAWYQQKPGKAPKV |
| | | | TGATCTATGCTGCATCCACTTTGCAAAGTGGGGTCCCATCAAGGTTC | | LIYAASTLQSGVPSRF |
| | | | AGCGGCAGTGGATCTGGGACAGAATTCACTCTCACAATCAGCAGCC | | SGSGSGTEFTLTISSLQ |
| | | | TGCAGCCTGAAGATTTTGCAACTTATTACTGTCAACAGCTTAATAGT | | PEDFATYYCQQLNSY |
| | | | TACCCTCCGTCCACTTTTGGCCAGGGGACCAAGCTGGAGATCAAA | | PPSTFGQGTKLEIK |
| 13 | 11G2_f3_K | 42 | GAAATTGTGTTGACGCAGTCTCCAGGCACCCTGTCTTTGTCTCCAGG | 100 | EIVLTQSPGTLSLSPGE |
| | | | GGAAAGAGCCACCCTCTCCTGCAGGGCCAGTCAGAGTGTTAGGAAC | | RATLSCRASQSVRNS |
| | | | AGTCACTTAGCCTGGTACCAGCAGAAACCTGGCCAGGCTCCCAGGC | | HLAWYQQKPGQAPR |
| | | | TCCTCATCTATGGTGCATCCAGCAGGGCCACTGGCATCCCAGACAGG | | LLIYGASSRATGIPDR |
| | | | TTCAGTGGCAGTGGGTCTGGGACAGACTTCACTCTCACCATCAGCAG | | FSGSGSGTDFTLTISRL |
| | | | ACTGGAGCCTGAAGATTTTGCAGTGTATTACTGTCAGCAGTTTGGTA | | EPEDFAVYYCQQFGS |
| | | | GCTCACCGATCACCTTCGGCCAAGGGACACGACTGGAGATTAAA | | SPITFGQGTRLEIK |
| 14 | 11G7_f2_K | 43 | GATATTGTGATGACTCAGTCTCCACTCTCCCTGCCCGTCACCCCTGG | 101 | DIVMTQSPLSLPVTPG |
| | | | AGAGCCGGCCTCCATCTCCTGCAGGTCTAGTCAGAGCCTCCTGCATA | | EPASISCRSSQSLLHSN |
| | | | GTAATGGATACAACTATTTGGATTGGTACCTGCAGAAGCCAGGGCA | | GYNYLDWYLQKPGQ |
| | | | GTCTCCACAGCTCCTGATCTATTTGGGTTCTAATCGGGCCTCCGGGG | | SPQLLIYLGSNRASGV |
| | | | TCCCTGACAGGTTCAGTGGCAGTGGATCAGGCACAGATTTTACACTG | | PDRFSGSGSGTDFTLK |
| | | | AAAATCAGCAGAGTGGAGGCTGAGGATGTTGGGGTTTATTACTGCA | | ISRVEAEDVGVYYCM |
| | | | TGCAAGCTCTACAAACTCCTCTCACTTTCGGCGGAGGGACCAAGGTG | | QALQTPLTFGGGTKV |
| | | | GAGATCAAA | | EIK |
| 15 | 16C5_f3_K | 44 | GAAATTGTGTTGACGCAGTCTCCAGGCACCCTGTCTTTGTCTCCAGG | 102 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCAGCAGAAACCTGGCCAGGCTCCCAGGC | | LAWYQQKPGQAPRLL |
| | | | TCCTCATCTATGGTGCATCCAGCAGGGCCACTGGCATCCCAGACAGG | | IYGASSRATGIPDRFS |
| | | | TTCAGTGGCAGTGGGTCTGGGACAGACTTCACTCTCACCATCAGCAG | | GSGSGTDFTLTISRLEP |
| | | | ACTGGAGCCTGAAGATTTTGCAGTGTATTACTGTCAGCAGTATAATA | | EDFAVYYCQQYNNSP |
| | | | ACTCACCGATCACCTTCGGCCAAGGGACACGACTGGAGATTAAA | | ITFGQGTRLEIK |
| 16 | 16C12_f3_K | 45 | GAAATTGTGTTGACGCAGTCTCCAGGCACCCTGTCTTTGTCTCCAGG | 103 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCAGCAGAAACCTGGCCAGGCTCCCAGGC | | LAWYQQKPGQAPRLL |
| | | | TCCTCATCTATGGTGCATCCAGCAGGGCCACTGGCATCCCAGACAGG | | IYGASSRATGIPDRFS |
| | | | TTCAGTGGCAGTGGGTCTGGGACAGACTTCACTCTCACCATCAGCAG | | GSGSGTDFTLTISRLEP |
| | | | ACTGGAGCCTGAAGATTTTGCAGTGTATTACTGTCAGCAGTATGGTA | | EDFAVYYCQQYGSSPI |
| | | | GCTCACCGATCACCTTCGGCCAAGGGACACGACTGGAGATTAAA | | TFGQGTRLEIK |
| 17 | 17A8_f3_K | 46 | GAAACTGTGTTGACGCAGTCTCCAGGCACTCTGTCTTTGTCTCCAGG | 104 | ETVLTQSPGTLSLSPG |
| | | | GGAAAGAGCCACCCTCTCCTGCAGGGCCAGTCAGAGTGTTAGGAGC | | ERATLSCRASQSVRSS |
| | | | AGTCACTTAGCCTGGTACCAGCAGAAACCTGGCCAGGCTCCCAGGC | | HLAWYQQKPGQAPR |
| | | | TCCTCATCTATGGTGCATCCAGTAGGGCCACTGGCATCCCAGACAGG | | LLIYGASSRATGIPDR |
| | | | TTCAGTGGCAGTGGGTCTGGGACAGACTTCACTCTCACCATCAGCAG | | FSGSGSGTDFTLTISRL |
| | | | ACTGGAGCCTGAAGATTTTGCAGTGTATTACTGTCAGCAGTATGGTA | | EPEDFAVYYCQQYGS |
| | | | GCTCACCGATCACCTTCGGCCAAGGGACACGACTGGAGATTAAA | | SPITFGQGTRLEIK |
| 18 | 17F2_f3_K | 47 | GAAATTGTGTTGACGCAGTCTCCAGGCACCCTGTCTTTGTCTCCAGG | 105 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCAGCAGAAACCTGGCCAGGCTCCCAGGC | | LAWYQQKPGQAPRLL |
| | | | TCCTCATCTATGGTGCATCCAGCAGGGCCACTGGCATCCCAGACAGG | | IYGASSRATGIPDRFS |
| | | | TTCAGTGGCAGTGGGTCTGGGACAGACTTCACTCTCACCATCAGCAG | | GSGSGTDFTLTISRLEP |
| | | | ACTGGAGCCTGAAGATTTTGCAGTGTATTACTGTCAGCAGTATAATA | | EDFAVYYCQQYNSSPI |
| | | | GCTCACCGATCACCTTCGGCCAAGGGACACGACTGGAGATAAAA | | TFGQGTRLEIK |
| 19 | 18E7_f3_K | 48 | GAAATTGTGTTGACGCAGTCTCCAGGCACCCTGTCTTTGTCTCCAGG | 106 | EIVLTQSPGTLSLSPGE |
| | | | GGAAAGAGCCACCCTCTCCTGCAGGGCCAGTCAGAGTGTTAGGAGC | | RATLSCRASQSVRSSH |
| | | | AGTCACTTAGCCTGGTACCAGCAGAAACCTGGCCAGGCTCCCAGGC | | LAWYQQKPGQAPRLL |
| | | | TCCTCATCTATGGTGCATCCAGCAGGGCCACTGGCATCCCAGACAGG | | IYGASSRATGIPDRFS |
| | | | TTCAGTGGCAGTGGGTCTGGGACAGACTTCACTCTCACCATCAGCAG | | GSGSGTDFTLTISRLEP |
| | | | ACTGGAGCCTGAAGATTTTGCAGTGTATTACTGTCAGCAGTATGGTA | | EDFAVYYCQQYGSSPI |
| | | | GCTCACCGATCACCTTCGGCCAAGGGACACGACTGGAGATTAAA | | TFGQGTRLEIK |
| 20 | 19C4_f3_K | 49 | GACATCCAGTTGACCCAGTCTCCATCCTTCCTGTCTGCATCTGTAGG | 107 | DIQLTQSPSFLSASVG |
| | | | AGACAGAGTCACCATCACTTGCCGGGCCAGTCAGGGCATTAGCAGT | | DRVTITCRASQGISSY |
| | | | TATTTAGCCTGGTATCAGCAAAAACCAGGGAAAGCCCCTAAGGTCC | | LAWYQQKPGKAPKV |
| | | | TGATCTATGCTGCATCCACTTTGCAAAGTGGGGTCCCATCAAGGTTC | | LIYAASTLQSGVPSRF |
| | | | AGCGGCAGTGGATCTGGGACAAAATTCACTCTCACAATCAGCAGCC | | SGSGSGTKFTLTISSLQ |
| | | | TGCAGCCTGAAGATTTTGCAACTTATTACTGTCAACAGCTTAATAGT | | PEDFATYYCQQLNSFP |
| | | | TTCCCTCCGTCCACTTTTGGCCAGGGGACCAAGCTGGAGATCAAA | | PSTFGQGTKLEIK |
| 21 | 2C1_f4_K | 50 | GAAATAGTGATGACGCAGTCTCCAGCCACCCTGTCTGTGTCTCCAGG | 108 | EIVMTQSPATLSVSPG |
| | | | GGAAAGAGCCACCCTCTCCTGCAGGGCCAGTCAGAGTGTTAGCATC | | ERATLSCRASQSVSIN |
| | | | AACTTAGCCTGGTACCAGCAGAAACCTGGCCAGGCTCCCAGGCTCC | | LAWYQQKPGQAPRLL |
| | | | TCATCTATGGTGCATCCACCAGGGCCACTGGTATCCCAGCCAGGTTC | | IYGASTRATGIPARFS |
| | | | AGTGGCAGTGGGTCTGGGACAGAGTTCACTCTCACCGTCAGCAGCC | | GSGSGTEFTLTVSSLQ |
| | | | TGCAGTCTGAAGATTTTGCAGTTTATTACTGTCAGCAGTATAATAAC | | SEDFAVYYCQQYNN |
| | | | TGGTGGACGTTCGGCCAAGGGACCAAGGTGGAAATCAAA | | WWTFGQGTKVEIK |
| 22 | 4E3_f4_K | 51 | GACATCCAGATGACCCAGTCTCCATCCTCCCTGTCTGCATCTGTAGG | 109 | DIQMTQSPSSLSASVG |
| | | | AGACAGAGTCACCATCACTTGCCGGGCAAGTCAGAGCATTCACAGC | | DRVTITCRASQSIHSFL |
| | | | TTTTTAAATTGGTATCAGCAGAAACCAGGGAAACCCCCTAAGCTCCT | | NWYQQKPGKPPKLLI |
| | | | GATCTATGCTGCATCCAGTTTGCCAAGTGGGCTCCCATCAAGGTTCA | | YAASSLPSGLPSRFSG |
| | | | GTGGCAGTGGATCTGGGACAGATTTCACTCTCACCATCAGCAGTCTG | | SGSGTDFTLTISSLQPE |
| | | | CAACCTGAAGATTTTGCAACTTACTACTGTCAACAGAGTTACATTAC | | DFATYYCQQSYITPPT |
| | | | CCCTCCGACGTTCGGCCAAGGGACCAAGGTGGAAATCAAA | | FGQGTKVEIK |
| 23 | 7D2_f4_K | 52 | GAAATAGTGATGACGCAGTCTCCAGCCACCCTGTCTGTGTCTCCAGG | 110 | EIVMTQSPATLSVSPG |
| | | | GGAAAGAGCCACCCTCTCCTGCAGGGCCAGTCAGAGTGTTAGCAGC | | ERATLSCRASQSVSSN |
| | | | AACTTAGCCTGGTACCAGCAGAAACCTGGCCAGGCTCCCAGGCTCC | | LAWYQQKPGQAPRLL |
| | | | TCATCTATGGTGCATCCACCAGGGCCACTGGTGTCCCAGCCAGGTTC | | IYGASTRATGVPARFS |
| | | | AGTGGCAGTGGGTCTGGGACAGAGTTCACTCTCACCATCAGCAGCC | | GSGSGTEFTLTISSLQS |
| | | | TGCAGTCTGAAGATTTTGCAGTTTATTTCTGTCAGCAGTATAATAAC | | EDFAVYFCQQYNNW |
| | | | TGGCCCCCTTTCGGCGGAGGGACCAAGGTGGAGATCAAA | | PPFGGGTKVEIK |
| 24 | 9C5_f4_K | 53 | GACATCCAGATGACCCAGTCTCCATCCTCCCTGTCTGCATCTGTAGG | 111 | DIQMTQSPSSLSASVG |
| | | | AGACAGAGTCACCATCACTTGCCGGGCAAGTCAGAGCATTCACAGC | | DRVTITCRASQSIHSFL |
| | | | TTTTTAAATTGGTATCAGCAGAAACCAGGGAAACCCCCTAAGCTCCT | | NWYQQKPGKPPKLLI |
| | | | GATCTATACTACATCCAGTTTGCAAAGTGGGCTCCCATCAAGGTTCA | | YTTSSLQSGLPSRFSG |
| | | | GTGGCAGTGGATCTGGGACAGATTTCACTCTCACCATCAGCAGTCTG | | SGSGTDFTLTISSLQPE |
| | | | CAACCTGAAGATTTTGCAACTTACTACTGTCAACAGAGTTACATTAC | | DFATYYCQQSYITPPT |
| | | | CCCTCCGACGTTCGGCCAAGGGACCAAGGTGGAAATCAAA | | FGQGTKVEIK |
| 25 | 10A3_f4_K | 54 | GACATCCAGATGACCCAGTCTCCATCCTCCCTGTCTGCATCTGTAGG | 112 | DIQMTQSPSSLSASVG |
| | | | AGACAGAGTCACCATCACTTGCCGGGCAAGTCAGAGCATTCACAGC | | DRVTITCRASQSIHSFL |
| | | | TTTTTAAATTGGTATCAGCAGAAACCAGGGAAACCCCCTAACCTCCT | | NWYQQKPGKPPNLLI |
| | | | GATCTATGCTGCATCCAGTTTGCAAAGTGGGCTCCCATCAAGGTTCA | | YAASSLQSGLPSRFSG |
| | | | GTGGCAGTGGATCTGGGACAGATTTCACTCTCACCATCAGCAGTCTG | | SGSGTDFTLTISSLQPE |
| | | | CAACCTGAAGATTTTGCAACTTACTACTGTCAACAGAGTTACATTAC | | DFATYYCQQSYITPPT |
| | | | CCCTCCGACGTTCGGCCATGGGACCAAGGTGGAAATCAAA | | FGHGTKVEIK |
| 26 | 14G5_f4_K | 55 | GAAATAGTGATGACGCAGTCTCCAGCCACCCTGTCTGTGTCTCCAGG | 113 | EIVMTQSPATLSVSPG |
| | | | GGAAAGAGCCACCCTCTCCTGCAGGGCCAGTCAGAGTGTTAGCAGC | | ERATLSCRASQSVSSN |
| | | | AACTTAGCCTGGTACCAGCAGAAACCTGGCCAGGCTCCCAGGCTCC | | LAWYQQKPGQAPRLL |
| | | | TCATCTATGGTGCATCCACCAGGGCCACTGGTATCCCAGCCAGGTTC | | IYGASTRATGIPARFS |
| | | | AGTGGCAGTGGGTCTGGGACAGAGTTCACTCTCACCATCAGCAGCC | | GSGSGTEFTLTISSLQS |
| | | | TGCAGTCTGAAGATTTTGCAGTTTATTACTGTCAGCAGTATAATAAC | | EDFAVYYCQQYNNW |
| | | | TGGCCTCCGACTTTTGGCCAGGGGACCAAGCTGGAGATCAAA | | PPTFGQGTKLEIK |
| 27 | 13A12_f3_K | 56 | GAAATAGTGATGACGCAGTCTCCAGCCACCCTGTCTGTGTCTCCAGG | 114 | EIVMTQSPATLSVSPG |
| | | | GGAAAGAGCCACCCTCTCCTGCAGGGCCAGTCAGAGTGTTAGCAGC | | ERATLSCRASQSVSSN |
| | | | AACTTAGCCTGGTACCAGCAGAAACCTGGCCAGGCTCCCAGGCTCC | | LAWYQQKPGQAPRLL |
| | | | TCATCTATGGTGCATCCACCAGGGCCACTGGTATCCCAGCCAGGTTC | | IYGASTRATGIPARFS |
| | | | AGTGGCAGTGGGTCTGGGACAGAGTTCACTCTCACCATCAGCAGCC | | GSGSGTEFTLTISSLQS |
| | | | TGCAGTCTGAAGATTTTGCAGTTTATTACTGTCAGCAGTATGATAAC | | EDFAVYYCQQYDNW |
| | | | TGGCCTCTCACTTTCGGCGGAGGGACCAAGGTGGAGATCAAA | | PLTFGGGTKVEIK |
| 28 | 5B6_f3_K | 57 | GACATCCAGATGACCCAGTCTCCATCCTCCCTGTCTGCATCTGTAGG | 115 | DIQMTQSPSSLSASVG |
| | | | AGACAGAGTCACCATCACTTGCCGGGCGAGTCAGGGCATTAGCAAT | | DRVTITCRASQGISNY |
| | | | TATTTAGCCTGGTATCAGCAGAAACCAGGGAAAGTTCCTAAGCTCCT | | LAWYQQKPGKVPKL |
| | | | GATCTATGCTGCATCCACTTTGCAATCAGGGGTCCCATCTCGGTTCA | | LIYAASTLQSGVPSRF |
| | | | GTGGCAGTGGATCTGGGACAGATTTCACTCTCACCATCAGCAGCCTG | | SGSGSGTDFTLTISSLQ |
| | | | CAGCCTGAAGATGTTGCAACTTATTACTGTCAAAAGTATAACAGTGC | | PEDVATYYCQKYNSA |
| | | | CCCTCACACTTTTGGCCAGGGGACCAAGCTGGAGATCAAA | | PHTFGQGTKLEIK |
| 29 | 2G6_f4_K | 58 | GACATCCAGTTGACCCAGTCTCCATCCTTCCTGTCTGCATCTGTAGG | 116 | DIQLTQSPSFLSASVG |
| | | | AGACAGAGTCACCATCACTTGCCGGGCCAGTCAGGGCATTAGCAGT | | DRVTITCRASQGISSY |
| | | | TATTTAGCCTGGTATCAGCAAAAACCAGGGAAAGCCCCTAAGCTCC | | LAWYQQKPGKAPKL |
| | | | TGATCTATGCTGCATCCACTTTGCAAAGTGGGGTCCCATCAAGGTTC | | LIYAASTLQSGVPSRF |
| | | | AGCGGCAGTGGATCTGGGACAGAATTCACTCTCACAATCAGCAGCC | | SGSGSGTEFTLTISSLQ |
| | | | TGCAGTCTGAAGATTTTGCAATTTATTACTGTCAACAGCTTAATAGT | | SEDFAIYYCQQLNSYP |
| | | | TACCCGCTCACTTTCGGCGGAGGGACCAAGGTGGAGATCAAA | | LTFGGGTKVEIK |

TABLE 3
Summary of anti-SARS-CoV-2 Monoclonal Antibody Function.

| | | | ACE2 Inhibition |
|---|---|---|---|
| Antibody # | Clone | EC50 (pM) | (%) |
| | 1B1 | 90 | 07 |
| | 1B9 | 240 | 95 |
| 1 | 1C12 | 75 | 88 |
| 3 | 1D10 | 47 | 95 |
| 2 | 1D5 | 49 | 94 |
| 4 | 1E2 | 218 | 86 |
| 5 | 1G9 | 145 | 96 |
| | 2E10 | 573 | 98 |
| | 3E1 | 362 | 99 |
| | 3E6 | 195 | 95 |
| | 3E7 | 8175 | 91 |
| | 4B12 | 139 | 99 |
| | 4D3 | 6800 | 91 |
| | 4H11 | 6800 | 91 |
| | 5B11 | 6800 | 95 |
| 6 | 5D7 | 284 | 97 |
| 7 | 5H12 | 377 | 100 |
| | 5H3 | 127 | 99 |
| | 5H5 | 94 | 83 |
| | 6D12 | 6800 | 93 |
| 8 | 7C10 | 152 | 85 |
| | 7F3 | 5075 | 99 |
| | 8A1 | 96 | 95 |
| | 8E11 | 65 | 91 |
| 9 | 8H4 | 100 | 93 |
| 10 | 9C6 | 105 | 85 |
| | 9D10 | 262 | 93 |
| | 9C6 | 105 | 85 |
| | 9D10 | 262 | 93 |
| | 10A8 | 68 | 88 |
| 11 | 10D6 | 1016 | 81 |
| | 11B5 | 2240 | 89 |
| 12 | 11D5 | 440 | 66 |
| 13 | 11G2 | 74 | 90 |
| | 12A12 | 6800 | 98 |
| | 13B4 | 1199 | 87 |
| | 15C2 | 6800 | 56 |
| 16 | 16C12 | 59 | 87 |
| 15 | 16C5 | 107 | 94 |
| | 16G5 | 5139 | 77 |
| | 16H4 | 239 | 70 |
| 17 | 17A8 | 59 | 98 |
| | 17E4 | 256 | 72 |
| 18 | 17F2 | 151 | 65 |
| | 18B6 | 968 | 59 |
| 19 | 18E7 | 62 | 82 |
| | 19B3 | 128 | 91 |
| 20 | 19C4 | 492 | 89 |

TABLE 4
Summary of anti-SARS-CoV-2 Monoclonal Antibody Function.

| | | ACE2 binding | Inhibition: EC50 |
|---|---|---|---|
| Antibody # | Clone name | [% inhibition] | [pM] |
| 1 | 1C12 | 88 | 75 |
| 2 | 1D5 | 94 | 94 |
| 3 | 1D10 | 95 | 47 |
| 4 | 1E2 | 86 | 218 |
| 5 | 1G9 | 96 | 145 |
| 6 | 5D7 | 97 | 284 |
| 7 | 5H12 | 100 | 377 |
| 8 | 7C10 | 85 | 152 |
| 9 | 8H4 | 93 | 100 |
| 10 | 9C6 | 85 | 105 |
| 11 | 10D6 | 81 | 1016 |
| 12 | 11D5 | 66 | 440 |
| 13 | 11G2 | 90 | 109 |
| 15 | 16C5 | 94 | 107 |
| 16 | 16C12 | 87 | 59 |
| 17 | 17A8 | 98 | 59 |
| 18 | 17F2 | 65 | 151 |
| 19 | 18E7 | 82 | 62 |
| 20 | 19C4 | 89 | 492 |
| | 19B3 | 91 | 123 |

TABLE 5
Heavy Chain CDR Sequences (Nucleic Acid Sequences).

| | | Variable Heavy Chain Nucleotide Sequence | | | | | |
|---|---|---|---|---|---|---|---|
| | | CDR1-IMGT | | CDR2-IMGT | | CDR3-IMGT | |
| | | SEQ | | SEQ | | SEQ | |
| Ab # | Name | ID NO | Sequence | ID NO | Sequence | ID NO | Sequence |
| 1 | 1C12_f3_H | 117 | GGATACACCTTCAC | 146 | ATGAGCCCTAAC | 175 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 2 | 1D5_f3_H | 118 | GGATACACCTTCAC | 147 | ATGAGCCCTAAC | 176 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 3 | 1D10_f3_H | 119 | GGATACACCTTCAC | 148 | ATGAGCCCTAAC | 177 | GCGAGATTCGGCTATGGTTCGGG |
| | | | CAGTTATGAT | | AGCGGTAACACA | | GGCCCTCGACTACTACTATTACG |
| | | | | | | | GTTTGGACGTC |
| 4 | 1E2_f3_H | 120 | GGATACACCTTCAC | 149 | ATGAGCCCTAAC | 178 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 5 | 1G9_f3_H | 121 | GGATACACCTTCAC | 150 | ATGAGCCCTAAC | 179 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 6 | 5D7_f3_H | 122 | GGATACACCTTCAC | 151 | ATGAGCCCTAAA | 180 | GCGAGATTCGGCTATGGTTCGGG |
| | | | CAGTTATGAT | | AATGGTAACACA | | GGCCCTCGACTACTACTACTACG |
| | | | | | | | GTTTGGACGTC |
| 7 | 5H12_f3_H | 123 | GGATACACCTTCCC | 152 | ATGAGCCCTAAA | 181 | GCGAGATTCGGCTATGGGTCGGG |
| | | | CAGTTATGAT | | AATGGTAACACA | | GGCCCTCGGATATTACTACTACG |
| | | | | | | | GTTTGGACGTC |
| 8 | 7C10_f3_H | 124 | GGATACACCTTCAC | 153 | ATGAGCCCTAAC | 182 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 9 | 8H4_f3_H | 125 | GGATACACCTTCAC | 154 | ATGAGCCCTAAC | 183 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 10 | 9C6_f3_H | 126 | GGATACACCTTCAC | 155 | ATGAGCCCTAAA | 184 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 11 | 10D6_f3_H | 127 | GGGATCACCGTCAG | 156 | ATTTATAGCGGT | 185 | GCGAGAGATTTAGAACTGGCTGG |
| | | | TAGTAACTAC | | GGTAGCACA | | AGCTTTTGATATC |
| 12 | 11D5_f3_H | 128 | GGGATCACCGTCAG | 157 | ATTTATAGCGGT | 186 | GCGAGAGATTTAGCAGTGGCTGG |
| | | | TAGTAACTAC | | GGTAGCACA | | AGCTTTTGATATC |
| 13 | 11G2_f3_H | 129 | GGATACACCTTCAC | 158 | ATGAGCCCTAAC | 187 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 14 | 11G7_f2_H | 130 | GGTGGCTCCATCAG | 159 | ATCTTTCATAGT | 188 | GCGAGATTCAGAAGGATAGTGGC |
| | | | CAGTAGTAACTGG | | GGGAGCACC | | TACGAGCTACTATTTTGACTAC |
| 15 | 16C5_f3_H | 131 | GGATACACCTTCAC | 160 | ATGAGCCCTAAT | 189 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 16 | 16C12_f3_H | 132 | GGATACACCTTCAC | 161 | ATGAGCCCTAAC | 190 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 17 | 17A8_f3_H | 133 | GGATACACCTTCAC | 162 | ATGAGCCCTAAC | 191 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 18 | 17F2_f3_H | 134 | GGATACACCTTCAC | 163 | ATGAGCCCTGAC | 192 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 19 | 18E7_f3_H | 135 | GGATACACCTTTAC | 164 | ATGAGCCCTAAC | 193 | GCGAGATTCGGCTATGGTTCGGG |
| | | | CAGTTATGAT | | AGTGGTAACACA | | GGCCCTCGACTACTACTACTACG |
| | | | | | | | GTTTGGACGTC |
| 20 | 19C4_f3_H | 136 | GGGATCACCGTCAG | 165 | ATTTATAGCGGT | 194 | GCGAGAGATTTAGAAGTGGCTGG |
| | | | TAGTAATTAC | | GGTAGCACA | | AGGTTTTGATATC |
| 21 | 2C1_f4_H | 137 | GGATTCACCTTTGG | 166 | ATTAGAAGCAAA | 195 | ACTAGAGTGTCCGGGTATAGCAA |
| | | | TGATTATACT | | CCTTTTGGTGGG | | CATCTGGTTCTTTGCCTAC |
| | | | | | ACAACA | | |
| 22 | 4E3_f4_H | 138 | GGATTCACCTTCAG | 167 | ATTTGGTATGAT | 196 | GCGAGAAAAGATTATTCGAAGAC |
| | | | TAGCTATGGC | | GGAAATAATAAA | | ATTTTATGGATACTACTTTGACTA |
| | | | | | | | T |
| 23 | 7D2_f4_H | 139 | GGGTTCACCGTCAG | 168 | ATTTATAGCGGT | 197 | GCGAGAGATCTAGTCATCTACGG |
| | | | TAGCAACTAC | | GGTAGCACA | | TATGGACGTC |
| 24 | 9C5_f4_H | 140 | GGATTCACCTTCAG | 169 | ATTTGGTATGAT | 198 | GCGAGAAAAGATGGTTCGAAGAC |
| | | | TAGCTATGGC | | GGAAATAATAAA | | ATATTATGGATACTACTTTGACTA |
| | | | | | | | T |
| 25 | 10A3_f4_H | 141 | GGATTCACCTTCAG | 170 | ATTTGGTATGAT | 199 | GCGAGAAAAGATGGTTCGAAGAC |
| | | | TAGTTATGGC | | GGAAATAATACA | | ATATTATGGATACTACTTTGACTA |
| | | | | | | | T |
| 26 | 14G5_f4_H | 142 | GGTGGCTCCATCAG | 171 | ATCTTGCATGGT | 200 | GCGAGAGATGCGAATTTCTATGG |
| | | | CAGTAATAACTGG | | GAGATCACC | | TTCGGGGAGTTCTTACTTTGACTA |
| | | | | | | | C |
| 27 | 13A12_f3_H | 143 | GGTGGCTCCATCTA | 172 | ATCTATCATAGT | 201 | GCGAGAGATCAAGATTACTATGG |
| | | | CAGTAGTAACTGC | | GGGGGCACC | | TTCGGGGAGTTCCCTCTTTGACTA |
| | | | | | | | C |
| 28 | 5B6_f3_H | 144 | GGATTCACTTTCAG | 173 | TTTAAAAGCAAA | 202 | ACCACCAGCAGTGGCTAC |
| | | | TAACGCCTGG | | ACTGATGGTGGG | | |
| | | | | | ACAACA | | |
| 29 | 2G6_f4_H | 145 | GGTGGGTCCTTCAG | 174 | ATCAATCATAGT | 203 | GCGAGGTATTACTATGATGGTAA |
| | | | TGTTTACTAC | | GGAAACACC | | TGGTTATTACCCC |

TABLE 6
Heavy Chain CDR Sequences (Amino Acid Sequences).

| | | Variable Heavy Chain Amino Acid Sequence | | | | | |
|---|---|---|---|---|---|---|---|
| | | CDR1-IMGT | | CDR2-IMGT | | CDR3-IMGT | |
| | | SEQ | | SEQ | | SEQ | |
| Ab # | Name | ID NO | Sequence | ID NO | Sequence | ID NO | Sequence |
| 1 | 1C12_f3_H | 204 | GYTFTSYD | 233 | MSPNSGNT | 262 | ARFGYGSGALDYYYYGLDV |
| 2 | 1D5_f3_H | 205 | GYTFTSYD | 234 | MSPNSGNT | 263 | ARFGYGSGALDYYYYGLDV |
| 3 | 1D10_f3_H | 206 | GYTFTSYD | 235 | MSPNSGNT | 264 | ARFGYGSGALDYYYYGLDV |
| 4 | 1E2_f3_H | 207 | GYTFTSYD | 236 | MSPNSGNT | 265 | ARFGYGSGALDYYYYGLDV |
| 5 | 1G9_f3_H | 208 | GYTFTSYD | 237 | MSPNSGNT | 266 | ARFGYGSGALDYYYYGLDV |
| 6 | 5D7_f3_H | 209 | GYTFTSYD | 238 | MSPKNGNT | 267 | ARFGYGSGALDYYYYGLDV |
| 7 | 5H12_f3_H | 210 | GYTFPSYD | 239 | MSPKNGNT | 268 | ARFGYGSGALGYYYYGLDV |
| 8 | 7C10_f3_H | 211 | GYTFTSYD | 240 | MSPNSGNT | 269 | ARFGYGSGALDYYYYGLDV |
| 9 | 8H4_f3_H | 212 | GYTFTSYD | 241 | MSPNSGNT | 270 | ARFGYGSGALDYYYYGLDV |
| 10 | 9C6_f3_H | 213 | GYTFTSYD | 242 | MSPKSGNT | 271 | ARFGYGSGALDYYYYGLDV |
| 11 | 10D6_f3_H | 214 | GITVSSNY | 243 | IYSGGST | 272 | ARDLELAGAFDI |
| 12 | 11D5_f3_H | 215 | GITVSSNY | 244 | IYSGGST | 273 | ARDLAVAGAFDI |
| 13 | 11G2_f3_H | 216 | GYTFTSYD | 245 | MSPNSGNT | 274 | ARFGYGSGALDYYYYGLDV |
| 14 | 11G7_f2_H | 217 | GGSISSSNW | 246 | IFHSGST | 275 | ARFRRIVATSYYFDY |
| 15 | 16C5_f3_H | 218 | GYTFTSYD | 247 | MSPNSGNT | 276 | ARFGYGSGALDYYYYGLDV |
| 16 | 16C12_f3_H | 219 | GYTFTSYD | 248 | MSPNSGNT | 277 | ARFGYGSGALDYYYYGLDV |
| 17 | 17A8_f3_H | 220 | GYTFTSYD | 249 | MSPNSGNT | 278 | ARFGYGSGALDYYYYGLDV |
| 18 | 17F2_f3_H | 221 | GYTFTSYD | 250 | MSPDSGNT | 279 | ARFGYGSGALDYYYYGLDV |
| 19 | 18E7_f3_H | 222 | GYTFTSYD | 251 | MSPNSGNT | 280 | ARFGYGSGALDYYYYGLDV |
| 20 | 19C4_f3_H | 223 | GITVSSNY | 252 | IYSGGST | 281 | ARDLEVAGGFDI |
| 21 | 2C1_f4_H | 224 | GFTFGDYT | 253 | IRSKPFGGTT | 282 | TRVSGYSNIWFFAY |
| 22 | 4E3_f4_H | 225 | GFTFSSYG | 254 | IWYDGNNK | 283 | ARKDYSKTFYGYYFDY |
| 23 | 7D2_f4_H | 226 | GFTVSSNY | 255 | IYSGGST | 284 | ARDLVIYGMDV |
| 24 | 9C5_f4_H | 227 | GFTFSSYG | 256 | IWYDGNNK | 285 | ARKDGSKTYYGYYFDY |
| 25 | 10A3_f4_H | 228 | GFTFSSYG | 257 | IWYDGNNT | 286 | ARKDGSKTYYGYYFDY |
| 26 | 14G5_f4_H | 229 | GGSISSNNW | 258 | ILHGEIT | 287 | ARDANFYGSGSSYFDY |
| 27 | 13A12_f3_H | 230 | GGSIYSSNC | 259 | IYHSGGT | 288 | ARDQDYYGSGSSLFDY |
| 28 | 5B6_f3_H | 231 | GFTFSNAW | 260 | FKSKTDGGTT | 289 | TTSSGY |
| 29 | 2G6_f4_H | 232 | GGSFSVYY | 261 | INHSGNT | 290 | ARYYYDGNGYYP |

TABLE 7
Light Chain CDR Sequences (Nucleic Acid Sequences)

| | | Variable Light Chain Nucleotide Sequence | | | | | |
|---|---|---|---|---|---|---|---|
| | | CDR1-IMGT | | CDR2-IMGT | | CDR3-IMGT | |
| | | SEQ | | SEQ | | SEQ | |
| Ab # | Name | ID NO | Sequence | ID NO | Sequence | ID NO | Sequence |
| 1 | 1C12_f3_K | 291 | CAGAGTGTTAGGAG | 320 | GGTGCATCC | 349 | CAGCAGTATAATAACTCACCGAT |
| | | | CAGTCAC | | | | CACC |
| 2 | 1D5_f3_K | 292 | CAGAGTGTTAGGAG | 321 | GGTGCATCC | 350 | CAGCAGTTTGGTAGCTCACCGAT |
| | | | CAGTCAC | | | | CACC |
| 3 | 1D10_f3_K | 293 | CAGAGTGTTAGCAG | 322 | GGTGCATCC | 35 | CAGCAGTATAATAGCTCACCGAT |
| | | | CAGCCAC | | | | CACC |
| 4 | 1E2_f3_K | 294 | CAGAGTGTTAGGAG | 323 | GGTGCATCC | 352 | CAGCAGTATGGTAGCTCACCGAT |
| | | | CAGTCAC | | | | CACC |
| 5 | 1G9_f3_K | 295 | CAGAGTGTTAGGAG | 324 | GGTGCATCC | 353 | CAGCAGTATGGTAGCTCACCGAT |
| | | | CAGTCAC | | | | CACC |
| 6 | 5D7_f3_K | 296 | CAGAGTGTTAGGAG | 325 | GGTGCCTCC | 354 | CAGCAGTATAATATCTCACCGAT |
| | | | CAGTCAC | | | | CACC |
| 7 | 5H12_f3_K | 297 | CAGAGTGTTAGGAG | 326 | GGTGCCTCC | 355 | CAGCAGTATAATTTCTCACCGAT |
| | | | CAGTCAC | | | | CACC |
| 8 | 7C10_f3_K | 298 | CAGAGTGTTAGGAG | 327 | GGTGCATCC | 356 | CAGCAGTTTGGTAGCTCACCGAT |
| | | | CAGTCAC | | | | CACC |
| 9 | 8H4_f3_K | 299 | CAGAGTGTTAGGAG | 328 | GGTGCATCC | 357 | CAGCAGTTTGGTAGCTCACCGAT |
| | | | CAGTCAC | | | | CACC |
| 10 | 9C6_f3_K | 300 | CAGAGTGTTAGGAG | 329 | GGTGCCTCC | 358 | CAGCAGTATAATAGCTCACCGAT |
| | | | CAGTCAC | | | | CACC |
| 11 | 10D6_f3_K | 301 | CAGGGCATTAGCAG | 330 | GCTGCATCC | 359 | CAACAGCTTAATAGTTACCCTCC |
| | | | TTAT | | | | GTCCACT |
| 12 | 11D5_f3_K | 302 | CAGGGCATTAGCAG | 331 | GCTGCATCC | 360 | CAACAGCTTAATAGTTACCCTCC |
| | | | TTAT | | | | GTCCACT |
| 13 | 11G2_f3_K | 303 | CAGAGTGTTAGGAA | 332 | GGTGCATCC | 36 | CAGCAGTTTGGTAGCTCACCGAT |
| | | | CAGTCAC | | | | CACC |
| 14 | 11G7_f2_K | 304 | CAGAGCCTCCTGCA | 333 | TTGGGTTCT | 362 | ATGCAAGCTCTACAAACTCCTCT |
| | | | TAGTAATGGATACA | | | | CACT |
| | | | ACTAT | | | | |
| 15 | 16C5_f3_K | 305 | CAGAGTGTTAGGAG | 334 | GGTGCATCC | 363 | CAGCAGTATAATAACTCACCGAT |
| | | | CAGTCAC | | | | CACC |
| 16 | 16C12_f3_K | 306 | CAGAGTGTTAGGAG | 335 | GGTGCATCC | 364 | CAGCAGTATGGTAGCTCACCGAT |
| | | | CAGTCAC | | | | CACC |
| 17 | 17A8_f3_K | 307 | CAGAGTGTTAGGAG | 336 | GGTGCATCC | 365 | CAGCAGTATGGTAGCTCACCGAT |
| | | | CAGTCAC | | | | CACC |
| 18 | 17F2_f3_K | 308 | CAGAGTGTTAGGAG | 337 | GGTGCATCC | 366 | CAGCAGTATAATAGCTCACCGAT |
| | | | CAGTCAC | | | | CACC |
| 19 | 18E7_f3_K | 309 | CAGAGTGTTAGGAG | 338 | GGTGCATCC | 367 | CAGCAGTATGGTAGCTCACCGAT |
| | | | CAGTCAC | | | | CACC |
| 20 | 19C4_f3_K | 310 | CAGGGCATTAGCAG | 339 | GCTGCATCC | 368 | CAACAGCTTAATAGTTTCCCTCC |
| | | | TTAT | | | | GTCCACT |
| 21 | 2C1_f4_K | 311 | CAGAGTGTTAGCAT | 340 | GGTGCATCC | 369 | CAGCAGTATAATAACTGGTGGAC |
| | | | CAAC | | | | G |
| 22 | 4E3_f4_K | 312 | CAGAGCATTCACAG | 341 | GCTGCATCC | 370 | CAACAGAGTTACATTACCCCTCC |
| | | | CTTT | | | | GACG |
| 23 | 7D2_f4_K | 313 | CAGAGTGTTAGCAG | 342 | GGTGCATCC | 371 | CAGCAGTATAATAACTGGCCCCC |
| | | | CAAC | | | | T |
| 24 | 9C5_f4_K | 314 | CAGAGCATTCACAG | 343 | ACTACATCC | 372 | CAACAGAGTTACATTACCCCTCC |
| | | | CTTT | | | | GACG |
| 25 | 10A3_f4_K | 315 | CAGAGCATTCACAG | 344 | GCTGCATCC | 373 | CAACAGAGTTACATTACCCCTCC |
| | | | CTTT | | | | GACG |
| 26 | 14G5_f4_K | 316 | CAGAGTGTTAGCAG | 345 | GGTGCATCC | 374 | CAGCAGTATAATAACTGGCCTCC |
| | | | CAAC | | | | GACT |
| 27 | 13A12_f3_K | 317 | CAGAGTGTTAGCAG | 346 | GGTGCATCC | 375 | CAGCAGTATGATAACTGGCCTCT |
| | | | CAAC | | | | CACT |
| 28 | 5B6_f3_K | 318 | CAGGGCATTAGCAA | 347 | GCTGCATCC | 376 | CAAAAGTATAACAGTGCCCCTCA |
| | | | TTAT | | | | CACT |
| 29 | 2G6_f4_K | 319 | CAGGGCATTAGCAG | 348 | GCTGCATCC | 377 | CAACAGCTTAATAGTTACCCGCT |
| | | | TTAT | | | | CACT |

TABLE 8
Light Chain CDR Sequences (Amino Acid Sequences)

| | | Variable Light Chain Amino Acid Sequence | | | | | |
|---|---|---|---|---|---|---|---|
| | | CDR1-IMGT | | CDR2-IMGT | | CDR3-IMGT | |
| Ab # | Name | SEQ ID NO | Sequence | SEQ ID NO | Sequence | SEQ ID NO | Sequence |
| 1 | 1C12_f3_K | 378 | QSVRSSH | n/a | GAS | 407 | QQYNNSPIT |
| 2 | 1D5_f3_K | 379 | QSVRSSH | n/a | GAS | 408 | QQFGSSPIT |
| 3 | 1D10_f3_K | 380 | QSVSSSH | n/a | GAS | 409 | QQYNSSPIT |
| 4 | 1E2_f3_K | 381 | QSVRSSH | n/a | GAS | 410 | QQYGSSPIT |
| 5 | 1G9_f3_K | 382 | QSVRSSH | n/a | GAS | 411 | QQYGSSPIT |
| 6 | 5D7_f3_K | 383 | QSVRSSH | n/a | GAS | 412 | QQYNISPIT |
| 7 | 5H12_f3_K | 384 | QSVRSSH | n/a | GAS | 413 | QQYNFSPIT |
| 8 | 7C10_f3_K | 385 | QSVRSSH | n/a | GAS | 414 | QQFGSSPIT |
| 9 | 8H4_f3_K | 386 | QSVRSSH | n/a | GAS | 415 | QQFGSSPIT |
| 10 | 9C6_f3_K | 387 | QSVRSSH | n/a | GAS | 416 | QQYNSSPIT |
| 11 | 10D6_f3_K | 388 | QGISSY | n/a | AAS | 417 | QQLNSYPPST |
| 12 | 11D5_f3_K | 389 | QGISSY | n/a | AAS | 418 | QQLNSYPPST |
| 13 | 11G2_f3_K | 390 | QSVRNSH | n/a | GAS | 419 | QQFGSSPIT |
| 14 | 11G7_f2_K | 391 | QSLLHSNGYNY | n/a | LGS | 420 | MQALQTPLT |
| 15 | 16C5_f3_K | 392 | QSVRSSH | n/a | GAS | 421 | QQYNNSPIT |
| 16 | 16C12_f3_K | 393 | QSVRSSH | n/a | GAS | 422 | QQYGSSPIT |
| 17 | 17A8_f3_K | 394 | QSVRSSH | n/a | GAS | 423 | QQYGSSPIT |
| 18 | 17F2_f3_K | 395 | QSVRSSH | n/a | GAS | 424 | QQYNSSPIT |
| 19 | 18E7_f3_K | 396 | QSVRSSH | n/a | GAS | 425 | QQYGSSPIT |
| 20 | 19C4_f3_K | 397 | QGISSY | n/a | AAS | 426 | QQLNSFPPST |
| 21 | 2C1_f4_K | 398 | QSVSIN | n/a | GAS | 427 | QQYNNWWT |
| 22 | 4E3_f4_K | 399 | QSIHSF | n/a | AAS | 428 | QQSYITPPT |
| 23 | 7D2_f4_K | 400 | QSVSSN | n/a | GAS | 429 | QQYNNWPP |
| 24 | 9C5_f4_K | 401 | QSIHSF | n/a | TTS | 430 | QQSYITPPT |
| 25 | 10A3_f4_K | 402 | QSIHSF | n/a | AAS | 431 | QQSYITPPT |
| 26 | 14G5_f4_K | 403 | QSVSSN | n/a | GAS | 432 | QQYNNWPPT |
| 27 | 13A12_f3_K | 404 | QSVSSN | n/a | GAS | 433 | QQYDNWPLT |
| 28 | 5B6_f3_K | 405 | QGISNY | n/a | AAS | 434 | QKYNSAPHT |
| 29 | 2G6_f4_K | 406 | QGISSY | n/a | AAS | 435 | QQLNSYPLT |

TABLE 9
Summary of Antibody Sequences (Nucleic Acid Sequences)

| | | | CDR1H- | CDR2H- | CDR3H- | | CDR1L- | CDR2L- | CDR3L- |
|---|---|---|---|---|---|---|---|---|---|
| | | VH | IMGT | IMGT | IMGT | VL | IMGT | IMGT | IMGT |
| Antibody | Antibody | SEQ ID | SEQ ID | SEQ ID | SEQ ID | SEQ ID | SEQ ID | SEQ ID | SEQ ID |
| # | name | NO | NO | NO | NO | NO | NO | NO | NO |
| 1 | 1C12_f3 | 1 | 117 | 146 | 175 | 30 | 291 | 320 | 349 |
| 2 | 1D5_f3 | 2 | 118 | 147 | 176 | 31 | 292 | 321 | 350 |
| 3 | 1D10_f3 | 3 | 119 | 148 | 177 | 32 | 293 | 322 | 351 |
| 4 | 1E2_f3 | 4 | 120 | 149 | 178 | 33 | 294 | 323 | 352 |
| 5 | 1G9_f3 | 5 | 121 | 150 | 179 | 34 | 295 | 324 | 353 |
| 6 | 5D7_f3 | 6 | 122 | 151 | 180 | 35 | 296 | 325 | 354 |
| 7 | 5H12_f3 | 7 | 123 | 152 | 181 | 36 | 297 | 326 | 355 |
| 8 | 7C10_f3 | 8 | 124 | 153 | 182 | 37 | 298 | 327 | 356 |
| 9 | 8H4_f3 | 9 | 125 | 154 | 183 | 38 | 299 | 328 | 357 |
| 10 | 9C6_f3 | 10 | 126 | 155 | 184 | 39 | 300 | 329 | 358 |
| 11 | 10D6_f3 | 11 | 127 | 156 | 185 | 40 | 301 | 330 | 359 |
| 12 | 11D5_f3 | 12 | 128 | 157 | 186 | 41 | 302 | 331 | 360 |
| 13 | 11G2_f3 | 13 | 129 | 158 | 187 | 42 | 303 | 332 | 361 |
| 14 | 11G7_f2 | 14 | 130 | 159 | 188 | 43 | 304 | 333 | 362 |
| 15 | 16C5_f3 | 15 | 131 | 160 | 189 | 44 | 305 | 334 | 363 |
| 16 | 16C12_f3 | 16 | 132 | 161 | 190 | 45 | 306 | 335 | 364 |
| 17 | 17A8_f3 | 17 | 133 | 162 | 191 | 46 | 307 | 336 | 365 |
| 18 | 17F2_f3 | 18 | 134 | 163 | 192 | 47 | 308 | 337 | 366 |
| 19 | 18E7_f3 | 19 | 135 | 164 | 193 | 48 | 309 | 338 | 367 |
| 20 | 19C4_f3 | 20 | 136 | 165 | 194 | 49 | 310 | 339 | 368 |
| 21 | 2C1_f4 | 21 | 137 | 166 | 195 | 50 | 311 | 340 | 369 |
| 22 | 4E3_f4 | 22 | 138 | 167 | 196 | 51 | 312 | 341 | 370 |
| 23 | 7D2_f4 | 23 | 139 | 168 | 197 | 52 | 313 | 342 | 371 |
| 24 | 9C5_f4 | 24 | 140 | 169 | 198 | 53 | 314 | 343 | 372 |
| 25 | 10A3_f4 | 25 | 141 | 170 | 199 | 54 | 315 | 344 | 373 |
| 26 | 14G5_f4 | 26 | 142 | 171 | 200 | 55 | 316 | 345 | 374 |
| 27 | 13A12_f3 | 27 | 143 | 172 | 201 | 56 | 317 | 346 | 375 |
| 28 | 5B6_f3 | 28 | 144 | 173 | 202 | 57 | 318 | 347 | 376 |
| 29 | 2G6_f4 | 29 | 145 | 174 | 203 | 58 | 319 | 348 | 377 |

TABLE 10
Summary of Antibody Sequences (Amino Acid Sequences)

| | | | CDR1H- | CDR2H- | CDR3H- | | CDR1L- | | CDR3L- |
|---|---|---|---|---|---|---|---|---|---|
| | | VH | IMGT | IMGT | IMGT | VL | IMGT | CDR2L- | IMGT |
| Antibody | Antibody | SEQ ID | SEQ ID | SEQ ID | SEQ ID | SEQ ID | SEQ ID | IMGT | SEQ ID |
| # | name | NO | NO | NO | NO | NO | NO | Sequence | NO |
| 1 | 1C12_f3 | 59 | 204 | 233 | 262 | 88 | 378 | GAS | 407 |
| 2 | 1D5_f3 | 60 | 205 | 234 | 263 | 89 | 379 | GAS | 408 |
| 3 | 1D10_f3 | 61 | 206 | 235 | 264 | 90 | 380 | GAS | 409 |
| 4 | 1E2_f3 | 62 | 207 | 236 | 265 | 91 | 381 | GAS | 410 |
| 5 | 1G9_f3 | 63 | 208 | 237 | 266 | 92 | 382 | GAS | 411 |
| 6 | 5D7_f3 | 64 | 209 | 238 | 267 | 93 | 383 | GAS | 412 |
| 7 | 5H12_f3 | 65 | 210 | 239 | 268 | 94 | 384 | GAS | 413 |
| 8 | 7C10_f3 | 66 | 211 | 240 | 269 | 95 | 385 | GAS | 414 |
| 9 | 8H4_f3 | 67 | 212 | 241 | 270 | 96 | 386 | GAS | 415 |
| 10 | 9C6_f3 | 68 | 213 | 242 | 271 | 97 | 387 | GAS | 416 |
| 11 | 10D6_f3 | 69 | 214 | 243 | 272 | 98 | 388 | AAS | 417 |
| 12 | 11D5_f3 | 70 | 215 | 244 | 273 | 99 | 389 | AAS | 418 |
| 13 | 11G2_f3 | 71 | 216 | 245 | 274 | 100 | 390 | GAS | 419 |
| 14 | 11G7_f2 | 72 | 217 | 246 | 275 | 101 | 391 | LGS | 420 |
| 15 | 16C5_f3 | 73 | 218 | 247 | 276 | 102 | 392 | GAS | 421 |
| 16 | 16C12_f3 | 74 | 219 | 248 | 277 | 103 | 393 | GAS | 422 |
| 17 | 17A8_f3 | 75 | 220 | 249 | 278 | 104 | 394 | GAS | 423 |
| 18 | 17F2_f3 | 76 | 221 | 250 | 279 | 105 | 395 | GAS | 424 |
| 19 | 18E7_f3 | 77 | 222 | 251 | 280 | 106 | 396 | GAS | 425 |
| 20 | 19C4_f3 | 78 | 223 | 252 | 28 | 107 | 397 | AAS | 426 |
| 21 | 2C1_f4 | 79 | 224 | 253 | 282 | 108 | 398 | GAS | 427 |
| 22 | 4E3_f4 | 80 | 225 | 254 | 283 | 109 | 399 | AAS | 428 |
| 23 | 7D2_f4 | 81 | 226 | 255 | 284 | 110 | 400 | GAS | 429 |
| 24 | 9C5_f4 | 82 | 227 | 256 | 285 | 111 | 401 | TTS | 430 |
| 25 | 10A3_f4 | 83 | 228 | 257 | 286 | 112 | 402 | AAS | 431 |
| 26 | 14G5_f4 | 84 | 229 | 258 | 287 | 113 | 403 | GAS | 432 |
| 27 | 13A12_f3 | 85 | 230 | 259 | 288 | 114 | 404 | GAS | 433 |
| 28 | 5B6_f3 | 86 | 231 | 260 | 289 | 115 | 405 | AAS | 434 |
| 29 | 2G6_f4 | 87 | 232 | 261 | 290 | 116 | 406 | AAS | 435 |

The foregoing is not to be limited in scope by the specific embodiments described herein. Indeed, various modifications of the antibodies and methods provided herein and their equivalents, in addition to those described herein will become apparent to those skilled in the art from the foregoing description and accompanying figures. Such modifications are intended to fall within the scope of the appended claims.

All references cited herein are incorporated herein by reference in their entireties and for all purposes to the same extent as if each individual publication or patent or patent application was specifically and individually indicated to be incorporated by reference in its entirety for all purposes.

1. An isolated antibody or antigen-binding fragment thereof that binds to SARS-CoV-2 spike protein, wherein the antibody or antigen-binding fragment thereof comprises a variable heavy chain region comprising a CDR1H, a CDR2H, and a CDR3H, and a variable light chain region comprising a CDR1L, a CDR2L, and a CDR3L, wherein:
a) CDR1H comprises SEQ ID NO:204, CDR2H comprises SEQ ID NO:233, CDR3H comprises SEQ ID NO:262, CDR1L comprises SEQ ID NO:378, CDR2L comprises sequence GAS, and CDR3L comprises SEQ ID NO:407;
b) CDR1H comprises SEQ ID NO:205, CDR2H comprises SEQ ID NO:234, CDR3H comprises SEQ ID NO:263, CDR1L comprises SEQ ID NO:379, CDR2L comprises sequence GAS, and CDR3L comprises SEQ ID NO:408;
c) CDR1H comprises SEQ ID NO:206, CDR2H comprises SEQ ID NO:235, CDR3H comprises SEQ ID NO:264, CDR1L comprises SEQ ID NO:380, CDR2L comprises sequence GAS, and CDR3L comprises SEQ ID NO:409;
d) CDR1H comprises SEQ ID NO:207, CDR2H comprises SEQ ID NO:236, CDR3H comprises SEQ ID NO:265, CDR1L comprises SEQ ID NO:381, CDR2L comprises sequence GAS, and CDR3L comprises SEQ ID NO:410;
e) CDR1H comprises SEQ ID NO:208, CDR2H comprises SEQ ID NO:237, CDR3H comprises SEQ ID NO:266, CDR1L comprises SEQ ID NO:382, CDR2L comprises sequence GAS, and CDR3L comprises SEQ ID NO:411;
f) CDR1H comprises SEQ ID NO:209, CDR2H comprises SEQ ID NO:238, CDR3H comprises SEQ ID NO:267, CDR1L comprises SEQ ID NO:383, CDR2L comprises sequence GAS, and CDR3L comprises SEQ ID NO:412;
g) CDR1H comprises SEQ ID NO:210, CDR2H comprises SEQ ID NO:239, CDR3H comprises SEQ ID NO:268, CDR1L comprises SEQ ID NO:384, CDR2L comprises sequence GAS, and CDR3L comprises SEQ ID NO:413;
h) CDR1H comprises SEQ ID NO:211, CDR2H comprises SEQ ID NO:240, CDR3H comprises SEQ ID NO:269, CDR1L comprises SEQ ID NO:385, CDR2L comprises sequence GAS, and CDR3L comprises SEQ ID NO:414;
i) CDR1H comprises SEQ ID NO:212, CDR2H comprises SEQ ID NO:241, CDR3H comprises SEQ ID NO:270, CDR1L comprises SEQ ID NO:386, CDR2L comprises sequence GAS, and CDR3L comprises SEQ ID NO:415;
j) CDR1H comprises SEQ ID NO:213, CDR2H comprises SEQ ID NO:242, CDR3H comprises SEQ ID NO:271, CDR1L comprises SEQ ID NO:387, CDR2L comprises sequence GAS, and CDR3L comprises SEQ ID NO:416;
k) CDR1H comprises SEQ ID NO:214, CDR2H comprises SEQ ID NO:243, CDR3H comprises SEQ ID NO:272, CDR1L comprises SEQ ID NO:388, CDR2L comprises sequence AAS, and CDR3L comprises SEQ ID NO:417;
l) CDR1H comprises SEQ ID NO:215, CDR2H comprises SEQ ID NO:244, CDR3H comprises SEQ ID NO:273, CDR1L comprises SEQ ID NO:389, CDR2L comprises sequence AAS, and CDR3L comprises SEQ ID NO:418;
m) CDR1H comprises SEQ ID NO:216, CDR2H comprises SEQ ID NO:245, CDR3H comprises SEQ ID NO:274, CDR1L comprises SEQ ID NO:390, CDR2L comprises sequence GAS, and CDR3L comprises SEQ ID NO:419;
n) CDR1H comprises SEQ ID NO:217, CDR2H comprises SEQ ID NO:246, CDR3H comprises SEQ ID NO:275, CDR1L comprises SEQ ID NO:391, CDR2L comprises sequence LGS, and CDR3L comprises SEQ ID NO:420;
o) CDR1H comprises SEQ ID NO:218, CDR2H comprises SEQ ID NO:247, CDR3H comprises SEQ ID NO:276, CDR1L comprises SEQ ID NO:392, CDR2L comprises sequence GAS, and CDR3L comprises SEQ ID NO:421;
p) CDR1H comprises SEQ ID NO:219, CDR2H comprises SEQ ID NO:248, CDR3H comprises SEQ ID NO:277, CDR1L comprises SEQ ID NO:393, CDR2L comprises sequence GAS, and CDR3L comprises SEQ ID NO:422;
q) CDR1H comprises SEQ ID NO:220, CDR2H comprises SEQ ID NO:249, CDR3H comprises SEQ ID NO:278, CDR1L comprises SEQ ID NO:394, CDR2L comprises sequence GAS, and CDR3L comprises SEQ ID NO:423;
r) CDR1H comprises SEQ ID NO:221, CDR2H comprises SEQ ID NO:250, CDR3H comprises SEQ ID NO:279, CDR1L comprises SEQ ID NO:395, CDR2L comprises sequence GAS, and CDR3L comprises SEQ ID NO:424;
s) CDR1H comprises SEQ ID NO:222, CDR2H comprises SEQ ID NO:251, CDR3H comprises SEQ ID NO:280, CDR1L comprises SEQ ID NO:396, CDR2L comprises sequence GAS, and CDR3L comprises SEQ ID NO:425;
t) CDR1H comprises SEQ ID NO:223, CDR2H comprises SEQ ID NO:252, CDR3H comprises SEQ ID NO:281, CDR1L comprises SEQ ID NO:397, CDR2L comprises sequence AAS, and CDR3L comprises SEQ ID NO:426;
u) CDR1H comprises SEQ ID NO:224, CDR2H comprises SEQ ID NO:253, CDR3H comprises SEQ ID NO:282, CDR1L comprises SEQ ID NO:398, CDR2L comprises sequence GAS, and CDR3L comprises SEQ ID NO:427;
v) CDR1H comprises SEQ ID NO:225, CDR2H comprises SEQ ID NO:254, CDR3H comprises SEQ ID NO:283, CDR1L comprises SEQ ID NO:399, CDR2L comprises sequence AAS, and CDR3L comprises SEQ ID NO:428;
w) CDR1H comprises SEQ ID NO:226, CDR2H comprises SEQ ID NO:255, CDR3H comprises SEQ ID NO:284, CDR1L comprises SEQ ID NO:400, CDR2L comprises sequence GAS, and CDR3L comprises SEQ ID NO:429;
x) CDR1H comprises SEQ ID NO:227, CDR2H comprises SEQ ID NO:256, CDR3H comprises SEQ ID NO:285, CDR1L comprises SEQ ID NO:401, CDR2L comprises sequence TTS, and CDR3L comprises SEQ ID NO:430;
y) CDR1H comprises SEQ ID NO:228, CDR2H comprises SEQ ID NO:257, CDR3H comprises SEQ ID NO:286, CDR1L comprises SEQ ID NO:402, CDR2L comprises sequence AAS, and CDR3L comprises SEQ ID NO:431;
z) CDR1H comprises SEQ ID NO:229, CDR2H comprises SEQ ID NO:258, CDR3H comprises SEQ ID NO:287, CDR1L comprises SEQ ID NO:403, CDR2L comprises sequence GAS, and CDR3L comprises SEQ ID NO:432;
aa) CDR1H comprises SEQ ID NO:230, CDR2H comprises SEQ ID NO:259, CDR3H comprises SEQ ID NO:288, CDR1L comprises SEQ ID NO:404, CDR2L comprises sequence GAS, and CDR3L comprises SEQ ID NO:433;
bb) CDR1H comprises SEQ ID NO:231, CDR2H comprises SEQ ID NO:260, CDR3H comprises SEQ ID NO:289, CDR1L comprises SEQ ID NO:405, CDR2L comprises sequence AAS, and CDR3L comprises SEQ ID NO:434; or
cc) CDR1H comprises SEQ ID NO:232, CDR2H comprises SEQ ID NO:261, CDR3H comprises SEQ ID NO:290, CDR1L comprises SEQ ID NO:406, CDR2L comprises sequence AAS, and CDR3L comprises SEQ ID NO:435.

2. The antibody or antigen-binding fragment thereof of claim 1, wherein:
a) the variable heavy chain region comprises a sequence that is at least 90% identical to SEQ ID NO:59 and the variable light chain region comprises a sequence that is at least 90% identical to SEQ ID NO:88;
b) the variable heavy chain region comprises a sequence that is at least 90% identical to SEQ ID NO:60 and the variable light chain region comprises a sequence that is at least 90% identical to SEQ ID NO:89;
c) the variable heavy chain region comprises a sequence that is at least 90% identical to SEQ ID NO:61 and the variable light chain region comprises a sequence that is at least 90% identical to SEQ ID NO:90;
d) the variable heavy chain region comprises a sequence that is at least 90% identical to SEQ ID NO:62 and the variable light chain region comprises a sequence that is at least 90% identical to SEQ ID NO:91;
e) the variable heavy chain region comprises a sequence that is at least 90% identical to SEQ ID NO:63 and the variable light chain region comprises a sequence that is at least 90% identical to SEQ ID NO:92;
f) the variable heavy chain region comprises a sequence that is at least 90% identical to SEQ ID NO:64 and the variable light chain region comprises a sequence that is at least 90% identical to SEQ ID NO:93;
g) the variable heavy chain region comprises a sequence that is at least 90% identical to SEQ ID NO:65 and the variable light chain region comprises a sequence that is at least 90% identical to SEQ ID NO:94;
h) the variable heavy chain region comprises a sequence that is at least 90% identical to SEQ ID NO:66 and the variable light chain region comprises a sequence that is at least 90% identical to SEQ ID NO:95;
i) the variable heavy chain region comprises a sequence that is at least 90% identical to SEQ ID NO:67 and the variable light chain region comprises a sequence that is at least 90% identical to SEQ ID NO:96;
j) the variable heavy chain region comprises a sequence that is at least 90% identical to SEQ ID NO:68 and the variable light chain region comprises a sequence that is at least 90% identical to SEQ ID NO:97;
k) the variable heavy chain region comprises a sequence that is at least 90% identical to SEQ ID NO:69 and the variable light chain region comprises a sequence that is at least 90% identical to SEQ ID NO:98;
l) the variable heavy chain region comprises a sequence that is at least 90% identical to SEQ ID NO:70 and the variable light chain region comprises a sequence that is at least 90% identical to SEQ ID NO:99;
m) the variable heavy chain region comprises a sequence that is at least 90% identical to SEQ ID NO:71 and the variable light chain region comprises a sequence that is at least 90% identical to SEQ ID NO:100;
n) the variable heavy chain region comprises a sequence that is at least 90% identical to SEQ ID NO:72 and the variable light chain region comprises a sequence that is at least 90% identical to SEQ ID NO:101;
o) the variable heavy chain region comprises a sequence that is at least 90% identical to SEQ ID NO:73 and the variable light chain region comprises a sequence that is at least 90% identical to SEQ ID NO:102;
p) the variable heavy chain region comprises a sequence that is at least 90% identical to SEQ ID NO:74 and the variable light chain region comprises a sequence that is at least 90% identical to SEQ ID NO:103;
q) the variable heavy chain region comprises a sequence that is at least 90% identical to SEQ ID NO:75 and the variable light chain region comprises a sequence that is at least 90% identical to SEQ ID NO:104;
r) the variable heavy chain region comprises a sequence that is at least 90% identical to SEQ ID NO:76 and the variable light chain region comprises a sequence that is at least 90% identical to SEQ ID NO:105;
s) the variable heavy chain region comprises a sequence that is at least 90% identical to SEQ ID NO:77 and the variable light chain region comprises a sequence that is at least 90% identical to SEQ ID NO:106;
t) the variable heavy chain region comprises a sequence that is at least 90% identical to SEQ ID NO:78 and the variable light chain region comprises a sequence that is at least 90% identical to SEQ ID NO:107;
u) the variable heavy chain region comprises a sequence that is at least 90% identical to SEQ ID NO:79 and the variable light chain region comprises a sequence that is at least 90% identical to SEQ ID NO:108;
v) the variable heavy chain region comprises a sequence that is at least 90% identical to SEQ ID NO:80 and the variable light chain region comprises a sequence that is at least 90% identical to SEQ ID NO:109;
w) the variable heavy chain region comprises a sequence that is at least 90% identical to SEQ ID NO:81 and the variable light chain region comprises a sequence that is at least 90% identical to SEQ ID NO:110;
x) the variable heavy chain region comprises a sequence that is at least 90% identical to SEQ ID NO:82 and the variable light chain region comprises a sequence that is at least 90% identical to SEQ ID NO:111;
y) the variable heavy chain region comprises a sequence that is at least 90% identical to SEQ ID NO:83 and the variable light chain region comprises a sequence that is at least 90% identical to SEQ ID NO:112;
z) the variable heavy chain region comprises a sequence that is at least 90% identical to SEQ ID NO:84 and the variable light chain region comprises a sequence that is at least 90% identical to SEQ ID NO:113;
aa) the variable heavy chain region comprises a sequence that is at least 90% identical to SEQ ID NO:85 and the variable light chain region comprises a sequence that is at least 90% identical to SEQ ID NO:114;
bb) the variable heavy chain region comprises a sequence that is at least 90% identical to SEQ ID NO:86 and the variable light chain region comprises a sequence that is at least 90% identical to SEQ ID NO:115; or
cc) the variable heavy chain region comprises a sequence that is at least 90% identical to SEQ ID NO:87 and the variable light chain region comprises a sequence that is at least 90% identical to SEQ ID NO:116.

3. The antibody or antigen-binding fragment thereof of claim 2, wherein:
a) the variable heavy chain region comprises SEQ ID NO:59 and the variable light chain region comprises SEQ ID NO:88;
b) the variable heavy chain region comprises SEQ ID NO:60 and the variable light chain region comprises SEQ ID NO:89;
c) the variable heavy chain region comprises SEQ ID NO:61 and the variable light chain region comprises SEQ ID NO:90;
d) the variable heavy chain region comprises SEQ ID NO:62 and the variable light chain region comprises SEQ ID NO:91;
e) the variable heavy chain region comprises SEQ ID NO:63 and the variable light chain region comprises SEQ ID NO:92;
f) the variable heavy chain region comprises SEQ ID NO:64 and the variable light chain region comprises SEQ ID NO:93;
g) the variable heavy chain region comprises SEQ ID NO:65 and the variable light chain region comprises SEQ ID NO:94;
h) the variable heavy chain region comprises SEQ ID NO:66 and the variable light chain region comprises SEQ ID NO:95;
i) the variable heavy chain region comprises SEQ ID NO:67 and the variable light chain region comprises SEQ ID NO:96;
j) the variable heavy chain region comprises SEQ ID NO:68 and the variable light chain region comprises SEQ ID NO:97;
k) the variable heavy chain region comprises SEQ ID NO:69 and the variable light chain region comprises SEQ ID NO:98;
l) the variable heavy chain region comprises SEQ ID NO:70 and the variable light chain region comprises SEQ ID NO:99;
m) the variable heavy chain region comprises SEQ ID NO:71 and the variable light chain region comprises SEQ ID NO:100;
n) the variable heavy chain region comprises SEQ ID NO:72 and the variable light chain region comprises SEQ ID NO:101;
o) the variable heavy chain region comprises SEQ ID NO:73 and the variable light chain region comprises SEQ ID NO:102;
p) the variable heavy chain region comprises SEQ ID NO:74 and the variable light chain region comprises SEQ ID NO:103;
q) the variable heavy chain region comprises SEQ ID NO:75 and the variable light chain region comprises SEQ ID NO:104;
r) the variable heavy chain region comprises SEQ ID NO:76 and the variable light chain region comprises SEQ ID NO:105;
s) the variable heavy chain region comprises SEQ ID NO:77 and the variable light chain region comprises SEQ ID NO:106;
t) the variable heavy chain region comprises SEQ ID NO:78 and the variable light chain region comprises SEQ ID NO:107;
u) the variable heavy chain region comprises SEQ ID NO:79 and the variable light chain region comprises SEQ ID NO:108;
v) the variable heavy chain region comprises SEQ ID NO:80 and the variable light chain region comprises SEQ ID NO:109;
w) the variable heavy chain region comprises SEQ ID NO:81 and the variable light chain region comprises SEQ ID NO:110;
x) the variable heavy chain region comprises SEQ ID NO:82 and the variable light chain region comprises SEQ ID NO:111;
y) the variable heavy chain region comprises SEQ ID NO:83 and the variable light chain region comprises SEQ ID NO:112;
z) the variable heavy chain region comprises SEQ ID NO:84 and the variable light chain region comprises SEQ ID NO:113;
aa) the variable heavy chain region comprises SEQ ID NO:85 and the variable light chain region comprises SEQ ID NO:114;
bb) the variable heavy chain region comprises SEQ ID NO:86 and the variable light chain region comprises SEQ ID NO:115; or
cc) the variable heavy chain region comprises SEQ ID NO:87 and the variable light chain region comprises SEQ ID NO:116.

4. The antibody or antigen-binding fragment thereof of claim 1, wherein the antibody comprises human-derived heavy and light chain constant regions.

5. The antibody or antigen-binding fragment thereof of claim 1, wherein the antibody is an immunoglobulin comprising two identical heavy chains and two identical light chains.

6. The antibody or antigen-binding fragment thereof of claim 1, wherein the antibody is a monoclonal antibody.

7. The antibody or antigen-binding fragment thereof of claim 1, wherein the antigen-binding fragment is a scFv.

8. The antibody or antigen-binding fragment thereof of claim 1, wherein the antibody is conjugated to a detectable agent or a therapeutic agent.

9. An isolated nucleic acid sequence comprising a nucleotide sequence encoding the antibody or antigen-binding fragment thereof of claim 1.

10. An expression vector comprising the nucleic acid sequence of claim 9.

11. The expression vector of claim 10, wherein the nucleotide sequence is operably linked to one or more regulatory regions.

12. A host cell comprising the nucleic acid sequence of claim 9.

13. A method for expressing the antibody or antigen-binding fragment thereof of claim 1, comprising:
a. culturing a host cell comprising a nucleotide sequence encoding the antibody or antigen-binding fragment thereof, and
b. isolating the antibody or antigen-binding fragment thereof from the host cell or cell culture.

14. A method for detecting SARS-CoV-2, comprising:
a. contacting cells or a biological sample with the antibody or antigen-binding fragment thereof of claim 1; and
b. detecting the binding of the antibody to SARS-CoV-2 spike protein, wherein SARS-CoV-2 is detected if the level of binding of the antibody or antigen-binding fragment thereof to SARS-CoV-2 protein is greater than the level of binding of the antibody or antigen-binding fragment thereof to non-SARS-CoV-2 infected cells or a biological sample not infected with SARS-CoV-2.

15. A pharmaceutical composition comprising the antibody or antigen-binding fragment thereof of claim 1, and pharmaceutically acceptable carrier.

16. A method for treating SARS-CoV-2 infection or COVID-19 in a subject, comprising administering to the subject an effective amount of the antibody or antigen-binding fragment thereof of any one of claim 1.

17. The method of claim 16, wherein the subject is diagnosed with SARS-CoV-2 infection or COVID-19.

18. A method for preventing COVID-19 in a subject, comprising administering to the subject to the subject an effective amount of the antibody or antigen-binding fragment thereof of claim 1.

19. A method for treating SARS-CoV-2 infection or COVID-19 in a subject, comprising administering to the subject an effective amount of the antibody or antigen-binding fragment thereof of claim 1.

20. The method of claim 16, wherein the subject is a human.

21. (canceled)

22. (canceled)

23. (canceled)


SEQUENCE LISTING (435 sequences)

SEQ ID NO: 1 - DNA, 378 bp, Artificial Sequence
  Description of Artificial Sequence Synthetic polynucleotide
  1C12_f3 variable heavy chain
caggtgcagctggtgcagtctggggctgaggtgaagaagcctggggcctcagtgaaggtctcctgcaaggcttctggatacaccttcaccagttatgatatcacctgggtgcgacaggccactggacaagggcttgagtggatgggatggatgagccctaacagtggtaacacaggctatacacagaagttccagggcagagtcaccatgaccaggaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 2 - DNA, 378 bp, Artificial Sequence
  Description of Artificial Sequence Synthetic polynucleotide
  1D5_f3 variable heavy chain
caggtgcagctggtgcagtctggggctgaggtgaagaagcctggggcctcagtgaaggtctcctgcaaggcttctggatacaccttcaccagttatgatatcacctgggtgcgacaggccactggacaagggcttgagtggatgggatggatgagccctaacagtggtaacacaggctatgcacagaagttccagggcagagtcaccatgaccaggaacacctccataagcacagcctacatggagctgaacagcctgagatctgaggacacggccgtgtattactgtgcgagattcggctatggttcgggggccctcgactactactactacggtttggacgtctggggccaagggaccacggtcaccgtctcctca

SEQ ID NO: 3 - DNA, 378 bp, Artificial Sequence
  Description of Artificial Sequence Synthetic polynucleotide
  1D10_f3 variable heavy chain
caggtgcagctggtgcagtctggggctgaagtgaagaagcctggggcctcagtgaaggtctcctgcaaggcttctggatacaccttcaccagttatgatatcatctgggtgcgacaggcctctggacaagggcttgagtggatgggatggatgagccctaacagcggtaacacaggctatgcacagaagttccagggcagagtcaccatgaccaggaacacctccataaacacagcctacatggagctgagtagcctgagatctgaggacacggccgtgtattattgtgcgagattcggctatggttcgggggccctcgactactactattacggtttggacgtctggggccaagggaccacggtcaccgtctcctca

SEQ ID NO: 4 - DNA, 378 bp, Artificial Sequence
  Description of Artificial Sequence Synthetic polynucleotide
  1E2_f3 variable heavy chain
caggtgcagctggtgcagtctggggctgaggtgaagaagcctggggcctcagtgaaggtctcctgcaaggcttctggatacaccttcaccagttatgatatcacctgggtgcgacaggccactggacaagggcttgagtggatgggatggatgagccctaacagtggtaacacaggctatgcacagaagttccagggcagagtcaccatgaccaggaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 5 - DNA, 378 bp, Artificial Sequence
  Description of Artificial Sequence Synthetic polynucleotide
  1G9_f3 variable heavy chain
caggtgcagctggtgcagtctggggctgaggtgaagaagcctggggcctcagtgaaggtctcctgcaaggcttctggatacaccttcaccagttatgatatcatttgggtgcgacaggccactggacaagggcttgagtggatgggatggatgagccctaacagtggtaacacaggctatgcacagaagttccagggcagagtcaccatgaccaggaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 6 - DNA, 378 bp, Artificial Sequence
  Description of Artificial Sequence Synthetic polynucleotide
  5D7_f3 variable heavy chain
caggtgcagctggtgcagtctggggctgaggtgaagaagcctgggacctcagtgaaagtctcctgcaagacttctggatacaccttcaccagttatgatatcatctgggtgcgacaggccactggacaagggcttgagtggatgggatggatgagccctaaaaatggtaacacaggctatgcacagaggttccagggcagagtcaccatgaccaggaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 7 - DNA, 378 bp, Artificial Sequence
  Description of Artificial Sequence Synthetic polynucleotide
  5H12_f3 variable heavy chain
caggtgcagctggtgcagtctggggctgaggtgaagaagcctgggacctcagtgaaagtctcctgcaagacttctggatacaccttccccagttatgatatcatctgggtgcgacaggccactggacaagggcttgagtggatgggatggatgagccctaaaaatggtaacacaggctttgcacagaggttccagggcagagtcaccatgaccaggaacacctccataagcacagcctacatggaactgagcagcctgagatctgaggacacggccgtgtattactgtgcgagattcggctatgggtcgggggccctcggatattactactacggtttggacgtctggggccaagggaccacggtcaccgtctcctca

SEQ ID NO: 8 - DNA, 378 bp, Artificial Sequence
  Description of Artificial Sequence Synthetic polynucleotide
  7C10_f3 variable heavy chain
caggtgcagctggtgcagtctggggctgaggtgaagaagcctggggcctcagtgaaggtctcctgcaaggcttctggatacaccttcaccagttatgatatcacctgggtgcgacaggccactggacaagggcttgagtggatggggtggatgagccctaacagtggtaacacaggctatgcacagaagttccagggcagagtcaccatgaccaggaacacctccataagcacagcctacatggagctgcgcagcctgaggtctgaggacacggccgtgtattactgtgcgagattcggctatggttcgggggccctcgactactactactacggtttggacgtctggggccaagggaccacggtcaccgtctcctca

SEQ ID NO: 9 - DNA, 378 bp, Artificial Sequence; identical to SEQ ID NO: 4
  Description of Artificial Sequence Synthetic polynucleotide
  8H4_f3 variable heavy chain

SEQ ID NO: 10 - DNA, 378 bp, Artificial Sequence
  Description of Artificial Sequence Synthetic polynucleotide
  9C6_f3 variable heavy chain
caggtgcagctggtgcagtctggggctgaggtgaagaagcctggggcctcagtgaaggtctcctgcaaggcttctggatacaccttcaccagttatgatatcacctgggtgcgacaggccactggacaagggcttgagtggatgggatggatgagccctaaaagtggtaacacaggctatgcacagaagttccagggcagagtcaccatgaccaggaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 11 - DNA, 354 bp, Artificial Sequence
  Description of Artificial Sequence Synthetic polynucleotide
  10D6_f3 variable heavy chain
gaggtgcagttggtggagactggaggaggcttgatccagcctggggggtccctgagactctcctgtgcagcctctgggatcaccgtcagtagtaactacatgaactgggtccgccaggctccagggaaggggctggagtgggtctcagttatttatagcggtggtagcacatactacgcagactccgtgaagggccgattcaccatctccagagacaattccaagaacacactgtatcttcaaatgaacagcctgagagccgaggacacggccgtgtattactgtgcgagagatttagaactggctggagcttttgatatctggggccaagggacaatggtcaccgtctcttta

SEQ ID NO: 12 - DNA, 354 bp, Artificial Sequence
  Description of Artificial Sequence Synthetic polynucleotide
  11D5_f3 variable heavy chain
gaggtgcagttggtggagactggaggaggcttgatccagcctggggggtccctgagactctcctgtgcagcctctgggatcaccgtcagtagtaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagatttagcagtggctggagcttttgatatctggggccaagggacaatggtcaccgtctcttta

SEQ ID NO: 13 - DNA, 378 bp, Artificial Sequence
  Description of Artificial Sequence Synthetic polynucleotide
  11G2_f3 variable heavy chain
caggtgcagctggtgcagtctggggctgaggtgaagaagcctggggcctcagtgaaggtctcctgcaaggcttctggatacaccttcaccagttatgatatcacctgggtgcgacaggccactggacaagggcttgagtggatgggatggatgagccctaacagtggtaacacaggctatgcacagaagttccagggcagagtctccatgaccaggaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 14 - DNA, 366 bp, Artificial Sequence
  Description of Artificial Sequence Synthetic polynucleotide
  11G7_f2 variable heavy chain
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctttcatagtgggagcaccaactacaacccgtccctcaagagtcgagtcaccatatcagtagacaagtccaagaaccagttctccctgaagctgaactctgtgaccgccgcggacacggccgtgtattactgtgcgagattcagaaggatagtggctacgagctactattttgactactggggccagggaaccctggtcaccgtctcctca

SEQ ID NO: 15 - DNA, 378 bp, Artificial Sequence
  Description of Artificial Sequence Synthetic polynucleotide
  16C5_f3 variable heavy chain
caggtgcagctggtgcagtctggggctgaggtgaagaagcctggggcctcagtgaaggtctcctgcaaggcttctggatacaccttcaccagttatgatatcatttgggtgcgacaggccactggacaagggcttgagtggatgggatggatgagccctaatagtggtaacacaggctatgcacagaagttccagggcagagtcaccatgaccaggaacacctccataggcacagcctacatggagctgagcagcctgagatctgaggacacggccgtgtatttctgtgcgagattcggctatggttcgggggccctcgactactactactacggtttggacgtctggggccaagggaccacggtcaccgtctcctca

SEQ ID NO: 16 - DNA, 378 bp, Artificial Sequence
  Description of Artificial Sequence Synthetic polynucleotide
  16C12_f3 variable heavy chain
caggtgcagctggtgcagtctggggctgaggtgaagaagcctggggcctcagtgaaggtctcctgcaaggcttctggatacaccttcaccagttatgatattatctgggtgcgacaggccactggacaagggcttgagtggatgggatggatgagccctaacagtggtaacacaggctatgcacagaagttccagggcagagtcaccatgaccaggaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 17 - DNA, 378 bp, Artificial Sequence
  Description of Artificial Sequence Synthetic polynucleotide
  17A8_f3 variable heavy chain
caggtgcagctggtgcagtctggggctgaggtgaagaagcctggggcctcagtgaaggtctcctgcaaggcttctggatacaccttcaccagttatgatatcatttgggtgcgacaggccactggacaagggcttgagtggatgggatggatgagccctaacagtggtaacacaggctatgcacagaagttccagggcagagtcaccatgaccaagaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 18 - DNA, 378 bp, Artificial Sequence
  Description of Artificial Sequence Synthetic polynucleotide
  17F2_f3 variable heavy chain
caggtgcagctggtgcagtctggggctgaggtgaagaggcctggggcctcagtgaaggtctcctgcaaggcttctggatacaccttcaccagttatgatatcacctgggtgcgacaggccactggacaagggcttgagtggatgggatggatgagccctgacagtggtaacacaggctatgcacagaggttccagggcagagtcaccatgaccaggaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 19 - DNA, 378 bp, Artificial Sequence
  Description of Artificial Sequence Synthetic polynucleotide
  18E7_f3 variable heavy chain
caggtgcagctggtgcagtctggggctgaggtgaagaagcctggggcctcagtgacggtctcctgcaaggcttctggatacacctttaccagttatgatatcatctgggtgcgacaggccactggacaagggcttgagtggatgggatggatgagccctaacagtggtaacacaggctatgcacagaggttccagggcagagtcaccatgaccaggaacacctccataagcacagcctacatggagctgagcagcctgagatctgaggacacggccgtgtattactgtgcgagattcggctatggttcgggggccctcgactactactactacggtttggacgtctggggccaagggaccacggtcaccgtctcctca

SEQ ID NO: 20 - DNA, 354 bp, Artificial Sequence
  Description of Artificial Sequence Synthetic polynucleotide
  19C4_f3 variable heavy chain
gaggtgcagttggtggagactggaggaggcttgatccagcctggggggtccctgagactctcctgtgcagcctctgggatcaccgtcagtagtaattacatgaactgggtccgccaggctccagggaaggggctggagtgggtctcagttatttatagcggtggtagcacattctacgcagactccgtgaagggccgattcaccatctccagagacaattccaagaacacgctgtatcttcaaatgaacagcctgagagccgaggacacggccgtatattactgtgcgagagatttagaagtggctggaggttttgatatctggggccaagggacaatggtcaccgtctcttta

SEQ ID NO: 21 - DNA, 369 bp, Artificial Sequence
  Description of Artificial Sequence Synthetic polynucleotide
  2C1_f4 variable heavy chain
gaggtgcagctggtggagtctgggggaggcttggtacagccaggacggtccctgagactctcctgtacagcttctggattcacctttggtgattatactttgagctggttccgccaggctccagggaaggggctggaatgggtaggtttcattagaagcaaaccttttggtgggacaacacaatacgccgcgtctgtgaaaggcagattcaccatctcaagggatgattccaaaagcatcgcctatctgcaaatgaacagcctgaaaaccgaggacacagccgtgtattactgtactagagtgtccgggtatagcaacatctggttctttgcctactggggccagggaaccctggtcaccgtctcctca

SEQ ID NO: 22 - DNA, 369 bp, Artificial Sequence
  Description of Artificial Sequence Synthetic polynucleotide
  4E3_f4 variable heavy chain
caggtgcaactggtggagtctgggggaggcgtggtccagcctgggaggtccctgagactctcctgtgcagcgtctggattcaccttcagtagctatggcatgaactgggtccgccaggctccaggcaaggggctggagtgggtggcagttatttggtatgatggaaataataaatactatgcagactccgtgaagggccgattcaccatctccagagacaattccaagaacacgttgtatctgcaaatgaacagcctgagagccgaggacacggctgtgtattactgtgcgagaaaagattattcgaagacattttatggatactactttgactattggggccagggaaccctggtcaccgtctcctca

SEQ ID NO: 23 - DNA, 351 bp, Artificial Sequence
  Description of Artificial Sequence Synthetic polynucleotide
  7D2_f4 variable heavy chain
gaggtgcagctggtggagactggaggaggcttgatccagcctggggggtccctgagactctcctgtgcagcctctgggttcaccgtcagtagcaactacatgaactgggtccgccaggctccagggaaggggctggagtgggtctcagttatttatagcggtggtagcacattctacgcagactccgtgaagggccgattcaccatctccagagacaattcctacaacacgctgtatcttcaaatgaacagcctgagagccgaggacacggccgtgtattactgtgcgagagatctagtcatctacggtatggacgtctggggccaagggaccacggtcaccgtctcctca

SEQ ID NO: 24 - DNA, 369 bp, Artificial Sequence
  Description of Artificial Sequence Synthetic polynucleotide
  9C5_f4 variable heavy chain
caggtgcaactggtggagtctgggggaggcgtggtccagcctgggaggtccctgagactctcctgtgtagcgtctggattcaccttcagtagctatggcatgaactgggtccgccaggctccaggcaaggggctggagtgggtggcagttatttggtatgatggaaataataaatactatgcagactccgtgaagggccgattcaccatctccagagacaattccaagaacacgctgtatctgcaaatgaacagtctgagagccgaggacacggctgtgtattactgtgcgagaaaagatggttcgaagacatattatggatactactttgactattggggccagggaaccctggtcaccgtctcctca

SEQ ID NO: 25 - DNA, 369 bp, Artificial Sequence
  Description of Artificial Sequence Synthetic polynucleotide
  10A3_f4 variable heavy chain
caggtgcaactggtggagtctgggggaggcgtggtccagcctgggaggtccctgagactctcctgtgcagcgtctggattcaccttcagtagttatggcatgaactgggtccgccaggctccaggcaaggggctggagtgggtggcaattatttggtatgatggaaataatacatactatgtagactccgtgaagggccgattcaccatctccagagacaattccaagaacacgctgtatctgcaaatgaacagcctgagagccgaagacacggctgtgtattactgtgcgagaaaagatggttcgaagacatattatggatactactttgactattggggccagggaaccctggtcaccgtctcctca

SEQ ID NO: 26 - DNA, 369 bp, Artificial Sequence
  Description of Artificial Sequence Synthetic polynucleotide
  14G5_f4 variable heavy chain
caggtgcagctgcaggagtcgggcccaggactggtgaagccttcggggaccctgtccctcacctgcgctgtctctggtggctccatcagcagtaataactggtggagttgggtccgccagcccccagggaaggggctggtgtggattggggaaatcttgcatggtgagatcaccaactacaacccgtccctcaagagtcgagtcaccatatcaatagacaagtccaagaaccagttctccctgaagctgagctctgtgaccgccgcggacacggccgtgtattactgtgcgagagatgcgaatttctatggttcggggagttcttactttgactactggggccagggaaccctggtcaccgtctcctca

SEQ ID NO: 27 - DNA, 369 bp, Artificial Sequence
  Description of Artificial Sequence Synthetic polynucleotide
  13A12_f3 variable heavy chain
caggtgcagctgcaggagtcgggcccaggactggtgaagccttcggggaccctgtccctcacctgcgctgtctcaggtggctccatctacagtagtaactgctggagttgggtccgccagcccccagggaaggggctggagtggattggggaaatctatcatagtgggggcaccaactacaacccgtccctcaagagtcgagtcaccatatcattagacaagtccaagaacaggttctccctgaggctgagctctgtgaccgccgcggacacggccgtgtattactgtgcgagagatcaagattactatggttcggggagttccctctttgactactggggccagggaaccctggtcaccgtctcctca

SEQ ID NO: 28 - DNA, 345 bp, Artificial Sequence
  Description of Artificial Sequence Synthetic polynucleotide
  5B6_f3 variable heavy chain
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgttttaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccaccagcagtggctactggggccagggaaccctggtcaccgtctcctca

SEQ ID NO: 29 - DNA, 354 bp, Artificial Sequence
  Description of Artificial Sequence Synthetic polynucleotide
  2G6_f4 variable heavy chain
caggtgcagctacagcagtggggcgcaggactgttgaagccttcggagaccctgtccctcacctgcactatctatggtgggtccttcagtgtttactactggaactggatccgccagcccccagagaaggggctggagtggattggggaaatcaatcatagtggaaacaccaactacaacccgtccctcaagagtcgagtcaccatatcagtagacacgtccaagaaccaattctccctgaagctgagctctgtgaccgccgcggacacggctgtgtattactgtgcgaggtattactatgatggtaatggttattacccctggggccagggaaccctggtcaccgtctcctca

SEQ ID NO: 30 - DNA, 324 bp, Artificial Sequence
  Description of Artificial Sequence Synthetic polynucleotide
  1C12_f3 variable light chain
gaaattgtgttgacgcagtctccaggcaccctgtctttgtctccaggggaaagagccaccctctcctgcagggccagtcagagtgttaggagcagtcacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtataataactcaccgatcaccttcggccaagggacacgactggagattaaa

SEQ ID NO: 31 - DNA, 324 bp, Artificial Sequence
  Description of Artificial Sequence Synthetic polynucleotide
  1D5_f3 variable light chain
gaaattgtgttgacgcagtctccaggcaccctgtctttgtctccaggggacagagccaccctctcctgcagggccagtcagagtgttaggagcagtcacttagcctggtaccagcagaaacctggccaggctcccaggctcctcatctatggtgcatccagcagggccactggcatcccagacaggttcagtggcagtgggtctgggacagacttctctctcaccatcagcagactggagcctgaggattttgcagtgtattactgtcagcagtttggtagctcaccgatcaccttcggccaagggacacggctggagattaaa

SEQ ID NO: 32 - DNA, 324 bp, Artificial Sequence
  Description of Artificial Sequence Synthetic polynucleotide
  1D10_f3 variable light chain
gaaattgtgttgacgcagtctccaggcaccctgtctttgtctccaggggaaagagccaccctctcctgcagggccagtcagagtgttagcagcagccacttagcctggtaccagcagaaacctggccaggctcccaggctcctcatctatggtgcatccagcagggccactggcatcccagacaggttcagtggcagtgggtctggaacagacttcactctcaccatcagcagactggagcctgaagattttgcagtgtattactgtcagcagtataatagctcaccgatcaccttcggccaagggacacgactggagattaaa

SEQ ID NO: 33 - DNA, 324 bp, Artificial Sequence
  Description of Artificial Sequence Synthetic polynucleotide
  1E2_f3 variable light chain
gaaattgtgttgacgcagtctccaggcaccctgtctttgtctccaggggaaagagccaccctctcctgcagggccagtcagagtgttaggagcagtcactttgcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtgttactgtcagcagtatggtagctcaccgatcaccttcggccaagggacacgactggagattaaa

SEQ ID NO: 34 - DNA, 324 bp, Artificial Sequence
  Description of Artificial Sequence Synthetic polynucleotide
  1G9_f3 variable light chain
gaaattgtgttgacgcagtctccaggcaccctgtctttgtctccaggggaaagagccaccctctcctgcagggccagtcagagtgttaggagcagtcacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgatcaccttcggccaagggacacgactggagattaaa

SEQ ID NO: 35 - DNA, 324 bp, Artificial Sequence
  Description of Artificial Sequence Synthetic polynucleotide
  5D7_f3 variable light chain
gaaattgtgttgacgcagtctccaggcaccctgtctttgtctccaggggaaagagccaccctctcctgcagggccagtcagagtgttaggagcagtcacttagcctggtaccggcagaaacctggccaggctcccaggctcctcatctatggtgcctccagcagggccactggcatcccagacaggttcagtggccgtgggtctgggacagacttcactctcaccatcagcagactggagcctgaagattttgcagtgtattactgtcagcagtataatatctcaccgatcaccttcggccaagggacacgactggagattaaa

SEQ ID NO: 36 - DNA, 324 bp, Artificial Sequence
  Description of Artificial Sequence Synthetic polynucleotide
  5H12_f3 variable light chain
gaaattgtgttgacgcagtctccaggcaccctgtctttgtctccaggggaaagagccaccctctcctgcagggccagtcagagtgttaggagcagtcacttagcctggtaccggcagaaacctggccaggctcccaggctcctcatctatggtgcctccagcagggccactggcatcccagacaggttcagtggccgtgggtctgggacagacttcactctcaccatcagcagactggagcctgaagattttgcagtgtattactgtcagcagtataatttctcaccgatcaccttcggccaagggacacgactggagattaaa

SEQ ID NO: 37 - DNA, 324 bp, Artificial Sequence
  Description of Artificial Sequence Synthetic polynucleotide
  7C10_f3 variable light chain
gaaattgtgttgacgcagtctccaggcaccctgtctttgtctccaggggaaagagccaccctctcctgcagggccagtcagagtgttaggagcagtcacttagcctggtcccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtttggtagctcaccgatcaccttcggccaagggacacgactggagattaaa

SEQ ID NO: 38 - DNA, 324 bp, Artificial Sequence
  Description of Artificial Sequence Synthetic polynucleotide
  8H4_f3 variable light chain
gaaattgtgttgacgcagtctccaggcaccctgtctttgtctccaggggaaagagccaccctctcctgcagggccagtcagagtgttaggagcagtcacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtttggtagctcaccgatcaccttcggccaagggacacgactggagattaag

SEQ ID NO: 39 - DNA, 324 bp, Artificial Sequence
  Description of Artificial Sequence Synthetic polynucleotide
  9C6_f3 variable light chain
gaaattgtgttgacgcagtctccaggcaccctgtctttgtctccaggggaaagagccaccctctcctgcagggccagtcagagtgttaggagcagtcacttagcctggtaccggcagaaacctggccaggctcccaggctcctcatctatggtgcctccagcagggccactggcatcccagacaggttcagtggccgtgggtctgggacagacttcactctcaccatcagcagactggagcctgaagattttgcagtgtattactgtcagcagtataatagctcaccgatcaccttcggccaagggacacgactggagataaaa

SEQ ID NO: 40 - DNA, 324 bp, Artificial Sequence
  Description of Artificial Sequence Synthetic polynucleotide
  10D6_f3 variable light chain
gacatccagttgacccagtctccatccttcctgtctgcatctgtaggagacagagtcaccatcacttgccgggccagtcagggcattagcagttatttagcctggtatcagcaaaaaccagggaaagcccctaaggtcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctccgtccacttttggccaggggaccaagctggagatcaaa

SEQ ID NO: 41 - DNA, 324 bp, Artificial Sequence; identical to SEQ ID NO: 40
  Description of Artificial Sequence Synthetic polynucleotide
  11D5_f3 variable light chain

SEQ ID NO: 42 - DNA, 324 bp, Artificial Sequence
  Description of Artificial Sequence Synthetic polynucleotide
  11G2_f3 variable light chain
gaaattgtgttgacgcagtctccaggcaccctgtctttgtctccaggggaaagagccaccctctcctgcagggccagtcagagtgttaggaacagtcacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtttggtagctcaccgatcaccttcggccaagggacacgactggagattaaa

SEQ ID NO: 43 - DNA, 336 bp, Artificial Sequence
  Description of Artificial Sequence Synthetic polynucleotide
  11G7_f2 variable light chain
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 44 - DNA, 324 bp, Artificial Sequence; identical to SEQ ID NO: 30
  Description of Artificial Sequence Synthetic polynucleotide
  16C5_f3 variable light chain

SEQ ID NO: 45 - DNA, 324 bp, Artificial Sequence; identical to SEQ ID NO: 34
  Description of Artificial Sequence Synthetic polynucleotide
  16C12_f3 variable light chain

SEQ ID NO: 46 - DNA, 324 bp, Artificial Sequence
  Description of Artificial Sequence Synthetic polynucleotide
  17A8_f3 variable light chain
gaaactgtgttgacgcagtctccaggcactctgtctttgtctccaggggaaagagccaccctctcctgcagggccagtcagagtgttaggagcagtcacttagcctggtaccagcagaaacctggccaggctcccaggctcctcatctatggtgcatccagtagggccactggcatcccagacaggttcagtggcagtgggtctgggacagacttcactctcaccatcagcagactggagcctgaagattttgcagtgtattactgtcagcagtatggtagctcaccgatcaccttcggccaagggacacgactggagattaaa

SEQ ID NO: 47 - DNA, 324 bp, Artificial Sequence
  Description of Artificial Sequence Synthetic polynucleotide
  17F2_f3 variable light chain
gaaattgtgttgacgcagtctccaggcaccctgtctttgtctccaggggaaagagccaccctctcctgcagggccagtcagagtgttaggagcagtcacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtataatagctcaccgatcaccttcggccaagggacacgactggagataaaa

SEQ ID NO: 48 - DNA, 324 bp, Artificial Sequence; identical to SEQ ID NO: 34
  Description of Artificial Sequence Synthetic polynucleotide
  18E7_f3 variable light chain

SEQ ID NO: 49 - DNA, 324 bp, Artificial Sequence
  Description of Artificial Sequence Synthetic polynucleotide
  19C4_f3 variable light chain
gacatccagttgacccagtctccatccttcctgtctgcatctgtaggagacagagtcaccatcacttgccgggccagtcagggcattagcagttatttagcctggtatcagcaaaaaccagggaaagcccctaaggtcctgatctatgctgcatccactttgcaaagtggggtcccatcaaggttcagcggcagtggatctgggacaaaattcactctcacaatcagcagcctgcagcctgaagattttgcaacttattactgtcaacagcttaatagtttccctccgtccacttttggccaggggaccaagctggagatcaaa

SEQ ID NO: 50 - DNA, 318 bp, Artificial Sequence
  Description of Artificial Sequence Synthetic polynucleotide
  2C1_f4 variable light chain
gaaatagtgatgacgcagtctccagccaccctgtctgtgtctccaggggaaagagccaccctctcctgcagggccagtcagagtgttagcatcaacttagcctggtaccagcagaaacctggccaggctcccaggctcctcatctatggtgcatccaccagggccactggtatcccagccaggttcagtggcagtgggtctgggacagagttcactctcaccgtcagcagcctgcagtctgaagattttgcagtttattactgtcagcagtataataactggtggacgttcggccaagggaccaaggtggaaatcaaa

SEQ ID NO: 51 - DNA, 321 bp, Artificial Sequence
  Description of Artificial Sequence Synthetic polynucleotide
  4E3_f4 variable light chain
gacatccagatgacccagtctccatcctccctgtctgcatctgtaggagacagagtcaccatcacttgccgggcaagtcagagcattcacagctttttaaattggtatcagcagaaaccagggaaaccccctaagctcctgatctatgctgcatccagtttgccaagtgggctcccatcaaggttcagtggcagtggatctgggacagatttcactctcaccatcagcagtctgcaacctgaagattttgcaacttactactgtcaacagagttacattacccctccgacgttcggccaagggaccaaggtggaaatcaaa

SEQ ID NO: 52 - DNA, 318 bp, Artificial Sequence
  Description of Artificial Sequence Synthetic polynucleotide
  7D2_f4 variable light chain
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtgtcccagccaggttcagtggcagtgggtctgggacagagttcactctcaccatcagcagcctgcagtctgaagattttgcagtttatttctgtcagcagtataataactggccccctttcggcggagggaccaaggtggagatcaaa

SEQ ID NO: 53 - DNA, 321 bp, Artificial Sequence
  Description of Artificial Sequence Synthetic polynucleotide
  9C5_f4 variable light chain
gacatccagatgacccagtctccatcctccctgtctgcatctgtaggagacagagtcaccatcacttgccgggcaagtcagagcattcacagctttttaaattggtatcagcagaaaccagggaaaccccctaagctcctgatctatactacatccagtttgcaaagtgggctcccatcaaggttcagtggcagtggatctgggacagatttcactctcaccatcagcagtctgcaacctgaagattttgcaacttactactgtcaacagagttacattacccctccgacgttcggccaagggaccaaggtggaaatcaaa

SEQ ID NO: 54 - DNA, 321 bp, Artificial Sequence
  Description of Artificial Sequence Synthetic polynucleotide
  10A3_f4 variable light chain
gacatccagatgacccagtctccatcctccctgtctgcatctgtaggagacagagtcaccatcacttgccgggcaagtcagagcattcacagctttttaaattggtatcagcagaaaccagggaaaccccctaacctcctgatctatgctgcatccagtttgcaaagtgggctcccatcaaggttcagtggcagtggatctgggacagatttcactctcaccatcagcagtctgcaacctgaagattttgcaacttactactgtcaacagagttacattacccctccgacgttcggccatgggaccaaggtggaaatcaaa

SEQ ID NO: 55 - DNA, 321 bp, Artificial Sequence
  Description of Artificial Sequence Synthetic polynucleotide
  14G5_f4 variable light chain
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgacttttggccaggggaccaagctggagatcaaa

SEQ ID NO: 56 - DNA, 321 bp, Artificial Sequence
  Description of Artificial Sequence Synthetic polynucleotide
  13A12_f3 variable light chain
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtatgataactggcctctcactttcggcggagggaccaaggtggagatcaaa

SEQ ID NO: 57 - DNA, 321 bp, Artificial Sequence
  Description of Artificial Sequence Synthetic polynucleotide
  5B6_f3 variable light chain
gacatccagatgacccagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgcccctcacacttttggccaggggaccaagctggagatcaaa

SEQ ID NO: 58 - DNA, 321 bp, Artificial Sequence
  Description of Artificial Sequence Synthetic polynucleotide
  2G6_f4 variable light chain
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagtctgaagattttgcaatttattactgtcaacagcttaatagttacccgctcactttcggcggagggaccaaggtggagatcaaa

SEQ ID NO: 59 - protein, 126 aa, Artificial Sequence
  Description of Artificial Sequence Synthetic polypeptide
  1C12_f3 variable heavy chain
QVQLVQSGAEVKKPGASVKVSCKASGYTFTSYDITWVRQATGQGLEWMGWMSPNSGNTGYTQKFQGRVTMTRNTSISTAYMELSSLRSEDTAVYYCARFGYGSGALDYYYYGLDVWGQGTTVTVSS

SEQ ID NO: 60 - protein, 126 aa, Artificial Sequence
  Description of Artificial Sequence Synthetic polypeptide
  1D5_f3 variable heavy chain
QVQLVQSGAEVKKPGASVKVSCKASGYTFTSYDITWVRQATGQGLEWMGWMSPNSGNTGYAQKFQGRVTMTRNTSISTAYMELNSLRSEDTAVYYCARFGYGSGALDYYYYGLDVWGQGTTVTVSS

SEQ ID NO: 61 - protein, 126 aa, Artificial Sequence
  Description of Artificial Sequence Synthetic polypeptide
  1D10_f3 variable heavy chain
QVQLVQSGAEVKKPGASVKVSCKASGYTFTSYDIIWVRQASGQGLEWMGWMSPNSGNTGYAQKFQGRVTMTRNTSINTAYMELSSLRSEDTAVYYCARFGYGSGALDYYYYGLDVWGQGTTVTVSS

SEQ ID NO: 62 - protein, 126 aa, Artificial Sequence
  Description of Artificial Sequence Synthetic polypeptide
  1E2_f3 variable heavy chain
QVQLVQSGAEVKKPGASVKVSCKASGYTFTSYDITWVRQATGQGLEWMGWMSPNSGNTGYAQKFQGRVTMTRNTSISTAYMELSSLRSEDTAVYYCARFGYGSGALDYYYYGLDVWGQGTTVTVSS

SEQ ID NO: 63 - protein, 126 aa, Artificial Sequence
  Description of Artificial Sequence Synthetic polypeptide
  1G9_f3 variable heavy chain
QVQLVQSGAEVKKPGASVKVSCKASGYTFTSYDIIWVRQATGQGLEWMGWMSPNSGNTGYAQKFQGRVTMTRNTSISTAYMELSSLRSEDTAVYYCARFGYGSGALDYYYYGLDVWGQGTTVTVSS

SEQ ID NO: 64 - protein, 126 aa, Artificial Sequence
  Description of Artificial Sequence Synthetic polypeptide
  5D7_f3 variable heavy chain
QVQLVQSGAEVKKPGTSVKVSCKTSGYTFTSYDIIWVRQATGQGLEWMGWMSPKNGNTGYAQRFQGRVTMTRNTSISTAYMELSSLRSEDTAVYYCARFGYGSGALDYYYYGLDVWGQGTTVTVSS

SEQ ID NO: 65 - protein, 126 aa, Artificial Sequence
  Description of Artificial Sequence Synthetic polypeptide
  5H12_f3 variable heavy chain
QVQLVQSGAEVKKPGTSVKVSCKTSGYTFPSYDIIWVRQATGQGLEWMGWMSPKNGNTGFAQRFQGRVTMTRNTSISTAYMELSSLRSEDTAVYYCARFGYGSGALGYYYYGLDVWGQGTTVTVSS

SEQ ID NO: 66 - protein, 126 aa, Artificial Sequence
  Description of Artificial Sequence Synthetic polypeptide
  7C10_f3 variable heavy chain
QVQLVQSGAEVKKPGASVKVSCKASGYTFTSYDITWVRQATGQGLEWMGWMSPNSGNTGYAQKFQGRVTMTRNTSISTAYMELRSLRSEDTAVYYCARFGYGSGALDYYYYGLDVWGQGTTVTVSS

SEQ ID NO: 67 - protein, 126 aa, Artificial Sequence; identical to SEQ ID NO: 62
  Description of Artificial Sequence Synthetic polypeptide
  8H4_f3 variable heavy chain

SEQ ID NO: 68 - protein, 126 aa, Artificial Sequence
  Description of Artificial Sequence Synthetic polypeptide
  9C6_f3 variable heavy chain
QVQLVQSGAEVKKPGASVKVSCKASGYTFTSYDITWVRQATGQGLEWMGWMSPKSGNTGYAQKFQGRVTMTRNTSISTAYMELSSLRSEDTAVYYCARFGYGSGALDYYYYGLDVWGQGTTVTVSS

SEQ ID NO: 69 - protein, 118 aa, Artificial Sequence
  Description of Artificial Sequence Synthetic polypeptide
  10D6_f3 variable heavy chain
EVQLVETGGGLIQPGGSLRLSCAASGITVSSNYMNWVRQAPGKGLEWVSVIYSGGSTYYADSVKGRFTISRDNSKNTLYLQMNSLRAEDTAVYYCARDLELAGAFDIWGQGTMVTVSL

SEQ ID NO: 70 - protein, 118 aa, Artificial Sequence
  Description of Artificial Sequence Synthetic polypeptide
  11D5_f3 variable heavy chain
EVQLVETGGGLIQPGGSLRLSCAASGITVSSNYMSWVRQAPGKGLEWVSVIYSGGSTYYADSVKGRFTISRDNSKNTLYLQMNSLRAEDTAVYYCARDLAVAGAFDIWGQGTMVTVSL

SEQ ID NO: 71 - protein, 126 aa, Artificial Sequence
  Description of Artificial Sequence Synthetic polypeptide
  11G2_f3 variable heavy chain
QVQLVQSGAEVKKPGASVKVSCKASGYTFTSYDITWVRQATGQGLEWMGWMSPNSGNTGYAQKFQGRVSMTRNTSISTAYMELSSLRSEDTAVYYCARFGYGSGALDYYYYGLDVWGQGTTVTVSS

SEQ ID NO: 72 - protein, 122 aa, Artificial Sequence
  Description of Artificial Sequence Synthetic polypeptide
  11G7_f2 variable heavy chain
QVQLQESGPGLVKPSGTLSLTCAVSGGSISSSNWWSWVRQPPGKGLEWIGEIFHSGSTNYNPSLKSRVTISVDKSKNQFSLKLNSVTAADTAVYYCARFRRIVATSYYFDYWGQGTLVTVSS

SEQ ID NO: 73 - protein, 126 aa, Artificial Sequence
  Description of Artificial Sequence Synthetic polypeptide
  16C5_f3 variable heavy chain
QVQLVQSGAEVKKPGASVKVSCKASGYTFTSYDIIWVRQATGQGLEWMGWMSPNSGNTGYAQKFQGRVTMTRNTSIGTAYMELSSLRSEDTAVYFCARFGYGSGALDYYYYGLDVWGQGTTVTVSS

SEQ ID NO: 74 - protein, 126 aa, Artificial Sequence; identical to SEQ ID NO: 63
  Description of Artificial Sequence Synthetic polypeptide
  16C12_f3 variable heavy chain

SEQ ID NO: 75 - protein, 126 aa, Artificial Sequence
  Description of Artificial Sequence Synthetic polypeptide
  17A8_f3 variable heavy chain
QVQLVQSGAEVKKPGASVKVSCKASGYTFTSYDIIWVRQATGQGLEWMGWMSPNSGNTGYAQKFQGRVTMTKNTSISTAYMELSSLRSEDTAVYYCARFGYGSGALDYYYYGLDVWGQGTTVTVSS

SEQ ID NO: 76 - protein, 126 aa, Artificial Sequence
  Description of Artificial Sequence Synthetic polypeptide
  17F2_f3 variable heavy chain
QVQLVQSGAEVKRPGASVKVSCKASGYTFTSYDITWVRQATGQGLEWMGWMSPDSGNTGYAQRFQGRVTMTRNTSISTAYMELSSLRSEDTAVYYCARFGYGSGALDYYYYGLDVWGQGTTVTVSS

SEQ ID NO: 77 - protein, 126 aa, Artificial Sequence
  Description of Artificial Sequence Synthetic polypeptide
  18E7_f3 variable heavy chain
QVQLVQSGAEVKKPGASVTVSCKASGYTFTSYDIIWVRQATGQGLEWMGWMSPNSGNTGYAQRFQGRVTMTRNTSISTAYMELSSLRSEDTAVYYCARFGYGSGALDYYYYGLDVWGQGTTVTVSS

SEQ ID NO: 78 - protein, 118 aa, Artificial Sequence
  Description of Artificial Sequence Synthetic polypeptide
  19C4_f3 variable heavy chain
EVQLVETGGGLIQPGGSLRLSCAASGITVSSNYMNWVRQAPGKGLEWVSVIYSGGSTFYADSVKGRFTISRDNSKNTLYLQMNSLRAEDTAVYYCARDLEVAGGFDIWGQGTMVTVSL

SEQ ID NO: 79 - protein, 123 aa, Artificial Sequence
  Description of Artificial Sequence Synthetic polypeptide
  2C1_f4 variable heavy chain
EVQLVESGGGLVQPGRSLRLSCTASGFTFGDYTLSWFRQAPGKGLEWVGFIRSKPFGGTTQYAASVKGRFTISRDDSKSIAYLQMNSLKTEDTAVYYCTRVSGYSNIWFFAYWGQGTLVTVSS

SEQ ID NO: 80 - protein, 123 aa, Artificial Sequence
  Description of Artificial Sequence Synthetic polypeptide
  4E3_f4 variable heavy chain
QVQLVESGGGVVQPGRSLRLSCAASGFTFSSYGMNWVRQAPGKGLEWVAVIWYDGNNKYYADSVKGRFTISRDNSKNTLYLQMNSLRAEDTAVYYCARKDYSKTFYGYYFDYWGQGTLVTVSS

SEQ ID NO: 81 - protein, 117 aa, Artificial Sequence
  Description of Artificial Sequence Synthetic polypeptide
  7D2_f4 variable heavy chain
EVQLVETGGGLIQPGGSLRLSCAASGFTVSSNYMNWVRQAPGKGLEWVSVIYSGGSTFYADSVKGRFTISRDNSYNTLYLQMNSLRAEDTAVYYCARDLVIYGMDVWGQGTTVTVSS

SEQ ID NO: 82 - protein, 123 aa, Artificial Sequence
  Description of Artificial Sequence Synthetic polypeptide
  9C5_f4 variable heavy chain
QVQLVESGGGVVQPGRSLRLSCVASGFTFSSYGMNWVRQAPGKGLEWVAVIWYDGNNKYYADSVKGRFTISRDNSKNTLYLQMNSLRAEDTAVYYCARKDGSKTYYGYYFDYWGQGTLVTVSS

SEQ ID NO: 83 - protein, 123 aa, Artificial Sequence
  Description of Artificial Sequence Synthetic polypeptide
  10A3_f4 variable heavy chain
QVQLVESGGGVVQPGRSLRLSCAASGFTFSSYGMNWVRQAPGKGLEWVAIIWYDGNNTYYVDSVKGRFTISRDNSKNTLYLQMNSLRAEDTAVYYCARKDGSKTYYGYYFDYWGQGTLVTVSS

SEQ ID NO: 84 - protein, 123 aa, Artificial Sequence
  Description of Artificial Sequence Synthetic polypeptide
  14G5_f4 variable heavy chain
QVQLQESGPGLVKPSGTLSLTCAVSGGSISSNNWWSWVRQPPGKGLVWIGEILHGEITNYNPSLKSRVTISIDKSKNQFSLKLSSVTAADTAVYYCARDANFYGSGSSYFDYWGQGTLVTVSS

SEQ ID NO: 85 - protein, 123 aa, Artificial Sequence
  Description of Artificial Sequence Synthetic polypeptide
  13A12_f3 variable heavy chain
QVQLQESGPGLVKPSGTLSLTCAVSGGSIYSSNCWSWVRQPPGKGLEWIGEIYHSGGTNYNPSLKSRVTISLDKSKNRFSLRLSSVTAADTAVYYCARDQDYYGSGSSLFDYWGQGTLVTVSS

SEQ ID NO: 86 - protein, 115 aa, Artificial Sequence
  Description of Artificial Sequence Synthetic polypeptide
  5B6_f3 variable heavy chain
EVQLVESGGGLVKPGGSLRLSCAASGFTFSNAWMSWVRQAPGKGLEWVGRFKSKTDGGTTDYAAPVKGRFTISRDDSKNTLYLQMNSLKTEDTAVYYCTTSSGYWGQGTLVTVSS

SEQ ID NO: 87 - protein, 118 aa, Artificial Sequence
  Description of Artificial Sequence Synthetic polypeptide
  2G6_f4 variable heavy chain
QVQLQQWGAGLLKPSETLSLTCTIYGGSFSVYYWNWIRQPPEKGLEWIGEINHSGNTNYNPSLKSRVTISVDTSKNQFSLKLSSVTAADTAVYYCARYYYDGNGYYPWGQGTLVTVSS

SEQ ID NO: 88 - protein, 108 aa, Artificial Sequence
  Description of Artificial Sequence Synthetic polypeptide
  1C12_f3 variable light chain
EIVLTQSPGTLSLSPGERATLSCRASQSVRSSHLAWYQQKPGQAPRLLIYGASSRATGIPDRFSGSGSGTDFTLTISRLEPEDFAVYYCQQYNNSPITFGQGTRLEIK

SEQ ID NO: 89 - protein, 108 aa, Artificial Sequence
  Description of Artificial Sequence Synthetic polypeptide
  1D5_f3 variable light chain
EIVLTQSPGTLSLSPGDRATLSCRASQSVRSSHLAWYQQKPGQAPRLLIYGASSRATGIPDRFSGSGSGTDFSLTISRLEPEDFAVYYCQQFGSSPITFGQGTRLEIK

SEQ ID NO: 90 - protein, 108 aa, Artificial Sequence
  Description of Artificial Sequence Synthetic polypeptide
  1D10_f3 variable light chain
EIVLTQSPGTLSLSPGERATLSCRASQSVSSSHLAWYQQKPGQAPRLLIYGASSRATGIPDRFSGSGSGTDFTLTISRLEPEDFAVYYCQQYNSSPITFGQGTRLEIK

SEQ ID NO: 91 - protein, 108 aa, Artificial Sequence
  Description of Artificial Sequence Synthetic polypeptide
  1E2_f3 variable light chain
EIVLTQSPGTLSLSPGERATLSCRASQSVRSSHFAWYQQKPGQAPRLLIYGASSRATGIPDRFSGSGSGTDFTLTISRLEPEDFAVCYCQQYGSSPITFGQGTRLEIK

SEQ ID NO: 92 - protein, 108 aa, Artificial Sequence
  Description of Artificial Sequence Synthetic polypeptide
  1G9_f3 variable light chain
EIVLTQSPGTLSLSPGERATLSCRASQSVRSSHLAWYQQKPGQAPRLLIYGASSRATGIPDRFSGSGSGTDFTLTISRLEPEDFAVYYCQQYGSSPITFGQGTRLEIK

SEQ ID NO: 93 - protein, 108 aa, Artificial Sequence
  Description of Artificial Sequence Synthetic polypeptide
  5D7_f3 variable light chain
EIVLTQSPGTLSLSPGERATLSCRASQSVRSSHLAWYRQKPGQAPRLLIYGASSRATGIPDRFSGRGSGTDFTLTISRLEPEDFAVYYCQQYNISPITFGQGTRLEIK

SEQ ID NO: 94 - protein, 108 aa, Artificial Sequence
  Description of Artificial Sequence Synthetic polypeptide
  5H12_f3 variable light chain
EIVLTQSPGTLSLSPGERATLSCRASQSVRSSHLAWYRQKPGQAPRLLIYGASSRATGIPDRFSGRGSGTDFTLTISRLEPEDFAVYYCQQYNFSPITFGQGTRLEIK

SEQ ID NO: 95 - protein, 108 aa, Artificial Sequence
  Description of Artificial Sequence Synthetic polypeptide
  7C10_f3 variable light chain
EIVLTQSPGTLSLSPGERATLSCRASQSVRSSHLAWSQQKPGQAPRLLIYGASSRATGIPDRFSGSGSGTDFTLTISRLEPEDFAVYYCQQFGSSPITFGQGTRLEIK

SEQ ID NO: 96 - protein, 108 aa, Artificial Sequence
  Description of Artificial Sequence Synthetic polypeptide
  8H4_f3 variable light chain
EIVLTQSPGTLSLSPGERATLSCRASQSVRSSHLAWYQQKPGQAPRLLIYGASSRATGIPDRFSGSGSGTDFTLTISRLEPEDFAVYYCQQFGSSPITFGQGTRLEIK

SEQ ID NO: 97 - protein, 108 aa, Artificial Sequence
  Description of Artificial Sequence Synthetic polypeptide
  9C6_f3 variable light chain
EIVLTQSPGTLSLSPGERATLSCRASQSVRSSHLAWYRQKPGQAPRLLIYGASSRATGIPDRFSGRGSGTDFTLTISRLEPEDFAVYYCQQYNSSPITFGQGTRLEIK

SEQ ID NO: 98 - protein, 108 aa, Artificial Sequence
  Description of Artificial Sequence Synthetic polypeptide
  10D6_f3 variable light chain
DIQLTQSPSFLSASVGDRVTITCRASQGISSYLAWYQQKPGKAPKVLIYAASTLQSGVPSRFSGSGSGTEFTLTISSLQPEDFATYYCQQLNSYPPSTFGQGTKLEIK

SEQ ID NO: 99 - protein, 108 aa, Artificial Sequence; identical to SEQ ID NO: 98
  Description of Artificial Sequence Synthetic polypeptide
  11D5_f3 variable light chain

SEQ ID NO: 100 - protein, 108 aa, Artificial Sequence
  Description of Artificial Sequence Synthetic polypeptide
  11G2_f3 variable light chain
EIVLTQSPGTLSLSPGERATLSCRASQSVRNSHLAWYQQKPGQAPRLLIYGASSRATGIPDRFSGSGSGTDFTLTISRLEPEDFAVYYCQQFGSSPITFGQGTRLEIK

SEQ ID NO: 101 - protein, 112 aa, Artificial Sequence
  Description of Artificial Sequence Synthetic polypeptide
  11G7_f2 variable light chain
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 102 - protein, 108 aa, Artificial Sequence; identical to SEQ ID NO: 88
  Description of Artificial Sequence Synthetic polypeptide
  16C5_f3 variable light chain

SEQ ID NO: 103 - protein, 108 aa, Artificial Sequence; identical to SEQ ID NO: 92
  Description of Artificial Sequence Synthetic polypeptide
  16C12_f3 variable light chain

SEQ ID NO: 104 - protein, 108 aa, Artificial Sequence
  Description of Artificial Sequence Synthetic polypeptide
  17A8_f3 variable light chain
ETVLTQSPGTLSLSPGERATLSCRASQSVRSSHLAWYQQKPGQAPRLLIYGASSRATGIPDRFSGSGSGTDFTLTISRLEPEDFAVYYCQQYGSSPITFGQGTRLEIK

SEQ ID NO: 105 - protein, 108 aa, Artificial Sequence
  Description of Artificial Sequence Synthetic polypeptide
  17F2_f3 variable light chain
EIVLTQSPGTLSLSPGERATLSCRASQSVRSSHLAWYQQKPGQAPRLLIYGASSRATGIPDRFSGSGSGTDFTLTISRLEPEDFAVYYCQQYNSSPITFGQGTRLEIK

SEQ ID NO: 106 - protein, 108 aa, Artificial Sequence; identical to SEQ ID NO: 92
  Description of Artificial Sequence Synthetic polypeptide
  18E7_f3 variable light chain

SEQ ID NO: 107 - protein, 108 aa, Artificial Sequence
  Description of Artificial Sequence Synthetic polypeptide
  19C4_f3 variable light chain
DIQLTQSPSFLSASVGDRVTITCRASQGISSYLAWYQQKPGKAPKVLIYAASTLQSGVPSRFSGSGSGTKFTLTISSLQPEDFATYYCQQLNSFPPSTFGQGTKLEIK

SEQ ID NO: 108 - protein, 106 aa, Artificial Sequence
  Description of Artificial Sequence Synthetic polypeptide
  2C1_f4 variable light chain
EIVMTQSPATLSVSPGERATLSCRASQSVSINLAWYQQKPGQAPRLLIYGASTRATGIPARFSGSGSGTEFTLTVSSLQSEDFAVYYCQQYNNWWTFGQGTKVEIK

SEQ ID NO: 109 - protein, 107 aa, Artificial Sequence
  Description of Artificial Sequence Synthetic polypeptide
  4E3_f4 variable light chain
DIQMTQSPSSLSASVGDRVTITCRASQSIHSFLNWYQQKPGKPPKLLIYAASSLPSGLPSRFSGSGSGTDFTLTISSLQPEDFATYYCQQSYITPPTFGQGTKVEIK

SEQ ID NO: 110 - protein, 106 aa, Artificial Sequence
  Description of Artificial Sequence Synthetic polypeptide
  7D2_f4 variable light chain
EIVMTQSPATLSVSPGERATLSCRASQSVSSNLAWYQQKPGQAPRLLIYGASTRATGVPARFSGSGSGTEFTLTISSLQSEDFAVYFCQQYNNWPPFGGGTKVEIK

SEQ ID NO: 111 - protein, 107 aa, Artificial Sequence
  Description of Artificial Sequence Synthetic polypeptide
  9C5_f4 variable light chain
DIQMTQSPSSLSASVGDRVTITCRASQSIHSFLNWYQQKPGKPPKLLIYTTSSLQSGLPSRFSGSGSGTDFTLTISSLQPEDFATYYCQQSYITPPTFGQGTKVEIK

SEQ ID NO: 112 - protein, 107 aa, Artificial Sequence
  Description of Artificial Sequence Synthetic polypeptide
  10A3_f4 variable light chain
DIQMTQSPSSLSASVGDRVTITCRASQSIHSFLNWYQQKPGKPPNLLIYAASSLQSGLPSRFSGSGSGTDFTLTISSLQPEDFATYYCQQSYITPPTFGHGTKVEIK

SEQ ID NO: 113 - protein, 107 aa, Artificial Sequence
  Description of Artificial Sequence Synthetic polypeptide
  14G5_f4 variable light chain
EIVMTQSPATLSVSPGERATLSCRASQSVSSNLAWYQQKPGQAPRLLIYGASTRATGIPARFSGSGSGTEFTLTISSLQSEDFAVYYCQQYNNWPPTFGQGTKLEIK

SEQ ID NO: 114 - protein, 107 aa, Artificial Sequence
  Description of Artificial Sequence Synthetic polypeptide
  13A12_f3 variable light chain
EIVMTQSPATLSVSPGERATLSCRASQSVSSNLAWYQQKPGQAPRLLIYGASTRATGIPARFSGSGSGTEFTLTISSLQSEDFAVYYCQQYDNWPLTFGGGTKVEIK

SEQ ID NO: 115 - protein, 107 aa, Artificial Sequence
  Description of Artificial Sequence Synthetic polypeptide
  5B6_f3 variable light chain
DIQMTQSPSSLSASVGDRVTITCRASQGISNYLAWYQQKPGKVPKLLIYAASTLQSGVPSRFSGSGSGTDFTLTISSLQPEDVATYYCQKYNSAPHTFGQGTKLEIK

SEQ ID NO: 116 - protein, 107 aa, Artificial Sequence
  Description of Artificial Sequence Synthetic polypeptide
  2G6_f4 variable light chain
DIQLTQSPSFLSASVGDRVTITCRASQGISSYLAWYQQKPGKAPKLLIYAASTLQSGVPSRFSGSGSGTEFTLTISSLQSEDFAIYYCQQLNSYPLTFGGGTKVEIK

SEQ ID NO: 117 - DNA, 24 bp, Artificial Sequence
  Description of Artificial Sequence Synthetic oligonucleotide
  1C12_f3 CDR1H
ggatacaccttcaccagttatgat

SEQ ID NO: 118 - DNA, 24 bp, Artificial Sequence
  Description of Artificial Sequence Synthetic oligonucleotide
  1D5_f3 CDR1H
ggatacaccttcaccagttatgat

SEQ ID NO: 119 - DNA, 24 bp, Artificial Sequence
  Description of Artificial Sequence Synthetic oligonucleotide
  1D10_f3 CDR1H
ggatacaccttcaccagttatgat

SEQ ID NO: 120 - DNA, 24 bp, Artificial Sequence
  Description of Artificial Sequence Synthetic oligonucleotide
  1E2_f3 CDR1H
ggatacaccttcaccagttatgat

SEQ ID NO: 121 - DNA, 24 bp, Artificial Sequence
  Description of Artificial Sequence Synthetic oligonucleotide
  1G9_f3 CDR1H
ggatacaccttcaccagttatgat

SEQ ID NO: 122 - DNA, 24 bp, Artificial Sequence
  Description of Artificial Sequence Synthetic oligonucleotide
  5D7_f3 CDR1H
ggatacaccttcaccagttatgat

SEQ ID NO: 123 - DNA, 24 bp, Artificial Sequence
  Description of Artificial Sequence Synthetic oligonucleotide
  5H12_f3 CDR1H
ggatacaccttccccagttatgat

SEQ ID NO: 124 - DNA, 24 bp, Artificial Sequence
  Description of Artificial Sequence Synthetic oligonucleotide
  7C10_f3 CDR1H
ggatacaccttcaccagttatgat

SEQ ID NO: 125 - DNA, 24 bp, Artificial Sequence
  Description of Artificial Sequence Synthetic oligonucleotide
  8H4_f3 CDR1H
ggatacaccttcaccagttatgat

SEQ ID NO: 126 - DNA, 24 bp, Artificial Sequence
  Description of Artificial Sequence Synthetic oligonucleotide
  9C6_f3 CDR1H
ggatacaccttcaccagttatgat

SEQ ID NO: 127 - DNA, 24 bp, Artificial Sequence
  Description of Artificial Sequence Synthetic oligonucleotide
  10D6_f3 CDR1H
gggatcaccgtcagtagtaactac

SEQ ID NO: 128 - DNA, 24 bp, Artificial Sequence
  Description of Artificial Sequence Synthetic oligonucleotide
  11D5_f3 CDR1H
gggatcaccgtcagtagtaactac

SEQ ID NO: 129 - DNA, 24 bp, Artificial Sequence
  Description of Artificial Sequence Synthetic oligonucleotide
  11G2_f3 CDR1H
ggatacaccttcaccagttatgat

SEQ ID NO: 130 - DNA, 27 bp, Artificial Sequence
  Description of Artificial Sequence Synthetic oligonucleotide
  11G7_f2 CDR1H
ggtggctccatcagcagtagtaactgg

SEQ ID NO: 131 - DNA, 24 bp, Artificial Sequence
  Description of Artificial Sequence Synthetic oligonucleotide
  16C5_f3 CDR1H
ggatacaccttcaccagttatgat

SEQ ID NO: 132 - DNA, 24 bp, Artificial Sequence
  Description of Artificial Sequence Synthetic oligonucleotide
  16C12_f3 CDR1H
ggatacaccttcaccagttatgat

SEQ ID NO: 133 - DNA, 24 bp, Artificial Sequence
  Description of Artificial Sequence Synthetic oligonucleotide
  17A8_f3 CDR1H
ggatacaccttcaccagttatgat

SEQ ID NO: 134 - DNA, 24 bp, Artificial Sequence
  Description of Artificial Sequence Synthetic oligonucleotide
  17F2_f3 CDR1H
ggatacaccttcaccagttatgat

SEQ ID NO: 135 - DNA, 24 bp, Artificial Sequence
  Description of Artificial Sequence Synthetic oligonucleotide
  18E7_f3 CDR1H
ggatacacctttaccagttatgat

SEQ ID NO: 136 - DNA, 24 bp, Artificial Sequence
  Description of Artificial Sequence Synthetic oligonucleotide
  19C4_f3 CDR1H
gggatcaccgtcagtagtaattac

SEQ ID NO: 137 - DNA, 24 bp, Artificial Sequence
  Description of Artificial Sequence Synthetic oligonucleotide
  2C1_f4 CDR1H
ggattcacctttggtgattatact

SEQ ID NO: 138 - DNA, 24 bp, Artificial Sequence
  Description of Artificial Sequence Synthetic oligonucleotide
  4E3_f4 CDR1H
ggattcaccttcagtagctatggc

SEQ ID NO: 139 - DNA, 24 bp, Artificial Sequence
  Description of Artificial Sequence Synthetic oligonucleotide
  7D2_f4 CDR1H
gggttcaccgtcagtagcaactac

SEQ ID NO: 140 - DNA, 24 bp, Artificial Sequence
  Description of Artificial Sequence Synthetic oligonucleotide
  9C5_f4 CDR1H
ggattcaccttcagtagctatggc

SEQ ID NO: 141 - DNA, 24 bp, Artificial Sequence
  Description of Artificial Sequence Synthetic oligonucleotide
  10A3_f4 CDR1H
ggattcaccttcagtagttatggc

SEQ ID NO: 142 - DNA, 27 bp, Artificial Sequence
  Description of Artificial Sequence Synthetic oligonucleotide
  14G5_f4 CDR1H
ggtggctccatcagcagtaataactgg

SEQ ID NO: 143 - DNA, 27 bp, Artificial Sequence
  Description of Artificial Sequence Synthetic oligonucleotide
  13A12_f3 CDR1H
ggtggctccatctacagtagtaactgc

SEQ ID NO: 144 - DNA, 24 bp, Artificial Sequence
  Description of Artificial Sequence Synthetic oligonucleotide
  5B6_f3 CDR1H
ggattcactttcagtaacgcctgg

SEQ ID NO: 145 - DNA, 24 bp, Artificial Sequence
  Description of Artificial Sequence Synthetic oligonucleotide
  2G6_f4 CDR1H
ggtgggtccttcagtgtttactac

SEQ ID NO: 146 - DNA, 24 bp, Artificial Sequence
  Description of Artificial Sequence Synthetic oligonucleotide
  1C12_f3 CDR2H
atgagccctaacagtggtaacaca

SEQ ID NO: 147 - DNA, 24 bp, Artificial Sequence
  Description of Artificial Sequence Synthetic oligonucleotide
  1D5_f3 CDR2H
atgagccctaacagtggtaacaca

SEQ ID NO: 148 - DNA, 24 bp, Artificial Sequence
  Description of Artificial Sequence Synthetic oligonucleotide
  1D10_f3 CDR2H
atgagccctaacagcggtaacaca

SEQ ID NO: 149 - DNA, 24 bp, Artificial Sequence
  Description of Artificial Sequence Synthetic oligonucleotide
  1E2_f3 CDR2H
atgagccctaacagtggtaacaca

SEQ ID NO: 150 - DNA, 24 bp, Artificial Sequence
  Description of Artificial Sequence Synthetic oligonucleotide
  1G9_f3 CDR2H
atgagccctaacagtggtaacaca

SEQ ID NO: 151 - DNA, 24 bp, Artificial Sequence
  Description of Artificial Sequence Synthetic oligonucleotide
  5D7_f3 CDR2H
atgagccctaaaaatggtaacaca

SEQ ID NO: 152 - DNA, 24 bp, Artificial Sequence
  Description of Artificial Sequence Synthetic oligonucleotide
  5H12_f3 CDR2H
atgagccctaaaaatggtaacaca

SEQ ID NO: 153 - DNA, 24 bp, Artificial Sequence
  Description of Artificial Sequence Synthetic oligonucleotide
  7C10_f3 CDR2H
atgagccctaacagtggtaacaca

SEQ ID NO: 154 - DNA, 24 bp, Artificial Sequence
  Description of Artificial Sequence Synthetic oligonucleotide
  8H4_f3 CDR2H
atgagccctaacagtggtaacaca

SEQ ID NO: 155 - DNA, 24 bp, Artificial Sequence
  Description of Artificial Sequence Synthetic oligonucleotide
  9C6_f3 CDR2H
atgagccctaaaagtggtaacaca

SEQ ID NO: 156 - DNA, 21 bp, Artificial Sequence
  Description of Artificial Sequence Synthetic oligonucleotide
  10D6_f3 CDR2H
atttatagcggtggtagcaca

SEQ ID NO: 157 - DNA, 21 bp, Artificial Sequence
  Description of Artificial Sequence Synthetic oligonucleotide
  11D5_f3 CDR2H
atttatagcggtggtagcaca

SEQ ID NO: 158 - DNA, 24 bp, Artificial Sequence
  Description of Artificial Sequence Synthetic oligonucleotide
  11G2_f3 CDR2H
atgagccctaacagtggtaacaca

SEQ ID NO: 159 - DNA, 21 bp, Artificial Sequence
  Description of Artificial Sequence Synthetic oligonucleotide
  11G7_f2 CDR2H
atctttcatagtgggagcacc

SEQ ID NO: 160 - DNA, 24 bp, Artificial Sequence
  Description of Artificial Sequence Synthetic oligonucleotide
  16C5_f3 CDR2H
atgagccctaatagtggtaacaca

SEQ ID NO: 161 - DNA, 24 bp, Artificial Sequence
  Description of Artificial Sequence Synthetic oligonucleotide
  16C12_f3 CDR2H
atgagccctaacagtggtaacaca

SEQ ID NO: 162 - DNA, 24 bp, Artificial Sequence
  Description of Artificial Sequence Synthetic oligonucleotide
  17A8_f3 CDR2H
atgagccctaacagtggtaacaca

SEQ ID NO: 163 - DNA, 24 bp, Artificial Sequence
  Description of Artificial Sequence Synthetic oligonucleotide
  17F2_f3 CDR2H
atgagccctgacagtggtaacaca

SEQ ID NO: 164 - DNA, 24 bp, Artificial Sequence
  Description of Artificial Sequence Synthetic oligonucleotide
  18E7_f3 CDR2H
atgagccctaacagtggtaacaca

SEQ ID NO: 165 - DNA, 21 bp, Artificial Sequence
  Description of Artificial Sequence Synthetic oligonucleotide
  19C4_f3 CDR2H
atttatagcggtggtagcaca

SEQ ID NO: 166 - DNA, 30 bp, Artificial Sequence
  Description of Artificial Sequence Synthetic oligonucleotide
  2C1_f4 CDR2H
attagaagcaaaccttttggtgggacaaca

SEQ ID NO: 167 - DNA, 24 bp, Artificial Sequence
  Description of Artificial Sequence Synthetic oligonucleotide
  4E3_f4 CDR2H
atttggtatgatggaaataataaa

SEQ ID NO: 168 - DNA, 21 bp, Artificial Sequence
  Description of Artificial Sequence Synthetic oligonucleotide
  7D2_f4 CDR2H
atttatagcggtggtagcaca

SEQ ID NO: 169 - DNA, 24 bp, Artificial Sequence
  Description of Artificial Sequence Synthetic oligonucleotide
  9C5_f4 CDR2H
atttggtatgatggaaataataaa

SEQ ID NO: 170 - DNA, 24 bp, Artificial Sequence
  Description of Artificial Sequence Synthetic oligonucleotide
  10A3_f4 CDR2H
atttggtatgatggaaataataca

SEQ ID NO: 171 - DNA, 21 bp, Artificial Sequence
  Description of Artificial Sequence Synthetic oligonucleotide
  14G5_f4 CDR2H
atcttgcatggtgagatcacc

SEQ ID NO: 172 - DNA, 21 bp, Artificial Sequence
  Description of Artificial Sequence Synthetic oligonucleotide
  13A12_f3 CDR2H
atctatcatagtgggggcacc

SEQ ID NO: 173 - DNA, 30 bp, Artificial Sequence
  Description of Artificial Sequence Synthetic oligonucleotide
  5B6_f3 CDR2H
tttaaaagcaaaactgatggtgggacaaca

SEQ ID NO: 174 - DNA, 21 bp, Artificial Sequence
  Description of Artificial Sequence Synthetic oligonucleotide
  2G6_f4 CDR2H
atcaatcatagtggaaacacc

SEQ ID NO: 175 - DNA, 57 bp, Artificial Sequence
  Description of Artificial Sequence Synthetic oligonucleotide
  1C12_f3 CDR3H
gcgagattcggctatggttcgggggccctcgactactactactacggtttggacgtc

SEQ ID NO: 176 - DNA, 57 bp, Artificial Sequence; identical to SEQ ID NO: 175
  Description of Artificial Sequence Synthetic oligonucleotide
  1D5_f3 CDR3H

SEQ ID NO: 177 - DNA, 57 bp, Artificial Sequence
  Description of Artificial Sequence Synthetic oligonucleotide
  1D10_f3 CDR3H
gcgagattcggctatggttcgggggccctcgactactactattacggtttggacgtc

SEQ ID NO: 178 - DNA, 57 bp, Artificial Sequence; identical to SEQ ID NO: 175
  Description of Artificial Sequence Synthetic oligonucleotide
  1E2_f3 CDR3H

SEQ ID NO: 179 - DNA, 57 bp, Artificial Sequence; identical to SEQ ID NO: 175
  Description of Artificial Sequence Synthetic oligonucleotide
  1G9_f3 CDR3H

SEQ ID NO: 180 - DNA, 57 bp, Artificial Sequence; identical to SEQ ID NO: 175
  Description of Artificial Sequence Synthetic oligonucleotide
  5D7_f3 CDR3H

SEQ ID NO: 181 - DNA, 57 bp, Artificial Sequence
  Description of Artificial Sequence Synthetic oligonucleotide
  5H12_f3 CDR3H
gcgagattcggctatgggtcgggggccctcggatattactactacggtttggacgtc

SEQ ID NO: 182 - DNA, 57 bp, Artificial Sequence; identical to SEQ ID NO: 175
  Description of Artificial Sequence Synthetic oligonucleotide
  7C10_f3 CDR3H

SEQ ID NO: 183 - DNA, 57 bp, Artificial Sequence; identical to SEQ ID NO: 175
  Description of Artificial Sequence Synthetic oligonucleotide
  8H4_f3 CDR3H

SEQ ID NO: 184 - DNA, 57 bp, Artificial Sequence; identical to SEQ ID NO: 175
  Description of Artificial Sequence Synthetic oligonucleotide
  9C6_f3 CDR3H

SEQ ID NO: 185 - DNA, 36 bp, Artificial Sequence
  Description of Artificial Sequence Synthetic oligonucleotide
  10D6_f3 CDR3H
gcgagagatttagaactggctggagcttttgatatc

SEQ ID NO: 186 - DNA, 36 bp, Artificial Sequence
  Description of Artificial Sequence Synthetic oligonucleotide
  11D5_f3 CDR3H
gcgagagatttagcagtggctggagcttttgatatc

SEQ ID NO: 187 - DNA, 57 bp, Artificial Sequence; identical to SEQ ID NO: 175
  Description of Artificial Sequence Synthetic oligonucleotide
  11G2_f3 CDR3H

SEQ ID NO: 188 - DNA, 45 bp, Artificial Sequence
  Description of Artificial Sequence Synthetic oligonucleotide
  11G7_f2 CDR3H
gcgagattcagaaggatagtggctacgagctactattttgactac

SEQ ID NO: 189 - DNA, 57 bp, Artificial Sequence; identical to SEQ ID NO: 175
  Description of Artificial Sequence Synthetic oligonucleotide
  16C5_f3 CDR3H

SEQ ID NO: 190 - DNA, 57 bp, Artificial Sequence; identical to SEQ ID NO: 175
  Description of Artificial Sequence Synthetic oligonucleotide
  16C12_f3 CDR3H

SEQ ID NO: 191 - DNA, 57 bp, Artificial Sequence; identical to SEQ ID NO: 175
  Description of Artificial Sequence Synthetic oligonucleotide
  17A8_f3 CDR3H

SEQ ID NO: 192 - DNA, 57 bp, Artificial Sequence; identical to SEQ ID NO: 175
  Description of Artificial Sequence Synthetic oligonucleotide
  17F2_f3 CDR3H

SEQ ID NO: 193 - DNA, 57 bp, Artificial Sequence; identical to SEQ ID NO: 175
  Description of Artificial Sequence Synthetic oligonucleotide
  18E7_f3 CDR3H

SEQ ID NO: 194 - DNA, 36 bp, Artificial Sequence
  Description of Artificial Sequence Synthetic oligonucleotide
  19C4_f3 CDR3H
gcgagagatttagaagtggctggaggttttgatatc

SEQ ID NO: 195 - DNA, 42 bp, Artificial Sequence
  Description of Artificial Sequence Synthetic oligonucleotide
  2C1_f4 CDR3H
actagagtgtccgggtatagcaacatctggttctttgcctac

SEQ ID NO: 196 - DNA, 48 bp, Artificial Sequence
  Description of Artificial Sequence Synthetic oligonucleotide
  4E3_f4 CDR3H
gcgagaaaagattattcgaagacattttatggatactactttgactat

SEQ ID NO: 197 - DNA, 33 bp, Artificial Sequence
  Description of Artificial Sequence Synthetic oligonucleotide
  7D2_f4 CDR3H
gcgagagatctagtcatctacggtatggacgtc

SEQ ID NO: 198 - DNA, 48 bp, Artificial Sequence
  Description of Artificial Sequence Synthetic oligonucleotide
  9C5_f4 CDR3H
gcgagaaaagatggttcgaagacatattatggatactactttgactat

SEQ ID NO: 199 - DNA, 48 bp, Artificial Sequence; identical to SEQ ID NO: 198
  Description of Artificial Sequence Synthetic oligonucleotide
  10A3_f4 CDR3H

SEQ ID NO: 200 - DNA, 48 bp, Artificial Sequence
  Description of Artificial Sequence Synthetic oligonucleotide
  14G5_f4 CDR3H
gcgagagatgcgaatttctatggttcggggagttcttactttgactac

SEQ ID NO: 201 - DNA, 48 bp, Artificial Sequence
  Description of Artificial Sequence Synthetic oligonucleotide
  13A12_f3 CDR3H
gcgagagatcaagattactatggttcggggagttccctctttgactac

SEQ ID NO: 202 - DNA, 18 bp, Artificial Sequence
  Description of Artificial Sequence Synthetic oligonucleotide
  5B6_f3 CDR3H
accaccagcagtggctac

SEQ ID NO: 203 - DNA, 36 bp, Artificial Sequence
  Description of Artificial Sequence Synthetic oligonucleotide
  2G6_f4 CDR3H
gcgaggtattactatgatggtaatggttattacccc

SEQ ID NO: 204 - protein, 8 aa, Artificial Sequence
  Description of Artificial Sequence Synthetic peptide
  1C12_f3 CDR1H
GYTFTSYD

SEQ ID NO: 205 - protein, 8 aa, Artificial Sequence
  Description of Artificial Sequence Synthetic peptide
  1D5_f3 CDR1H
GYTFTSYD

SEQ ID NO: 206 - protein, 8 aa, Artificial Sequence
  Description of Artificial Sequence Synthetic peptide
  1D10_f3 CDR1H
GYTFTSYD

SEQ ID NO: 207 - protein, 8 aa, Artificial Sequence
  Description of Artificial Sequence Synthetic peptide
  1E2_f3 CDR1H
GYTFTSYD

SEQ ID NO: 208 - protein, 8 aa, Artificial Sequence
  Description of Artificial Sequence Synthetic peptide
  1G9_f3 CDR1H
GYTFTSYD

SEQ ID NO: 209 - protein, 8 aa, Artificial Sequence
  Description of Artificial Sequence Synthetic peptide
  5D7_f3 CDR1H
GYTFTSYD

SEQ ID NO: 210 - protein, 8 aa, Artificial Sequence
  Description of Artificial Sequence Synthetic peptide
  5H12_f3 CDR1H
GYTFPSYD

SEQ ID NO: 211 - protein, 8 aa, Artificial Sequence
  Description of Artificial Sequence Synthetic peptide
  7C10_f3 CDR1H
GYTFTSYD

SEQ ID NO: 212 - protein, 8 aa, Artificial Sequence
  Description of Artificial Sequence Synthetic peptide
  8H4_f3 CDR1H
GYTFTSYD

SEQ ID NO: 213 - protein, 8 aa, Artificial Sequence
  Description of Artificial Sequence Synthetic peptide
  9C6_f3 CDR1H
GYTFTSYD

SEQ ID NO: 214 - protein, 8 aa, Artificial Sequence
  Description of Artificial Sequence Synthetic peptide
  10D6_f3 CDR1H
GITVSSNY

SEQ ID NO: 215 - protein, 8 aa, Artificial Sequence
  Description of Artificial Sequence Synthetic peptide
  11D5_f3 CDR1H
GITVSSNY

SEQ ID NO: 216 - protein, 8 aa, Artificial Sequence
  Description of Artificial Sequence Synthetic peptide
  11G2_f3 CDR1H
GYTFTSYD

SEQ ID NO: 217 - protein, 9 aa, Artificial Sequence
  Description of Artificial Sequence Synthetic peptide
  11G7_f2 CDR1H
GGSISSSNW

SEQ ID NO: 218 - protein, 8 aa, Artificial Sequence
  Description of Artificial Sequence Synthetic peptide
  16C5_f3 CDR1H
GYTFTSYD

SEQ ID NO: 219 - protein, 8 aa, Artificial Sequence
  Description of Artificial Sequence Synthetic peptide
  16C12_f3 CDR1H
GYTFTSYD

SEQ ID NO: 220 - protein, 8 aa, Artificial Sequence
  Description of Artificial Sequence Synthetic peptide
  17A8_f3 CDR1H
GYTFTSYD

SEQ ID NO: 221 - protein, 8 aa, Artificial Sequence
  Description of Artificial Sequence Synthetic peptide
  17F2_f3 CDR1H
GYTFTSYD

SEQ ID NO: 222 - protein, 8 aa, Artificial Sequence
  Description of Artificial Sequence Synthetic peptide
  18E7_f3 CDR1H
GYTFTSYD

SEQ ID NO: 223 - protein, 8 aa, Artificial Sequence
  Description of Artificial Sequence Synthetic peptide
  19C4_f3 CDR1H
GITVSSNY

SEQ ID NO: 224 - protein, 8 aa, Artificial Sequence
  Description of Artificial Sequence Synthetic peptide
  2C1_f4 CDR1H
GFTFGDYT

SEQ ID NO: 225 - protein, 8 aa, Artificial Sequence
  Description of Artificial Sequence Synthetic peptide
  4E3_f4 CDR1H
GFTFSSYG

SEQ ID NO: 226 - protein, 8 aa, Artificial Sequence
  Description of Artificial Sequence Synthetic peptide
  7D2_f4 CDR1H
GFTVSSNY

SEQ ID NO: 227 - protein, 8 aa, Artificial Sequence
  Description of Artificial Sequence Synthetic peptide
  9C5_f4 CDR1H
GFTFSSYG

SEQ ID NO: 228 - protein, 8 aa, Artificial Sequence
  Description of Artificial Sequence Synthetic peptide
  10A3_f4 CDR1H
GFTFSSYG

SEQ ID NO: 229 - protein, 9 aa, Artificial Sequence
  Description of Artificial Sequence Synthetic peptide
  14G5_f4 CDR1H
GGSISSNNW

SEQ ID NO: 230 - protein, 9 aa, Artificial Sequence
  Description of Artificial Sequence Synthetic peptide
  13A12_f3 CDR1H
GGSIYSSNC

SEQ ID NO: 231 - protein, 8 aa, Artificial Sequence
  Description of Artificial Sequence Synthetic peptide
  5B6_f3 CDR1H
GFTFSNAW

SEQ ID NO: 232 - protein, 8 aa, Artificial Sequence
  Description of Artificial Sequence Synthetic peptide
  2G6_f4 CDR1H
GGSFSVYY

SEQ ID NO: 233 - protein, 8 aa, Artificial Sequence
  Description of Artificial Sequence Synthetic peptide
  1C12_f3 CDR2H
MSPNSGNT

SEQ ID NO: 234 - protein, 8 aa, Artificial Sequence
  Description of Artificial Sequence Synthetic peptide
  1D5_f3 CDR2H
MSPNSGNT

SEQ ID NO: 235 - protein, 8 aa, Artificial Sequence
  Description of Artificial Sequence Synthetic peptide
  1D10_f3 CDR2H
MSPNSGNT

SEQ ID NO: 236 - protein, 8 aa, Artificial Sequence
  Description of Artificial Sequence Synthetic peptide
  1E2_f3 CDR2H
MSPNSGNT

SEQ ID NO: 237 - protein, 8 aa, Artificial Sequence
  Description of Artificial Sequence Synthetic peptide
  1G9_f3 CDR2H
MSPNSGNT

SEQ ID NO: 238 - protein, 8 aa, Artificial Sequence
  Description of Artificial Sequence Synthetic peptide
  5D7_f3 CDR2H
MSPKNGNT

SEQ ID NO: 239 - protein, 8 aa, Artificial Sequence
  Description of Artificial Sequence Synthetic peptide
  5H12_f3 CDR2H
MSPKNGNT

SEQ ID NO: 240 - protein, 8 aa, Artificial Sequence
  Description of Artificial Sequence Synthetic peptide
  7C10_f3 CDR2H
MSPNSGNT

SEQ ID NO: 241 - protein, 8 aa, Artificial Sequence
  Description of Artificial Sequence Synthetic peptide
  8H4_f3 CDR2H
MSPNSGNT

SEQ ID NO: 242 - protein, 8 aa, Artificial Sequence
  Description of Artificial Sequence Synthetic peptide
  9C6_f3 CDR2H
MSPKSGNT

SEQ ID NO: 243 - protein, 7 aa, Artificial Sequence
  Description of Artificial Sequence Synthetic peptide
  10D6_f3 CDR2H
IYSGGST

SEQ ID NO: 244 - protein, 7 aa, Artificial Sequence
  Description of Artificial Sequence Synthetic peptide
  11D5_f3 CDR2H
IYSGGST

SEQ ID NO: 245 - protein, 8 aa, Artificial Sequence
  Description of Artificial Sequence Synthetic peptide
  11G2_f3 CDR2H
MSPNSGNT

SEQ ID NO: 246 - protein, 7 aa, Artificial Sequence
  Description of Artificial Sequence Synthetic peptide
  11G7_f2 CDR2H
IFHSGST

SEQ ID NO: 247 - protein, 8 aa, Artificial Sequence
  Description of Artificial Sequence Synthetic peptide
  16C5_f3 CDR2H
MSPNSGNT

SEQ ID NO: 248 - protein, 8 aa, Artificial Sequence
  Description of Artificial Sequence Synthetic peptide
  16C12_f3 CDR2H
MSPNSGNT

SEQ ID NO: 249 - protein, 8 aa, Artificial Sequence
  Description of Artificial Sequence Synthetic peptide
  17A8_f3 CDR2H
MSPNSGNT

SEQ ID NO: 250 - protein, 8 aa, Artificial Sequence
  Description of Artificial Sequence Synthetic peptide
  17F2_f3 CDR2H
MSPDSGNT

SEQ ID NO: 251 - protein, 8 aa, Artificial Sequence
  Description of Artificial Sequence Synthetic peptide
  18E7_f3 CDR2H
MSPNSGNT

SEQ ID NO: 252 - protein, 7 aa, Artificial Sequence
  Description of Artificial Sequence Synthetic peptide
  19C4_f3 CDR2H
IYSGGST

SEQ ID NO: 253 - protein, 10 aa, Artificial Sequence
  Description of Artificial Sequence Synthetic peptide
  2C1_f4 CDR2H
IRSKPFGGTT

SEQ ID NO: 254 - protein, 8 aa, Artificial Sequence
  Description of Artificial Sequence Synthetic peptide
  4E3_f4 CDR2H
IWYDGNNK

SEQ ID NO: 255 - protein, 7 aa, Artificial Sequence
  Description of Artificial Sequence Synthetic peptide
  7D2_f4 CDR2H
IYSGGST

SEQ ID NO: 256 - protein, 8 aa, Artificial Sequence
  Description of Artificial Sequence Synthetic peptide
  9C5_f4 CDR2H
IWYDGNNK

SEQ ID NO: 257 - protein, 8 aa, Artificial Sequence
  Description of Artificial Sequence Synthetic peptide
  10A3_f4 CDR2H
IWYDGNNT

SEQ ID NO: 258 - protein, 7 aa, Artificial Sequence
  Description of Artificial Sequence Synthetic peptide
  14G5_f4 CDR2H
ILHGEIT

SEQ ID NO: 259 - protein, 7 aa, Artificial Sequence
  Description of Artificial Sequence Synthetic peptide
  13A12_f3 CDR2H
IYHSGGT

SEQ ID NO: 260 - protein, 10 aa, Artificial Sequence
  Description of Artificial Sequence Synthetic peptide
  5B6_f3 CDR2H
FKSKTDGGTT

SEQ ID NO: 261 - protein, 7 aa, Artificial Sequence
  Description of Artificial Sequence Synthetic peptide
  2G6_f4 CDR2H
INHSGNT

SEQ ID NO: 262 - protein, 19 aa, Artificial Sequence
  Description of Artificial Sequence Synthetic peptide
  1C12_f3 CDR3H
ARFGYGSGALDYYYYGLDV

SEQ ID NO: 263 - protein, 19 aa, Artificial Sequence
  Description of Artificial Sequence Synthetic peptide
  1D5_f3 CDR3H
ARFGYGSGALDYYYYGLDV

SEQ ID NO: 264 - protein, 19 aa, Artificial Sequence
  Description of Artificial Sequence Synthetic peptide
  1D10_f3 CDR3H
ARFGYGSGALDYYYYGLDV

SEQ ID NO: 265 - protein, 19 aa, Artificial Sequence
  Description of Artificial Sequence Synthetic peptide
  1E2_f3 CDR3H
ARFGYGSGALDYYYYGLDV

SEQ ID NO: 266 - protein, 19 aa, Artificial Sequence
  Description of Artificial Sequence Synthetic peptide
  1G9_f3 CDR3H
ARFGYGSGALDYYYYGLDV

SEQ ID NO: 267 - protein, 19 aa, Artificial Sequence
  Description of Artificial Sequence Synthetic peptide
  5D7_f3 CDR3H
ARFGYGSGALDYYYYGLDV

SEQ ID NO: 268 - protein, 19 aa, Artificial Sequence
  Description of Artificial Sequence Synthetic peptide
  5H12_f3 CDR3H
ARFGYGSGALGYYYYGLDV

SEQ ID NO: 269 - protein, 19 aa, Artificial Sequence
  Description of Artificial Sequence Synthetic peptide
  7C10_f3 CDR3H
ARFGYGSGALDYYYYGLDV

SEQ ID NO: 270 - protein, 19 aa, Artificial Sequence
  Description of Artificial Sequence Synthetic peptide
  8H4_f3 CDR3H
ARFGYGSGALDYYYYGLDV

SEQ ID NO: 271 - protein, 19 aa, Artificial Sequence
  Description of Artificial Sequence Synthetic peptide
  9C6_f3 CDR3H
ARFGYGSGALDYYYYGLDV

SEQ ID NO: 272 - protein, 12 aa, Artificial Sequence
  Description of Artificial Sequence Synthetic peptide
  10D6_f3 CDR3H
ARDLELAGAFDI

SEQ ID NO: 273 - protein, 12 aa, Artificial Sequence
  Description of Artificial Sequence Synthetic peptide
  11D5_f3 CDR3H
ARDLAVAGAFDI

SEQ ID NO: 274 - protein, 19 aa, Artificial Sequence
  Description of Artificial Sequence Synthetic peptide
  11G2_f3 CDR3H
ARFGYGSGALDYYYYGLDV

SEQ ID NO: 275 - protein, 15 aa, Artificial Sequence
  Description of Artificial Sequence Synthetic peptide
  11G7_f2 CDR3H
ARFRRIVATSYYFDY

SEQ ID NO: 276 - protein, 19 aa, Artificial Sequence
  Description of Artificial Sequence Synthetic peptide
  16C5_f3 CDR3H
ARFGYGSGALDYYYYGLDV

SEQ ID NO: 277 - protein, 19 aa, Artificial Sequence
  Description of Artificial Sequence Synthetic peptide
  16C12_f3 CDR3H
ARFGYGSGALDYYYYGLDV

SEQ ID NO: 278 - protein, 19 aa, Artificial Sequence
  Description of Artificial Sequence Synthetic peptide
  17A8_f3 CDR3H
ARFGYGSGALDYYYYGLDV

SEQ ID NO: 279 - protein, 19 aa, Artificial Sequence
  Description of Artificial Sequence Synthetic peptide
  17F2_f3 CDR3H
ARFGYGSGALDYYYYGLDV

SEQ ID NO: 280 - protein, 19 aa, Artificial Sequence
  Description of Artificial Sequence Synthetic peptide
  18E7_f3 CDR3H
ARFGYGSGALDYYYYGLDV

SEQ ID NO: 281 - protein, 12 aa, Artificial Sequence
  Description of Artificial Sequence Synthetic peptide
  19C4_f3 CDR3H
ARDLEVAGGFDI

SEQ ID NO: 282 - protein, 14 aa, Artificial Sequence
  Description of Artificial Sequence Synthetic peptide
  2C1_f4 CDR3H
TRVSGYSNIWFFAY

SEQ ID NO: 283 - protein, 16 aa, Artificial Sequence
  Description of Artificial Sequence Synthetic peptide
  4E3_f4 CDR3H
ARKDYSKTFYGYYFDY

SEQ ID NO: 284 - protein, 11 aa, Artificial Sequence
  Description of Artificial Sequence Synthetic peptide
  7D2_f4 CDR3H
ARDLVIYGMDV

SEQ ID NO: 285 - protein, 16 aa, Artificial Sequence
  Description of Artificial Sequence Synthetic peptide
  9C5_f4 CDR3H
ARKDGSKTYYGYYFDY

SEQ ID NO: 286 - protein, 16 aa, Artificial Sequence
  Description of Artificial Sequence Synthetic peptide
  10A3_f4 CDR3H
ARKDGSKTYYGYYFDY

SEQ ID NO: 287 - protein, 16 aa, Artificial Sequence
  Description of Artificial Sequence Synthetic peptide
  14G5_f4 CDR3H
ARDANFYGSGSSYFDY

SEQ ID NO: 288 - protein, 16 aa, Artificial Sequence
  Description of Artificial Sequence Synthetic peptide
  13A12_f3 CDR3H
ARDQDYYGSGSSLFDY

SEQ ID NO: 289 - protein, 6 aa, Artificial Sequence
  Description of Artificial Sequence Synthetic peptide
  5B6_f3 CDR3H
TTSSGY

SEQ ID NO: 290 - protein, 12 aa, Artificial Sequence
  Description of Artificial Sequence Synthetic peptide
  2G6_f4 CDR3H
ARYYYDGNGYYP

SEQ ID NO: 291 - DNA, 21 bp, Artificial Sequence
  Description of Artificial Sequence Synthetic oligonucleotide
  1C12_f3 CDR1L
cagagtgttaggagcagtcac

SEQ ID NO: 292 - DNA, 21 bp, Artificial Sequence
  Description of Artificial Sequence Synthetic oligonucleotide
  1D5_f3 CDR1L
cagagtgttaggagcagtcac

SEQ ID NO: 293 - DNA, 21 bp, Artificial Sequence
  Description of Artificial Sequence Synthetic oligonucleotide
  1D10_f3 CDR1L
cagagtgttagcagcagccac

SEQ ID NO: 294 - DNA, 21 bp, Artificial Sequence
  Description of Artificial Sequence Synthetic oligonucleotide
  1E2_f3 CDR1L
cagagtgttaggagcagtcac

SEQ ID NO: 295 - DNA, 21 bp, Artificial Sequence
  Description of Artificial Sequence Synthetic oligonucleotide
  1G9_f3 CDR1L
cagagtgttaggagcagtcac

SEQ ID NO: 296 - DNA, 21 bp, Artificial Sequence
  Description of Artificial Sequence Synthetic oligonucleotide
  5D7_f3 CDR1L
cagagtgttaggagcagtcac

SEQ ID NO: 297 - DNA, 21 bp, Artificial Sequence
  Description of Artificial Sequence Synthetic oligonucleotide
  5H12_f3 CDR1L
cagagtgttaggagcagtcac

SEQ ID NO: 298 - DNA, 21 bp, Artificial Sequence
  Description of Artificial Sequence Synthetic oligonucleotide
  7C10_f3 CDR1L
cagagtgttaggagcagtcac

SEQ ID NO: 299 - DNA, 21 bp, Artificial Sequence
  Description of Artificial Sequence Synthetic oligonucleotide
  8H4_f3 CDR1L
cagagtgttaggagcagtcac

SEQ ID NO: 300 - DNA, 21 bp, Artificial Sequence
  Description of Artificial Sequence Synthetic oligonucleotide
  9C6_f3 CDR1L
cagagtgttaggagcagtcac

SEQ ID NO: 301 - DNA, 18 bp, Artificial Sequence
  Description of Artificial Sequence Synthetic oligonucleotide
  10D6_f3 CDR1L
cagggcattagcagttat

SEQ ID NO: 302 - DNA, 18 bp, Artificial Sequence
  Description of Artificial Sequence Synthetic oligonucleotide
  11D5_f3 CDR1L
cagggcattagcagttat

SEQ ID NO: 303 - DNA, 21 bp, Artificial Sequence
  Description of Artificial Sequence Synthetic oligonucleotide
  11G2_f3 CDR1L
cagagtgttaggaacagtcac

SEQ ID NO: 304 - DNA, 33 bp, Artificial Sequence
  Description of Artificial Sequence Synthetic oligonucleotide
  11G7_f2 CDR1L
cagagcctcctgcatagtaatggatacaactat

SEQ ID NO: 305 - DNA, 21 bp, Artificial Sequence
  Description of Artificial Sequence Synthetic oligonucleotide
  16C5_f3 CDR1L
cagagtgttaggagcagtcac

SEQ ID NO: 306 - DNA, 21 bp, Artificial Sequence
  Description of Artificial Sequence Synthetic oligonucleotide
  16C12_f3 CDR1L
cagagtgttaggagcagtcac

SEQ ID NO: 307 - DNA, 21 bp, Artificial Sequence
  Description of Artificial Sequence Synthetic oligonucleotide
  17A8_f3 CDR1L
cagagtgttaggagcagtcac

SEQ ID NO: 308 - DNA, 21 bp, Artificial Sequence
  Description of Artificial Sequence Synthetic oligonucleotide
  17F2_f3 CDR1L
cagagtgttaggagcagtcac

SEQ ID NO: 309 - DNA, 21 bp, Artificial Sequence
  Description of Artificial Sequence Synthetic oligonucleotide
  18E7_f3 CDR1L
cagagtgttaggagcagtcac

SEQ ID NO: 310 - DNA, 18 bp, Artificial Sequence
  Description of Artificial Sequence Synthetic oligonucleotide
  19C4_f3 CDR1L
cagggcattagcagttat

SEQ ID NO: 311 - DNA, 18 bp, Artificial Sequence
  Description of Artificial Sequence Synthetic oligonucleotide
  2C1_f4 CDR1L
cagagtgttagcatcaac

SEQ ID NO: 312 - DNA, 18 bp, Artificial Sequence
  Description of Artificial Sequence Synthetic oligonucleotide
  4E3_f4 CDR1L
cagagcattcacagcttt

SEQ ID NO: 313 - DNA, 18 bp, Artificial Sequence
  Description of Artificial Sequence Synthetic oligonucleotide
  7D2_f4 CDR1L
cagagtgttagcagcaac

SEQ ID NO: 314 - DNA, 18 bp, Artificial Sequence
  Description of Artificial Sequence Synthetic oligonucleotide
  9C5_f4 CDR1L
cagagcattcacagcttt

SEQ ID NO: 315 - DNA, 18 bp, Artificial Sequence
  Description of Artificial Sequence Synthetic oligonucleotide
  10A3_f4 CDR1L
cagagcattcacagcttt

SEQ ID NO: 316 - DNA, 18 bp, Artificial Sequence
  Description of Artificial Sequence Synthetic oligonucleotide
  14G5_f4 CDR1L
cagagtgttagcagcaac

SEQ ID NO: 317 - DNA, 18 bp, Artificial Sequence
  Description of Artificial Sequence Synthetic oligonucleotide
  13A12_f3 CDR1L
cagagtgttagcagcaac

SEQ ID NO: 318 - DNA, 18 bp, Artificial Sequence
  Description of Artificial Sequence Synthetic oligonucleotide
  5B6_f3 CDR1L
cagggcattagcaattat

SEQ ID NO: 319 - DNA, 18 bp, Artificial Sequence
  Description of Artificial Sequence Synthetic oligonucleotide
  2G6_f4 CDR1L
cagggcattagcagttat

SEQ ID NO: 320 - DNA, 9 bp, Artificial Sequence
  Description of Artificial Sequence Synthetic oligonucleotide
  1C12_f3 CDR2L
ggtgcatcc

SEQ ID NO: 321 - DNA, 9 bp, Artificial Sequence
  Description of Artificial Sequence Synthetic oligonucleotide
  1D5_f3 CDR2L
ggtgcatcc

SEQ ID NO: 322 - DNA, 9 bp, Artificial Sequence
  Description of Artificial Sequence Synthetic oligonucleotide
  1D10_f3 CDR2L
ggtgcatcc

SEQ ID NO: 323 - DNA, 9 bp, Artificial Sequence
  Description of Artificial Sequence Synthetic oligonucleotide
  1E2_f3 CDR2L
ggtgcatcc

SEQ ID NO: 324 - DNA, 9 bp, Artificial Sequence
  Description of Artificial Sequence Synthetic oligonucleotide
  1G9_f3 CDR2L
ggtgcatcc

SEQ ID NO: 325 - DNA, 9 bp, Artificial Sequence
  Description of Artificial Sequence Synthetic oligonucleotide
  5D7_f3 CDR2L
ggtgcctcc

SEQ ID NO: 326 - DNA, 9 bp, Artificial Sequence
  Description of Artificial Sequence Synthetic oligonucleotide
  5H12_f3 CDR2L
ggtgcctcc

SEQ ID NO: 327 - DNA, 9 bp, Artificial Sequence
  Description of Artificial Sequence Synthetic oligonucleotide
  7C10_f3 CDR2L
ggtgcatcc

SEQ ID NO: 328 - DNA, 9 bp, Artificial Sequence
  Description of Artificial Sequence Synthetic oligonucleotide
  8H4_f3 CDR2L
ggtgcatcc

SEQ ID NO: 329 - DNA, 9 bp, Artificial Sequence
  Description of Artificial Sequence Synthetic oligonucleotide
  9C6_f3 CDR2L
ggtgcctcc

SEQ ID NO: 330 - DNA, 9 bp, Artificial Sequence
  Description of Artificial Sequence Synthetic oligonucleotide
  10D6_f3 CDR2L
gctgcatcc

SEQ ID NO: 331 - DNA, 9 bp, Artificial Sequence
  Description of Artificial Sequence Synthetic oligonucleotide
  11D5_f3 CDR2L
gctgcatcc

SEQ ID NO: 332 - DNA, 9 bp, Artificial Sequence
  Description of Artificial Sequence Synthetic oligonucleotide
  11G2_f3 CDR2L
ggtgcatcc

SEQ ID NO: 333 - DNA, 9 bp, Artificial Sequence
  Description of Artificial Sequence Synthetic oligonucleotide
  11G7_f2 CDR2L
ttgggttct

SEQ ID NO: 334 - DNA, 9 bp, Artificial Sequence
  Description of Artificial Sequence Synthetic oligonucleotide
  16C5_f3 CDR2L
ggtgcatcc

SEQ ID NO: 335 - DNA, 9 bp, Artificial Sequence
  Description of Artificial Sequence Synthetic oligonucleotide
  16C12_f3 CDR2L
ggtgcatcc

SEQ ID NO: 336 - DNA, 9 bp, Artificial Sequence
  Description of Artificial Sequence Synthetic oligonucleotide
  17A8_f3 CDR2L
ggtgcatcc

SEQ ID NO: 337 - DNA, 9 bp, Artificial Sequence
  Description of Artificial Sequence Synthetic oligonucleotide
  17F2_f3 CDR2L
ggtgcatcc

SEQ ID NO: 338 - DNA, 9 bp, Artificial Sequence
  Description of Artificial Sequence Synthetic oligonucleotide
  18E7_f3 CDR2L
ggtgcatcc

SEQ ID NO: 339 - DNA, 9 bp, Artificial Sequence
  Description of Artificial Sequence Synthetic oligonucleotide
  19C4_f3 CDR2L
gctgcatcc

SEQ ID NO: 340 - DNA, 9 bp, Artificial Sequence
  Description of Artificial Sequence Synthetic oligonucleotide
  2C1_f4 CDR2L
ggtgcatcc

SEQ ID NO: 341 - DNA, 9 bp, Artificial Sequence
  Description of Artificial Sequence Synthetic oligonucleotide
  4E3_f4 CDR2L
gctgcatcc

SEQ ID NO: 342 - DNA, 9 bp, Artificial Sequence
  Description of Artificial Sequence Synthetic oligonucleotide
  7D2_f4 CDR2L
ggtgcatcc

SEQ ID NO: 343 - DNA, 9 bp, Artificial Sequence
  Description of Artificial Sequence Synthetic oligonucleotide
  9C5_f4 CDR2L
actacatcc

SEQ ID NO: 344 - DNA, 9 bp, Artificial Sequence
  Description of Artificial Sequence Synthetic oligonucleotide
  10A3_f4 CDR2L
gctgcatcc

SEQ ID NO: 345 - DNA, 9 bp, Artificial Sequence
  Description of Artificial Sequence Synthetic oligonucleotide
  14G5_f4 CDR2L
ggtgcatcc

SEQ ID NO: 346 - DNA, 9 bp, Artificial Sequence
  Description of Artificial Sequence Synthetic oligonucleotide
  13A12_f3 CDR2L
ggtgcatcc

SEQ ID NO: 347 - DNA, 9 bp, Artificial Sequence
  Description of Artificial Sequence Synthetic oligonucleotide
  5B6_f3 CDR2L
gctgcatcc

SEQ ID NO: 348 - DNA, 9 bp, Artificial Sequence
  Description of Artificial Sequence Synthetic oligonucleotide
  2G6_f4 CDR2L
gctgcatcc

SEQ ID NO: 349 - DNA, 27 bp, Artificial Sequence
  Description of Artificial Sequence Synthetic oligonucleotide
  1C12_f3 CDR3L
cagcagtataataactcaccgatcacc

SEQ ID NO: 350 - DNA, 27 bp, Artificial Sequence
  Description of Artificial Sequence Synthetic oligonucleotide
  1D5_f3 CDR3L
cagcagtttggtagctcaccgatcacc

SEQ ID NO: 351 - DNA, 27 bp, Artificial Sequence
  Description of Artificial Sequence Synthetic oligonucleotide
  1D10_f3 CDR3L
cagcagtataatagctcaccgatcacc

SEQ ID NO: 352 - DNA, 27 bp, Artificial Sequence
  Description of Artificial Sequence Synthetic oligonucleotide
  1E2_f3 CDR3L
cagcagtatggtagctcaccgatcacc

SEQ ID NO: 353 - DNA, 27 bp, Artificial Sequence
  Description of Artificial Sequence Synthetic oligonucleotide
  1G9_f3 CDR3L
cagcagtatggtagctcaccgatcacc

SEQ ID NO: 354 - DNA, 27 bp, Artificial Sequence
  Description of Artificial Sequence Synthetic oligonucleotide
  5D7_f3 CDR3L
cagcagtataatatctcaccgatcacc

SEQ ID NO: 355 - DNA, 27 bp, Artificial Sequence
  Description of Artificial Sequence Synthetic oligonucleotide
  5H12_f3 CDR3L
cagcagtataatttctcaccgatcacc

SEQ ID NO: 356 - DNA, 27 bp, Artificial Sequence
  Description of Artificial Sequence Synthetic oligonucleotide
  7C10_f3 CDR3L
cagcagtttggtagctcaccgatcacc

SEQ ID NO: 357 - DNA, 27 bp, Artificial Sequence
  Description of Artificial Sequence Synthetic oligonucleotide
  8H4_f3 CDR3L
cagcagtttggtagctcaccgatcacc

SEQ ID NO: 358 - DNA, 27 bp, Artificial Sequence
  Description of Artificial Sequence Synthetic oligonucleotide
  9C6_f3 CDR3L
cagcagtataatagctcaccgatcacc

SEQ ID NO: 359 - DNA, 30 bp, Artificial Sequence
  Description of Artificial Sequence Synthetic oligonucleotide
  10D6_f3 CDR3L
caacagcttaatagttaccctccgtccact

SEQ ID NO: 360 - DNA, 30 bp, Artificial Sequence; identical to SEQ ID NO: 359
  Description of Artificial Sequence Synthetic oligonucleotide
  11D5_f3 CDR3L

SEQ ID NO: 361 - DNA, 27 bp, Artificial Sequence
  Description of Artificial Sequence Synthetic oligonucleotide
  11G2_f3 CDR3L
cagcagtttggtagctcaccgatcacc

SEQ ID NO: 362 - DNA, 27 bp, Artificial Sequence
  Description of Artificial Sequence Synthetic oligonucleotide
  11G7_f2 CDR3L
atgcaagctctacaaactcctctcact

SEQ ID NO: 363 - DNA, 27 bp, Artificial Sequence
  Description of Artificial Sequence Synthetic oligonucleotide
  16C5_f3 CDR3L
cagcagtataataactcaccgatcacc

SEQ ID NO: 364 - DNA, 27 bp, Artificial Sequence
  Description of Artificial Sequence Synthetic oligonucleotide
  16C12_f3 CDR3L
cagcagtatggtagctcaccgatcacc

SEQ ID NO: 365 - DNA, 27 bp, Artificial Sequence
  Description of Artificial Sequence Synthetic oligonucleotide
  17A8_f3 CDR3L
cagcagtatggtagctcaccgatcacc

SEQ ID NO: 366 - DNA, 27 bp, Artificial Sequence
  Description of Artificial Sequence Synthetic oligonucleotide
  17F2_f3 CDR3L
cagcagtataatagctcaccgatcacc

SEQ ID NO: 367 - DNA, 27 bp, Artificial Sequence
  Description of Artificial Sequence Synthetic oligonucleotide
  18E7_f3 CDR3L
cagcagtatggtagctcaccgatcacc

SEQ ID NO: 368 - DNA, 30 bp, Artificial Sequence
  Description of Artificial Sequence Synthetic oligonucleotide
  19C4_f3 CDR3L
caacagcttaatagtttccctccgtccact

SEQ ID NO: 369 - DNA, 24 bp, Artificial Sequence
  Description of Artificial Sequence Synthetic oligonucleotide
  2C1_f4 CDR3L
cagcagtataataactggtggacg

SEQ ID NO: 370 - DNA, 27 bp, Artificial Sequence
  Description of Artificial Sequence Synthetic oligonucleotide
  4E3_f4 CDR3L
caacagagttacattacccctccgacg

SEQ ID NO: 371 - DNA, 24 bp, Artificial Sequence
  Description of Artificial Sequence Synthetic oligonucleotide
  7D2_f4 CDR3L
cagcagtataataactggccccct

SEQ ID NO: 372 - DNA, 27 bp, Artificial Sequence
  Description of Artificial Sequence Synthetic oligonucleotide
  9C5_f4 CDR3L
caacagagttacattacccctccgacg

SEQ ID NO: 373 - DNA, 27 bp, Artificial Sequence
  Description of Artificial Sequence Synthetic oligonucleotide
  10A3_f4 CDR3L
caacagagttacattacccctccgacg

SEQ ID NO: 374 - DNA, 27 bp, Artificial Sequence
  Description of Artificial Sequence Synthetic oligonucleotide
  14G5_f4 CDR3L
cagcagtataataactggcctccgact

SEQ ID NO: 375 - DNA, 27 bp, Artificial Sequence
  Description of Artificial Sequence Synthetic oligonucleotide
  13A12_f3 CDR3L
cagcagtatgataactggcctctcact

SEQ ID NO: 376 - DNA, 27 bp, Artificial Sequence
  Description of Artificial Sequence Synthetic oligonucleotide
  5B6_f3 CDR3L
caaaagtataacagtgcccctcacact

SEQ ID NO: 377 - DNA, 27 bp, Artificial Sequence
  Description of Artificial Sequence Synthetic oligonucleotide
  2G6_f4 CDR3L
caacagcttaatagttacccgctcact

SEQ ID NO: 378 - protein, 7 aa, Artificial Sequence
  Description of Artificial Sequence Synthetic peptide
  1C12_f3 CDR1L
QSVRSSH

SEQ ID NO: 379 - protein, 7 aa, Artificial Sequence
  Description of Artificial Sequence Synthetic peptide
  1D5_f3 CDR1L
QSVRSSH

SEQ ID NO: 380 - protein, 7 aa, Artificial Sequence
  Description of Artificial Sequence Synthetic peptide
  1D10_f3 CDR1L
QSVSSSH

SEQ ID NO: 381 - protein, 7 aa, Artificial Sequence
  Description of Artificial Sequence Synthetic peptide
  1E2_f3 CDR1L
QSVRSSH

SEQ ID NO: 382 - protein, 7 aa, Artificial Sequence
  Description of Artificial Sequence Synthetic peptide
  1G9_f3 CDR1L
QSVRSSH

SEQ ID NO: 383 - protein, 7 aa, Artificial Sequence
  Description of Artificial Sequence Synthetic peptide
  5D7_f3 CDR1L
QSVRSSH

SEQ ID NO: 384 - protein, 7 aa, Artificial Sequence
  Description of Artificial Sequence Synthetic peptide
  5H12_f3 CDR1L
QSVRSSH

SEQ ID NO: 385 - protein, 7 aa, Artificial Sequence
  Description of Artificial Sequence Synthetic peptide
  7C10_f3 CDR1L
QSVRSSH

SEQ ID NO: 386 - protein, 7 aa, Artificial Sequence
  Description of Artificial Sequence Synthetic peptide
  8H4_f3 CDR1L
QSVRSSH

SEQ ID NO: 387 - protein, 7 aa, Artificial Sequence
  Description of Artificial Sequence Synthetic peptide
  9C6_f3 CDR1L
QSVRSSH

SEQ ID NO: 388 - protein, 6 aa, Artificial Sequence
  Description of Artificial Sequence Synthetic peptide
  10D6_f3 CDR1L
QGISSY

SEQ ID NO: 389 - protein, 6 aa, Artificial Sequence
  Description of Artificial Sequence Synthetic peptide
  11D5_f3 CDR1L
QGISSY

SEQ ID NO: 390 - protein, 7 aa, Artificial Sequence
  Description of Artificial Sequence Synthetic peptide
  11G2_f3 CDR1L
QSVRNSH

SEQ ID NO: 391 - protein, 11 aa, Artificial Sequence
  Description of Artificial Sequence Synthetic peptide
  11G7_f2 CDR1L
QSLLHSNGYNY

SEQ ID NO: 392 - protein, 7 aa, Artificial Sequence
  Description of Artificial Sequence Synthetic peptide
  16C5_f3 CDR1L
QSVRSSH

SEQ ID NO: 393 - protein, 7 aa, Artificial Sequence
  Description of Artificial Sequence Synthetic peptide
  16C12_f3 CDR1L
QSVRSSH

SEQ ID NO: 394 - protein, 7 aa, Artificial Sequence
  Description of Artificial Sequence Synthetic peptide
  17A8_f3 CDR1L
QSVRSSH

SEQ ID NO: 395 - protein, 7 aa, Artificial Sequence
  Description of Artificial Sequence Synthetic peptide
  17F2_f3 CDR1L
QSVRSSH

SEQ ID NO: 396 - protein, 7 aa, Artificial Sequence
  Description of Artificial Sequence Synthetic peptide
  18E7_f3 CDR1L
QSVRSSH

SEQ ID NO: 397 - protein, 6 aa, Artificial Sequence
  Description of Artificial Sequence Synthetic peptide
  19C4_f3 CDR1L
QGISSY

SEQ ID NO: 398 - protein, 6 aa, Artificial Sequence
  Description of Artificial Sequence Synthetic peptide
  2C1_f4 CDR1L
QSVSIN

SEQ ID NO: 399 - protein, 6 aa, Artificial Sequence
  Description of Artificial Sequence Synthetic peptide
  4E3_f4 CDR1L
QSIHSF

SEQ ID NO: 400 - protein, 6 aa, Artificial Sequence
  Description of Artificial Sequence Synthetic peptide
  7D2_f4 CDR1L
QSVSSN

SEQ ID NO: 401 - protein, 6 aa, Artificial Sequence
  Description of Artificial Sequence Synthetic peptide
  9C5_f4 CDR1L
QSIHSF

SEQ ID NO: 402 - protein, 6 aa, Artificial Sequence
  Description of Artificial Sequence Synthetic peptide
  10A3_f4 CDR1L
QSIHSF

SEQ ID NO: 403 - protein, 6 aa, Artificial Sequence
  Description of Artificial Sequence Synthetic peptide
  14G5_f4 CDR1L
QSVSSN

SEQ ID NO: 404 - protein, 6 aa, Artificial Sequence
  Description of Artificial Sequence Synthetic peptide
  13A12_f3 CDR1L
QSVSSN

SEQ ID NO: 405 - protein, 6 aa, Artificial Sequence
  Description of Artificial Sequence Synthetic peptide
  5B6_f3 CDR1L
QGISNY

SEQ ID NO: 406 - protein, 6 aa, Artificial Sequence
  Description of Artificial Sequence Synthetic peptide
  2G6_f4 CDR1L
QGISSY

SEQ ID NO: 407 - protein, 9 aa, Artificial Sequence
  Description of Artificial Sequence Synthetic peptide
  1C12_f3 CDR3L
QQYNNSPIT

SEQ ID NO: 408 - protein, 9 aa, Artificial Sequence
  Description of Artificial Sequence Synthetic peptide
  1D5_f3 CDR3L
QQFGSSPIT

SEQ ID NO: 409 - protein, 9 aa, Artificial Sequence
  Description of Artificial Sequence Synthetic peptide
  1D10_f3 CDR3L
QQYNSSPIT

SEQ ID NO: 410 - protein, 9 aa, Artificial Sequence
  Description of Artificial Sequence Synthetic peptide
  1E2_f3 CDR3L
QQYGSSPIT

SEQ ID NO: 411 - protein, 9 aa, Artificial Sequence
  Description of Artificial Sequence Synthetic peptide
  1G9_f3 CDR3L
QQYGSSPIT

SEQ ID NO: 412 - protein, 9 aa, Artificial Sequence
  Description of Artificial Sequence Synthetic peptide
  5D7_f3 CDR3L
QQYNISPIT

SEQ ID NO: 413 - protein, 9 aa, Artificial Sequence
  Description of Artificial Sequence Synthetic peptide
  5H12_f3 CDR3L
QQYNFSPIT

SEQ ID NO: 414 - protein, 9 aa, Artificial Sequence
  Description of Artificial Sequence Synthetic peptide
  7C10_f3 CDR3L
QQFGSSPIT

SEQ ID NO: 415 - protein, 9 aa, Artificial Sequence
  Description of Artificial Sequence Synthetic peptide
  8H4_f3 CDR3L
QQFGSSPIT

SEQ ID NO: 416 - protein, 9 aa, Artificial Sequence
  Description of Artificial Sequence Synthetic peptide
  9C6_f3 CDR3L
QQYNSSPIT

SEQ ID NO: 417 - protein, 10 aa, Artificial Sequence
  Description of Artificial Sequence Synthetic peptide
  10D6_f3 CDR3L
QQLNSYPPST

SEQ ID NO: 418 - protein, 10 aa, Artificial Sequence
  Description of Artificial Sequence Synthetic peptide
  11D5_f3 CDR3L
QQLNSYPPST

SEQ ID NO: 419 - protein, 9 aa, Artificial Sequence
  Description of Artificial Sequence Synthetic peptide
  11G2_f3 CDR3L
QQFGSSPIT

SEQ ID NO: 420 - protein, 9 aa, Artificial Sequence
  Description of Artificial Sequence Synthetic peptide
  11G7_f2 CDR3L
MQALQTPLT

SEQ ID NO: 421 - protein, 9 aa, Artificial Sequence
  Description of Artificial Sequence Synthetic peptide
  16C5_f3 CDR3L
QQYNNSPIT

SEQ ID NO: 422 - protein, 9 aa, Artificial Sequence
  Description of Artificial Sequence Synthetic peptide
  16C12_f3 CDR3L
QQYGSSPIT

SEQ ID NO: 423 - protein, 9 aa, Artificial Sequence
  Description of Artificial Sequence Synthetic peptide
  17A8_f3 CDR3L
QQYGSSPIT

SEQ ID NO: 424 - protein, 9 aa, Artificial Sequence
  Description of Artificial Sequence Synthetic peptide
  17F2_f3 CDR3L
QQYNSSPIT

SEQ ID NO: 425 - protein, 9 aa, Artificial Sequence
  Description of Artificial Sequence Synthetic peptide
  18E7_f3 CDR3L
QQYGSSPIT

SEQ ID NO: 426 - protein, 10 aa, Artificial Sequence
  Description of Artificial Sequence Synthetic peptide
  19C4_f3 CDR3L
QQLNSFPPST

SEQ ID NO: 427 - protein, 8 aa, Artificial Sequence
  Description of Artificial Sequence Synthetic peptide
  2C1_f4 CDR3L
QQYNNWWT

SEQ ID NO: 428 - protein, 9 aa, Artificial Sequence
  Description of Artificial Sequence Synthetic peptide
  4E3_f4 CDR3L
QQSYITPPT

SEQ ID NO: 429 - protein, 8 aa, Artificial Sequence
  Description of Artificial Sequence Synthetic peptide
  7D2_f4 CDR3L
QQYNNWPP

SEQ ID NO: 430 - protein, 9 aa, Artificial Sequence
  Description of Artificial Sequence Synthetic peptide
  9C5_f4 CDR3L
QQSYITPPT

SEQ ID NO: 431 - protein, 9 aa, Artificial Sequence
  Description of Artificial Sequence Synthetic peptide
  10A3_f4 CDR3L
QQSYITPPT

SEQ ID NO: 432 - protein, 9 aa, Artificial Sequence
  Description of Artificial Sequence Synthetic peptide
  14G5_f4 CDR3L
QQYNNWPPT

SEQ ID NO: 433 - protein, 9 aa, Artificial Sequence
  Description of Artificial Sequence Synthetic peptide
  13A12_f3 CDR3L
QQYDNWPLT

SEQ ID NO: 434 - protein, 9 aa, Artificial Sequence
  Description of Artificial Sequence Synthetic peptide
  5B6_f3 CDR3L
QKYNSAPHT

SEQ ID NO: 435 - protein, 9 aa, Artificial Sequence
  Description of Artificial Sequence Synthetic peptide
  2G6_f4 CDR3L
QQLNSYPLT